(19) United States
(12) Patent Application Publication
(10) Pub. No.: US 2023/0371373 A1
(43) Pub. Date: Nov. 16, 2023

(54) ORGANIC ELECTROLUMINESCENT ELEMENT, COMPOUND, MATERIAL FOR ORGANIC ELECTROLUMINESCENT ELEMENT, AND ELECTRONIC DEVICE

(71) Applicant: ldemitsu Kosan Co.,Ltd., Tokyo (JP)

(72) Inventors: Takushi SHIOMI, Tokyo (JP); Hisato MATSUMOTO, Tokyo (JP)

(73) Assignee: ldemitsu Kosan Co.,Ltd., Tokyo (JP)

(21) Appl. No.: 18/029,977

(22) PCT Filed: Oct. 4, 2021
(86) PCT No.: PCT/JP2021/036657

§ 371 Date: Apr. 3, 2023

(30) Foreign Application Priority Data
Oct. 5, 2020 (JP) 2020-168512
Jan. 22, 2021 (JP) 2021-008796

(51) Int. Cl. H10K 85/60

(52) U.S. Cl. CPC H10K 85/6572; H10K 85/6574; H10K 85/6576; H10K 85/654; H10K 2101/20

(57) ABSTRACT

An organic electroluminescence device includes an anode, a cathode, and an emitting layer provided between the anode and the cathode, in which the emitting layer contains a compound M3 represented by a formula (11), (12) or (13) and a delayed fluorescent compound M2, the compound M3 and the compound M2 are different in structure, and a singlet energy S₁(M3) of the compound M3 and a singlet energy S₁(M2) of the compound M2 satisfy a relationship of a numerical formula (Numerical Formula 1) below. In the formulae (11), (12) and (13), one of carbon atoms banded to R₂₅, R₂₇ and R₂₈ is bonded to *1. One of carbon atoms and the like bonded to R₃₁ to R₃₈ and the like is bonded to *. (Numerical Formula 1): S₁(M3)>S₁(M2)

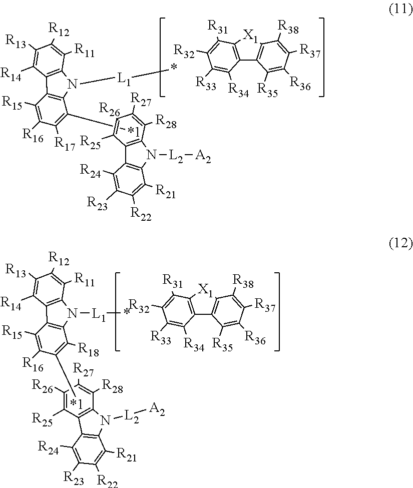

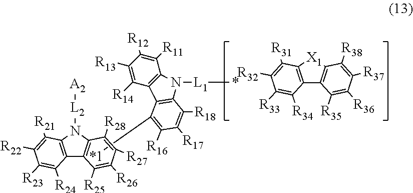

### TECHNICAL FIELD

The present invention relates to an organic electroluminescence device, a compound, an organic-electroluminescence-device material, and an electronic device.

### BACKGROUND ART

When a voltage is applied to an organic electroluminescence device (hereinafter, occasionally referred to as “organic EL device”), holes are injected from an anode and electrons are injected from a cathode into an emitting layer. The injected electrons and holes are recombined in the emitting layer to form excitons. Specifically, according to the electron spin statistics theory, singlet excitons and triplet excitons are generated at a ratio of 25%:75%.

A fluorescent organic EL device using light emission from singlet excitons has been applied to a full-color display such as a mobile phone and a television set, but an internal quantum efficiency is said to be at a limit of 25%. Studies have thus been made to improve performance of the organic EL device.

For instance, the organic EL device is expected to emit light more efficiently using triplet excitons in addition to singlet excitons. In view of the above, a highly-efficient fluorescent organic EL device using thermally activated delayed fluorescence (hereinafter simply referred to as “delayed fluorescence” in some cases) has been proposed and studied.

A thermally activated delayed fluorescence (TADF) mechanism uses such a phenomenon in which inverse intersystem crossing from triplet excitons to singlet excitons thermally occurs when a material having a small energy difference (ΔST) between singlet energy level and triplet energy level is used. Thermally activated delayed fluorescence is explained in “Yuki Hando-tai no Debaisu Bussei (Device Physics of Organic Semiconductors)” (edited by ADACHI Chihaya, published by Kodansha, issued on Apr. 1, 2012, on pages 261-268).

Patent Literature 1 discloses an organic EL device containing a delayed fluorescent compound and a biscarbazole compound substituted by an aryl group in an emitting layer.

Patent Literature 2 discloses an organic EL device containing a delayed fluorescent compound, a compound having a carbazole group and a dibenzothiophene group or a dibenzofuran group in an emitting layer.

### CITATION LIST

### Patent Literature(s)

Patent Literature 1: WO2019/013063

Patent Literature 2: WO2020/122118

### SUMMARY OF THE INVENTION

### Problem(s) to be Solved by the Invention

A further improvement in performance of the organic EL device has been demanded for an improvement in performance of an electronic device such as a display.

An object of the invention is to provide an organic electroluminescence device excellent in performance, particularly, having an improved luminous efficiency, and an electronic device including the organic electroluminescence device.

Another object of the invention is to provide a compound capable of achieving an organic electroluminescence device excellent in performance, particularly, having an improved luminous efficiency, and an organic-electroluminescence-device material containing the compound.

### Means for Solving the Problem(s)

According to an aspect of the invention, there is provided an organic electroluminescence device including: an anode; a cathode; an emitting layer provided between the anode and the cathode, in which the emitting layer contains a compound M3 represented by a formula (11), a formula (12) or a formula (13) below and a delayed fluorescent compound M2, the compound M3 and the compound M2 are different in structure, and a singlet energy S₁(M3) of the compound M3 and a singlet energy S₁(M2) of the compound M2 satisfy a relationship of a numerical formula (Numerical Formula 1) below.

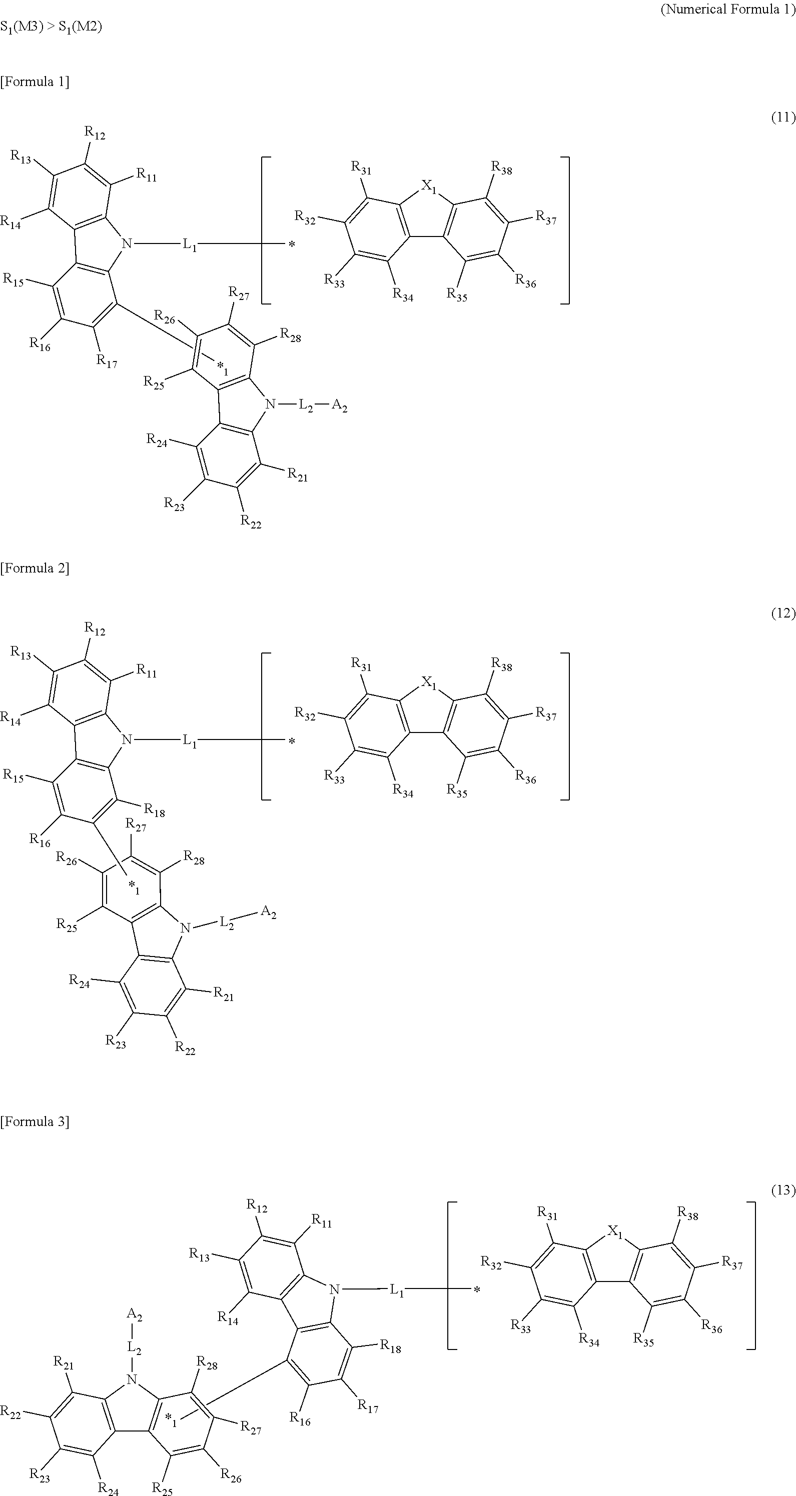

In the formulae (11) to (13):
A₂ is a substituted or unsubstituted aryl group having 6 to 30 ring carbon atoms, or a substituted or unsubstituted heterocyclic group having 5 to 30 ring atoms;
L₁ and L₂ are each independently a single bond, a substituted or unsubstituted arylene group having 6 to 30 ring carbon atoms, a substituted or unsubstituted divalent heterocyclic group having 5 to 30 ring atoms, a group obtained by bonding two groups selected from the group consisting of a substituted or unsubstituted arylene group having 6 to 30 ring carbon atoms and a substituted or unsubstituted divalent heterocyclic group having 5 to 30 ring atoms, or a group obtained by bonding three groups selected from the group consisting of a substituted or unsubstituted arylene group having 6 to 30 ring carbon atoms and a substituted or unsubstituted divalent heterocyclic group having 5 to 30 ring atoms;
the two groups are mutually the same or different;
the three groups are mutually the same or different;
R₁₁ to R₁₈ are each independently a hydrogen atom or a substituent, or at least one combination of a combination of R₁₁ and R₁₂, a combination of R₁₂ and R₁₃, a combination of R₁₃ and R₁₄, a combination of R₁₅ and R₁₆, a combination of R₁₆ and R₁₇, or a combination of R₁₇ and R₁₈ are mutually bonded to form a ring;
R₂₁ to R₂₈ are each independently a hydrogen atom or a substituent, or at least one combination of a combination of R₂₁ and R₂₂, a combination of R₂₂ and R₂₃, a combination of R₂₃ and R₂₄, a combination of R₂₅ and R₂₆, a combination of R₂₆ and R₂₇, or a combination of R₂₇ and R₂₈ are mutually bonded to form a ring;
one of carbon atoms bonded to R₂₅, R₂₇ and R₂₈ is bonded to *1;
X₁ is an oxygen atom, a sulfur atom, or NR₃₉;
R₃₉ is a substituent;
R₃₁ to R₃₈ are each independently a hydrogen atom or a substituent, or at least one combination of a combination of R₃₁ and R₃₂, a combination of R₃₂ and R₃₃, a combination of R₃₃ and R₃₄, a combination of R₃₅ and R₃₆, a combination of R₃₆ and R₃₇, or a combination of R₃₇ and R₃₈ are mutually bonded to form a ring;
one selected from carbon atoms bonded to R₃₁ to R₃₈ and a nitrogen atom bonded to R₃₉ is bonded to *;
R₁₁ to R₁₈, R₂₁ to R₂₈, and R₃₁ to R₃₉ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphory group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, a group represented by —N(Rz)₂, a thiol group, a substituted or unsubstituted alkylthio group having 1 to 30 carbon atoms, a substituted or unsubstituted aralkyl group having 7 to 30 ring carbon atoms, a substituted germanium group, a substituted phosphine oxide group, a nitro group, a substituted boryl group, or a substituted or unsubstituted arylthio group having 6 to 30 ring carbon atoms;
Rz is a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms; and
two Rz in —N(Rz)₂ are mutually the same or different.

According to another aspect of the invention, there is provided an electronic device including the organic electroluminescence device according to the aspect of the invention.

According to still another aspect of the invention, there is provided a compound represented by a formula (121) or (122) below.

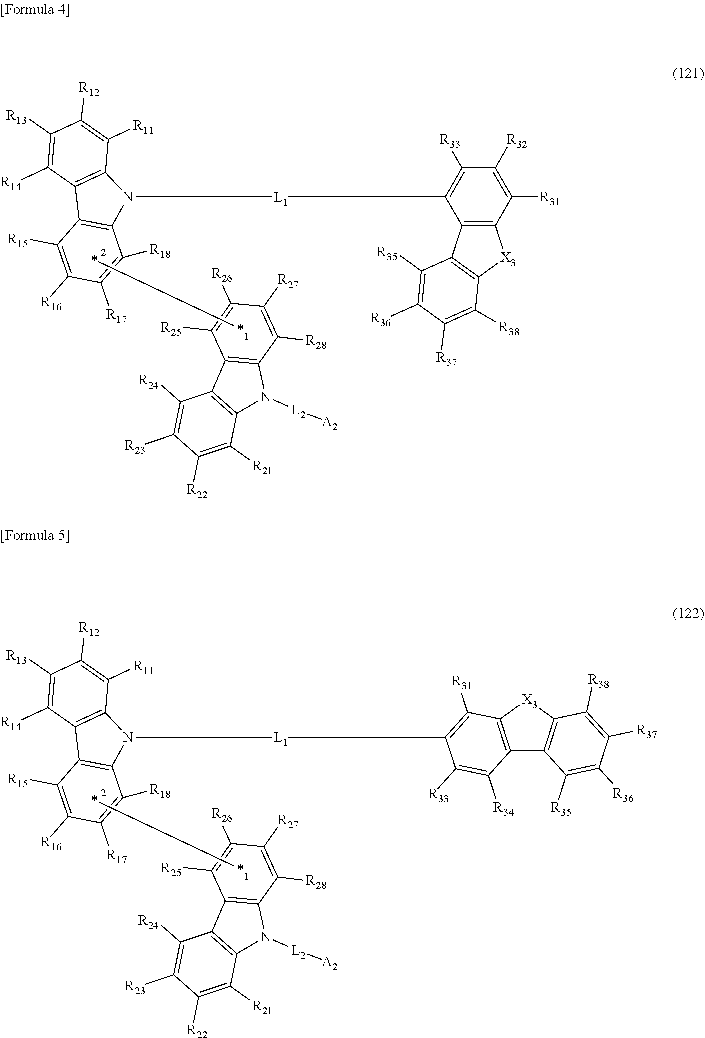

In the formulae (121) and (122):
A₂ is a substituted or unsubstituted aryl group having 6 to 30 ring carbon atoms, or a substituted or unsubstituted heterocyclic group having 5 to 30 ring atoms;
L₁ and L₂ are each independently a single bond, a substituted or unsubstituted arylene group having 6 to 30 ring carbon atoms, a substituted or unsubstituted divalent heterocyclic group having 5 to 30 ring atoms, a group obtained by bonding two groups selected from the group consisting of a substituted or unsubstituted arylene group having 6 to 30 ring carbon atoms and a substituted or unsubstituted divalent heterocyclic group having 5 to 30 ring atoms, or a group obtained by bonding three groups selected from the group consisting of a substituted or unsubstituted arylene group having 6 to 30 ring carbon atoms and a substituted or unsubstituted divalent heterocyclic group having 5 to 30 ring atoms; the two groups are mutually the same or different;
the three groups are mutually the same or different;
R₁₁ to R₁₈ are each independently a hydrogen atom or a substituent, or at least one combination of a combination of R₁₁ and R₁₂, a combination of R₁₂ and R₁₃, a combination of R₁₃ and R₁₄, a combination of R₁₅ and R₁₁, a combination of R₁₆ and R₁₇, or a combination of R₁₇ and R₁₈ are mutually bonded to form a ring;
R₂₁ to R₂₈ are each independently a hydrogen atom or a substituent, or at least one combination of a combination of R₂₁ and R₂₂, a combination of R₂₂ and R₂₃, a combination of R₂₃ and R₂₄, a combination of R₂₅ and R₂₆, a combination of R₂₆ and R₂₇, or a combination of R₂₇ and R₂₈ are mutually bonded to form a ring;
one of carbon atoms bonded to R₁₅, R₁₇ and R₁₈ is bonded to *2 and one of carbon atoms bonded to R₂₅, R₂₇ and R₂₈ is bonded to *1;
a carbon atom bonded to R₁₇ and a carbon atom bonded to R₂₇ are not simultaneously bonded to *2 and *1, respectively;
X₃ is an oxygen atom or a sulfur atom;
R₃₁ to R₃₈ are each independently a hydrogen atom or a substituent, or at least one combination of a combination of R₃₁ and R₃₂, a combination of R₃₂ and R₃₃, a combination of R₃₃ and R₃₄, a combination of R₃₅ and R₃₆, a combination of R₃₆ and R₃₇, or a combination of R₃₇ and R₃₈ are mutually bonded to form a ring;
R₁₁ to R₁₈, R₂₁ to R₂₈, and R₃₁ to R₃₈ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphory group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, a group represented by —N(Rz)₂, a thiol group, a substituted or unsubstituted alkylthio group having 1 to 30 carbon atoms, a substituted or unsubstituted aralkyl group having 7 to 30 ring carbon atoms, a substituted germanium group, a substituted phosphine oxide group, a nitro group, a substituted boryl group, or a substituted or unsubstituted arylthio group having 6 to 30 ring carbon atoms;
Rz is a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms; and
two Rz in —N(Rz)₂ are mutually the same or different.

According to a further aspect of the invention, there is provided a compound represented by one of formulae (131) to (134) below.

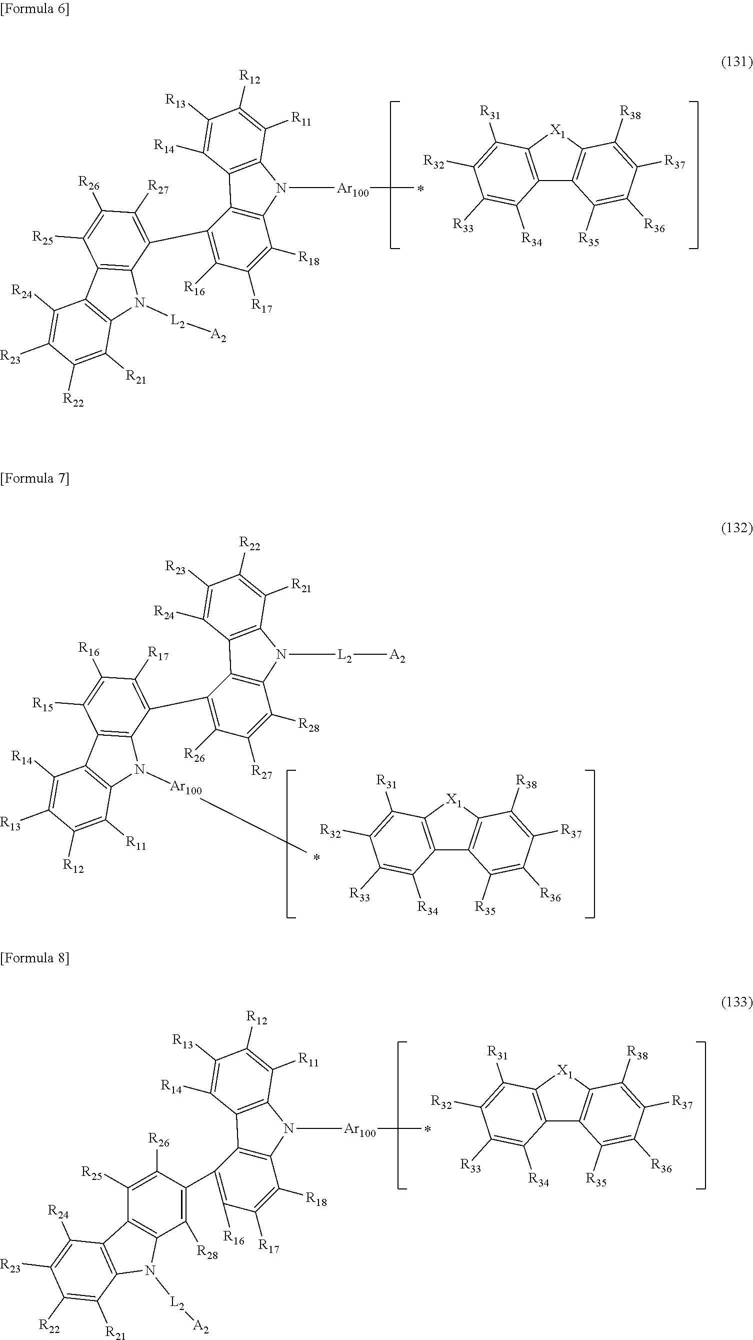

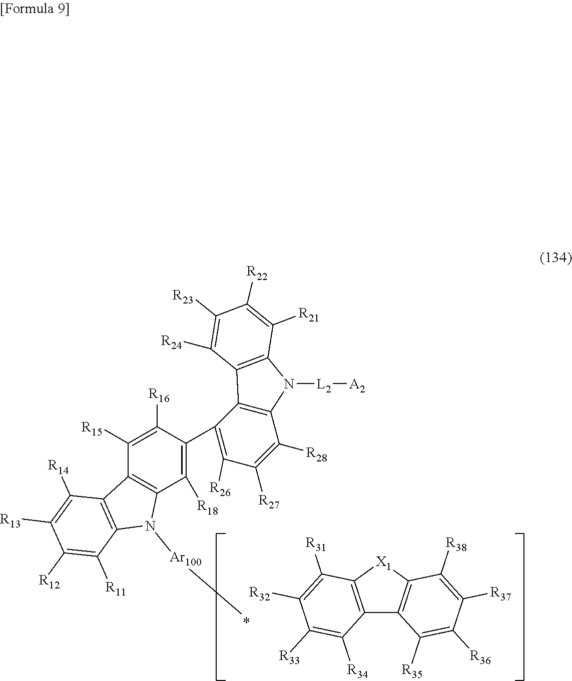

In the formulae (131) to (134):
A₂ is a substituted or unsubstituted aryl group having 6 to 30 ring carbon atoms, a substituted or unsubstituted heterocyclic group having 5 to 30 ring atoms;
Ar₁₀₀ is a substituted or unsubstituted arylene group having 6 to 30 ring carbon atoms;
L₂ is a single bond, a substituted or unsubstituted arylene group having 6 to 30 ring carbon atoms, a substituted or unsubstituted divalent heterocyclic group having 5 to 30 ring atoms, a group obtained by bonding two groups selected from the group consisting of a substituted or unsubstituted arylene group having 6 to 30 ring carbon atoms and a substituted or unsubstituted divalent heterocyclic group having 5 to 30 ring atoms, or a group obtained by bonding three groups selected from the group consisting of a substituted or unsubstituted arylene group having 6 to 30 ring carbon atoms and a substituted or unsubstituted divalent heterocyclic group having 5 to 30 ring atoms;
the two groups are mutually the same or different;
the three groups are mutually the same or different;
R₁₁ to R₁₈ are each independently a hydrogen atom or a substituent, or at least one combination of a combination of R₁₁ and R₁₂, a combination of R₁₂ and R₁₃, a combination of R₁₃ and R₁₄, a combination of R₁₅ and R₁₆, a combination of R₁₆ and R₁₇, or a combination of R₁₇ and R₁₈ are mutually bonded to form a ring;
R₂₁ to R₂₈ are each independently a hydrogen atom or a substituent, or at least one combination of a combination of R₂₁ and R₂₂, a combination of R₂₂ and R₂₃, a combination of R₂₃ and R₂₄, a combination of R₂₅ and R₂₆, a combination of R₂₆ and R₂₇, or a combination of R₂₇ and R₂₈ are mutually bonded to form a ring;
X₁ is an oxygen atom, a sulfur atom, or NR₃₉;
R₃₉ is a substituent;
R₃₁ to R₃₈ are each independently a hydrogen atom or a substituent, or at least one combination of a combination of R₃₁ and R₃₂, a combination of R₃₂ and R₃₃, a combination of R₃₃ and R₃₄, a combination of R₃₅ and R₃₆, a combination of R₃₆ and R₃₇, or a combination of R₃₇ and R₃₈ are mutually bonded to form a ring;
one selected from carbon atoms bonded to R₃₁ to R₃₈ and a nitrogen atom bonded to R₃₉ is bonded to *;
R₁₁ to R₁₈, R₂₁ to R₂₈, and R₃₁ to R₃₉ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, a group represented by —N(Rz)₂, a thiol group, a substituted or unsubstituted alkylthio group having 1 to 30 carbon atoms, a substituted or unsubstituted aralkyl group having 7 to 30 ring carbon atoms, a substituted germanium group, a substituted phosphine oxide group, a nitro group, a substituted boryl group, or a substituted or unsubstituted arylthio group having 6 to 30 ring carbon atoms;
Rz is a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms;
two Rz in —N(Rz)₂ are mutually the same or different; and
when Ar₁₀₀ has a substituent, the substituent is each independently a halogen atom, a cyano group, an unsubstituted aryl group having 6 to 30 ring carbon atoms, an unsubstituted heterocyclic group having 5 to 30 ring atoms, an unsubstituted alkyl group having 1 to 30 carbon atoms, an unsubstituted alkyl halide group having 1 to 30 carbon atoms, an unsubstituted alkenyl group having 2 to 30 carbon atoms, an unsubstituted alkynyl group having 2 to 30 carbon atoms, an unsubstituted alkylsilyl group having 3 to 30 carbon atoms, an unsubstituted arylsilyl group having 6 to 60 ring carbon atoms, an unsubstituted arylphosphoryl group having 6 to 60 ring carbon atoms, a hydroxy group, an unsubstituted alkoxy group having 1 to 30 carbon atoms, an unsubstituted aryloxy group having 6 to 30 ring carbon atoms, a thiol group, an unsubstituted alkylthio group having 1 to 30 carbon atoms, an unsubstituted aralkyl group having 7 to 30 ring carbon atoms, a substituted germanium group, a substituted phosphine oxide group, a nitro group, a substituted boryl group, or an unsubstituted arylthio group having 6 to 30 ring carbon atoms.

According to a still further aspect of the invention, there is provided a compound represented by a formula (141), (142) or (143) below

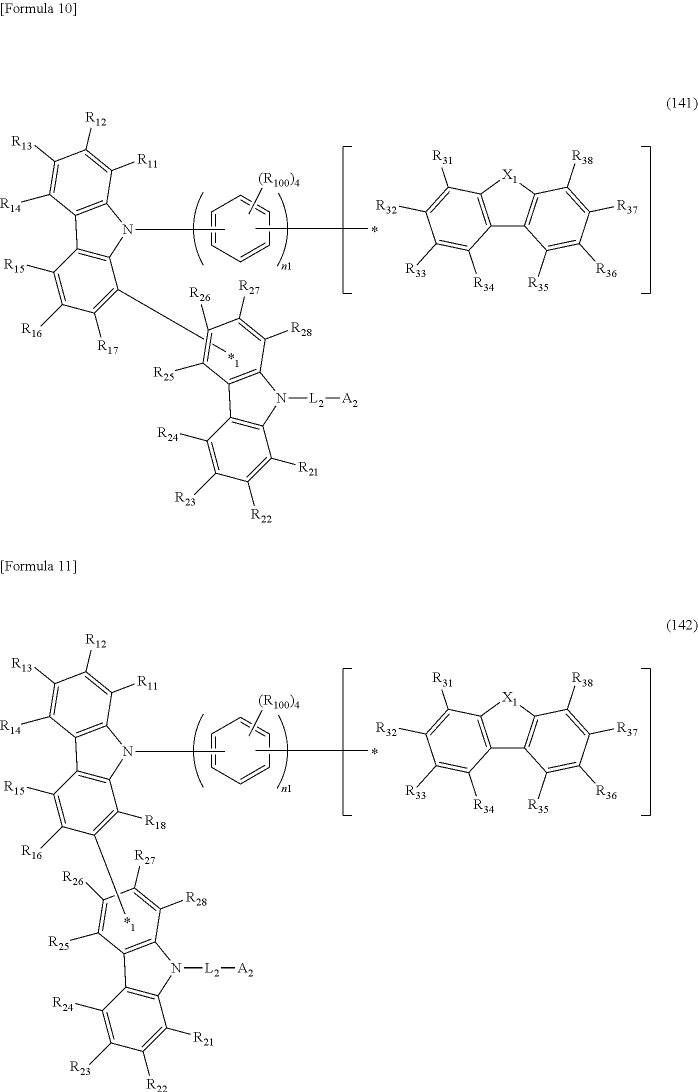

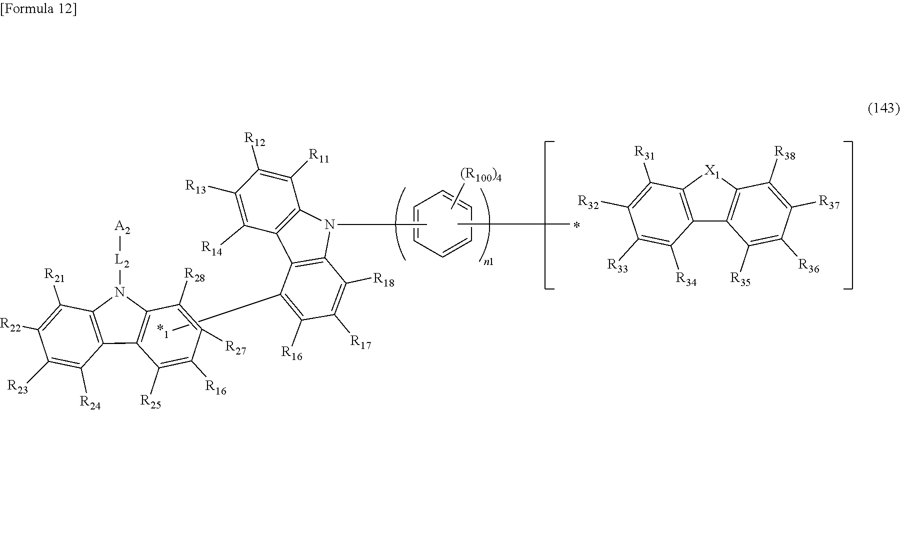

In the formulae (141) to (143):
A₂ is a substituted or unsubstituted aryl group having 6 to 30 ring carbon atoms, or a substituted or unsubstituted heterocyclic group having 5 to 30 ring atoms;
L₂ are each independently a single bond, a substituted or unsubstituted arylene group having 6 to 30 ring carbon atoms, a substituted or unsubstituted divalent heterocyclic group having 5 to 30 ring atoms, a group obtained by bonding two groups selected from the group consisting of a substituted or unsubstituted arylene group having 6 to 30 ring carbon atoms and a substituted or unsubstituted divalent heterocyclic group having 5 to 30 ring atoms, or a group obtained by bonding three groups selected from the group consisting of a substituted or unsubstituted arylene group having 6 to 30 ring carbon atoms and a substituted or unsubstituted divalent heterocyclic group having 5 to 30 ring atoms;
the two groups are mutually the same or different;
the three groups are mutually the same or different;
R₁₀₀ is each independently a hydrogen atom or a substituent;
n1 is 2, 3, or 4;
four R₁₀₀ are mutually the same or different;
R₁₁ to R₁₈ are each independently a hydrogen atom or a substituent, or at least one combination of a combination of R₁₁ and R₁₂, a combination of R₁₂ and R₁₃, a combination of R₁₃ and R₁₄, a combination of R₁₅ and R₁₆, a combination of R₁₆ and R₁₇, or a combination of R₁₇ and R₁₈ are mutually bonded to form a ring;
R₂₁ to R₂₈ are each independently a hydrogen atom or a substituent, or at least one combination of a combination of R₂₁ and R₂₂, a combination of R₂₂ and R₂₃, a combination of R₂₃ and R₂₄, a combination of R₂₅ and R₂₆, a combination of R₂₆ and R₂₇, or a combination of R₂₇ and R₂₈ are mutually bonded to form a ring;
one of carbon atoms bonded to R₂₅, R₂₇ and R₂₈ is bonded to *1;
X₁ is an oxygen atom, a sulfur atom, or NR₃₉;
R₃₉ is a substituent;
R₃₁ to R₃₈ are each independently a hydrogen atom or a substituent, or at least one combination of a combination of R₃₁ and R₃₂, a combination of R₃₂ and R₃₃, a combination of R₃₃ and R₃₄, a combination of R₃₅ and R₃₆, a combination of R₃₆ and R₃₇, or a combination of R₃₇ and R₃₈ are mutually bonded to form a ring;
one selected from carbon atoms bonded to R₃₁ to R₃₈ and a nitrogen atom bonded to R₃₉ is bonded to *;
R₁₀₀, R₁₁ to R₁₈, R₂₁ to R₂₈ and R₃₁ to R₃₉ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, a group represented by —N(Rz)₂, a thiol group, a substituted or unsubstituted alkylthio group having 1 to 30 carbon atoms, a substituted or unsubstituted aralkyl group having 7 to 30 ring carbon atoms, a substituted germanium group, a substituted phosphine oxide group, a nitro group, a substituted boryl group, or a substituted or unsubstituted arylthio group having 6 to 30 ring carbon atoms;
Rz is a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms;
two Rz in —N(Rz)₂ are mutually the same or different;
R₁₀₀ is not a group represented by —N(Rz)₂; and
when R₁₀₀ has a substituent, the substituent is each independently a halogen atom, a cyano group, an unsubstituted aryl group having 6 to 30 ring carbon atoms, an unsubstituted heterocyclic group having 5 to 30 ring atoms, an unsubstituted alkyl group having 1 to 30 carbon atoms, an unsubstituted alkyl halide group having 1 to 30 carbon atoms, an unsubstituted alkenyl group having 2 to 30 carbon atoms, an unsubstituted alkynyl group having 2 to 30 carbon atoms, an unsubstituted alkylsilyl group having 3 to 30 carbon atoms, an unsubstituted arylsilyl group having 6 to 60 ring carbon atoms, an unsubstituted arylphosphoryl group having 6 to 60 ring carbon atoms, a hydroxy group, an unsubstituted alkoxy group having 1 to 30 carbon atoms, an unsubstituted aryloxy group having 6 to 30 ring carbon atoms, a thiol group, an unsubstituted alkylthio group having 1 to 30 carbon atoms, an unsubstituted aralkyl group having 7 to 30 ring carbon atoms, a substituted germanium group, a substituted phosphine oxide group, a nitro group, a substituted boryl group, or an unsubstituted arylthio group having 6 to 30 ring carbon atoms.

According to a still further aspect of the invention, there is provided an organic-electroluminescence-device material containing the compound according to the aspect of the invention.

According to the above aspect of the invention, there can be provided an organic electroluminescence device excellent in performance, particularly, having an improved luminous efficiency, and an electronic device including the organic electroluminescence device.

According to the above aspect of the invention, there can be provided a compound capable of achieving an organic electroluminescence device excellent in performance, particularly, having an improved luminous efficiency, and an organic-electroluminescence-device material containing the compound.

### BRIEF EXPLANATION OF DRAWINGS

FIG. 1 schematically shows an exemplary arrangement of an organic electroluminescence device according to a first exemplary embodiment of the invention.

FIG. 2 schematically shows a device for measuring transient PL.

FIG. 3 shows an example of decay curves of the transient PL.

FIG. 4 schematically shows a relationship in energy level between a compound M3 and a compound M2 in an emitting layer of an exemplary organic electroluminescence device according to the first exemplary embodiment of the invention.

FIG. 5 schematically shows a relationship in energy level and energy transfer between the compound M3, the compound M2 and a compound M1 in an emitting layer of an exemplary organic electroluminescence device according to a second exemplary embodiment of the invention.

### DESCRIPTION OF EMBODIMENT(S)

### First Exemplary Embodiment

An arrangement of an organic EL device according to a first exemplary embodiment of the invention will be described below.

The organic EL device includes an anode, a cathode, and an organic layer between the anode and the cathode. The organic layer includes at least one layer formed from an organic compound(s). Alternatively, the organic layer includes a plurality of layers formed from an organic compound(s). The organic layer may further contain an inorganic compound(s). In the organic EL device of the exemplary embodiment, at least one layer of the organic layer is an emitting layer. For instance, the organic layer may be one emitting layer, or may further include a layer(s) usable in the organic EL device. Examples of the layer usable in the organic EL device, which are not particularly limited, include at least one selected from the group consisting of a hole injecting layer, a hole transporting layer, an electron injecting layer, an electron transporting layer, and a blocking layer.

The organic EL device of the exemplary embodiment includes the emitting layer between the anode and the cathode.

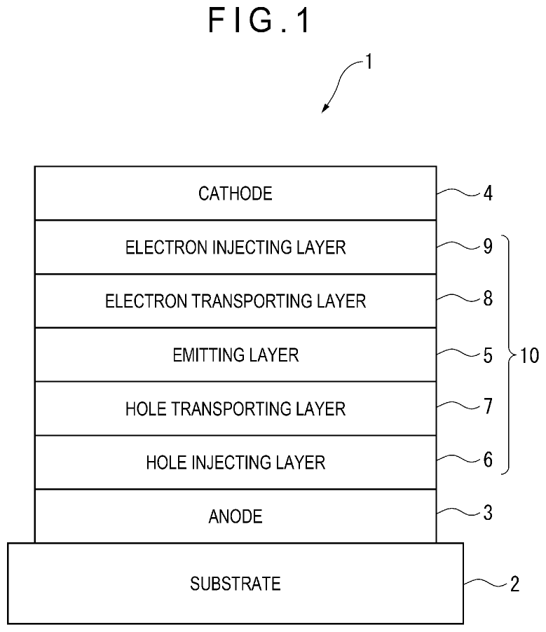

FIG. 1 schematically shows an exemplary arrangement of an organic EL device according to the exemplary embodiment.

An organic EL device 1 includes a light-transmissive substrate 2, an anode 3, a cathode 4, and an organic layer 10 provided between the anode 3 and the cathode 4. The organic layer 10 includes a hole injecting layer 6, a hole transporting layer 7, an emitting layer 5, an electron transporting layer 8, and an electron injecting layer 9 that are layered on the anode 3 in this order.

In an exemplary arrangement of the exemplary embodiment, the emitting layer may contain a metal complex.

In an exemplary arrangement of the exemplary embodiment, the emitting layer preferably does not contain a phosphorescent material (dopant material).

In an exemplary arrangement of the exemplary embodiment, the emitting layer preferably does not contain a heavy-metal complex and a phosphorescent rare earth metal complex. Examples of the heavy-metal complex herein include iridium complex, osmium complex, and platinum complex.

In an exemplary arrangement of the exemplary embodiment, the emitting layer also preferably does not contain a metal complex.

In the organic EL device of the exemplary embodiment, the emitting layer contains a compound M3 represented by a formula (11), (12) or (13) below and a delayed fluorescent compound M2.

In this arrangement, the compound M2 is preferably a dopant material (also referred to as a guest material, emitter or luminescent material), and the compound M3 is preferably a host material (also referred to as a matrix material).

The inventors have found that a high-performance organic EL device is achievable by using the compound M3 represented by the formula (11), (12) or (13) with the delayed fluorescent compound M2.

In general, a moiety having a large absolute value of an energy level of LUMO (lowest unoccupied molecular orbital) is often introduced into a molecule of a delayed fluorescent compound to reduce ΔST, which would also often increase an absolute value of an energy level of HOMO (highest occupied molecular orbital) of the entire molecule thereof. However, having a large absolute value of an energy level of HOMO may hinder hole injection into the emitting layer from the hole transporting layer, decreasing the luminous efficiency.

The compound M3 has a “biscarbazole structure excellent in hole injectability and hole transportability” and a “three-fused ring structure containing a hetero atom” (specifically, dibenzofuran, dibenzothiophen or carbazole) exhibiting a high heat stability contributing to a stable formation of an amorphous thin film.

In the exemplary embodiment, the emitting layer is made to contain the delayed fluorescent compound M2 and the compound M3 having the two structures (“biscarbazole structure” and “three-fused ring structure containing a hetero atom”), whereby the emitting layer can reduce hindrance to hole injection from the hole transporting layer to the emitting layer and can be obtained in a form of an amorphous thin film that is unlikely to be crystallized. As a result, luminous efficiency is considered to be improved.

Therefore, according to the exemplary embodiment, the organic EL device having a high performance, particularly, an improved luminous efficiency is achievable.

The wording “a high performance” in the exemplary embodiment means at least one of improving the luminous efficiency, emitting light for a long life, or decreasing drive voltage.

Patent Literature 2 discloses a biscarbazole structure in which a position 3 of one carbazole is bonded to the other carbazole. However, the luminous efficiency is decreased in the organic EL device in which the compound having the biscarbazole structure of Patent Literature 2 (e.g., a compound Ref-1 in Table 1 below) and the delayed fluorescent compound M2 are contained in the emitting layer. This is considered to be because an absolute value of an HOMO energy level is too small.

For instance, if the compound M3 having too small absolute value of the HOMO energy level and the delayed fluorescent compound M2 are contained in the emitting layer, hindrance to hole injection from the compound M3 to the compound M2, and exciplex formation of the compound M3 and the compound M2 are likely to occur, thereby causing decrease in efficiency.

Table 1 shows absolute values of HOMO energy level obtained by molecular orbital calculation for a compound M3-1 and a compound M3-5 as the compound M3 and a compound Ref-1 as the compound M3.

The molecular orbital calculation was performed using Gaussian98 at the B3LYP/6-31G* level.

In Table 1, the compound M3-1 and the compound M3-5 are compounds used in Examples 1 and 2 described later. The compound Ref-1 is a compound used in Comparative 1 described later and the compound having the biscarbazole structure in Patent Literature 2.

TABLE 1

| | 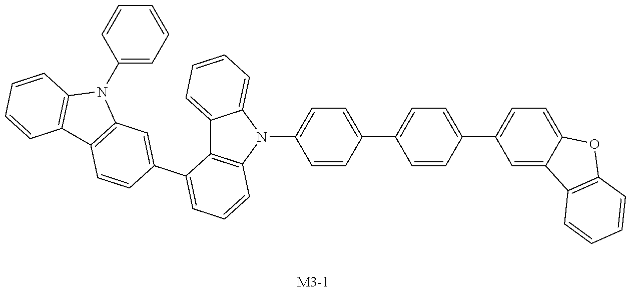 [SMILES: c1ccc(-n2c3ccccc3c3ccc(-c4cccc5c4c4ccccc4n5-c4ccc(-c5ccc(-c6ccc7oc8ccccc8c7c6)cc5)cc4)cc32)cc1] | 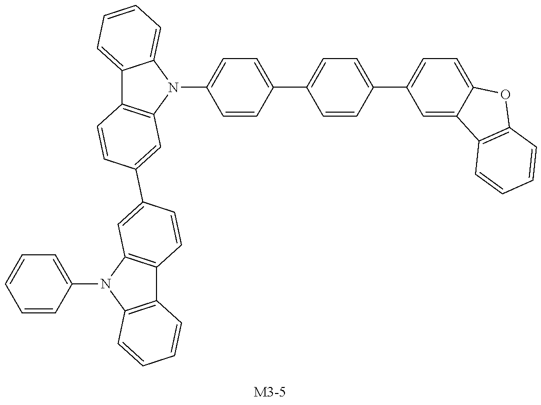 [SMILES: c1ccc(-n2c3ccccc3c3ccc(-c4ccc5c6ccccc6n(-c6ccc(-c7ccc(-c8ccc9oc%10ccccc%10c9c8)cc7)cc6)c5c4)cc32)cc1] |
|---|---|---|
| HOMO Calculation | 5.20 eV | 5.28 eV |
| Value | | |

| | 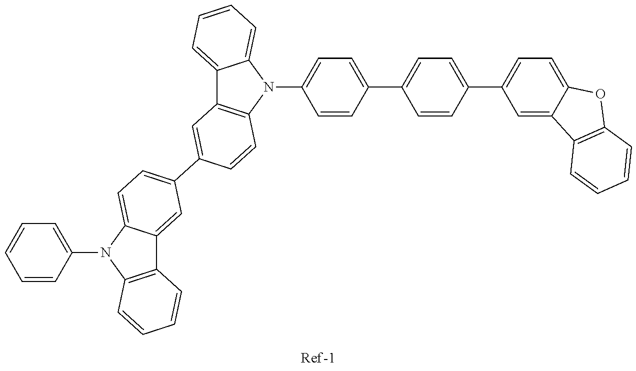 [SMILES: c1ccc(-n2c3ccccc3c3cc(-c4ccc5c(c4)c4ccccc4n5-c4ccc(-c5ccc(-c6ccc7oc8ccccc8c7c6)cc5)cc4)ccc32)cc1] |
|---|---|
| HOMOE Calculation | 4.97 eV |
| Value | |

### Emitting Layer: Compound M3

The emitting layer of the exemplary embodiment contains the compound M3 represented by the formula (11), (12) or (13) below.

The compound M3 of the exemplary embodiment may be a thermally activated delayed fluorescent compound or a compound exhibiting no thermally activated delayed fluorescence. However, the compound M3 is preferably a compound exhibiting no thermally activated delayed fluorescence.

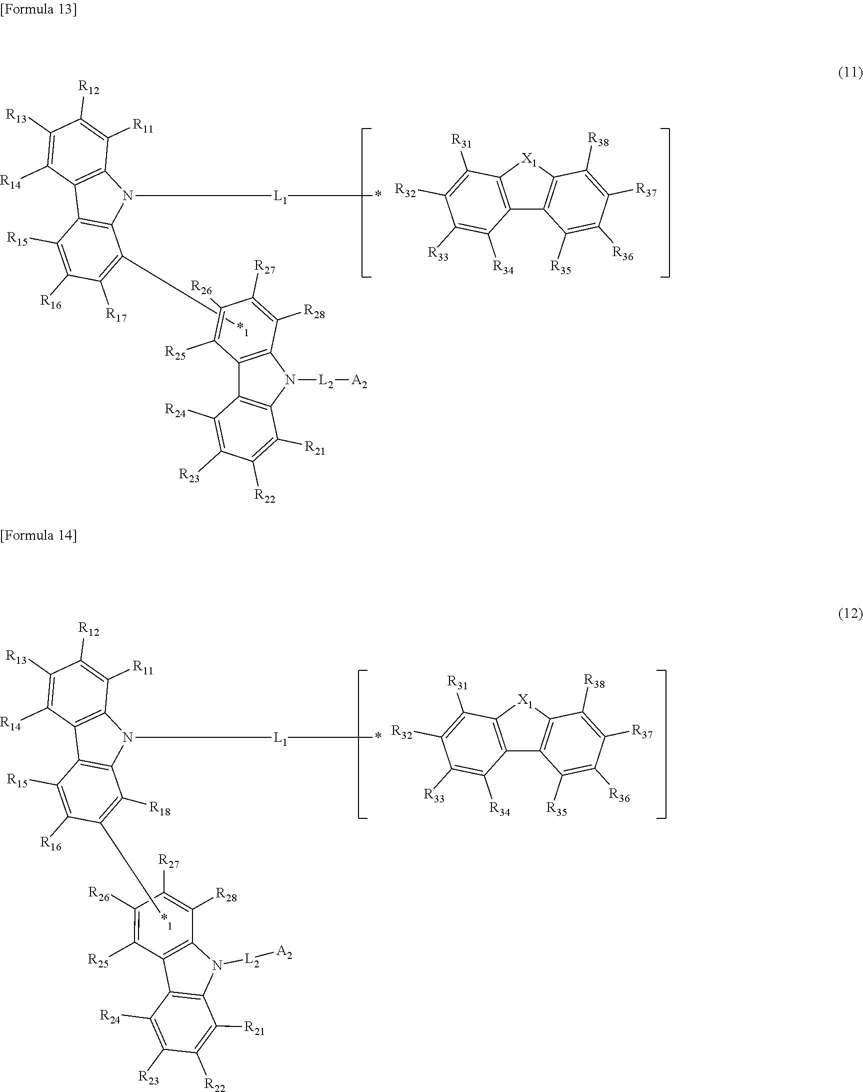

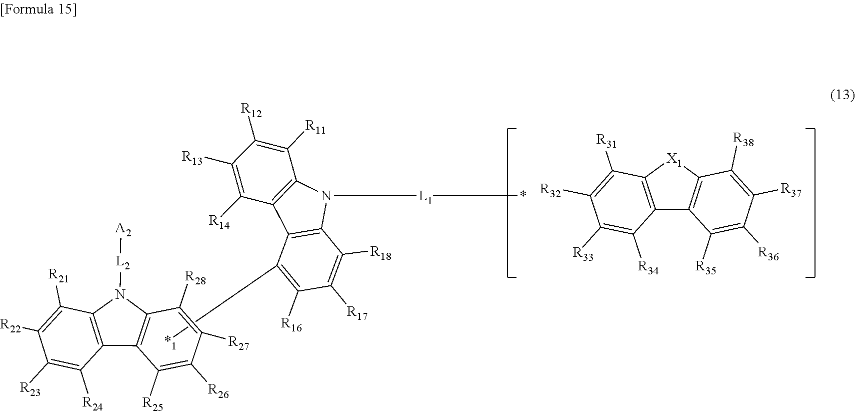

In the formulae (11) to (13):
A₂ is a substituted or unsubstituted aryl group having 6 to 30 ring carbon atoms, or a substituted or unsubstituted heterocyclic group having 5 to 30 ring atoms;
L₁ and L₂ are each independently a single bond, a substituted or unsubstituted arylene group having 6 to 30 ring carbon atoms, a substituted or unsubstituted divalent heterocyclic group having 5 to 30 ring atoms, a group obtained by bonding two groups selected from the group consisting of a substituted or unsubstituted arylene group having 6 to 30 ring carbon atoms and a substituted or unsubstituted divalent heterocyclic group having 5 to 30 ring atoms, or a group obtained by bonding three groups selected from the group consisting of a substituted or unsubstituted arylene group having 6 to 30 ring carbon atoms and a substituted or unsubstituted divalent heterocyclic group having 5 to 30 ring atoms;
the two groups are mutually the same or different;
the three groups are mutually the same or different;
R₁₁ to R₁₈ are each independently a hydrogen atom or a substituent, or at least one combination of a combination of R₁₁ and R₁₂, a combination of R₁₂ and R₁₃, a combination of R₁₃ and R₁₄, a combination of R₁₅ and R₁₆, a combination of R₁₆ and R₁₇, or a combination of R₁₇ and R₁₈ are mutually bonded to form a ring;
R₂₁ to R₂₈ are each independently a hydrogen atom or a substituent, or at least one combination of a combination of R₂₁ and R₂₂, a combination of R₂₂ and R₂₃, a combination of R₂₃ and R₂₄, a combination of R₂₅ and R₂₈, a combination of R₂₆ and R₂₇, or a combination of R₂₇ and R₂₈ are mutually bonded to form a ring;
one of carbon atoms bonded to R₂₅, R₂₇ and R₂₆ is bonded to *1;
X₁ is an oxygen atom, a sulfur atom, or NR₃₉;
R₃₉ is a substituent;
R₃₁ to R₃₈ are each independently a hydrogen atom or a substituent, or at least one combination of a combination of R₃₁ and R₃₂, a combination of R₃₂ and R₃₃, a combination of R₃₃ and R₃₄, a combination of R₃₅ and R₃₆, a combination of R₃₆ and R₃₇, or a combination of R₃₇ and R₃₈ are mutually bonded to form a ring;
one selected from carbon atoms bonded to R₃₁ to R₃₈ and a nitrogen atom bonded to R₃₉ is bonded to *;
R₁₁ to R₁₈, R₂₁ to R₂₈, and R₃₁ to R₃₉ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, a group represented by —N(Rz)₂, a thiol group, a substituted or unsubstituted alkylthio group having 1 to 30 carbon atoms, a substituted or unsubstituted aralkyl group having 7 to 30 ring carbon atoms, a substituted germanium group, a substituted phosphine oxide group, a nitro group, a substituted boryl group, or a substituted or unsubstituted arylthio group having 6 to 30 ring carbon atoms;
Rz is a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms;
two Rz in —N(Rz)₂ are mutually the same or different.

In the formulae (11) to (13): when a carbon atom bonded to R₃₁ is bonded to *, a hydrogen atom represented by R₃₁, a substituent represented by R₃₁, and a ring formed by involving R₃₁ are not present.

In the formulae (11) to (13), when a carbon atom bonded to R₃₂ is bonded to *, a hydrogen atom represented by R₃₂, a substituent represented by R₃₂, and a ring formed by involving R₃₂ are not present.

In the formulae (11) to (13), when a carbon atom bonded to R₃₃ is bonded to *, a hydrogen atom represented by R₃₃, a substituent represented by R₃₃, and a ring formed by involving R₃₃ are not present.

In the formulae (11) to (13), when a carbon atom bonded to R₃₄ is bonded to *, a hydrogen atom represented by R₃₄, a substituent represented by R₃₄, and a ring formed by involving R₃₄ are not present.

In the formulae (11) to (13), when a carbon atom bonded to R₃₅ is bonded to *, a hydrogen atom represented by R₃₅, a substituent represented by R₃₅, and a ring formed by involving R₃₅ are not present.

In the formulae (11) to (13), when a carbon atom bonded to R₃₆ is bonded to *, a hydrogen atom represented by R₃₆, a substituent represented by R₃₆, and a ring formed by involving R₃₆ are not present.

In the formulae (11) to (13), when a carbon atom bonded to R₃₇ is bonded to *, a hydrogen atom represented by R₃₇, a substituent represented by R₃₇, and a ring formed by involving R₃₇ are not present.

In the formulae (11) to (13), when a carbon atom bonded to R₃₈ is bonded to *, a hydrogen atom represented by R₃₈, a substituent represented by R₃₈, and a ring formed by involving R₃₈ are not present.

In the formulae (11) to (13), when a nitrogen atom bonded to R₃₉ is bonded to *, a substituent represented by R₃₉ is not present.

In the formulae (11) to (13), when a carbon atom bonded to R₂₅ is bonded to *1, a hydrogen atom represented by R₂₅, a substituent represented by R₂₅, and a ring formed by involving R₂₅ are not present.

In the formulae (11) to (13), when a carbon atom bonded to R₂₇ is bonded to *1, a hydrogen atom represented by R₂₇, a substituent represented by R₂₇, and a ring formed by involving R₂₇ are not present.

In the formulae (11) to (13): when a carbon atom bonded to R₂₈ is bonded to *1, a hydrogen atom represented by R₂₈, a substituent represented by R₂₈, and a ring formed by involving R₂₈ are not present.

In the compound M3, it is preferable that a combination of R₁₁ and R₁₂, a combination of R₁₂ and R₁₃, a combination of R₁₃ and R₁₄, a combination of R₁₅ and R₁₁, a combination of R₁₆ and R₁₇, and a combination of R₁₇ and R₁₈ are not mutually bonded. In other words, it is preferable that R₁₁ to R₁₈ in the compound M3 are each independently a hydrogen atom or a substituent.

In the compound M3, it is also preferable that at least one combination of a combination of R₁₁ and R₁₂, a combination of R₁₂ and R₁₃, a combination of R₁₃ and R₁₄, a combination of R₁₅ and R₁₆, a combination of R₁₆ and R₁₇, or a combination of R₁₇ and R₁₈ are mutually bonded to form a ring.

In the compound M3, it is preferable that a combination of R₂₁ and R₂₂, a combination of R₂₂ and R₂₃, a combination of R₂₃ and R₂₄, a combination of R₂₅ and R₂₆, a combination of R₂₆ and R₂₇, and a combination of R₂₇ and R₂₆ are not mutually bonded. In other words, it is preferable that R₂₁ to R₂₈ in the compound M3 are each independently a hydrogen atom or a substituent.

In the compound M3, it is also preferable that at least one combination of a combination of R₂₁ and R₂₂, a combination of R₂₂ and R₂₃, a combination of R₂₃ and R₂₄, a combination of R₂₅ and R₂₆, a combination of R₂₆ and R₂₇, or a combination of R₂₇ and R₂₈ are mutually bonded to form a ring.

In the compound M3, it is preferable that a combination of R₃₁ and R₃₂, a combination of R₃₂ and R₃₃, a combination of R₃₃ and R₃₄, a combination of R₃₅ and R₃₆, a combination of R₃₆ and R₃₇, and a combination of R₃₇ and R₃₈ are not mutually bonded. In other words, it is preferable that R₃₁ to R₃₈ in the compound M3 are each independently a hydrogen atom or a substituent.

In the compound M3, it is also preferable that at least one combination of a combination of R₃₁ and R₃₂, a combination of R₃₂ and R₃₃, a combination of R₃₃ and R₃₄, a combination of R₃₅ and R₃₆, a combination of R₃₆ and R₃₇, or a combination of R₃₇ and R₃₈ are mutually bonded to form a ring.

The compound M3 is preferably a compound represented by one of formulae (111) to (114) below.

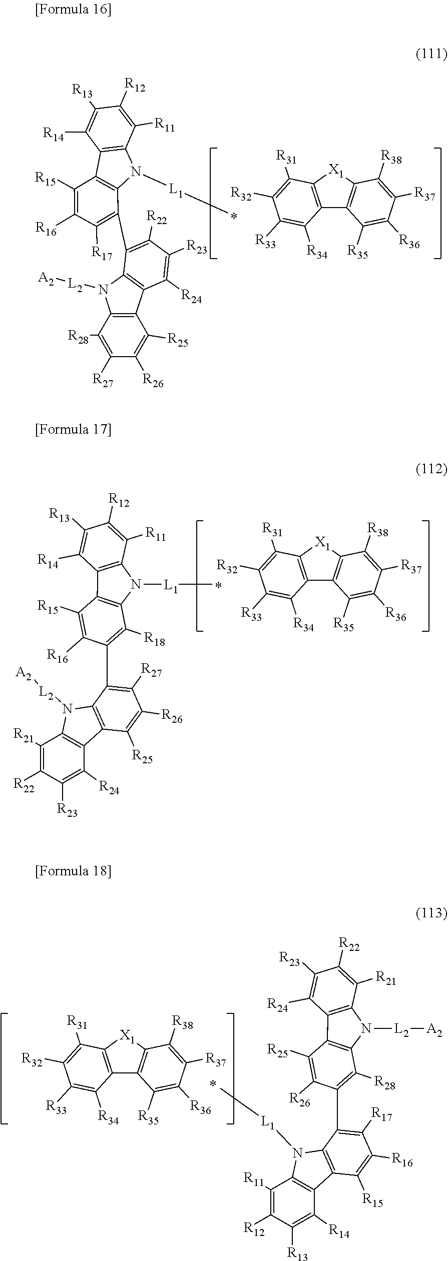

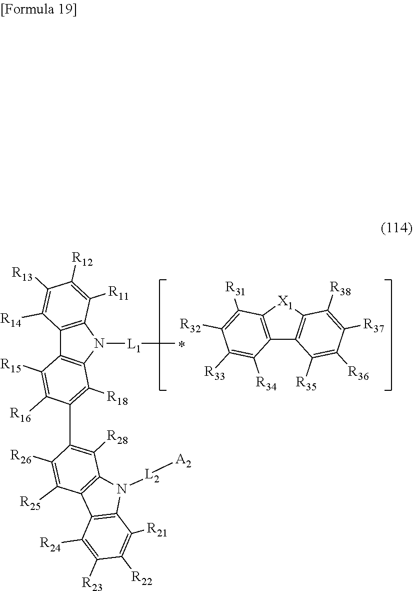

In the formulae (111) to (114), A₂, L₁, L₂, X₁, R₁₁ to R₁₈, R₂₁ to R₂₈ and R₃₁ to R₃₉ each independently represent the same as A₂, L₁, L₂, X₁, R₁₁ to R₁₈, R₂₁ to R₂₈ and R₃₁ to R₃₉ in the formulae (11) to (13) and one selected from carbon atoms bonded to R₃₁ to R₃₈ and a nitrogen atom bonded to R₃₉ is bonded to *.

The compound M3 is also preferably a compound represented by one of formulae (115) to (119) below.

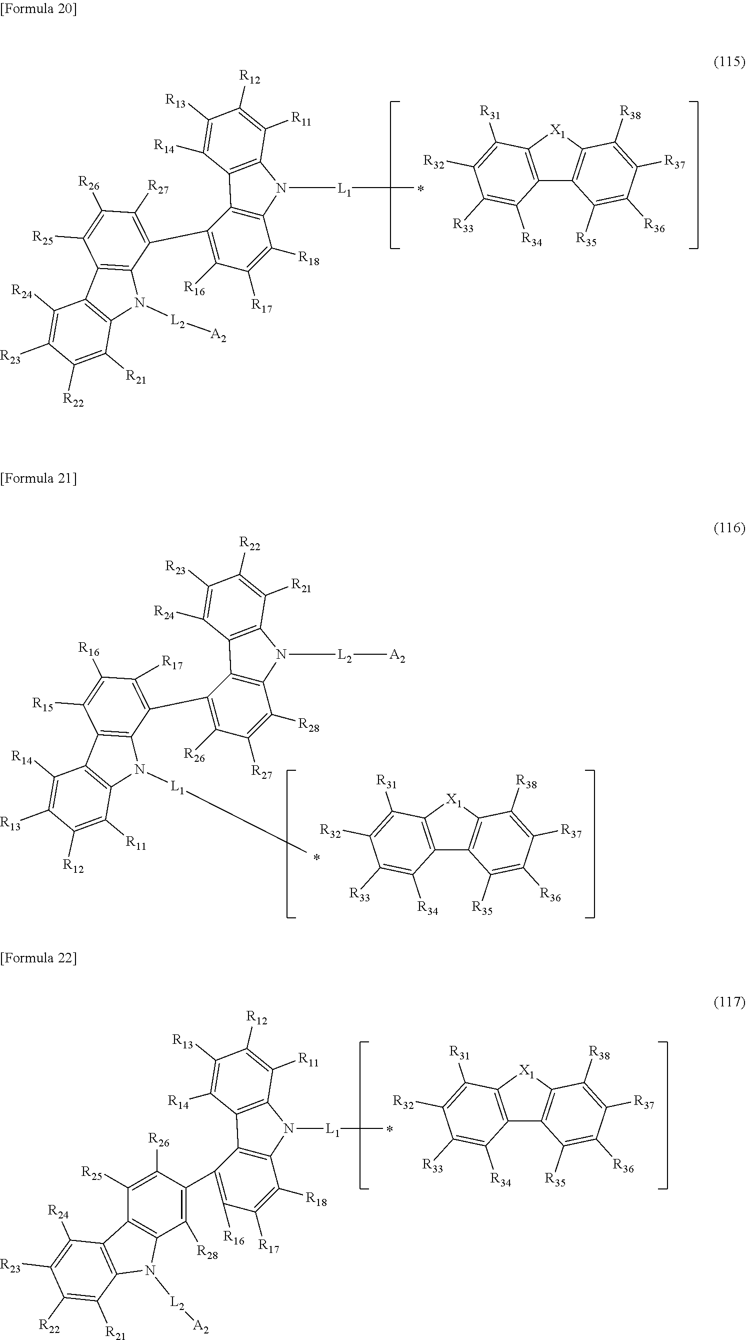

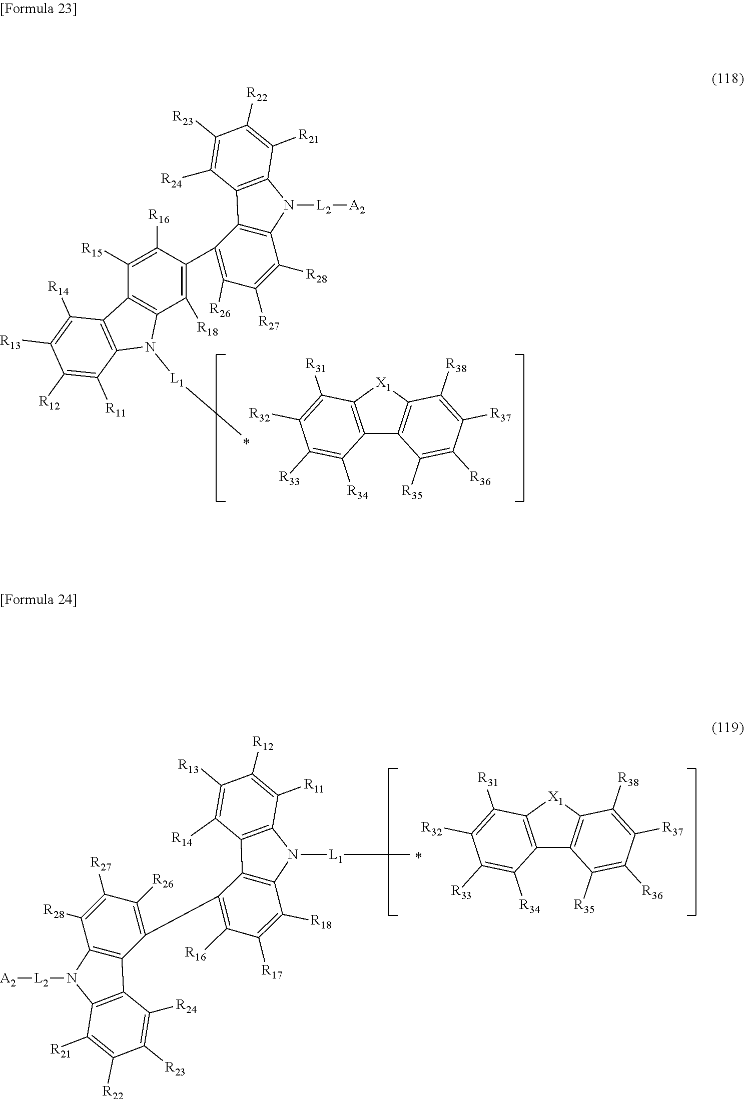

In the formulae (115) to (119), A₂, L₁, L₂, X₁, R₁₁ to R₁₈, R₂₁ to R₂₈ and R₃₁ to R₃₉ each independently represent the same as A₂, L₁, L₂, X₁, R₁₁ to R₁₈, R₂₁ to R₂₈ and R₃₁ to R₃₉ in the formulae (11) to (13) and one selected from carbon atoms bonded to R₃₁ to R₃₈ and a nitrogen atom bonded to R₃₉ is bonded to *.

The compound M3 is also preferably a compound represented by one of the formulae (115), (117) and (119).

The compound M3 is also preferably a compound represented by one of the formulae (111) to (113) and (115) to (119).

The compound M3 is also preferably a compound represented by one of the formulae (111), (115) to (116) and (119).

In the compound M3, A₂ is preferably a substituted or unsubstituted aryl group having 6 to 18 ring carbon atoms or a substituted or unsubstituted heterocyclic group having 5 to 18 ring atoms.

In the compound M3, A₂ is more preferably a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, or a group represented by a formula (14) below.

In the compound M3, it is preferable that L₂ is a single bond and A₂ is a substituted or unsubstituted aryl group having 6 to 18 ring carbon atoms or a substituted or unsubstituted heterocyclic group having 5 to 18 ring atoms.

In the compound M3, it is more preferable that L₂ is a single bond and A₂ is a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, or a group represented by the formula (14).

In the compound M3, it is still more preferable that L₂ is a single bond and A₂ is an unsubstituted phenyl group.

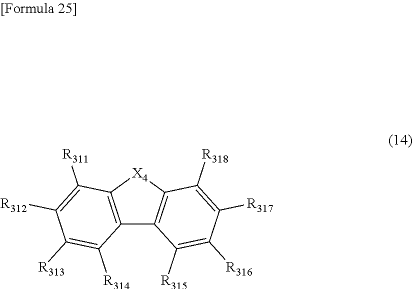

In the formula (14): X₄ is an oxygen atom, a sulfur atom, or NR₃₁₉;

R₃₁₁ to R₃₁₈ each independently represent the same as R₃₁ to R₃₈ in the formulae (11) to (13); R₃₁₉ each independently represents the same as R₃9 in the formulae (11) to (13); and one selected from carbon atoms bonded to R₃₁₁ to R₃₁₈ and a nitrogen atom bonded to R₃₁₉ is bonded to L₂.

In the formula (14), X₄ is preferably an oxygen atom or a sulfur atom.

In the formula (14), it is preferable that R₃₁₁ to R₃₁₈ are each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms; and R₃₁₉ is a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the formula (14), it is more preferable that R₃₁₁ to R₃₁₈ are each independently a hydrogen atom or a substituted or unsubstituted aryl group having 6 to 30 ring carbon atoms, and R₃₁₉ is a substituted or unsubstituted aryl group having 6 to 30 ring carbon atoms.

In the formula (14), it is still more preferable that R₃₁₁ to R₃₁₈ are each a hydrogen atom and R₃₁₉ is a substituted or unsubstituted phenyl group.

In the compound M3, it is preferable that R₁ to R₁₈, R₂₁ to R₂₈ and R₃₁ to R₃₈ are each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms; and R₃₉ is a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the compound M3, it is more preferable that R₁ to R₁₈, R₂₁ to R₂₈ and R₃₁ to R₃₈ are each independently a hydrogen atom or a substituted or unsubstituted aryl group having 6 to 30 ring carbon atoms; and R₃₉ is a substituted or unsubstituted aryl group having 6 to 30 ring carbon atoms.

In the formula (14), it is still more preferable that R₁₁ to R₁₈, R₂₁ to R₂₈ and R₃₁ to R₃₈ are each a hydrogen atom and R₃₉ is a substituted or unsubstituted phenyl group.

In the compound M3, it is preferable that L₁ and L₂ are each independently a single bond or a substituted or unsubstituted arylene group having 6 to 30 ring carbon atoms.

In the compound M3, it is more preferable that L₁ and L₂ are each independently a substituted or unsubstituted phenylene group, a substituted or unsubstituted parabiphenylene group, or a substituted or unsubstituted paraterphenylene group.

In the compound M3, it is still more preferable that L₁ and L₂ are each independently a substituted or unsubstituted parabiphenylene group, or a substituted or unsubstituted paraterphenylene group.

In the compound M3, it is also preferable that L₁ and L₂ are each independently a single bond or at least one group selected from the group consisting of groups represented by formulae (L1) to (L7) below.

In the compound M3, it is also preferable that L₂ is a single bond or a group represented by one of the formulae (L3), (L4) and (L6).

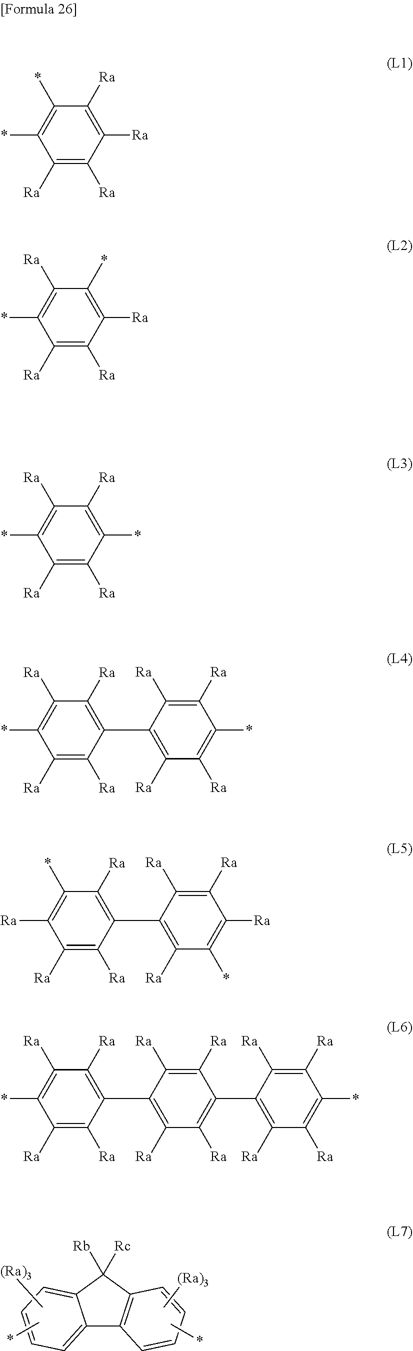

In the formulae (L1) to (L7): Ra is a hydrogen atom or a substituent; a plurality of Ra are mutually the same or different; Rb and Rc are each independently a hydrogen atom or a substituent, or a combination of Rb and Rc are mutually bonded to form a ring; and Ra, Rb and Rc as a substituent each independently represent the same as R₁₁ to R₁₈ as a substituent in the formulae (11) to (13).

In the formulae (L1) to (L7), Ra is each independently preferably a hydrogen atom, a substituted or unsubstituted aryl group having 6 to 30 ring carbon atoms, or a substituted or unsubstituted alkyl group having 1 to 30 carbon atoms, more preferably a hydrogen atom.

In the formula (L7), it is preferable that Rb and Rc are each independently a substituted or unsubstituted aryl group having 6 to 30 ring carbon atoms or a substituted or unsubstituted alkyl group having 1 to 30 carbon atoms, or a combination of Rb and Rc are mutually bonded to form a ring.

In the compound M3, it is preferable that X₁ is an oxygen atom or a sulfur atom, or X₁ is NR₃₉ and a nitrogen atom bonded to NR₃₉ is bonded to * in the formulae (11) to (13).

In the compound M3, it is more preferable that X₁ is an oxygen atom or a sulfur atom.

In the compound M3, it is still more preferable that X₁ is an oxygen atom.

In the compound M3, it is also preferable that X₁ is an oxygen atom or a sulfur atom, and a carbon atom bonded to R₃₁ is bonded to * in the formulae (11) to (13).

In the compound M3, it is also preferable that X₁ is an oxygen atom or a sulfur atom, and a carbon atom bonded to R₃₂ is bonded to * in the formulae (11) to (13).

In the compound M3, it is also preferable that X₁ is an oxygen atom or a sulfur atom, and a carbon atom bonded to R₃₃ is bonded to * in the formulae (11) to (13).

In the compound M3, it is also preferable that X₁ is an oxygen atom or a sulfur atom, and a carbon atom bonded to R₃₄ is bonded to * in the formulae (11) to (13).

In the compound M3, a substituent for “substituted or unsubstituted” group is each independently preferably a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon, more preferably an unsubstituted aryl group having 6 to 30 ring carbon atoms, an unsubstituted heterocyclic group having 5 to 30 ring atoms, or an unsubstituted alkyl group having 1 to 30 carbon atoms.

### Embodiment of Compound M3 Containing at Least One Deuterium Atom

A “hydrogen atom” used herein encompasses a protium atom, a deuterium atom, and a tritium atom. Accordingly, the compound M3 (compound represented by the formula (11), (12) or (13)) used in the organic EL device of the exemplary embodiment may contain a naturally occurring deuterium atom. Alternatively, a deuterium atom may be intentionally introduced into the compound M3 by using a starting compound in which a part or all of hydrogen atoms are deuterium atoms.

Accordingly, the compound M3 used in the organic EL device of the exemplary embodiment may contain at least one deuterium atom. Specifically, the compound M3 is a compound represented by the formula (11), (12) or (13) and at least one of hydrogen atom(s) contained in the compound M3 may be a deuterium atom.

When the compound M3 contains at least one deuterium atom, for instance, at least one hydrogen atom selected from hydrogen atoms below may be a deuterium atom:
hydrogen atom(s) included in A₂ that is a substituted or unsubstituted aryl group having 6 to 30 ring carbon atoms;
hydrogen atom(s) included in A₂ that is a substituted or unsubstituted heterocyclic group having 5 to 30 ring atoms;
hydrogen atom(s) that is included, when L₁ and L₂ are each independently a linking group (hereinafter, also referred to as a linking group L₁₀), in the linking group L₁₀, the linking group L₁₀ being each independently a substituted or unsubstituted arylene group having 6 to 30 ring carbon atoms, a substituted or unsubstituted divalent heterocyclic group having 5 to 30 ring atoms, a group obtained by bonding two groups selected from the group consisting of a substituted or unsubstituted arylene group having 6 to 30 ring carbon atoms and a substituted or unsubstituted divalent heterocyclic group having 5 to 30 ring atoms, or a group obtained by bonding three groups selected from the group consisting of a substituted or unsubstituted arylene group having 6 to 30 ring carbon atoms and a substituted or unsubstituted divalent heterocyclic group having 5 to 30 ring atoms;
hydrogen atom(s) that is at least one of R₁₁ to R₁₈, R₂₁ to R₂₈, or R₃₁ to R₃₈ Hydrogen atom that is included, when R₁₁ to R₁₈, R₂₁ to R₂₈, and R₃₁ to R₃₉ are each independently a substituent (hereinafter, also referred to as a substituent E), in the substituent E, the substituent E being each independently a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a substituted or unsubstituted alkoxy group having 1 to 30 carbon atoms, a substituted or unsubstituted aryloxy group having 6 to 30 ring carbon atoms, a group represented by —N(Rz)₂, a thiol group, a substituted or unsubstituted alkylthio group having 1 to 30 carbon atoms, a substituted or unsubstituted aralkyl group having 7 to 30 ring carbon atoms, a substituted germanium group, a substituted phosphine oxide group, a substituted boryl group, or a substituted or unsubstituted arylthio group having 6 to 30 ring carbon atoms;
hydrogen atom(s) that is at least one of R₃₁₁ to R₃₁₈ when A₂ is a group represented by the formula (14);
hydrogen atom(s) included in the substituent E when A₂ is a group represented by the formula (14) and R₃₁₁ to R₃₁₉ are each independently the substituent E;
hydrogen atom(s) that is at least one of a plurality of Ra when L₂ is a group represented by one of the formulae (L1) to (L7);
hydrogen atom(s) that is at least one of a plurality of Rb when L₂ is a group represented by one of the formulae (L1) to (L7);
hydrogen atom(s) that is at least one of a plurality of Rc when L₂ is a group represented by one of the formulae (L1) to (L7);
hydrogen atom(s) included in the substituent E when L₂ is a group represented by one of the formulae (L1) to (L7) and the plurality of Ra, the plurality of Rb, and the plurality of Rc are each independently the substituent E; and
hydrogen atom(s) included in at least one substituent of the substituents for “substituted or unsubstituted” groups in the compound M3.

### Manufacturing Method of Compound M3 in Embodiment

The compound M3 of the exemplary embodiment can be manufactured, for instance, by a method described in Example described later. The compound M3 of the exemplary embodiment can be manufactured by application of known substitution reactions and materials depending on a target compound according to reactions described in Example described later.

### Specific Examples of Compound M3

Specific examples of the compound M3 in the exemplary embodiment include compounds below. However, the invention is by no means limited to the specifically listed compounds.

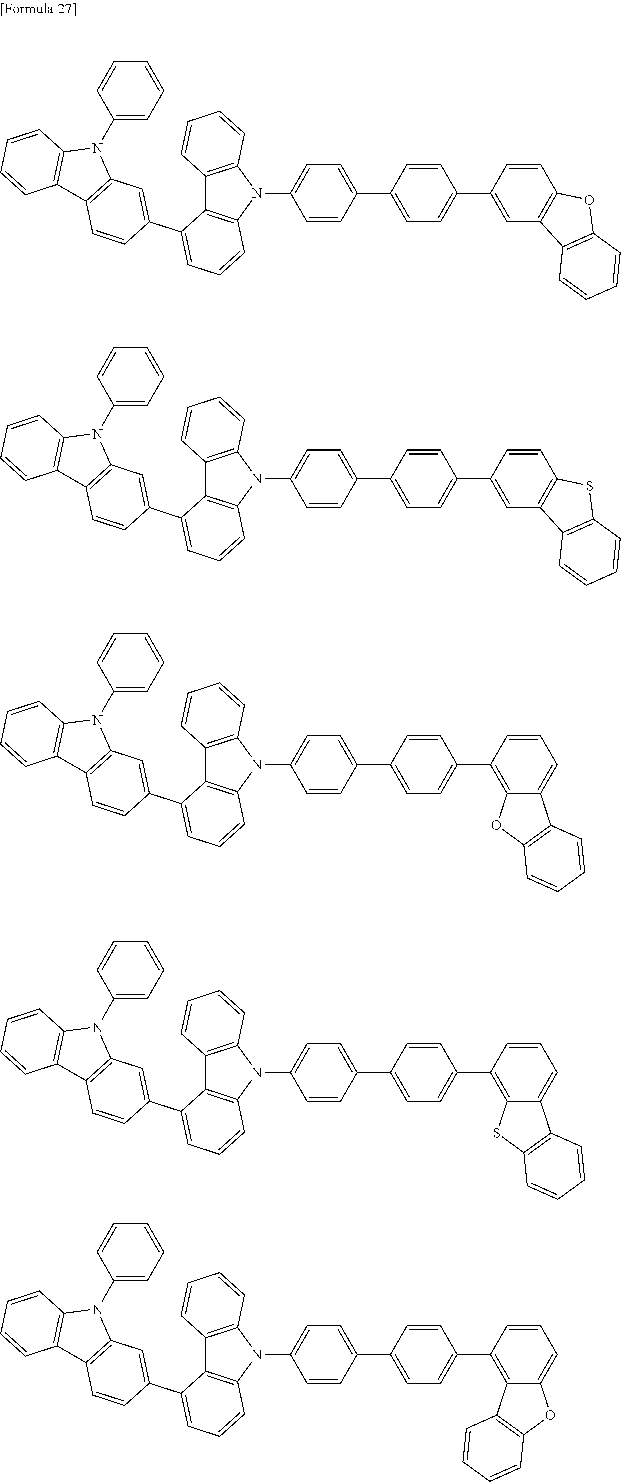 [SMILES: c1ccc(-n2c3ccccc3c3ccc(-c4cccc5c4c4ccccc4n5-c4ccc(-c5ccc(-c6ccc7oc8ccccc8c7c6)cc5)cc4)cc32)cc1 ; c1ccc(-n2c3ccccc3c3ccc(-c4cccc5c4c4ccccc4n5-c4ccc(-c5ccc(-c6ccc7sc8ccccc8c7c6)cc5)cc4)cc32)cc1 ; c1ccc(-n2c3ccccc3c3ccc(-c4cccc5c4c4ccccc4n5-c4ccc(-c5ccc(-c6cccc7c6oc6ccccc67)cc5)cc4)cc32)cc1 ; c1ccc(-n2c3ccccc3c3ccc(-c4cccc5c4c4ccccc4n5-c4ccc(-c5ccc(-c6cccc7c6sc6ccccc67)cc5)cc4)cc32)cc1 ; c1ccc(-n2c3ccccc3c3ccc(-c4cccc5c4c4ccccc4n5-c4ccc(-c5ccc(-c6cccc7oc8ccccc8c67)cc5)cc4)cc32)cc1]

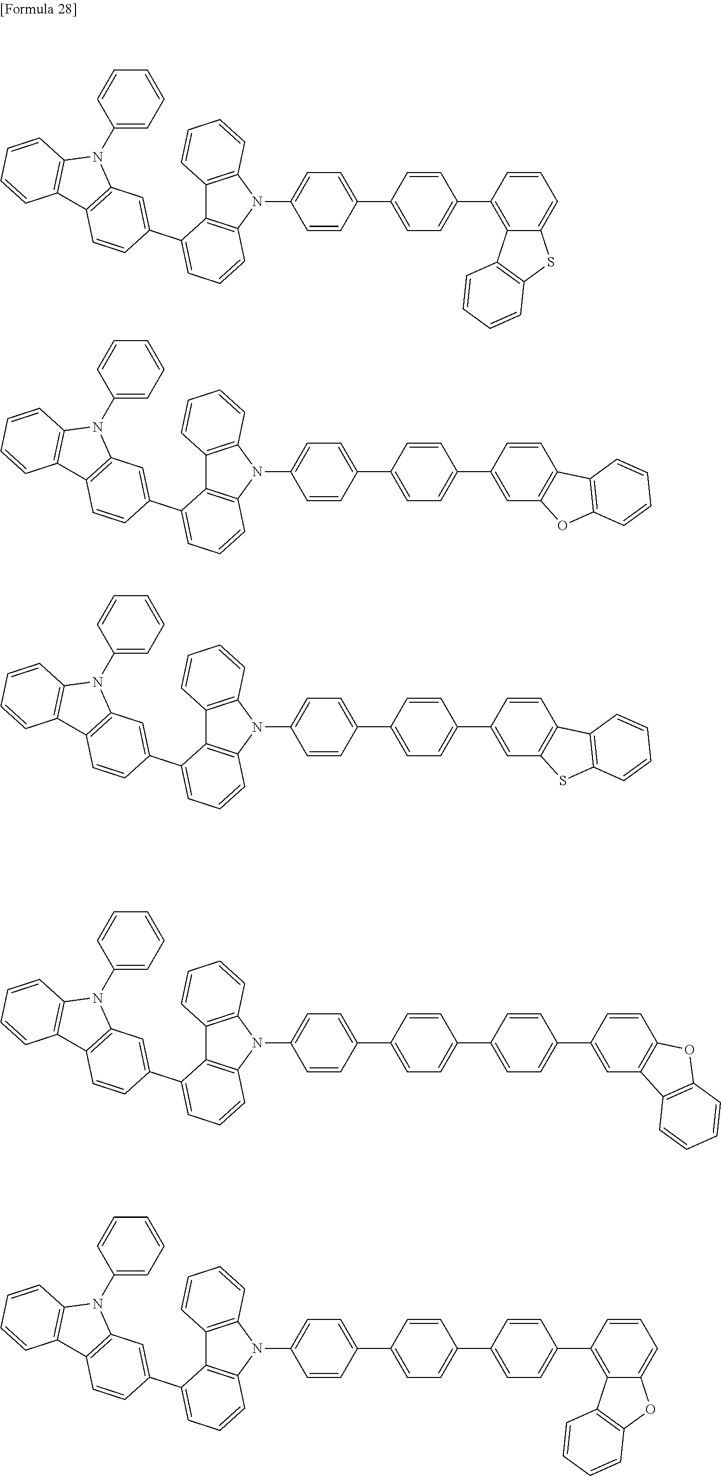

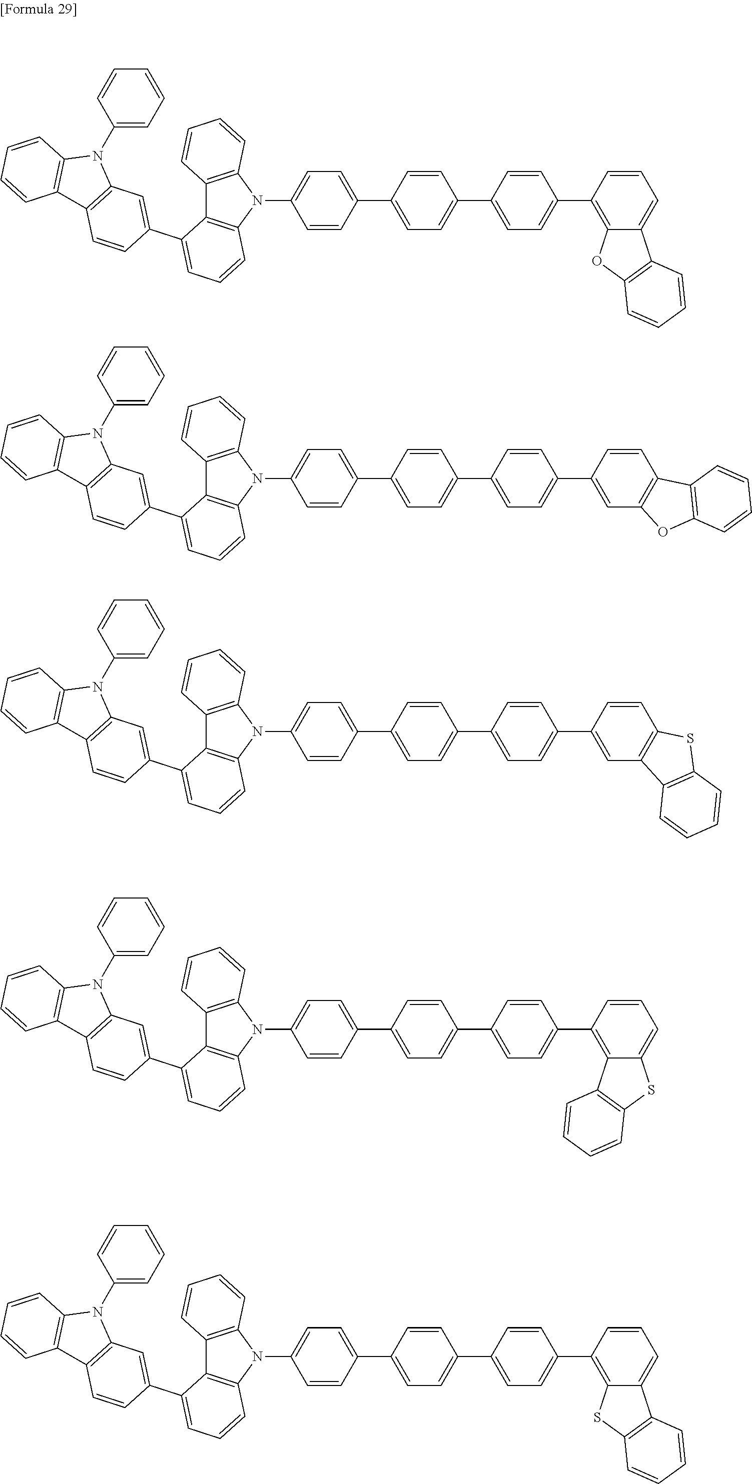

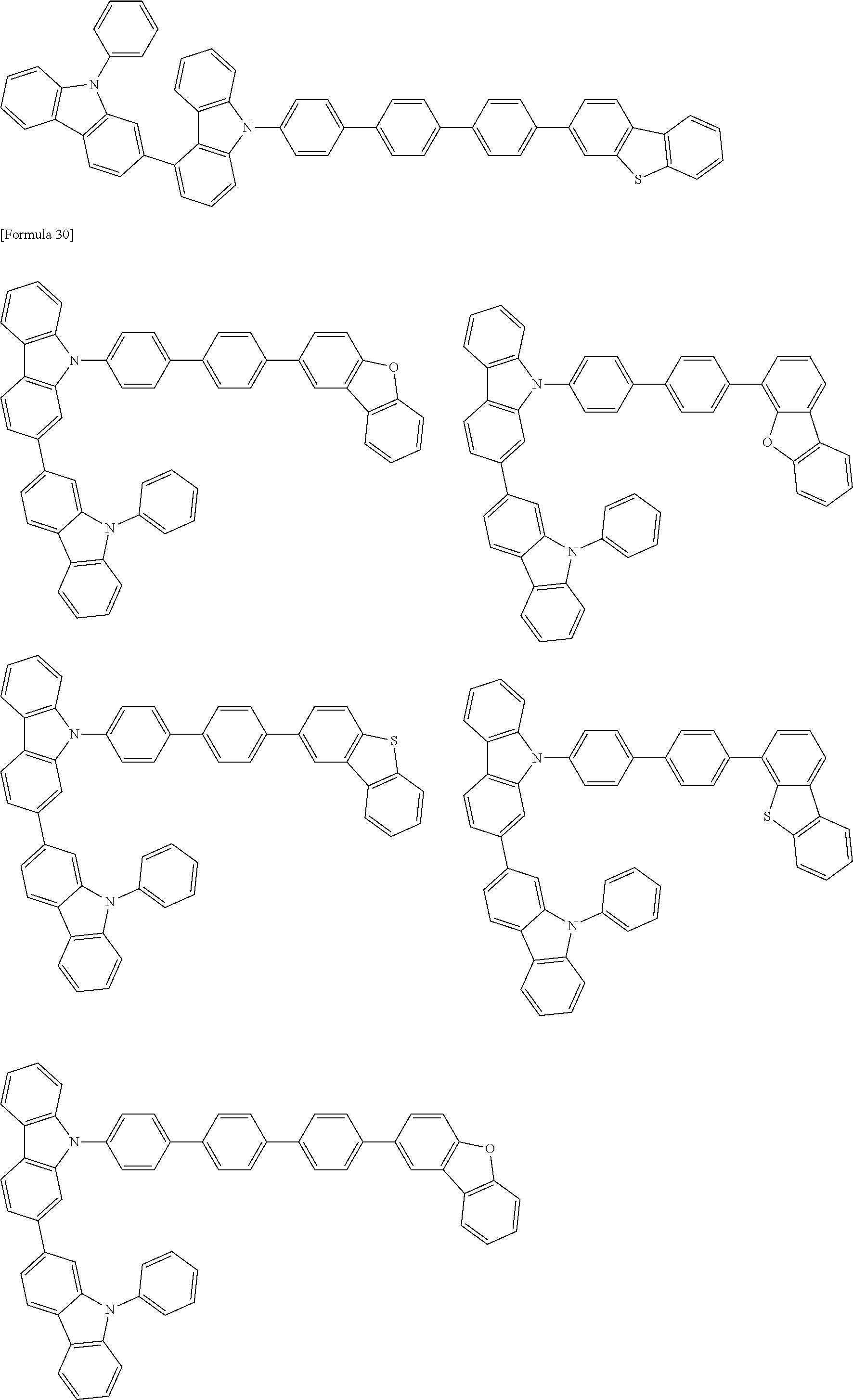

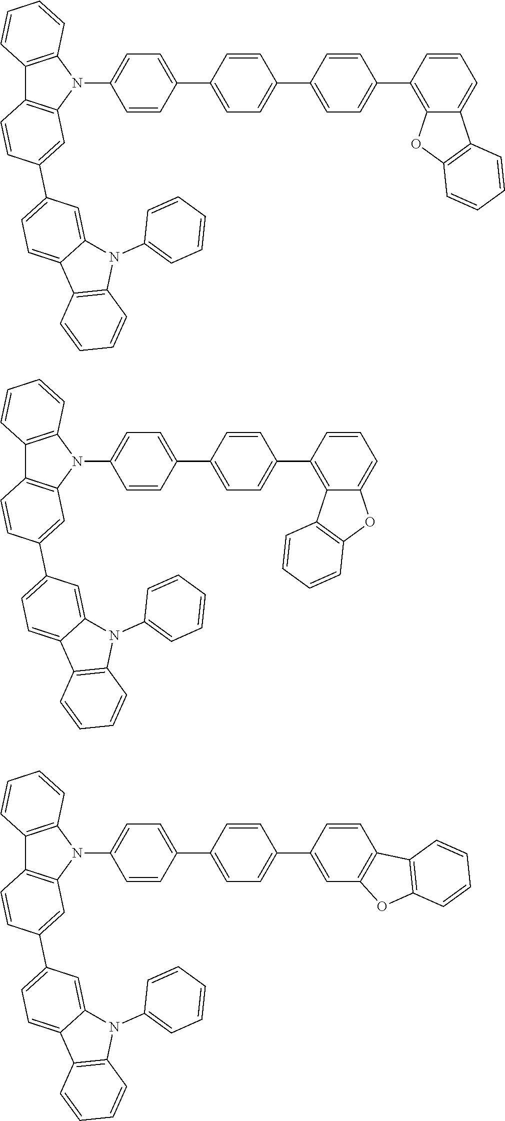

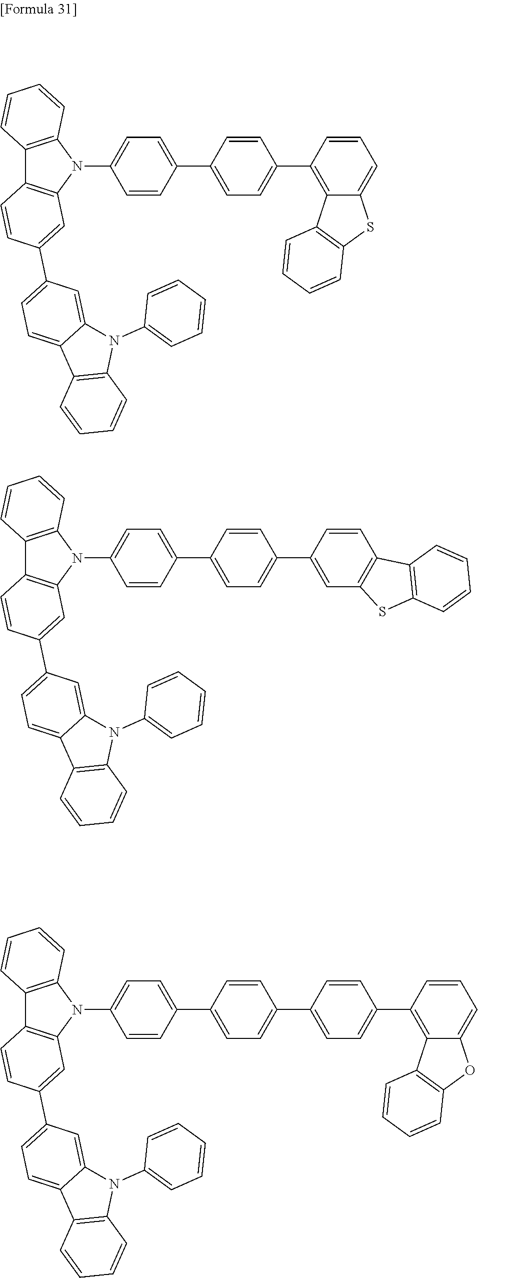

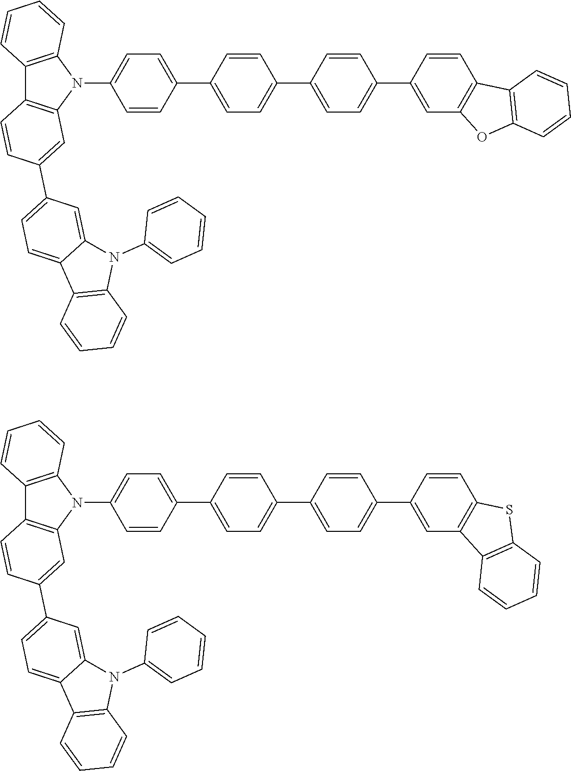 [SMILES: c1ccc(-n2c3ccccc3c3ccc(-c4ccc5c6ccccc6n(-c6ccc(-c7ccc(-c8ccc(-c9ccc%10c(c9)oc9ccccc9%10)cc8)cc7)cc6)c5c4)cc32)cc1 ; c1ccc(-n2c3ccccc3c3ccc(-c4ccc5c6ccccc6n(-c6ccc(-c7ccc(-c8ccc(-c9ccc%10sc%11ccccc%11c%10c9)cc8)cc7)cc6)c5c4)cc32)cc1]

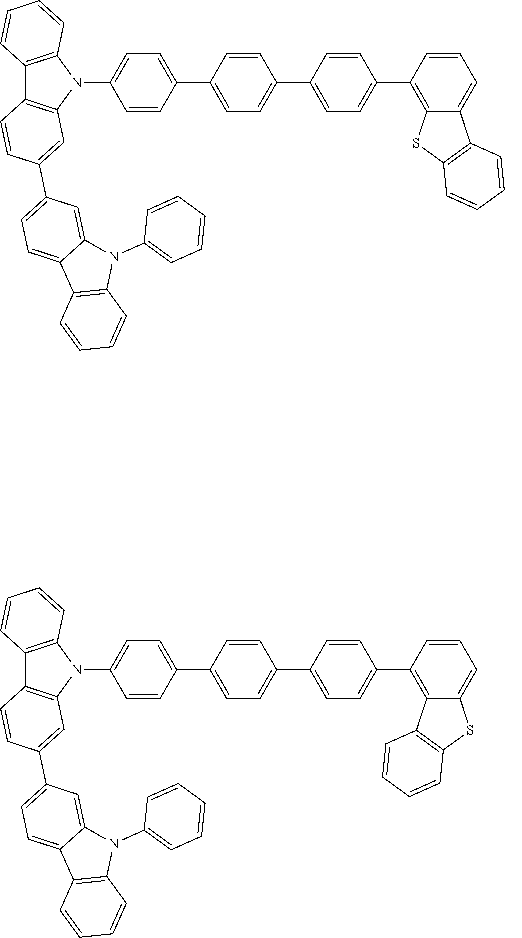

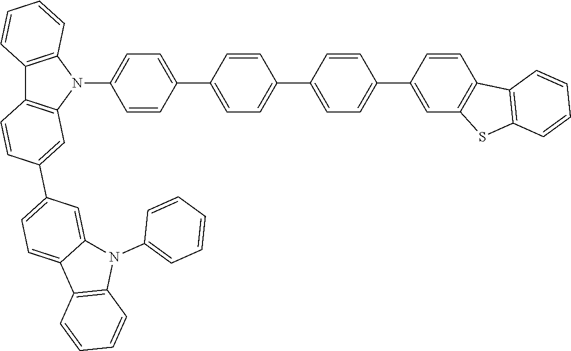 [SMILES: c1ccc(-n2c3ccccc3c3ccc(-c4ccc5c6ccccc6n(-c6ccc(-c7ccc(-c8ccc(-c9ccc%10c(c9)sc9ccccc9%10)cc8)cc7)cc6)c5c4)cc32)cc1]

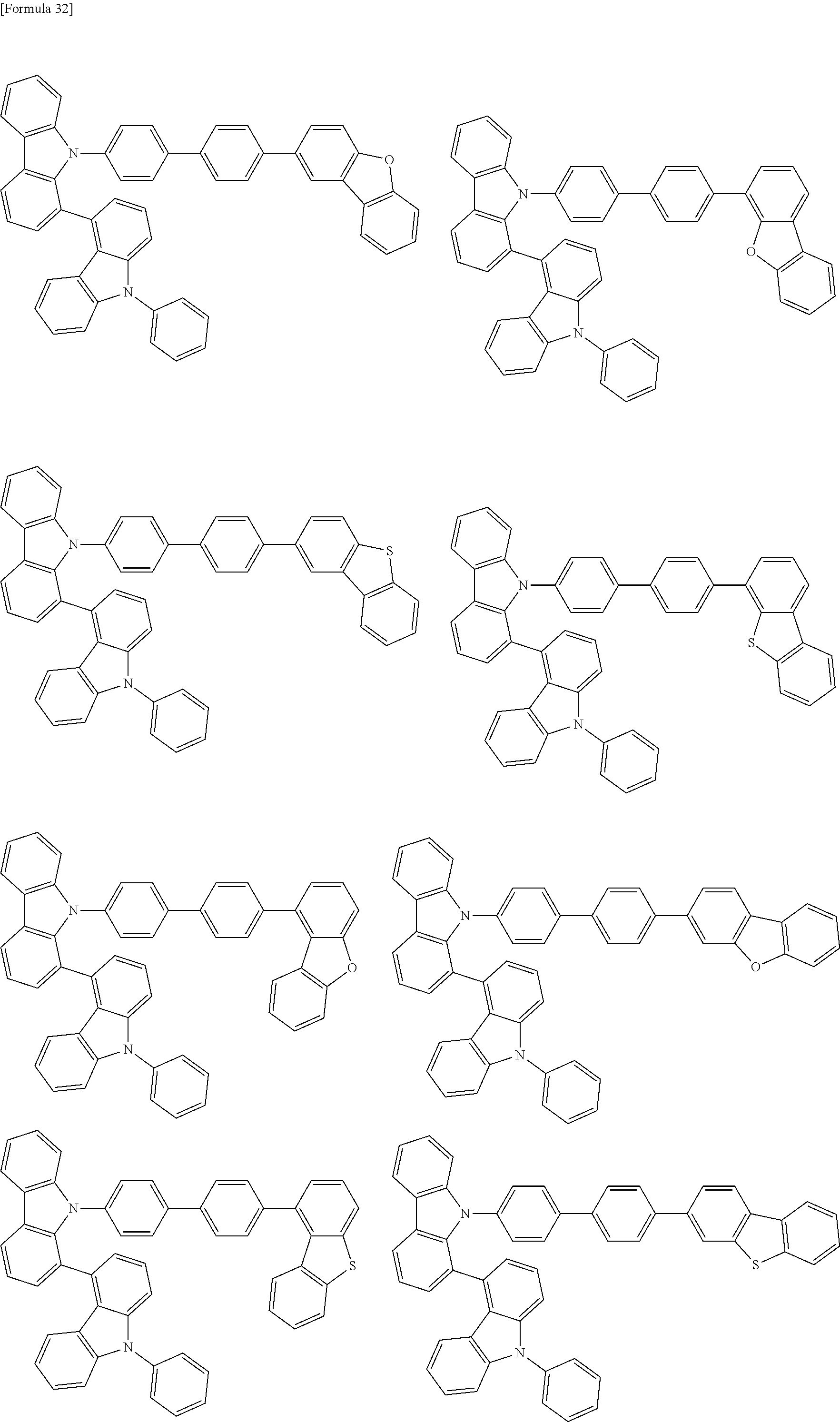

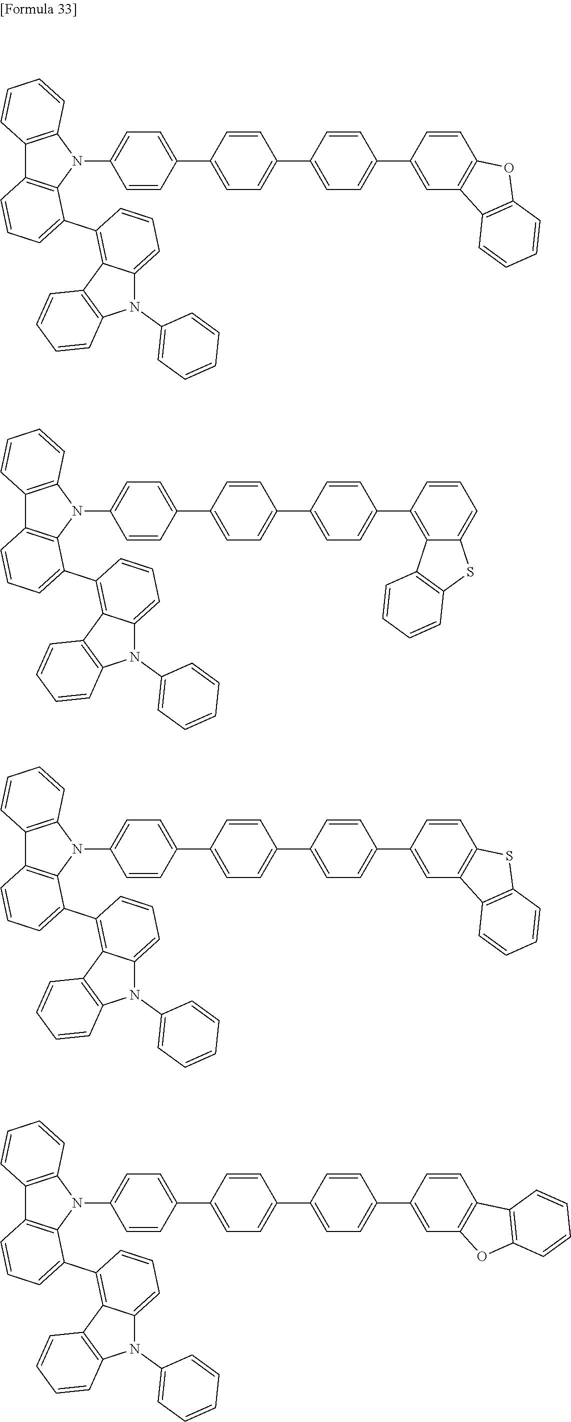 [SMILES: c1ccc(-n2c3ccccc3c3c(-c4cccc5c6ccccc6n(-c6ccc(-c7ccc(-c8ccc(-c9ccc%10oc%11ccccc%11c%10c9)cc8)cc7)cc6)c45)cccc32)cc1 ; c1ccc(-n2c3ccccc3c3c(-c4cccc5c6ccccc6n(-c6ccc(-c7ccc(-c8ccc(-c9cccc%10sc%11ccccc%11c9%10)cc8)cc7)cc6)c45)cccc32)cc1 ; c1ccc(-n2c3ccccc3c3c(-c4cccc5c6ccccc6n(-c6ccc(-c7ccc(-c8ccc(-c9ccc%10sc%11ccccc%11c%10c9)cc8)cc7)cc6)c45)cccc32)cc1 ; c1ccc(-n2c3ccccc3c3c(-c4cccc5c6ccccc6n(-c6ccc(-c7ccc(-c8ccc(-c9ccc%10c(c9)oc9ccccc9%10)cc8)cc7)cc6)c45)cccc32)cc1]

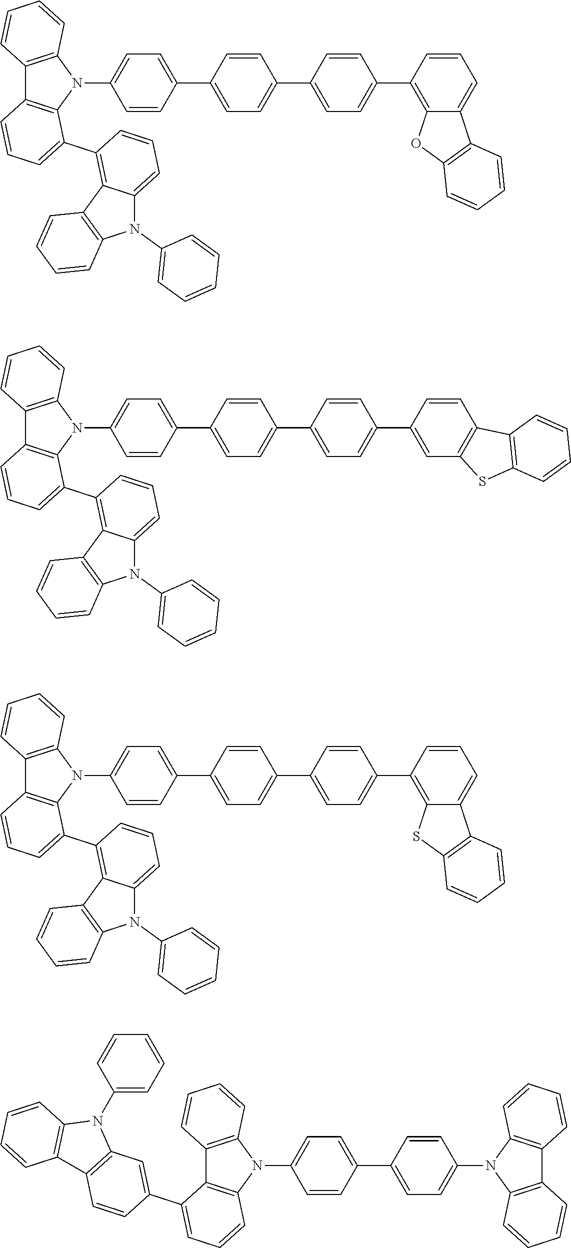 [SMILES: c1ccc(-n2c3ccccc3c3c(-c4cccc5c6ccccc6n(-c6ccc(-c7ccc(-c8ccc(-c9cccc%10c9oc9ccccc9%10)cc8)cc7)cc6)c45)cccc32)cc1 ; c1ccc(-n2c3ccccc3c3c(-c4cccc5c6ccccc6n(-c6ccc(-c7ccc(-c8ccc(-c9ccc%10c(c9)sc9ccccc9%10)cc8)cc7)cc6)c45)cccc32)cc1 ; c1ccc(-n2c3ccccc3c3c(-c4cccc5c6ccccc6n(-c6ccc(-c7ccc(-c8ccc(-c9cccc%10c9sc9ccccc9%10)cc8)cc7)cc6)c45)cccc32)cc1 ; c1ccc(-n2c3ccccc3c3ccc(-c4cccc5c4c4ccccc4n5-c4ccc(-c5ccc(-n6c7ccccc7c7ccccc76)cc5)cc4)cc32)cc1]

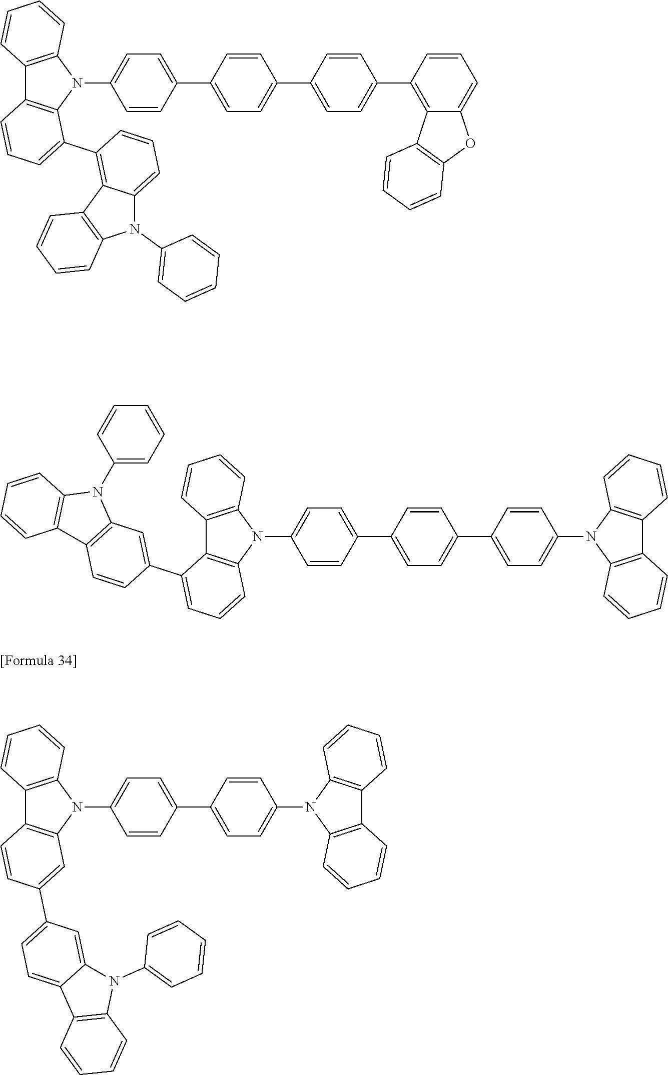 [SMILES: c1ccc(-n2c3ccccc3c3c(-c4cccc5c6ccccc6n(-c6ccc(-c7ccc(-c8ccc(-c9cccc%10oc%11ccccc%11c9%10)cc8)cc7)cc6)c45)cccc32)cc1 ; c1ccc(-n2c3ccccc3c3ccc(-c4cccc5c4c4ccccc4n5-c4ccc(-c5ccc(-c6ccc(-n7c8ccccc8c8ccccc87)cc6)cc5)cc4)cc32)cc1 ; c1ccc(-n2c3ccccc3c3ccc(-c4ccc5c6ccccc6n(-c6ccc(-c7ccc(-n8c9ccccc9c9ccccc98)cc7)cc6)c5c4)cc32)cc1]

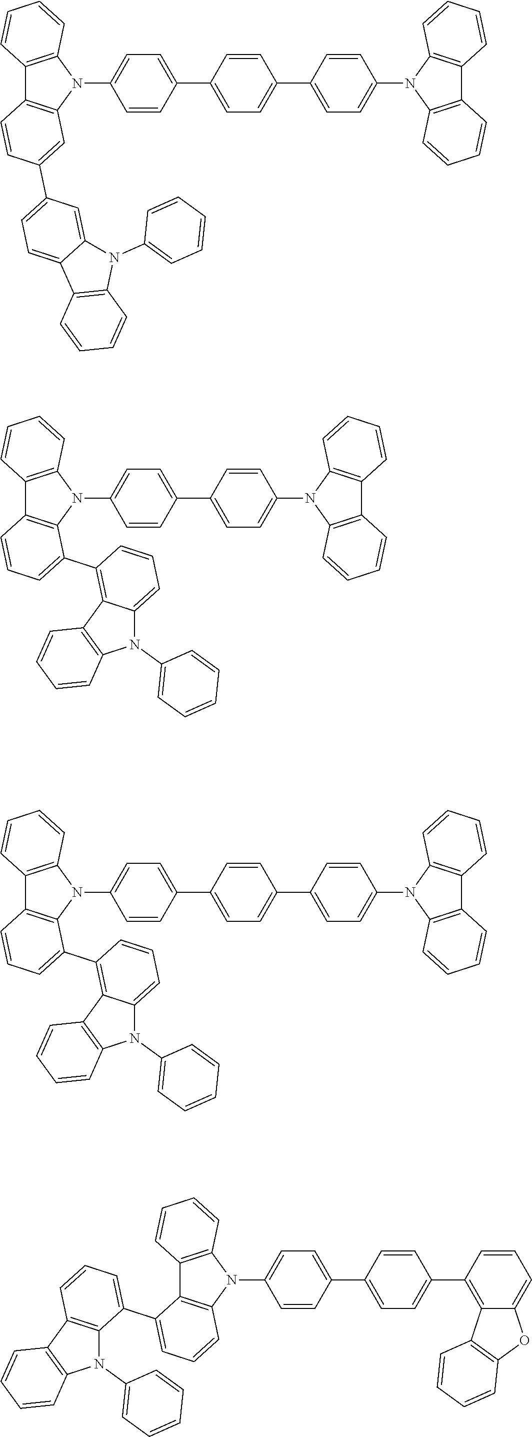 [SMILES: c1ccc(-n2c3ccccc3c3ccc(-c4ccc5c6ccccc6n(-c6ccc(-c7ccc(-c8ccc(-n9c%10ccccc%10c%10ccccc%109)cc8)cc7)cc6)c5c4)cc32)cc1 ; c1ccc(-n2c3ccccc3c3c(-c4cccc5c6ccccc6n(-c6ccc(-c7ccc(-n8c9ccccc9c9ccccc98)cc7)cc6)c45)cccc32)cc1 ; c1ccc(-n2c3ccccc3c3c(-c4cccc5c6ccccc6n(-c6ccc(-c7ccc(-c8ccc(-n9c%10ccccc%10c%10ccccc%109)cc8)cc7)cc6)c45)cccc32)cc1 ; c1ccc(-n2c3ccccc3c3cccc(-c4cccc5c4c4ccccc4n5-c4ccc(-c5ccc(-c6cccc7oc8ccccc8c67)cc5)cc4)c32)cc1]

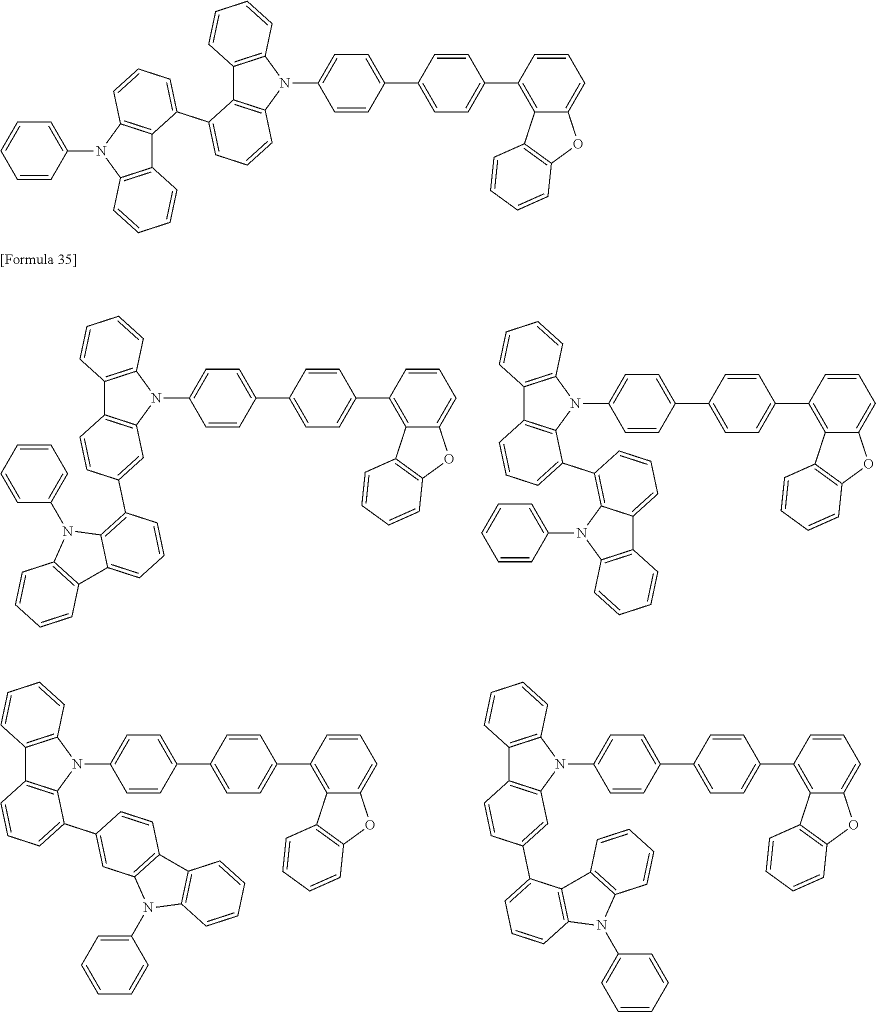

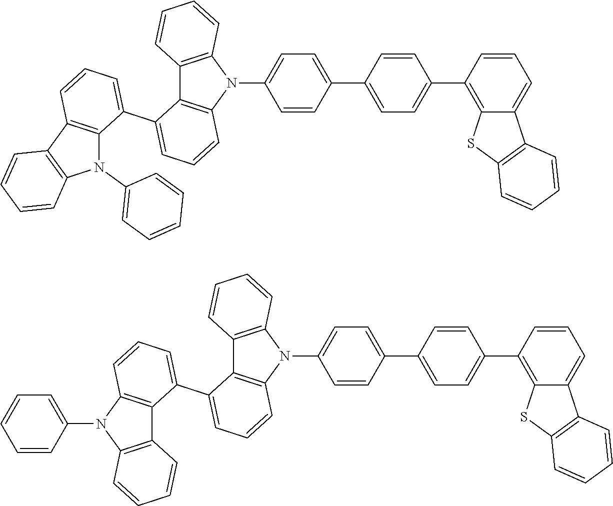 [SMILES: c1ccc(-n2c3ccccc3c3cccc(-c4cccc5c4c4ccccc4n5-c4ccc(-c5ccc(-c6cccc7c6sc6ccccc67)cc5)cc4)c32)cc1 ; c1ccc(-n2c3ccccc3c3c(-c4cccc5c4c4ccccc4n5-c4ccc(-c5ccc(-c6cccc7c6sc6ccccc67)cc5)cc4)cccc32)cc1]

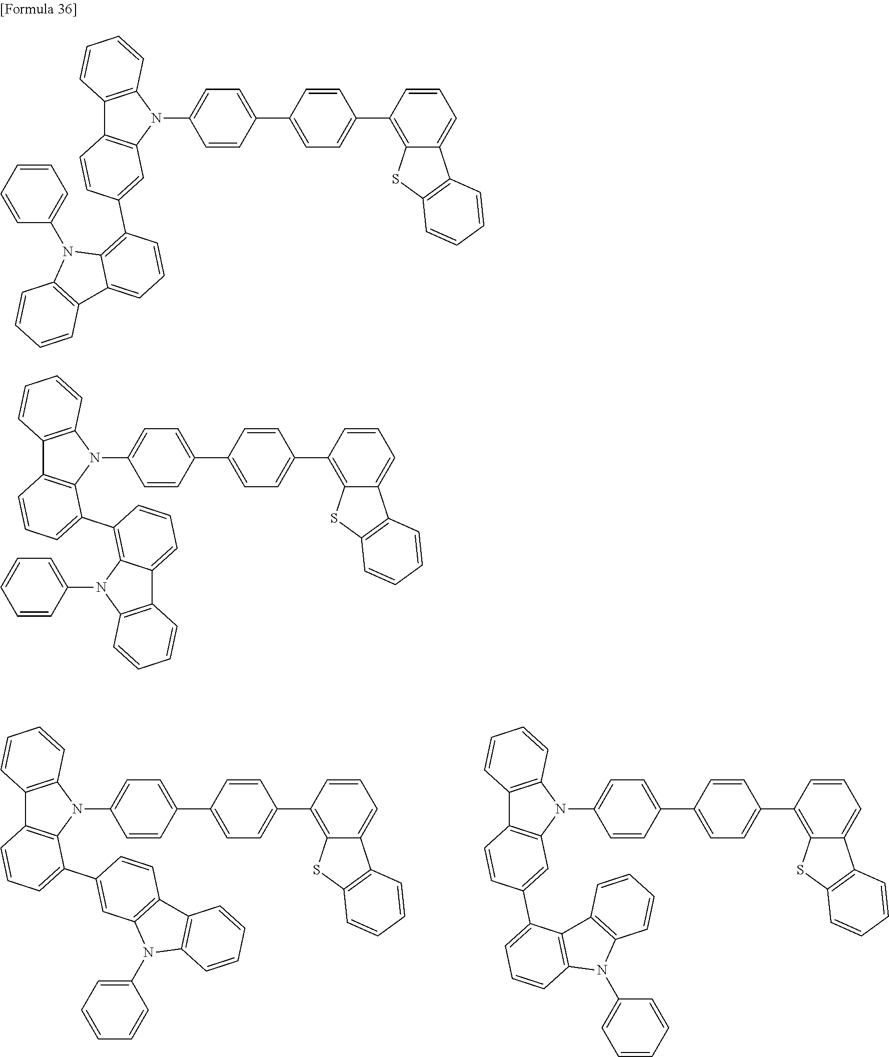

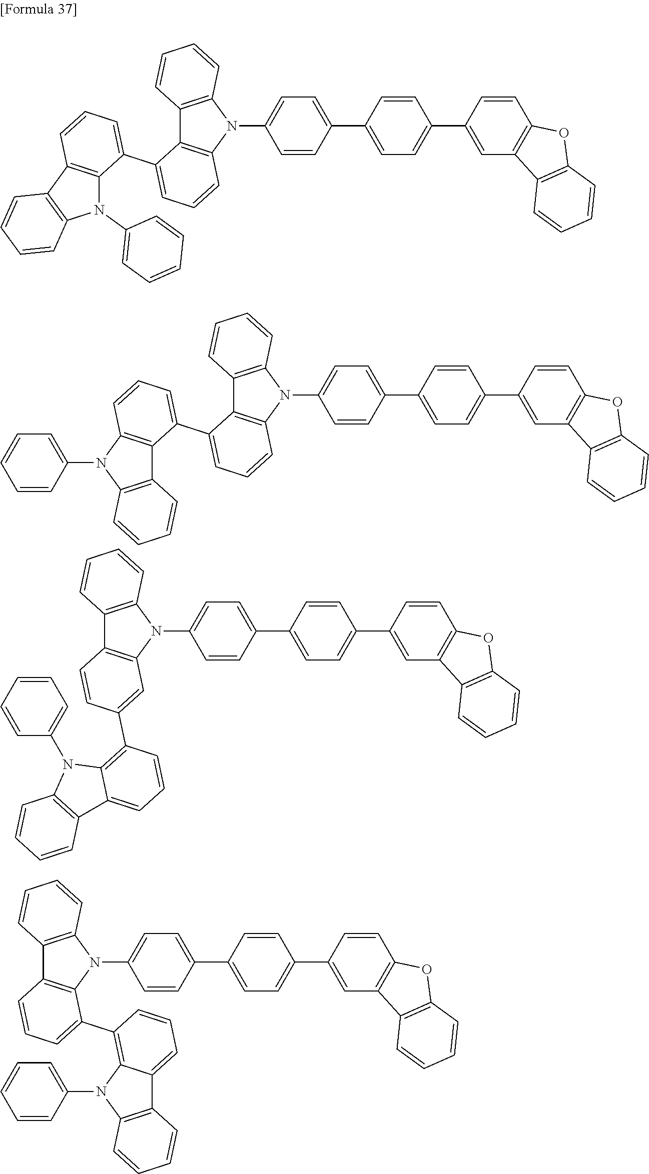

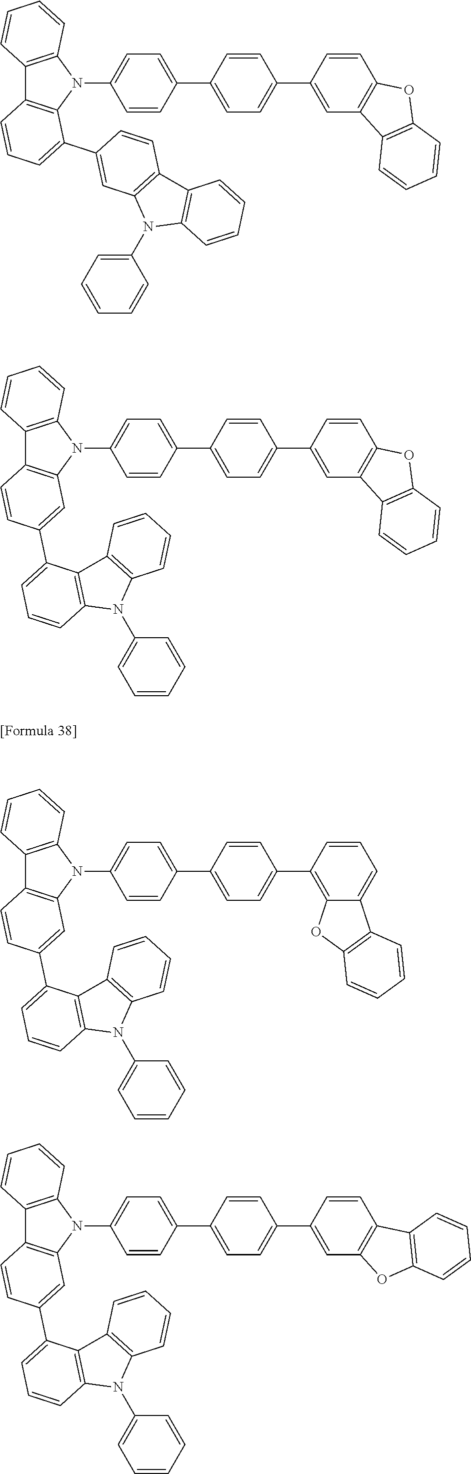 [SMILES: c1ccc(-n2c3ccccc3c3ccc(-c4cccc5c6ccccc6n(-c6ccc(-c7ccc(-c8ccc9oc%10ccccc%10c9c8)cc7)cc6)c45)cc32)cc1 ; c1ccc(-n2c3ccccc3c3c(-c4ccc5c6ccccc6n(-c6ccc(-c7ccc(-c8ccc9oc%10ccccc%10c9c8)cc7)cc6)c5c4)cccc32)cc1 ; c1ccc(-n2c3ccccc3c3c(-c4ccc5c6ccccc6n(-c6ccc(-c7ccc(-c8cccc9c8oc8ccccc89)cc7)cc6)c5c4)cccc32)cc1 ; c1ccc(-n2c3ccccc3c3c(-c4ccc5c6ccccc6n(-c6ccc(-c7ccc(-c8ccc9c(c8)oc8ccccc89)cc7)cc6)c5c4)cccc32)cc1]

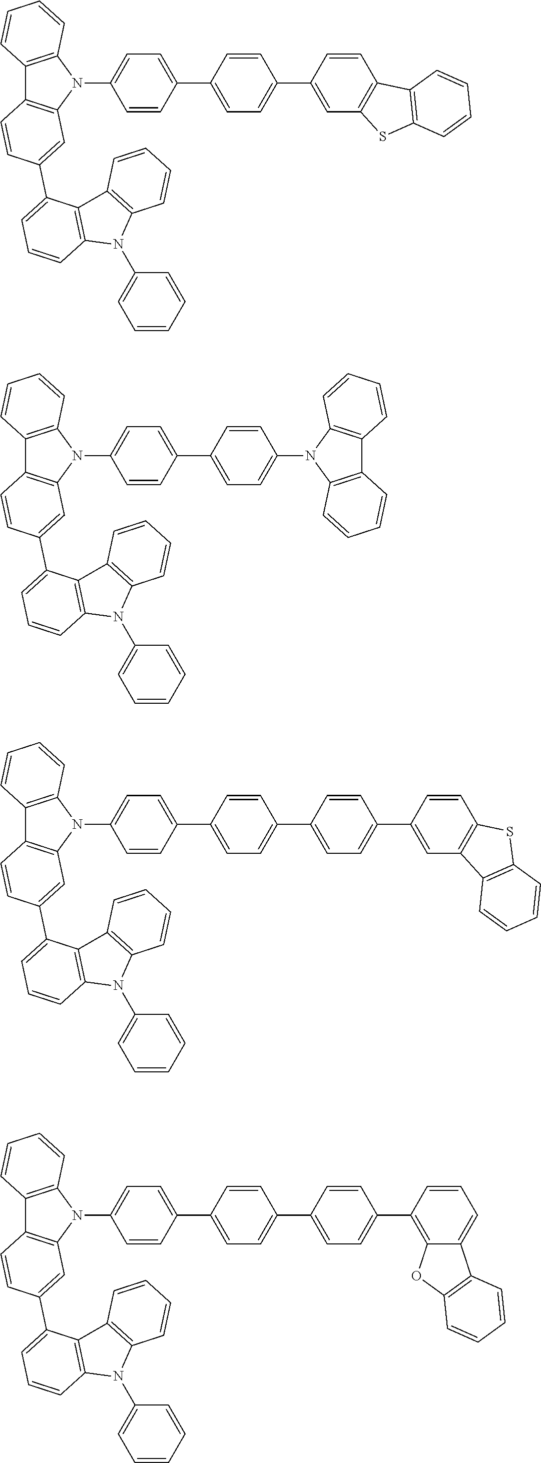 [SMILES: c1ccc(-n2c3ccccc3c3c(-c4ccc5c6ccccc6n(-c6ccc(-c7ccc(-c8ccc9c(c8)sc8ccccc89)cc7)cc6)c5c4)cccc32)cc1 ; c1ccc(-n2c3ccccc3c3c(-c4ccc5c6ccccc6n(-c6ccc(-c7ccc(-n8c9ccccc9c9ccccc98)cc7)cc6)c5c4)cccc32)cc1 ; c1ccc(-n2c3ccccc3c3c(-c4ccc5c6ccccc6n(-c6ccc(-c7ccc(-c8ccc(-c9ccc%10sc%11ccccc%11c%10c9)cc8)cc7)cc6)c5c4)cccc32)cc1 ; c1ccc(-n2c3ccccc3c3c(-c4ccc5c6ccccc6n(-c6ccc(-c7ccc(-c8ccc(-c9cccc%10c9oc9ccccc9%10)cc8)cc7)cc6)c5c4)cccc32)cc1]

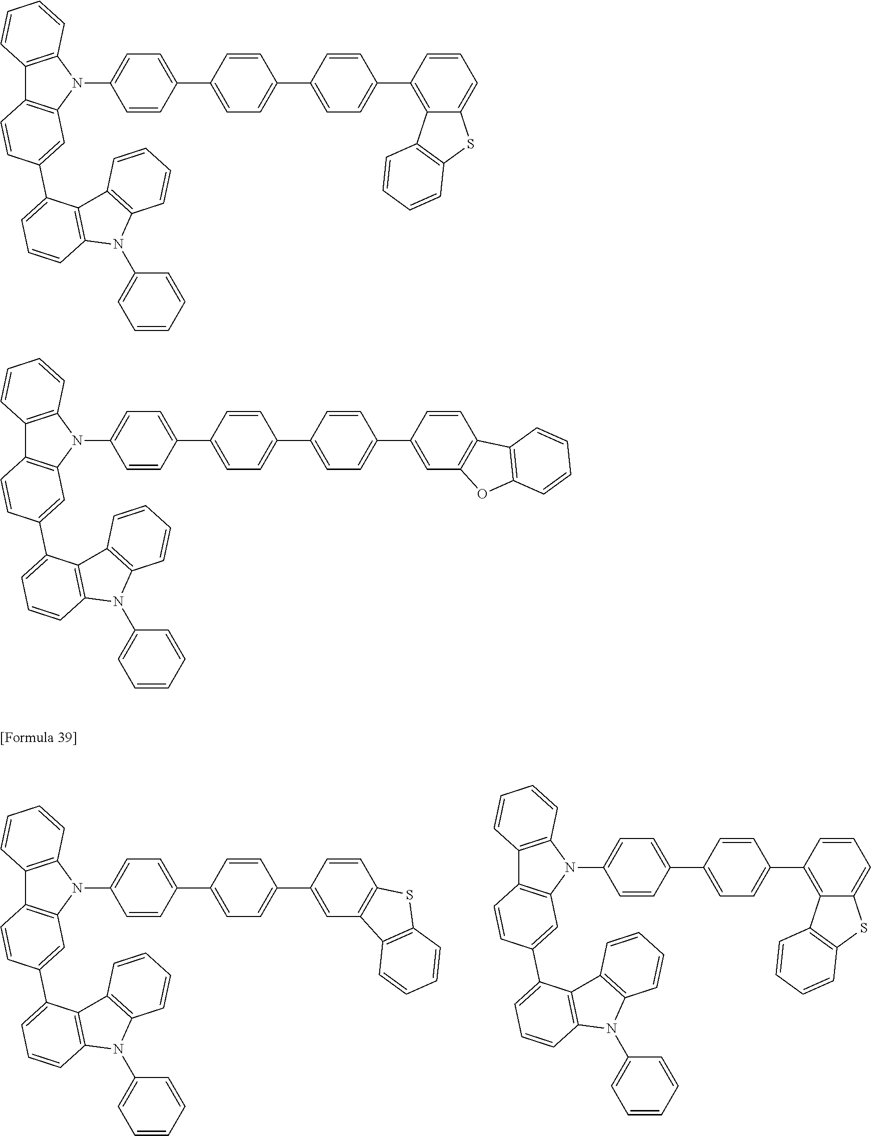 [SMILES: c1ccc(-n2c3ccccc3c3c(-c4ccc5c6ccccc6n(-c6ccc(-c7ccc(-c8ccc(-c9cccc%10sc%11ccccc%11c9%10)cc8)cc7)cc6)c5c4)cccc32)cc1 ; c1ccc(-n2c3ccccc3c3c(-c4ccc5c6ccccc6n(-c6ccc(-c7ccc(-c8ccc(-c9ccc%10c(c9)oc9ccccc9%10)cc8)cc7)cc6)c5c4)cccc32)cc1 ; c1ccc(-n2c3ccccc3c3c(-c4ccc5c6ccccc6n(-c6ccc(-c7ccc(-c8ccc9sc%10ccccc%10c9c8)cc7)cc6)c5c4)cccc32)cc1 ; c1ccc(-n2c3ccccc3c3c(-c4ccc5c6ccccc6n(-c6ccc(-c7ccc(-c8cccc9sc%10ccccc%10c89)cc7)cc6)c5c4)cccc32)cc1]

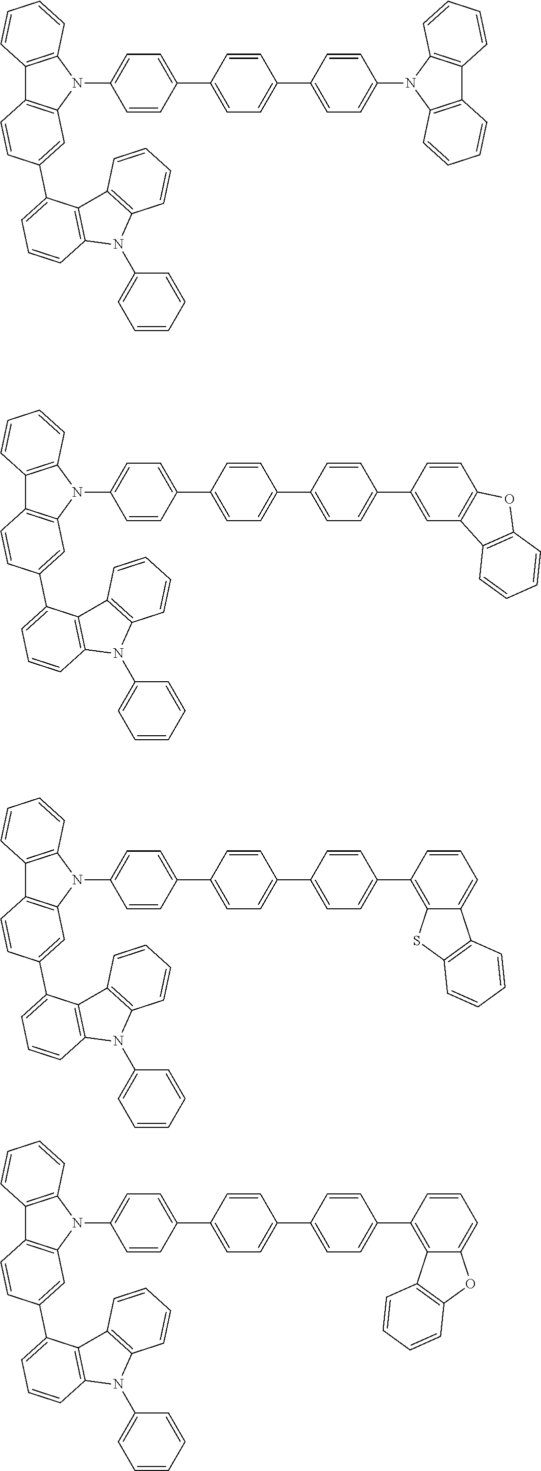 [SMILES: c1ccc(-n2c3ccccc3c3c(-c4ccc5c6ccccc6n(-c6ccc(-c7ccc(-c8ccc(-n9c%10ccccc%10c%10ccccc%109)cc8)cc7)cc6)c5c4)cccc32)cc1 ; c1ccc(-n2c3ccccc3c3c(-c4ccc5c6ccccc6n(-c6ccc(-c7ccc(-c8ccc(-c9ccc%10oc%11ccccc%11c%10c9)cc8)cc7)cc6)c5c4)cccc32)cc1 ; c1ccc(-n2c3ccccc3c3c(-c4ccc5c6ccccc6n(-c6ccc(-c7ccc(-c8ccc(-c9cccc%10c9sc9ccccc9%10)cc8)cc7)cc6)c5c4)cccc32)cc1 ; c1ccc(-n2c3ccccc3c3c(-c4ccc5c6ccccc6n(-c6ccc(-c7ccc(-c8ccc(-c9cccc%10oc%11ccccc%11c9%10)cc8)cc7)cc6)c5c4)cccc32)cc1]

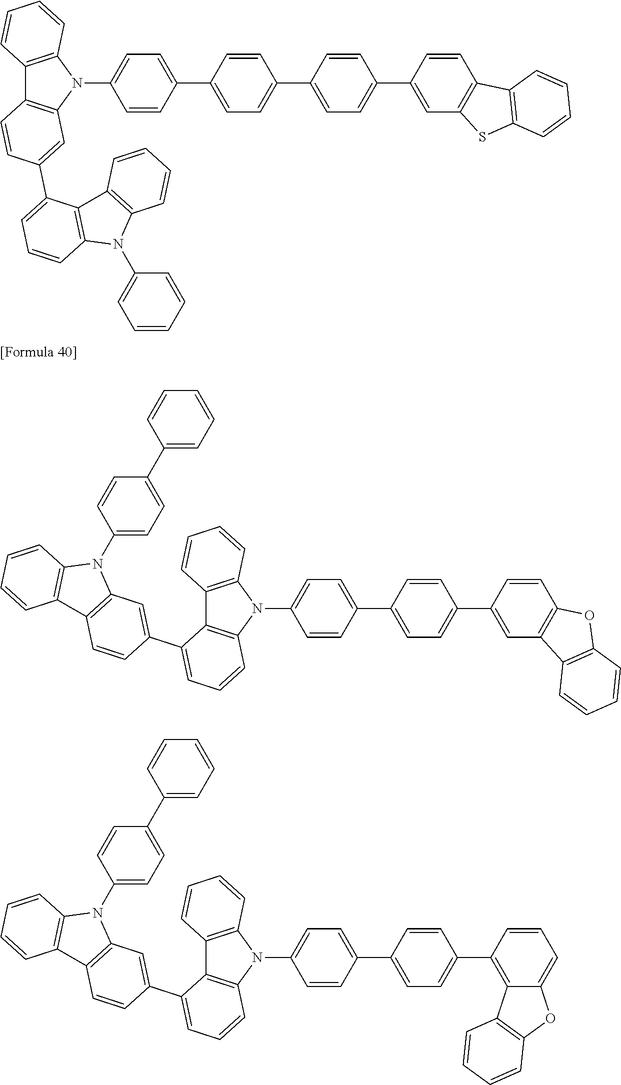 [SMILES: c1ccc(-n2c3ccccc3c3c(-c4ccc5c6ccccc6n(-c6ccc(-c7ccc(-c8ccc(-c9ccc%10c(c9)sc9ccccc9%10)cc8)cc7)cc6)c5c4)cccc32)cc1 ; c1ccc(-c2ccc(-n3c4ccccc4c4ccc(-c5cccc6c5c5ccccc5n6-c5ccc(-c6ccc(-c7ccc8oc9ccccc9c8c7)cc6)cc5)cc43)cc2)cc1 ; c1ccc(-c2ccc(-n3c4ccccc4c4ccc(-c5cccc6c5c5ccccc5n6-c5ccc(-c6ccc(-c7cccc8oc9ccccc9c78)cc6)cc5)cc43)cc2)cc1]

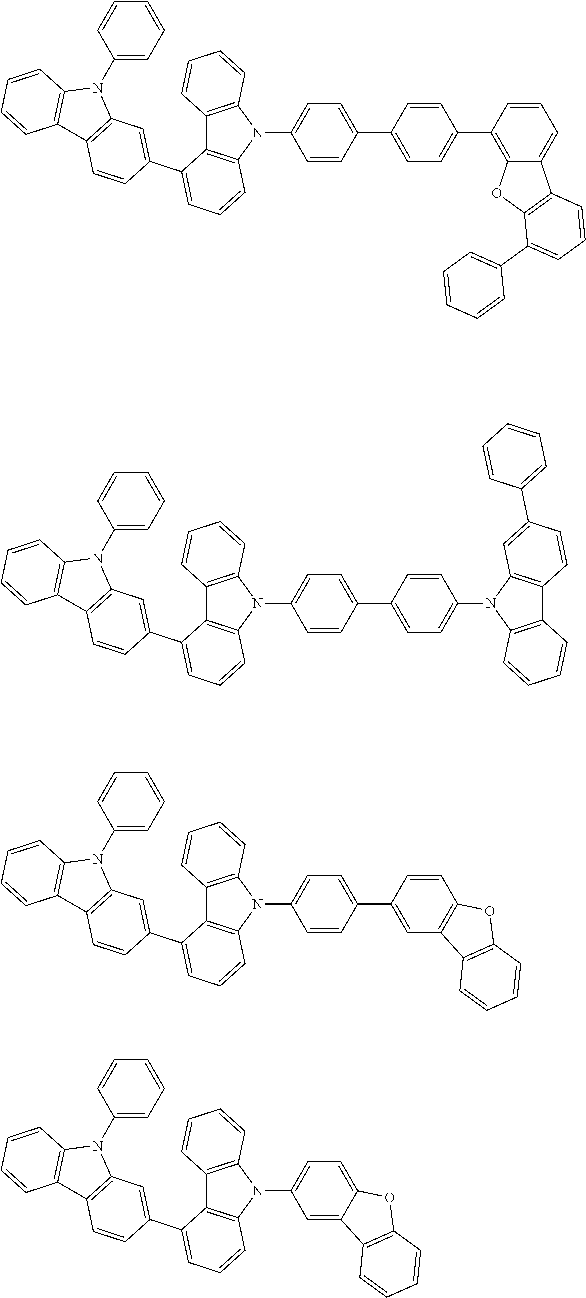 [SMILES: c1ccc(-c2cccc3c2oc2c(-c4ccc(-c5ccc(-n6c7ccccc7c7c(-c8ccc9c%10ccccc%10n(-c%10ccccc%10)c9c8)cccc76)cc5)cc4)cccc23)cc1 ; c1ccc(-c2ccc3c4ccccc4n(-c4ccc(-c5ccc(-n6c7ccccc7c7c(-c8ccc9c%10ccccc%10n(-c%10ccccc%10)c9c8)cccc76)cc5)cc4)c3c2)cc1 ; c1ccc(-n2c3ccccc3c3ccc(-c4cccc5c4c4ccccc4n5-c4ccc(-c5ccc6oc7ccccc7c6c5)cc4)cc32)cc1 ; c1ccc(-n2c3ccccc3c3ccc(-c4cccc5c4c4ccccc4n5-c4ccc5oc6ccccc6c5c4)cc32)cc1]

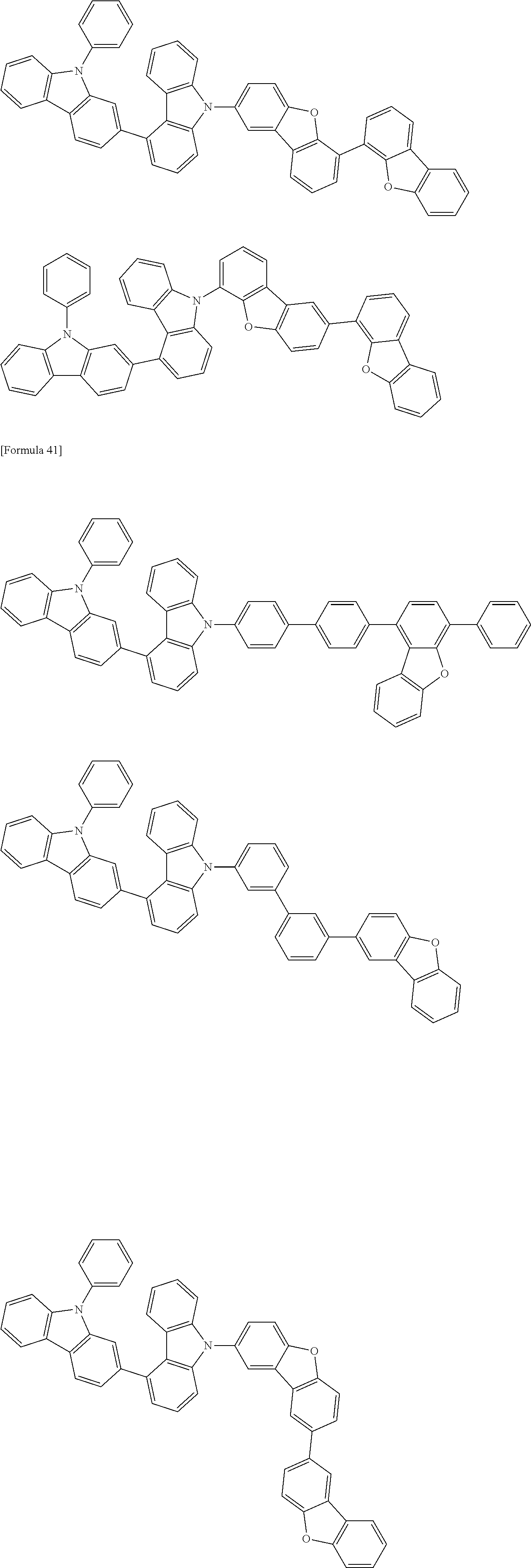 [SMILES: c1ccc(-n2c3ccccc3c3ccc(-c4cccc5c4c4ccccc4n5-c4ccc5oc6c(-c7cccc8c7oc7ccccc78)cccc6c5c4)cc32)cc1 ; c1ccc(-n2c3ccccc3c3ccc(-c4cccc5c4c4ccccc4n5-c4cccc5c4oc4ccc(-c6cccc7c6oc6ccccc67)cc45)cc32)cc1 ; c1ccc(-c2ccc(-c3ccc(-c4ccc(-n5c6ccccc6c6c(-c7ccc8c9ccccc9n(-c9ccccc9)c8c7)cccc65)cc4)cc3)c3c2oc2ccccc23)cc1 ; c1ccc(-n2c3ccccc3c3ccc(-c4cccc5c4c4ccccc4n5-c4cccc(-c5cccc(-c6ccc7oc8ccccc8c7c6)c5)c4)cc32)cc1 ; c1ccc(-n2c3ccccc3c3ccc(-c4cccc5c4c4ccccc4n5-c4ccc5oc6ccc(-c7ccc8oc9ccccc9c8c7)cc6c5c4)cc32)cc1]

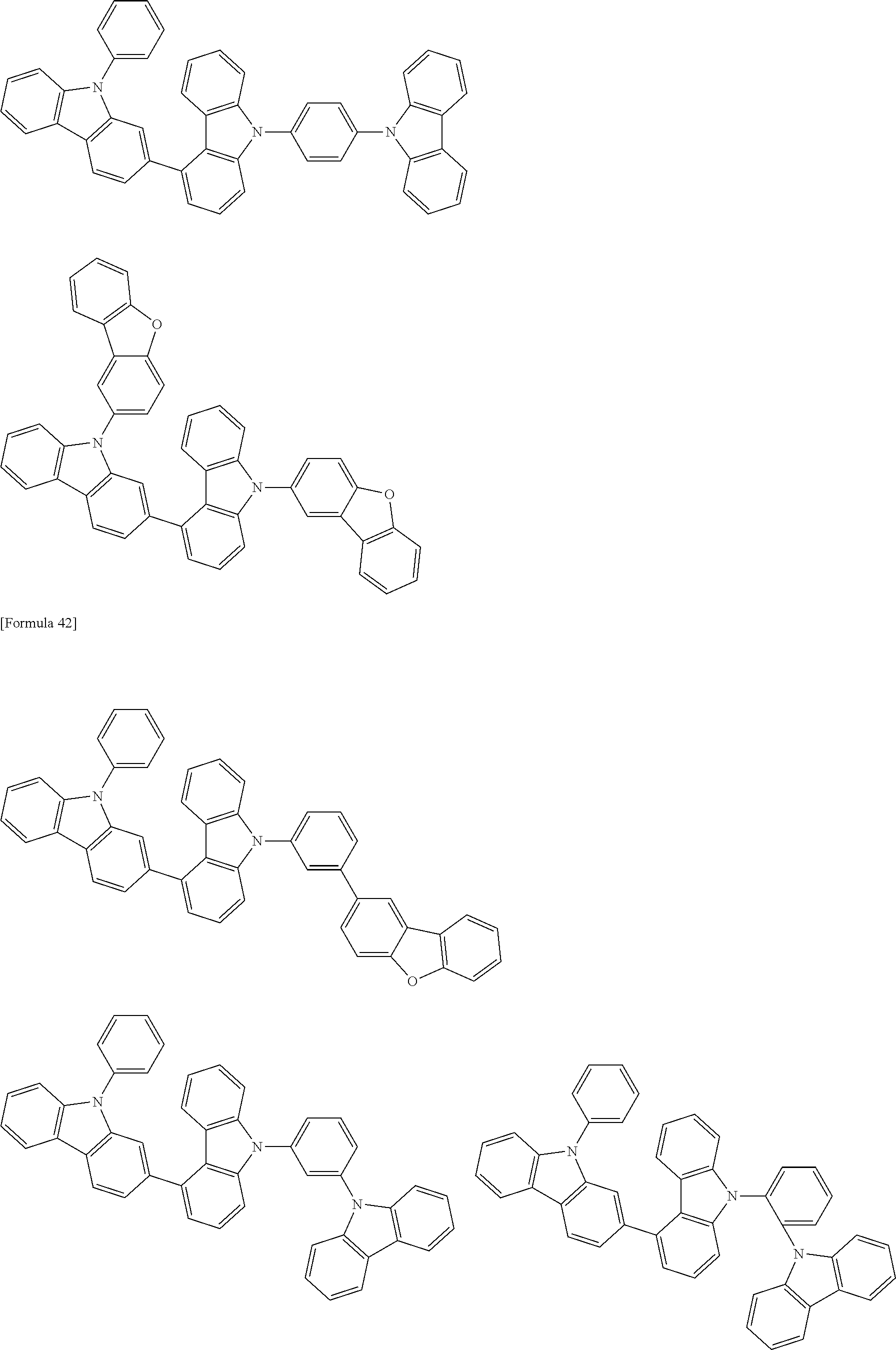

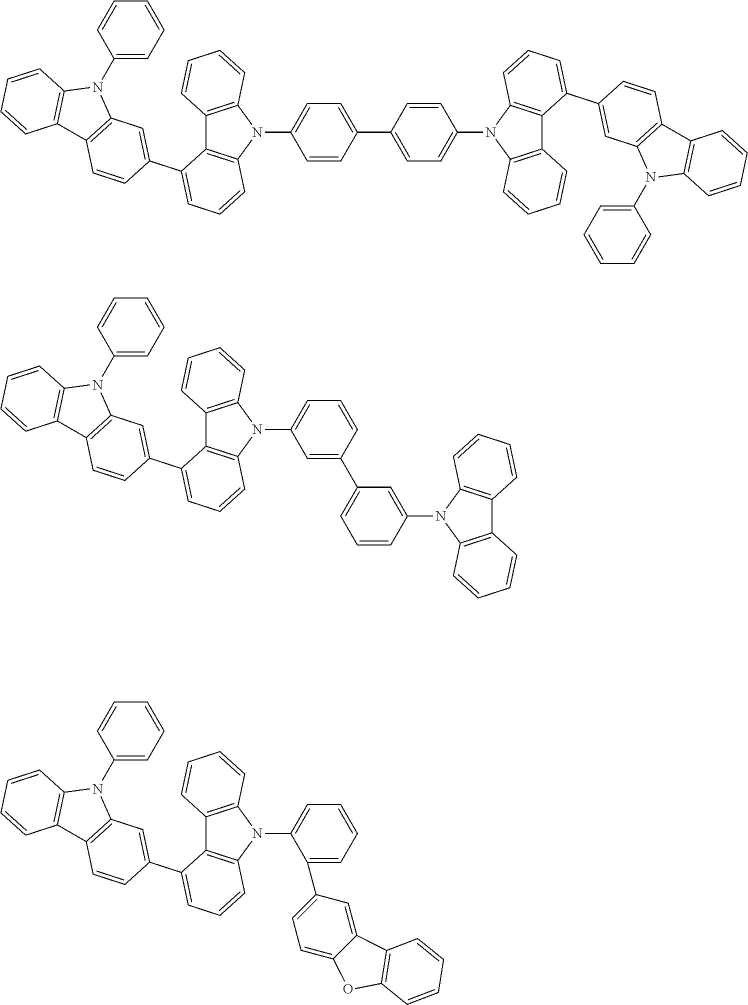 [SMILES: c1ccc(-n2c3ccccc3c3ccc(-c4cccc5c4c4ccccc4n5-c4ccc(-c5ccc(-n6c7ccccc7c7c(-c8ccc9c%10ccccc%10n(-c%10ccccc%10)c9c8)cccc76)cc5)cc4)cc32)cc1 ; c1ccc(-n2c3ccccc3c3ccc(-c4cccc5c4c4ccccc4n5-c4cccc(-c5cccc(-n6c7ccccc7c7ccccc76)c5)c4)cc32)cc1 ; c1ccc(-n2c3ccccc3c3ccc(-c4cccc5c4c4ccccc4n5-c4ccccc4-c4ccc5oc6ccccc6c5c4)cc32)cc1]

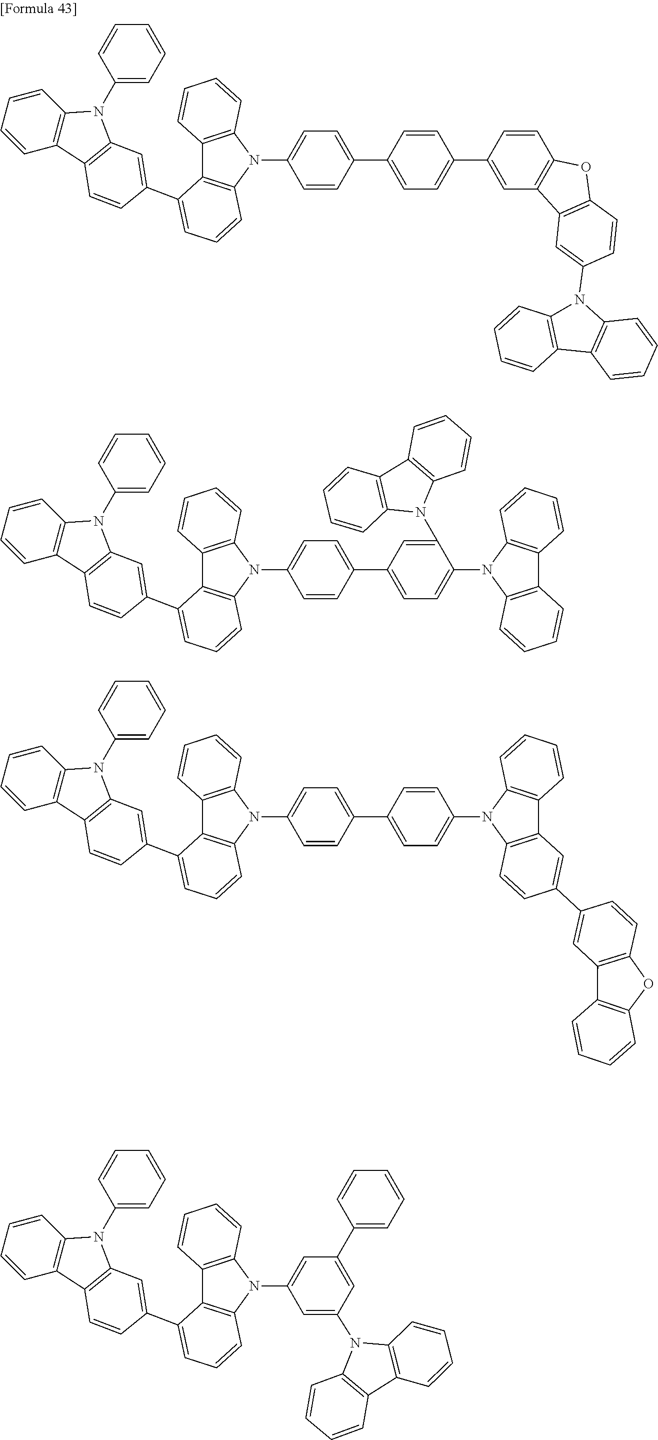 [SMILES: c1ccc(-n2c3ccccc3c3ccc(-c4cccc5c4c4ccccc4n5-c4ccc(-c5ccc(-c6ccc7oc8ccc(-n9c%10ccccc%10c%10ccccc%109)cc8c7c6)cc5)cc4)cc32)cc1 ; c1ccc(-n2c3ccccc3c3ccc(-c4cccc5c4c4ccccc4n5-c4ccc(-c5ccc(-n6c7ccccc7c7ccccc76)c(-n6c7ccccc7c7ccccc76)c5)cc4)cc32)cc1 ; c1ccc(-n2c3ccccc3c3ccc(-c4cccc5c4c4ccccc4n5-c4ccc(-c5ccc(-n6c7ccccc7c7cc(-c8ccc9oc%10ccccc%10c9c8)ccc76)cc5)cc4)cc32)cc1 ; c1ccc(-c2cc(-n3c4ccccc4c4ccccc43)cc(-n3c4ccccc4c4c(-c5ccc6c7ccccc7n(-c7ccccc7)c6c5)cccc43)c2)cc1]

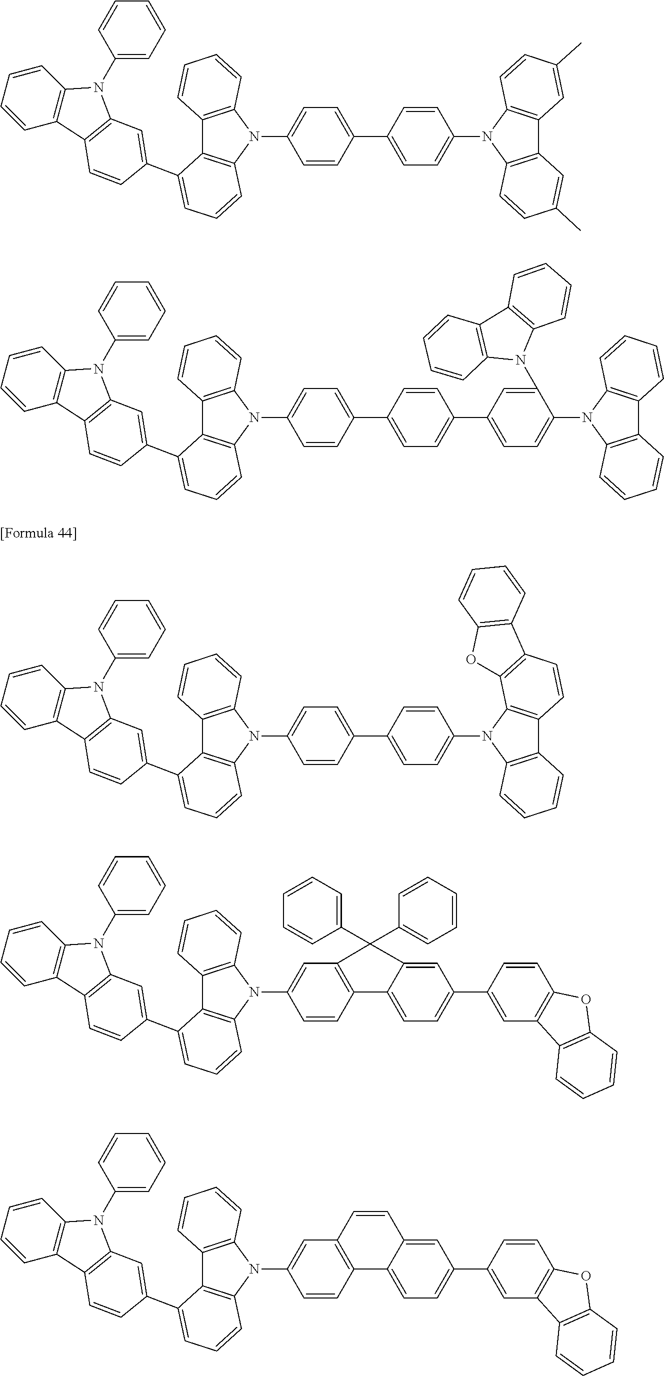

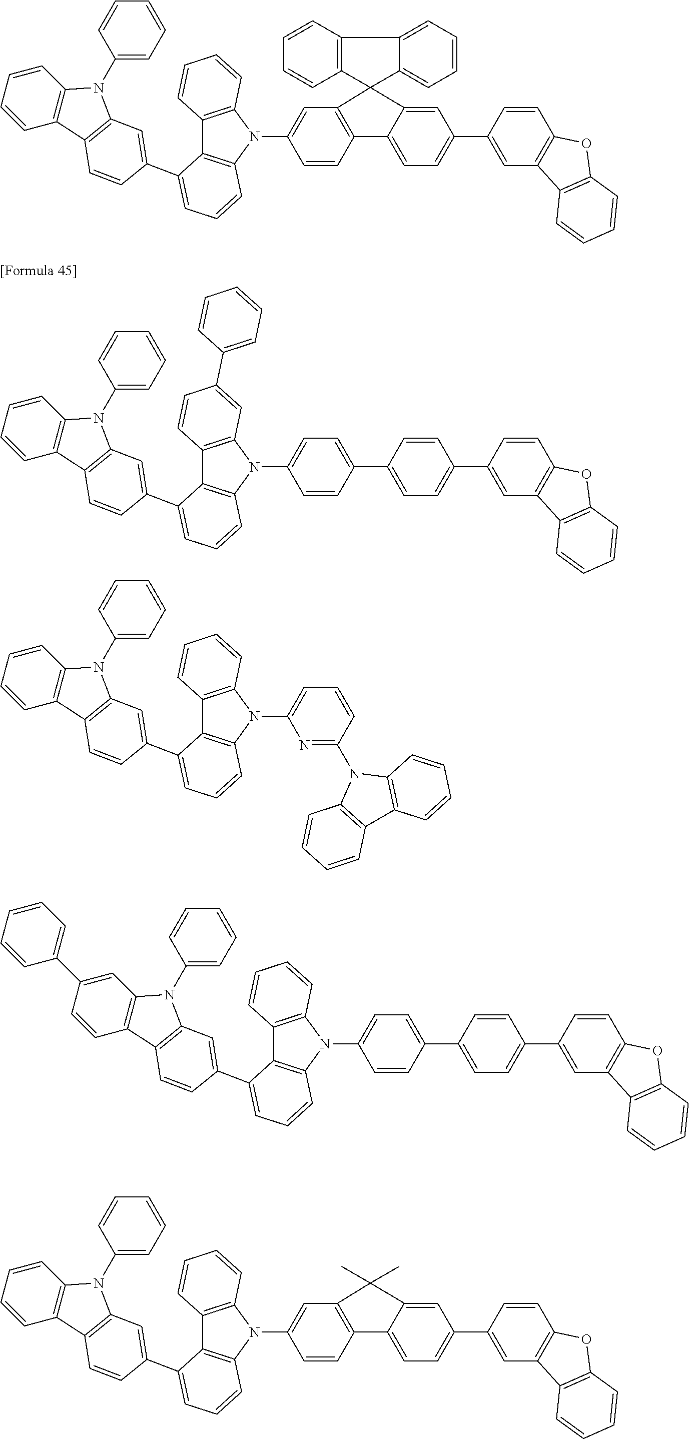

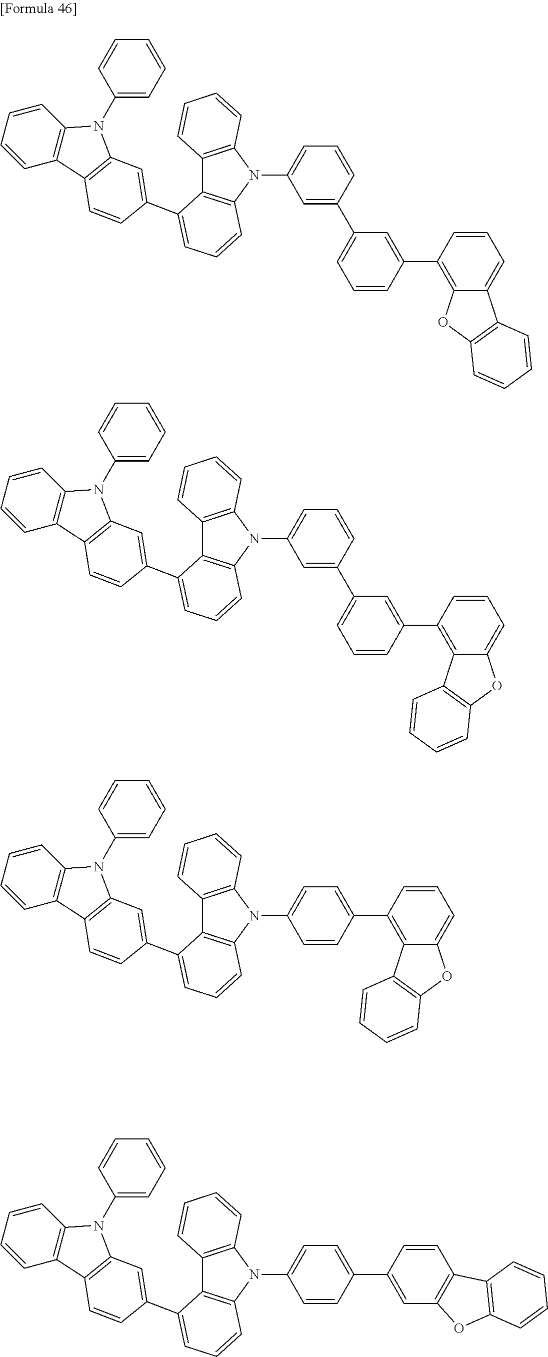 [SMILES: c1ccc(-n2c3ccccc3c3ccc(-c4cccc5c4c4ccccc4n5-c4cccc(-c5cccc(-c6cccc7c6oc6ccccc67)c5)c4)cc32)cc1 ; c1ccc(-n2c3ccccc3c3ccc(-c4cccc5c4c4ccccc4n5-c4cccc(-c5cccc(-c6cccc7oc8ccccc8c67)c5)c4)cc32)cc1 ; c1ccc(-n2c3ccccc3c3ccc(-c4cccc5c4c4ccccc4n5-c4ccc(-c5cccc6oc7ccccc7c56)cc4)cc32)cc1 ; c1ccc(-n2c3ccccc3c3ccc(-c4cccc5c4c4ccccc4n5-c4ccc(-c5ccc6c(c5)oc5ccccc56)cc4)cc32)cc1]

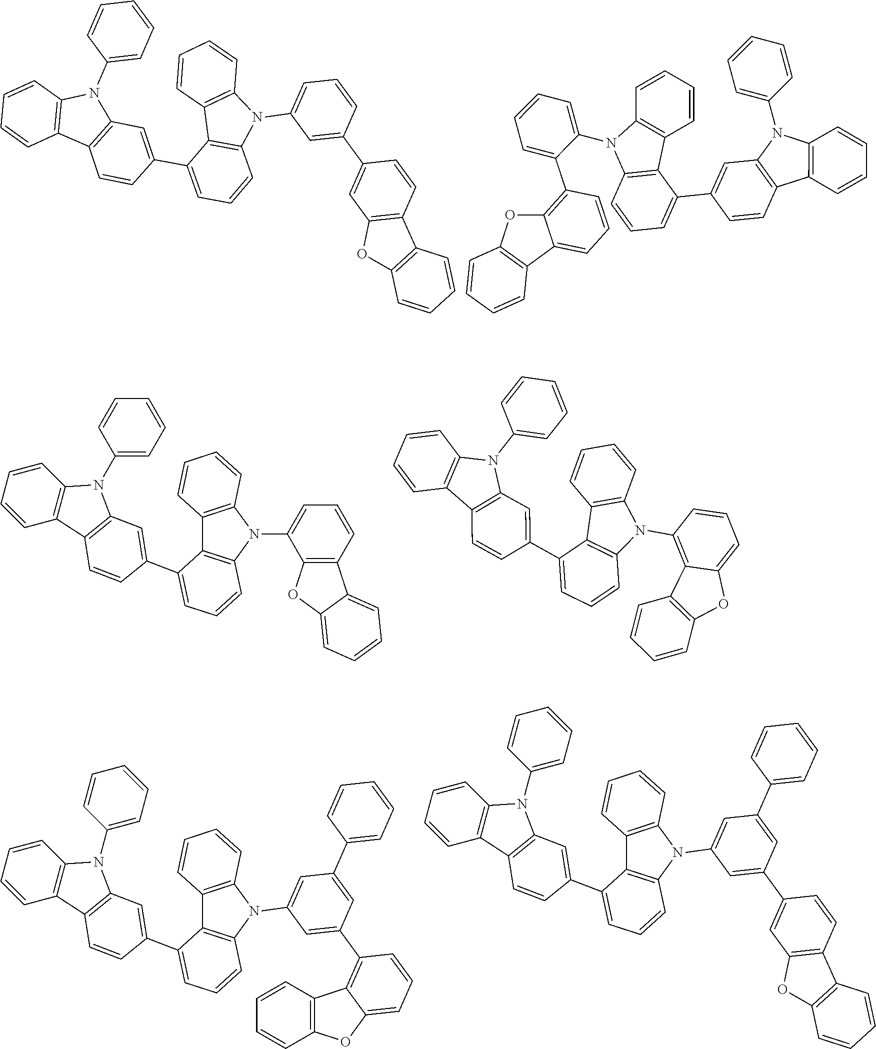

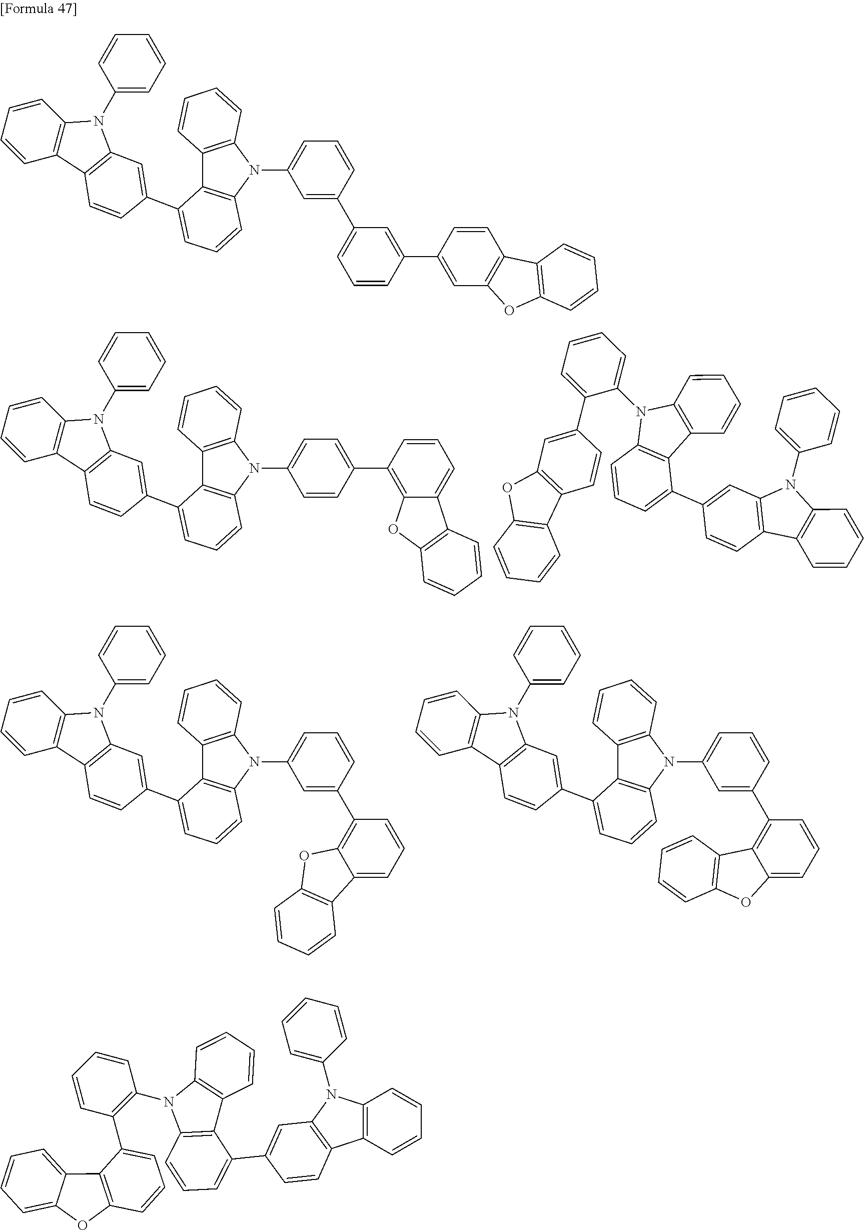

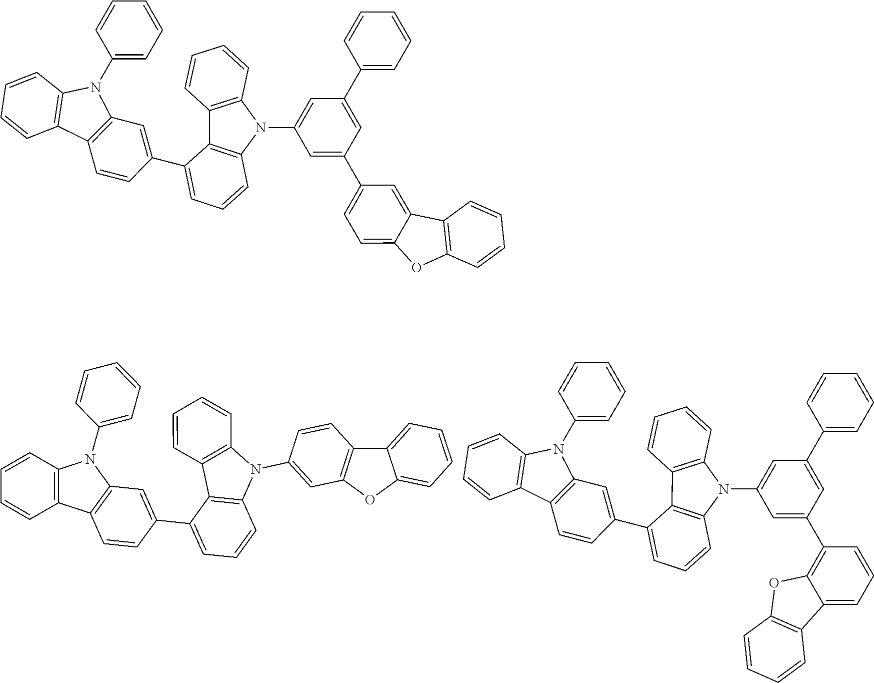

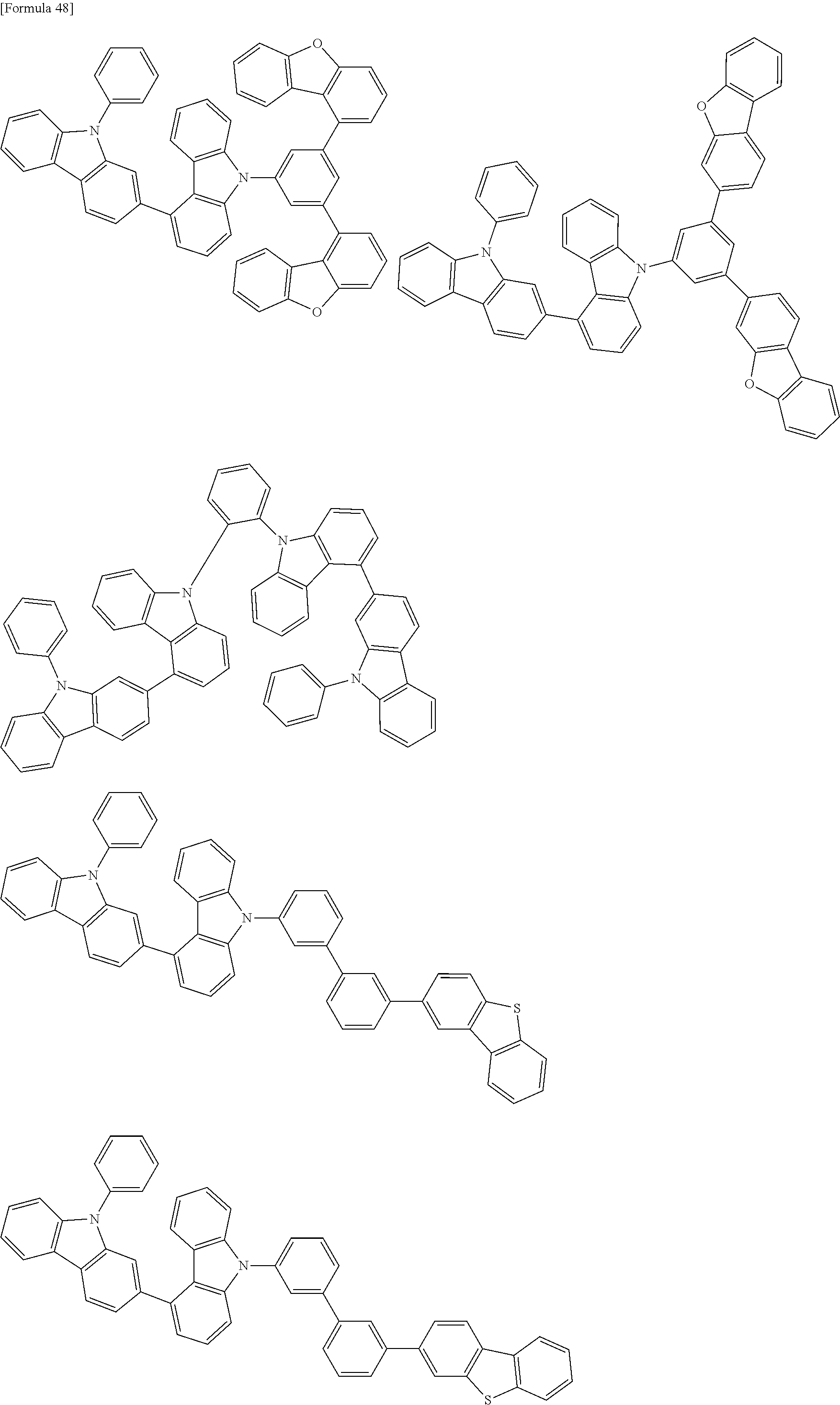

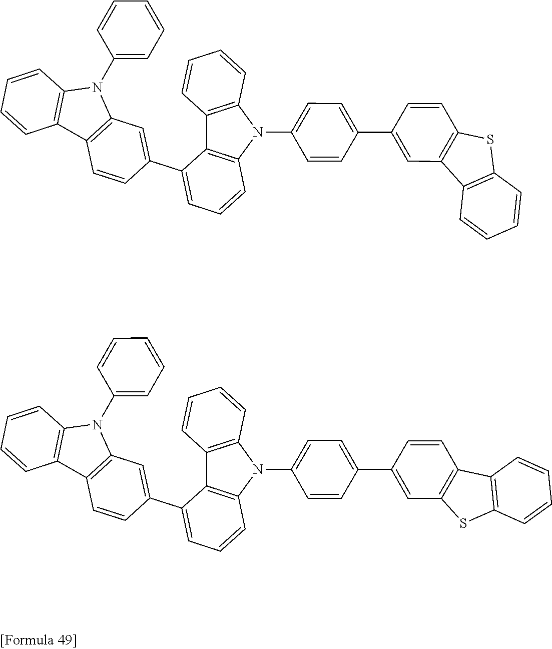 [SMILES: c1ccc(-n2c3ccccc3c3ccc(-c4cccc5c4c4ccccc4n5-c4ccc(-c5ccc6sc7ccccc7c6c5)cc4)cc32)cc1 ; c1ccc(-n2c3ccccc3c3ccc(-c4cccc5c4c4ccccc4n5-c4ccc(-c5ccc6c(c5)sc5ccccc56)cc4)cc32)cc1]

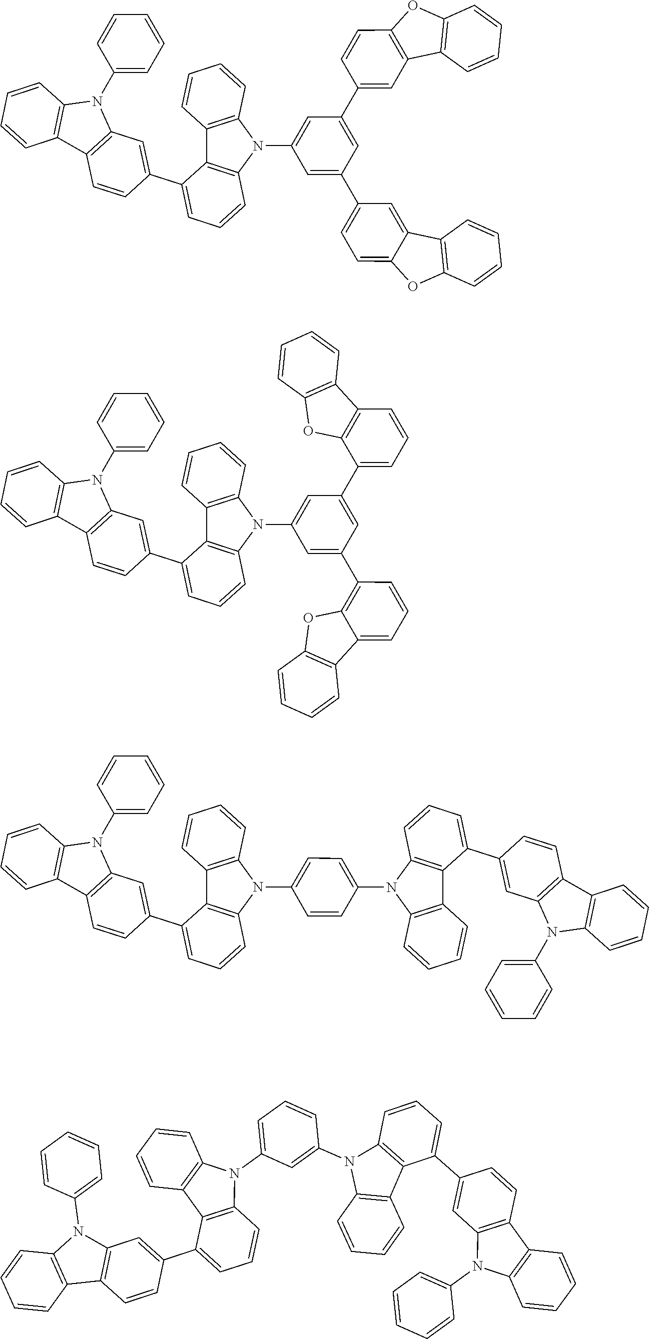 [SMILES: c1ccc(-n2c3ccccc3c3ccc(-c4cccc5c4c4ccccc4n5-c4cc(-c5ccc6oc7ccccc7c6c5)cc(-c5ccc6oc7ccccc7c6c5)c4)cc32)cc1 ; c1ccc(-n2c3ccccc3c3ccc(-c4cccc5c4c4ccccc4n5-c4cc(-c5cccc6c5oc5ccccc56)cc(-c5cccc6c5oc5ccccc56)c4)cc32)cc1 ; c1ccc(-n2c3ccccc3c3ccc(-c4cccc5c4c4ccccc4n5-c4ccc(-n5c6ccccc6c6c(-c7ccc8c9ccccc9n(-c9ccccc9)c8c7)cccc65)cc4)cc32)cc1 ; c1ccc(-n2c3ccccc3c3ccc(-c4cccc5c4c4ccccc4n5-c4cccc(-n5c6ccccc6c6c(-c7ccc8c9ccccc9n(-c9ccccc9)c8c7)cccc65)c4)cc32)cc1]

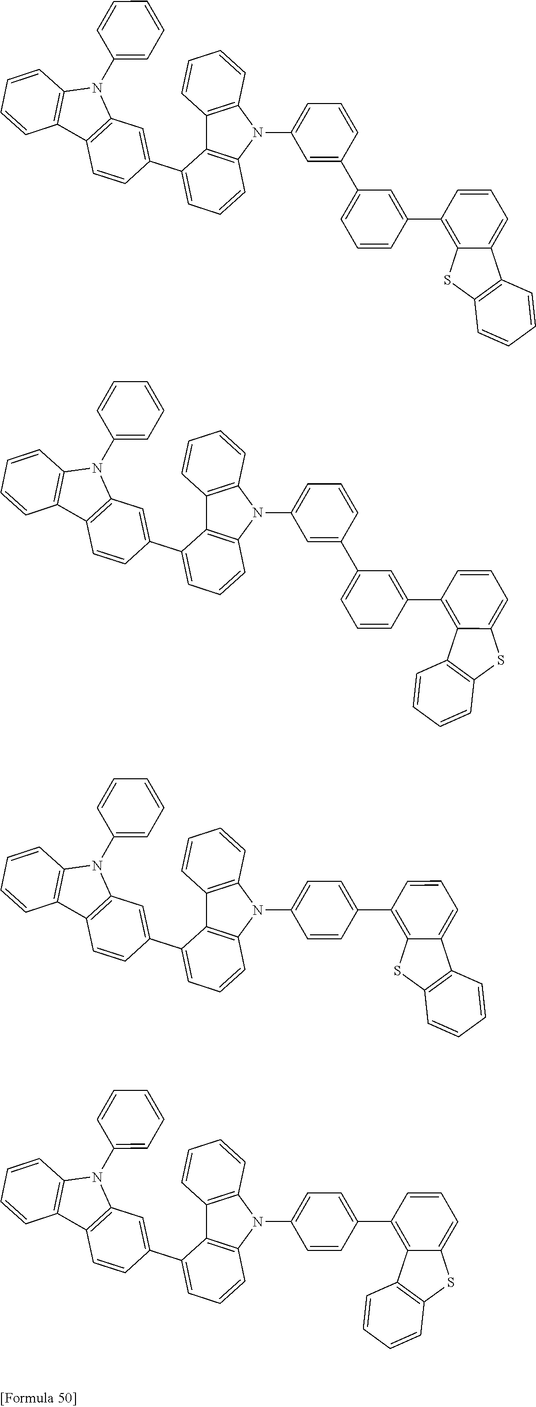 [SMILES: c1ccc(-n2c3ccccc3c3ccc(-c4cccc5c4c4ccccc4n5-c4cccc(-c5cccc(-c6cccc7c6sc6ccccc67)c5)c4)cc32)cc1 ; c1ccc(-n2c3ccccc3c3ccc(-c4cccc5c4c4ccccc4n5-c4cccc(-c5cccc(-c6cccc7sc8ccccc8c67)c5)c4)cc32)cc1 ; c1ccc(-n2c3ccccc3c3ccc(-c4cccc5c4c4ccccc4n5-c4ccc(-c5cccc6c5sc5ccccc56)cc4)cc32)cc1 ; c1ccc(-n2c3ccccc3c3ccc(-c4cccc5c4c4ccccc4n5-c4ccc(-c5cccc6sc7ccccc7c56)cc4)cc32)cc1]

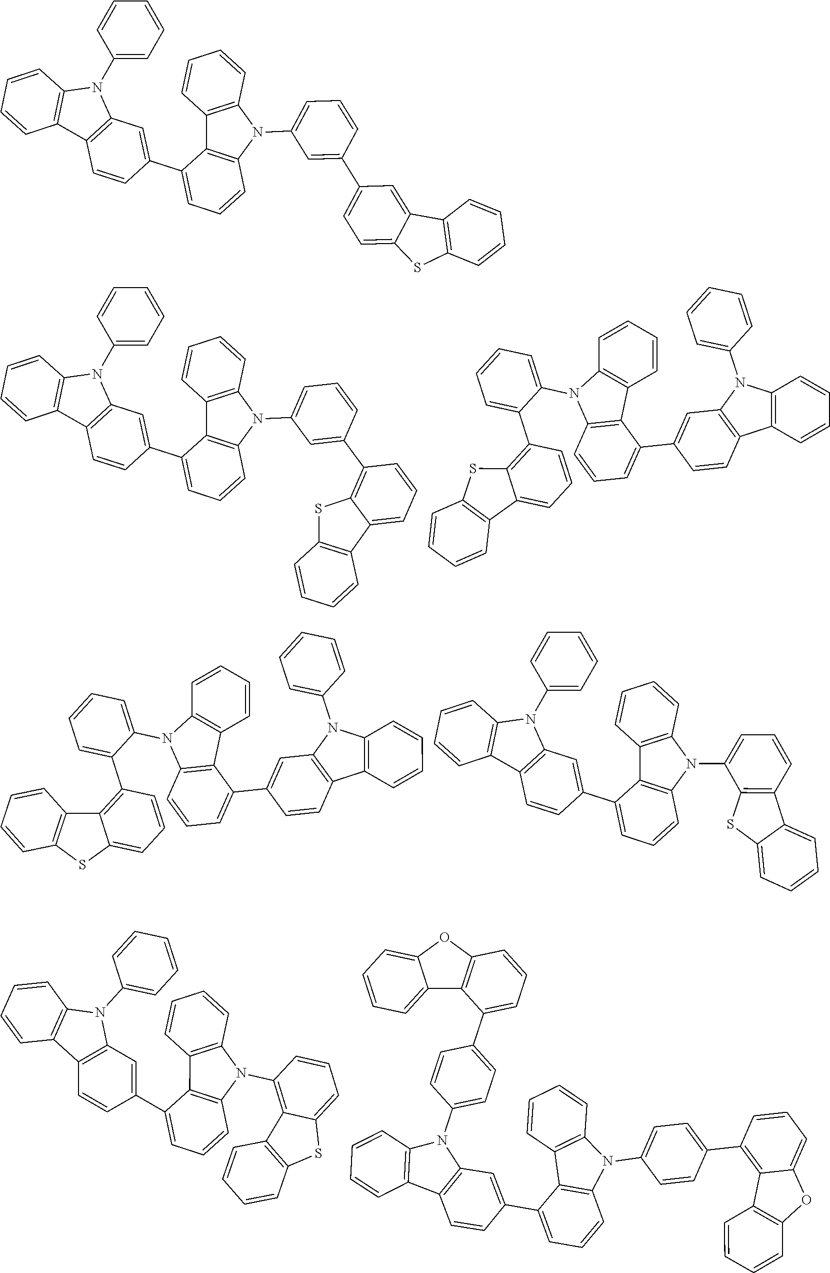

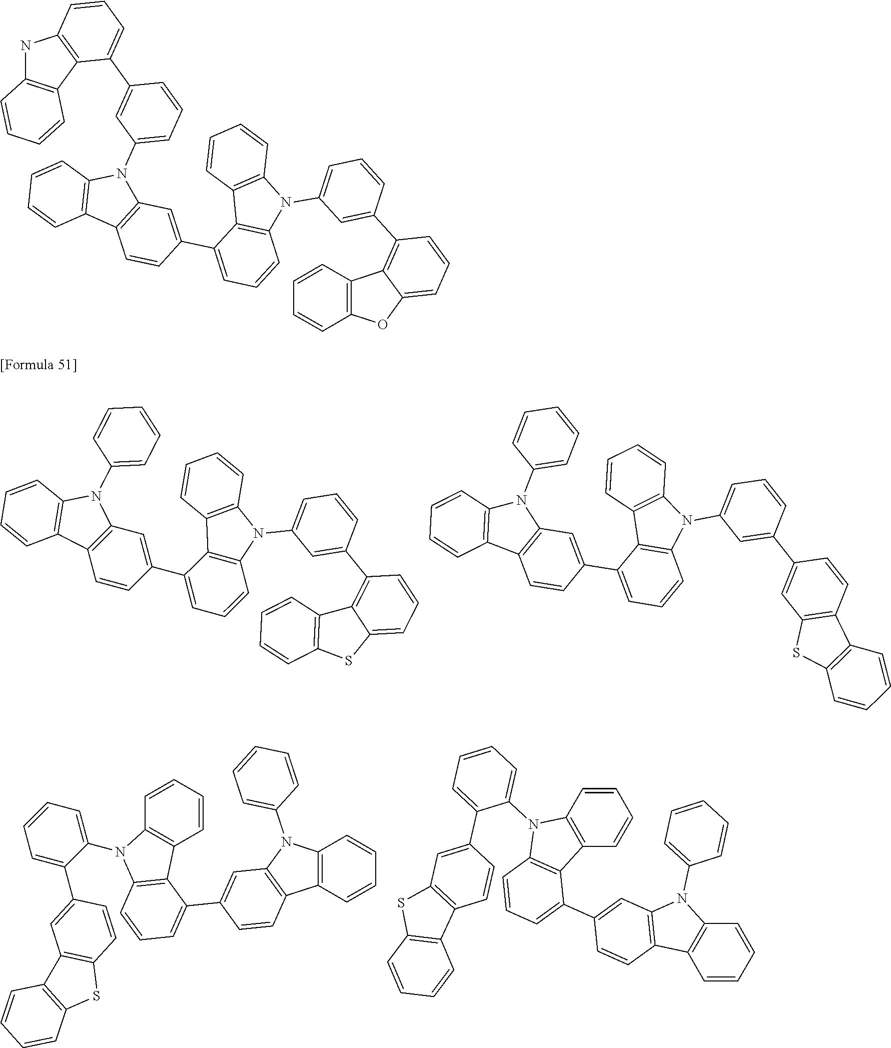

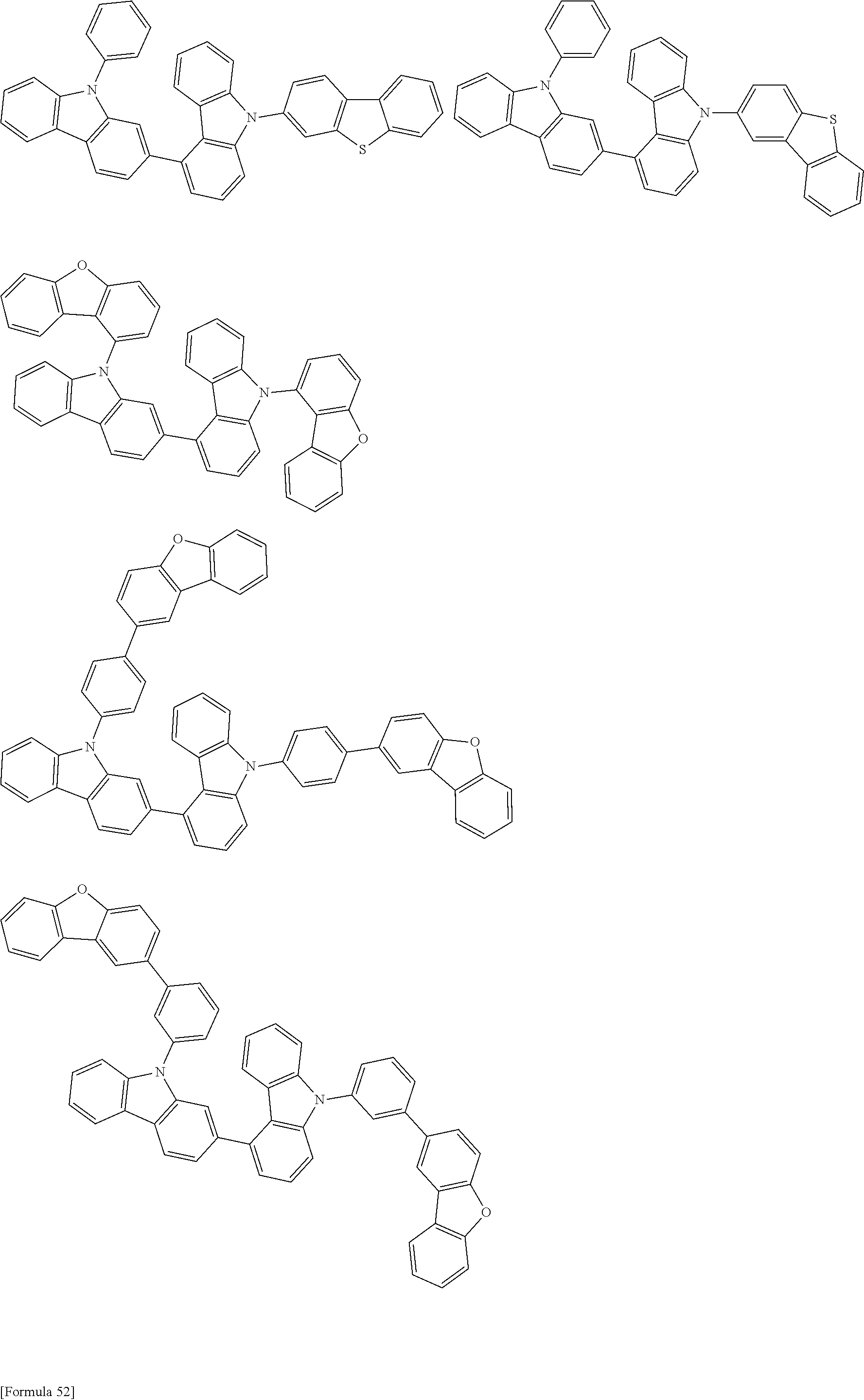

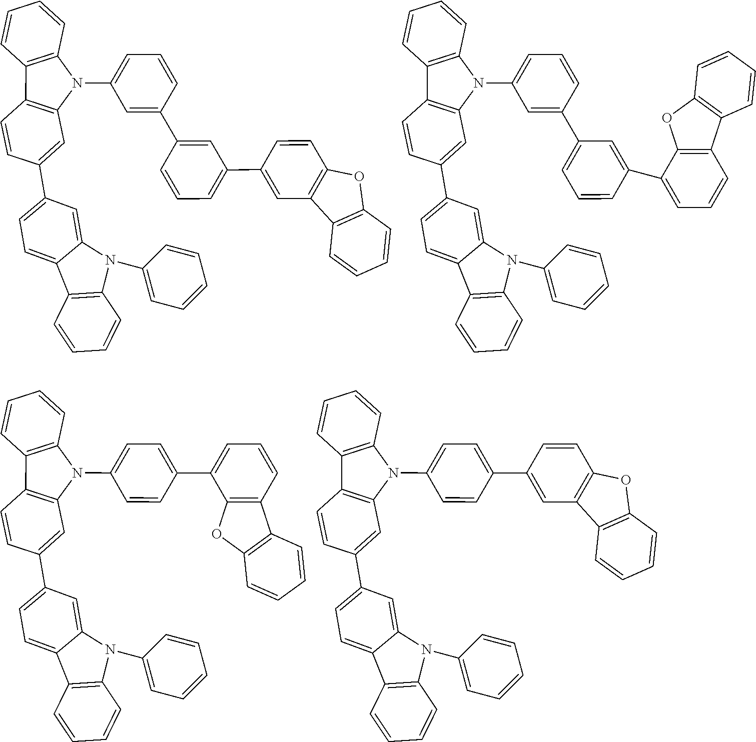

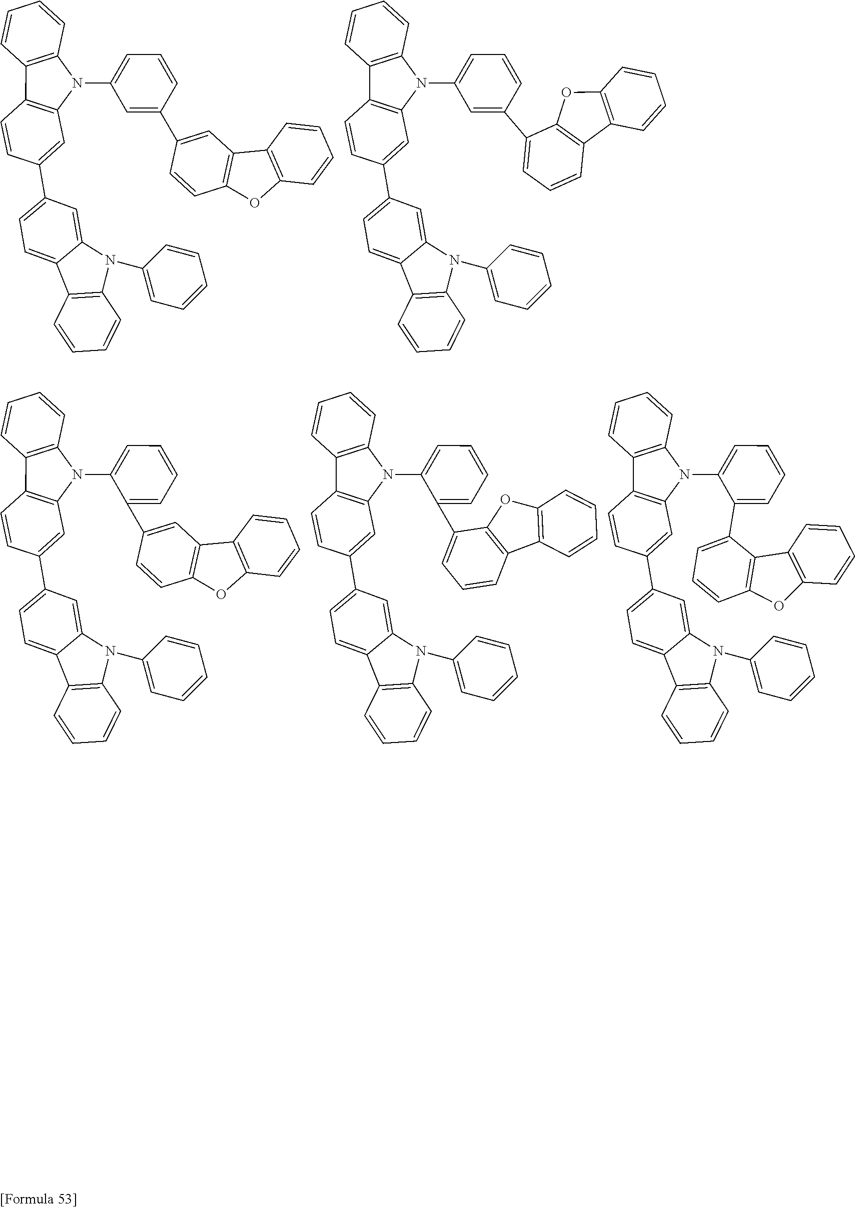

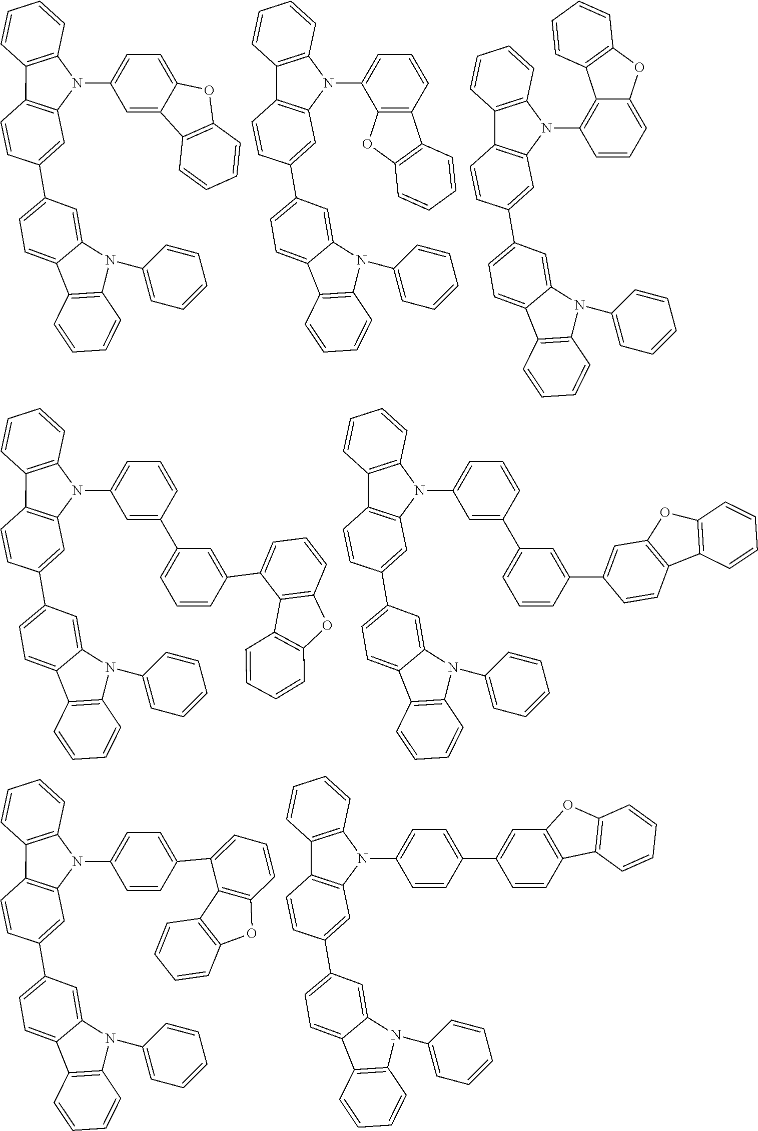

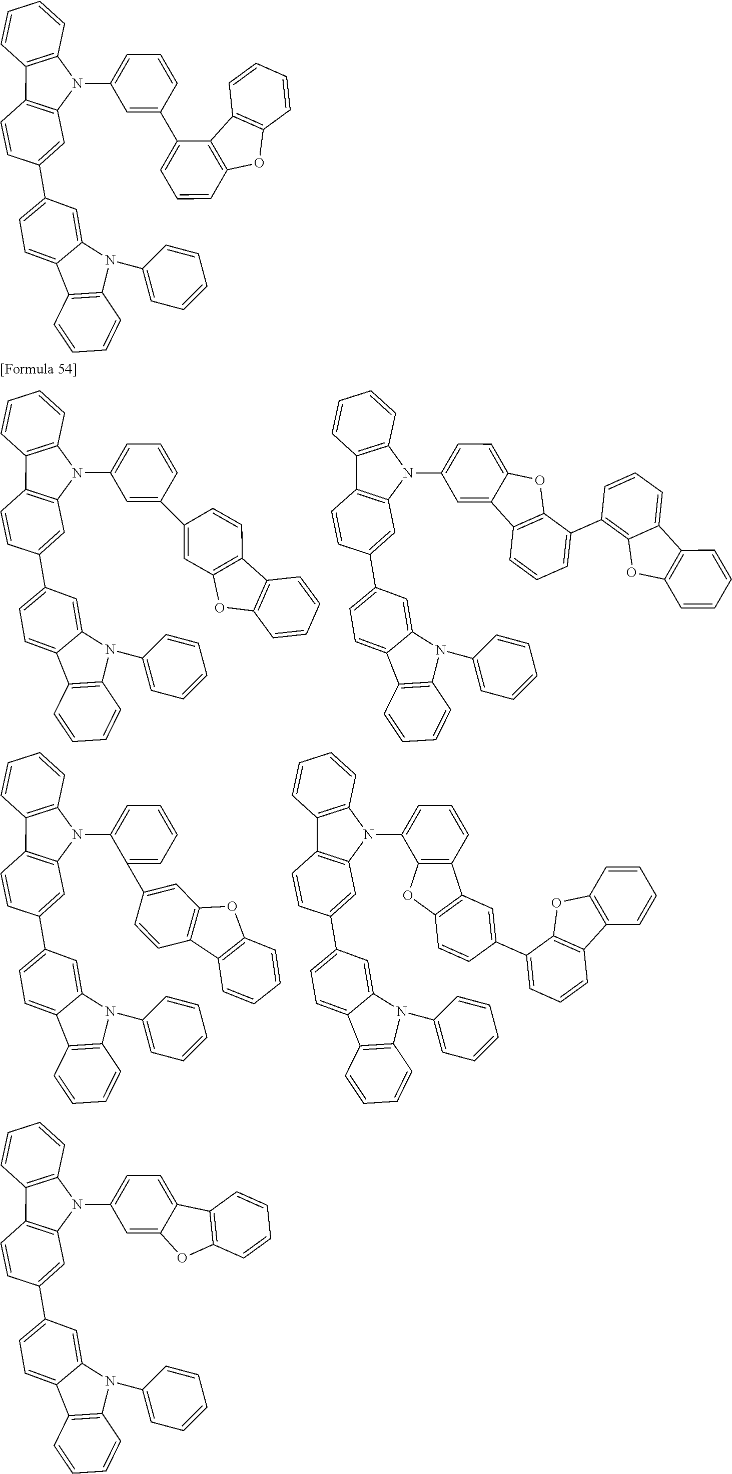

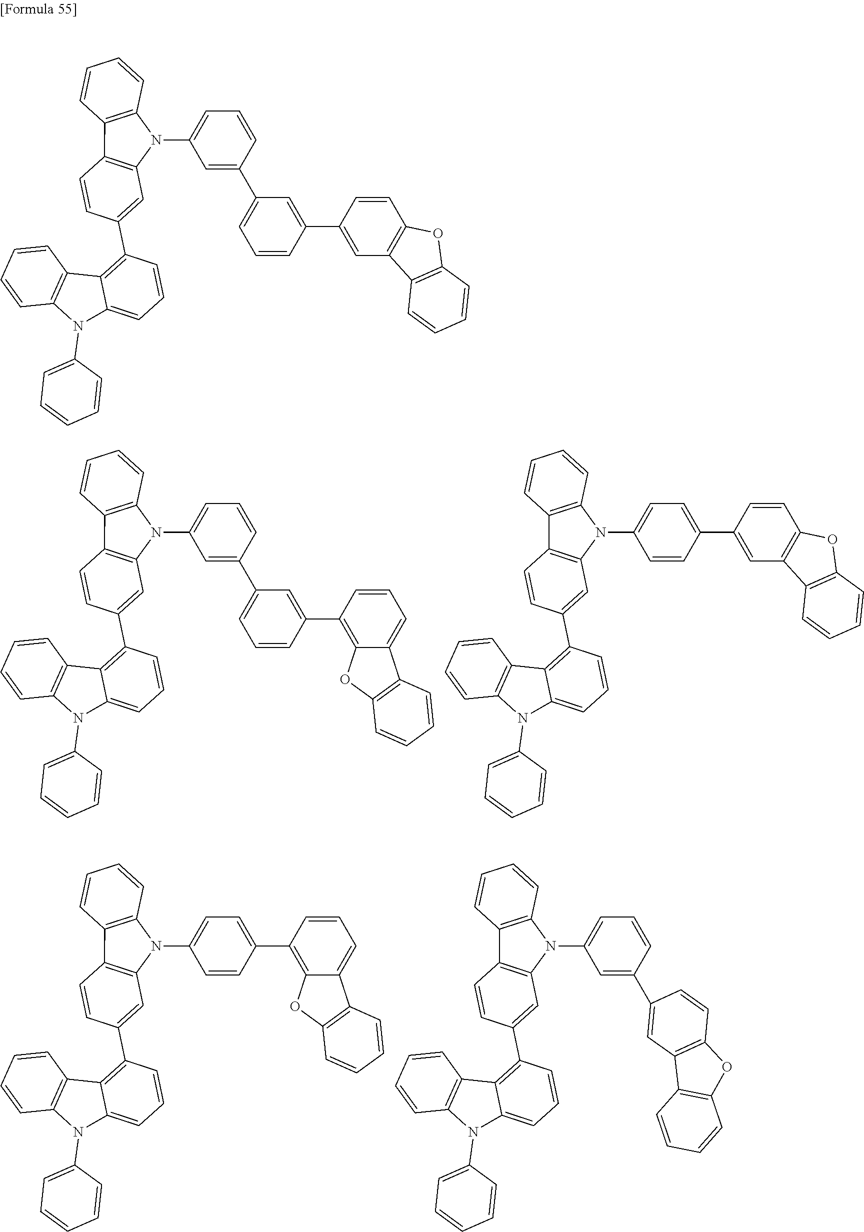

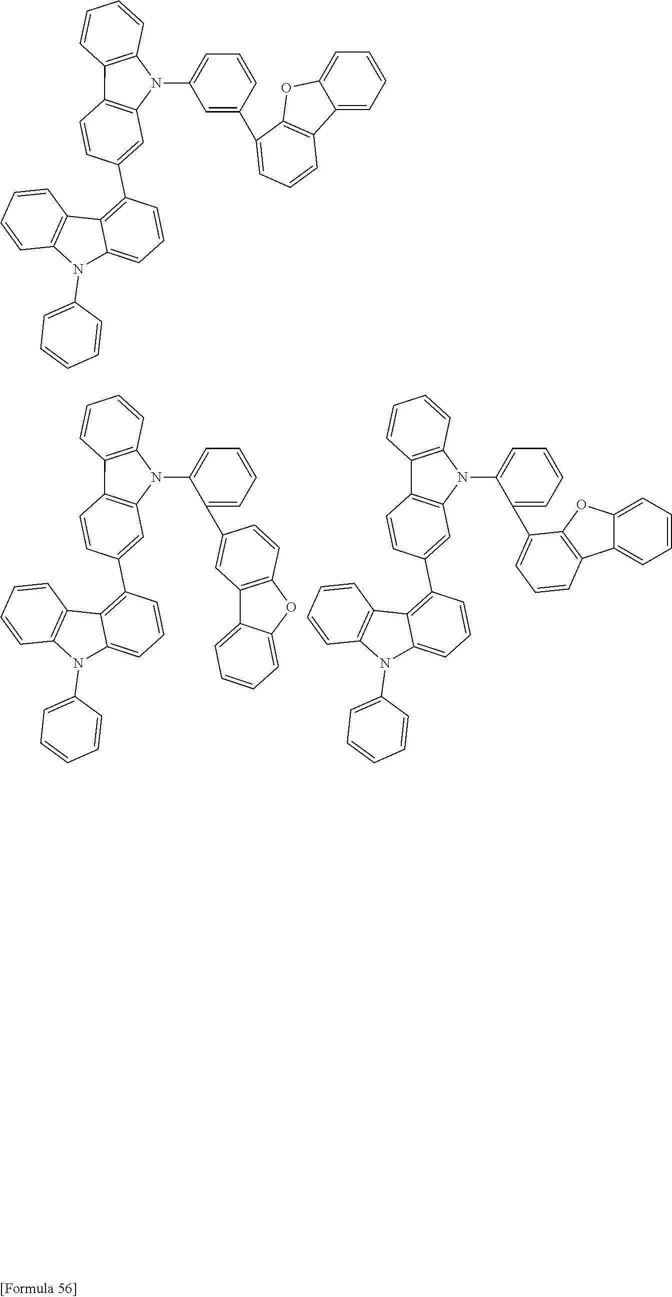

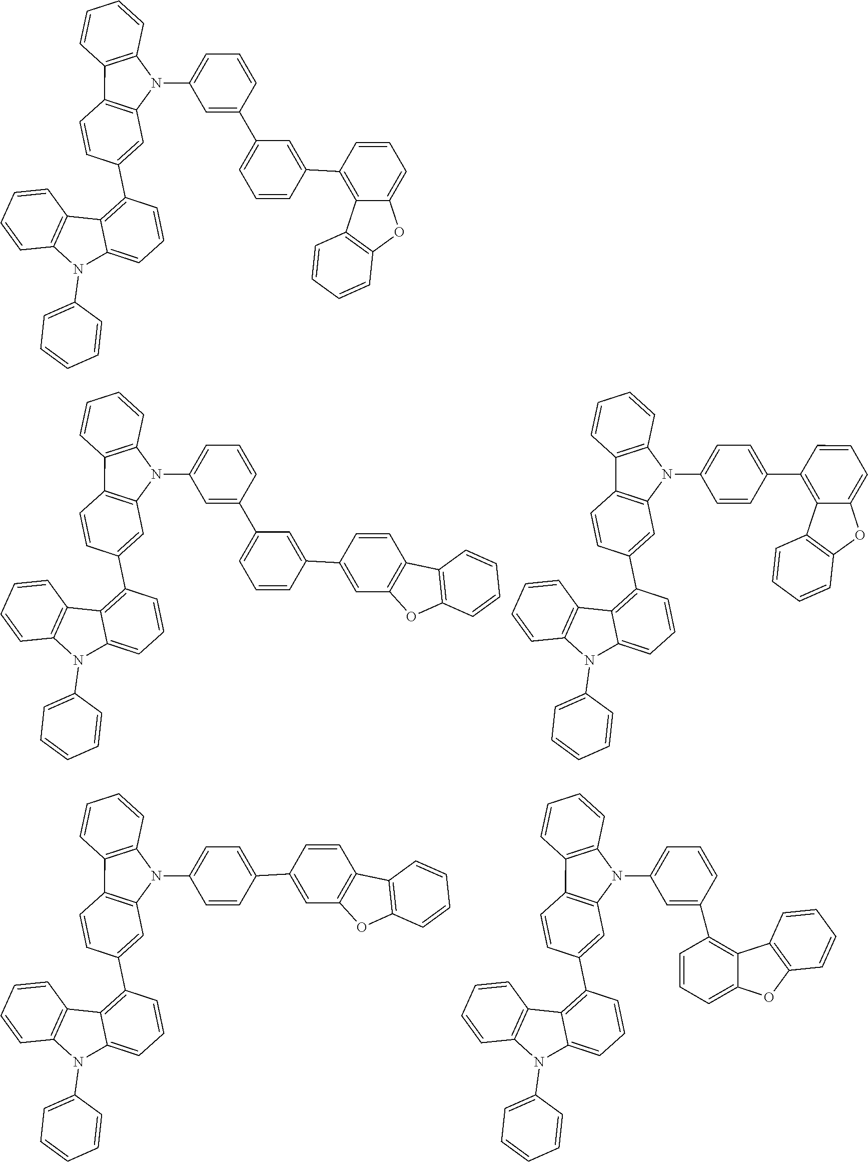

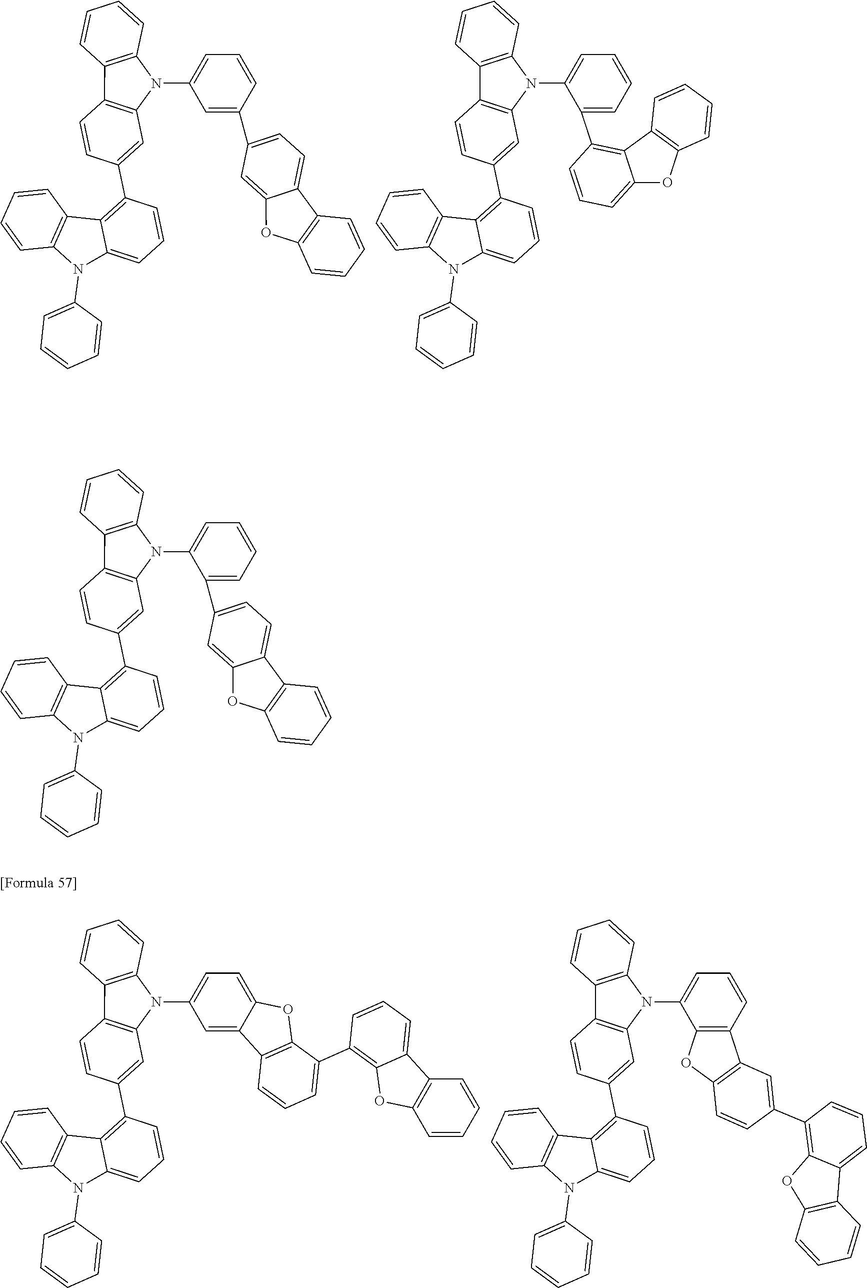

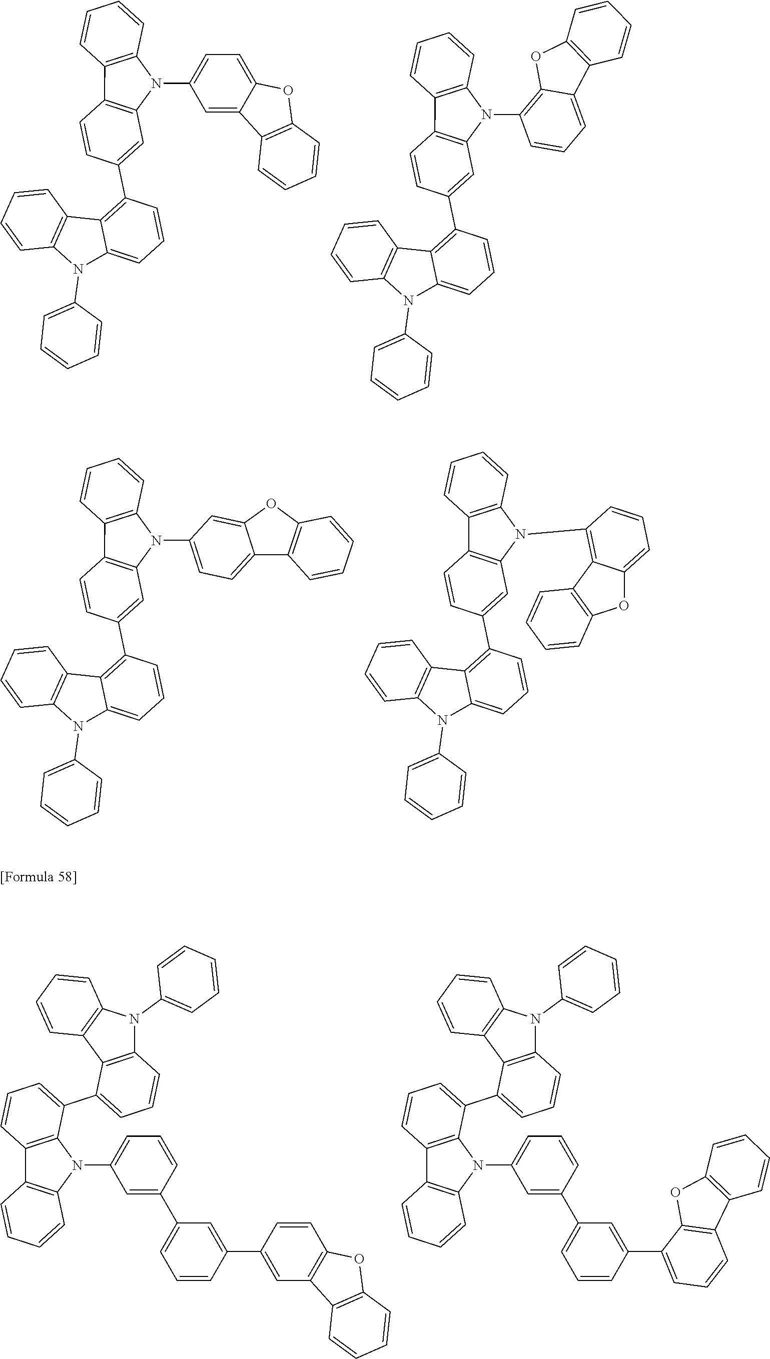

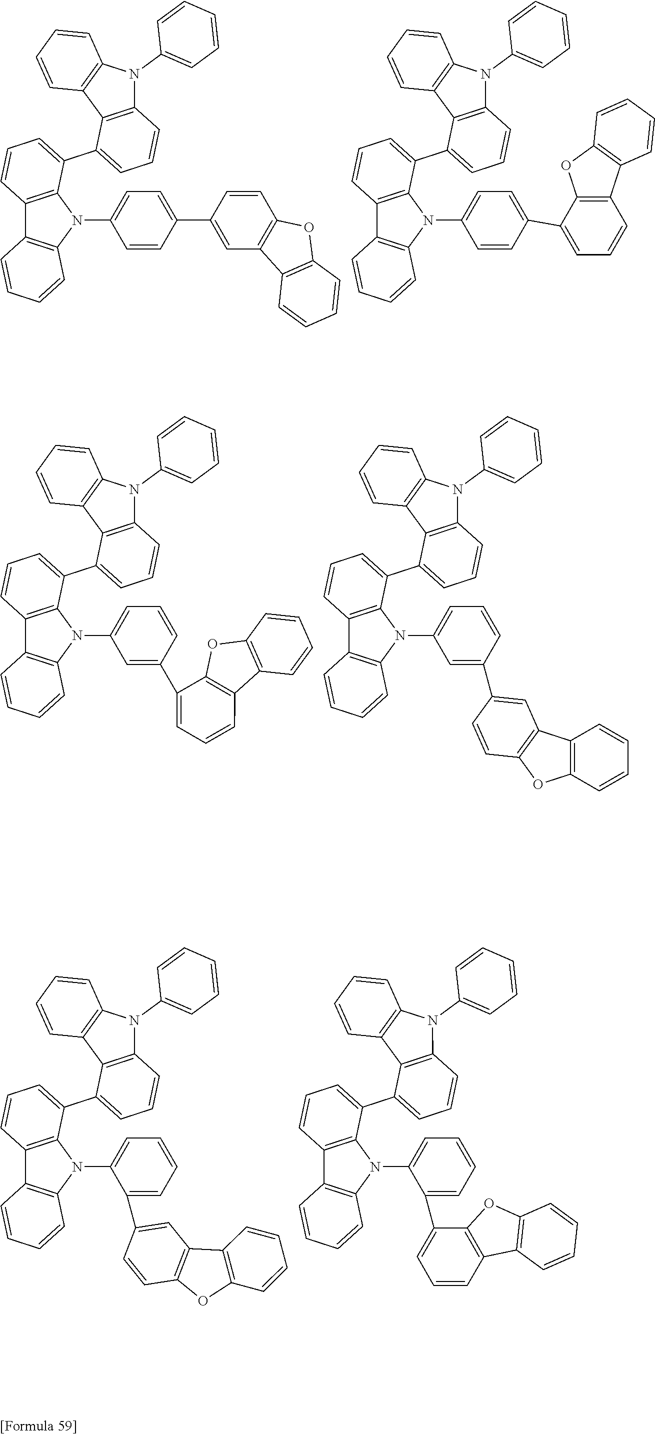

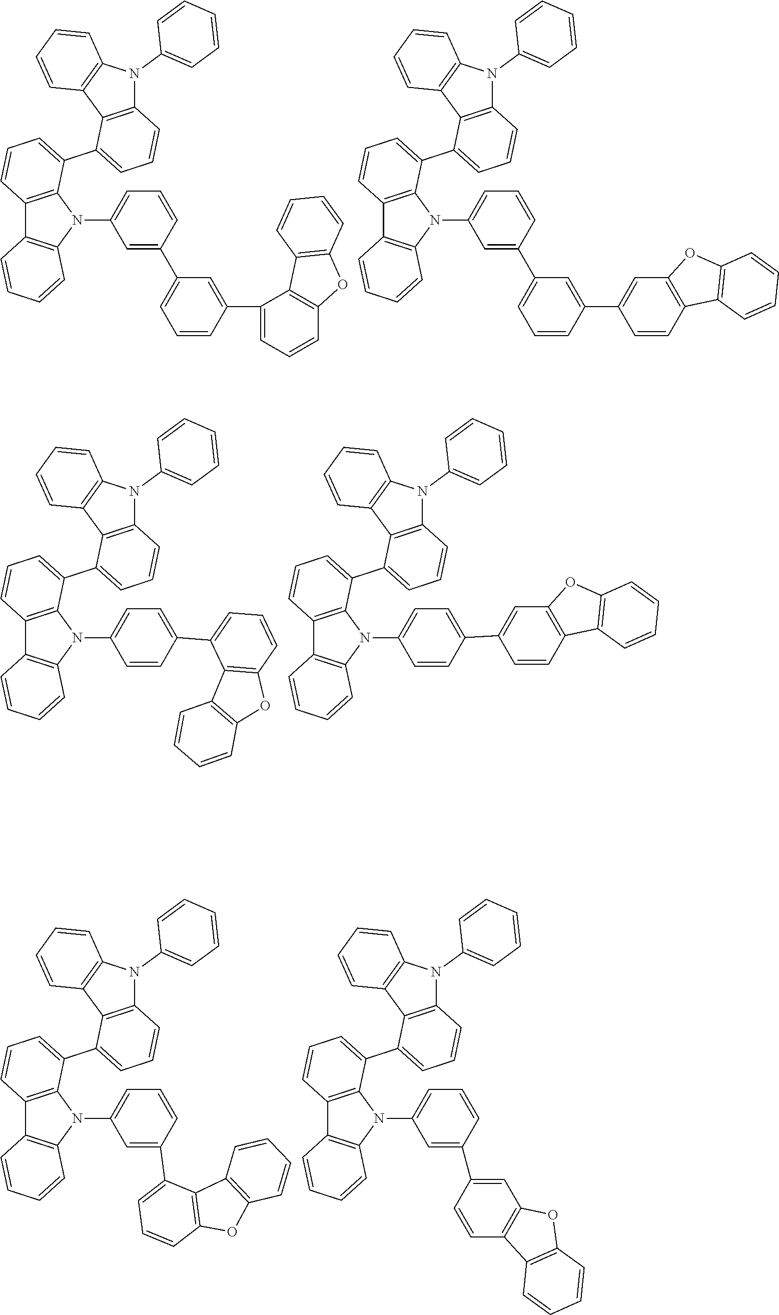

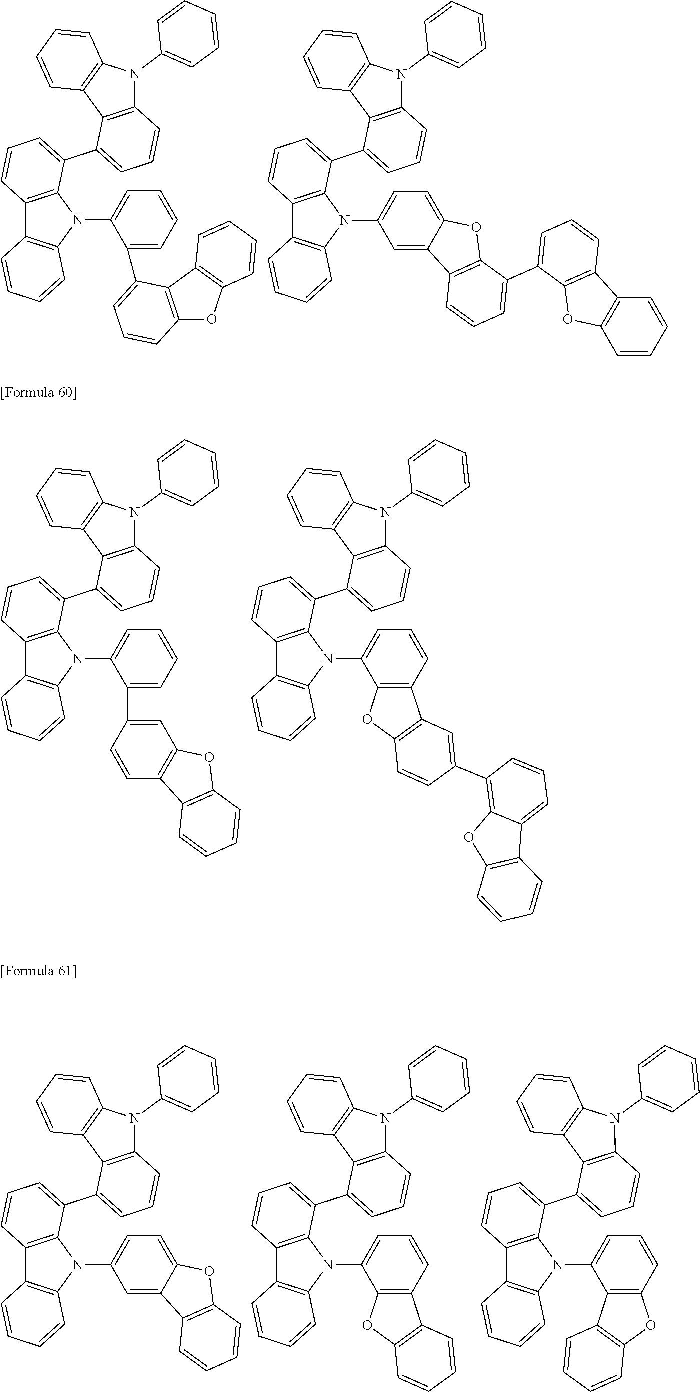

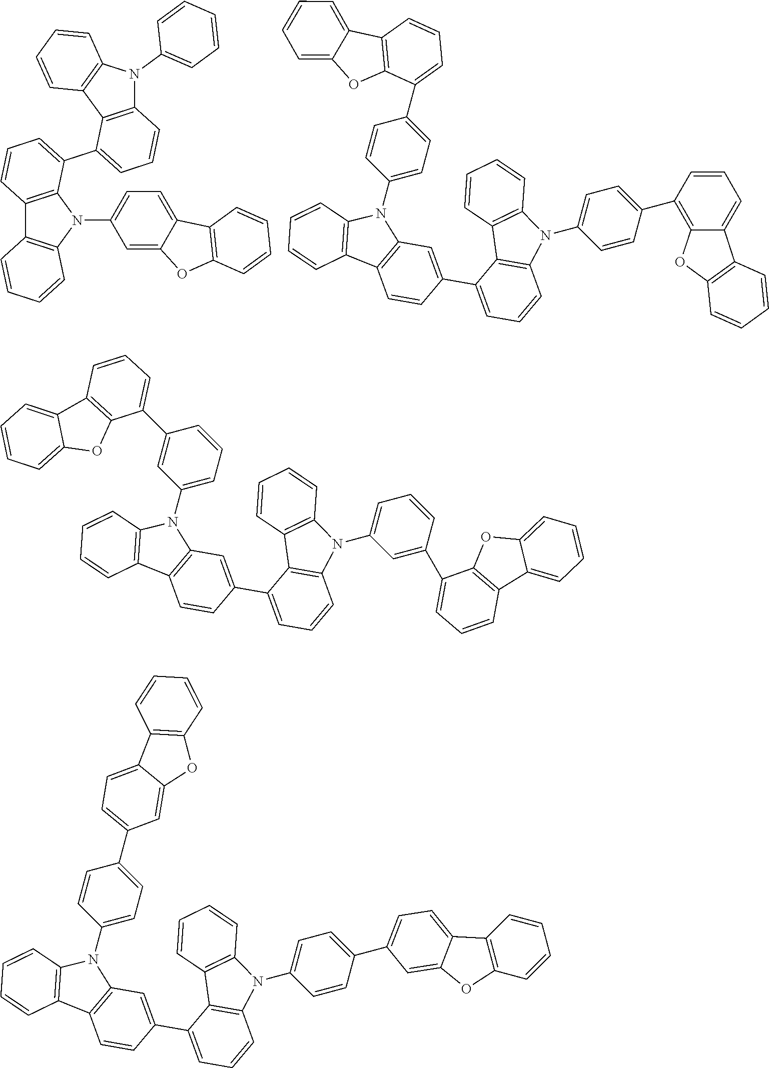

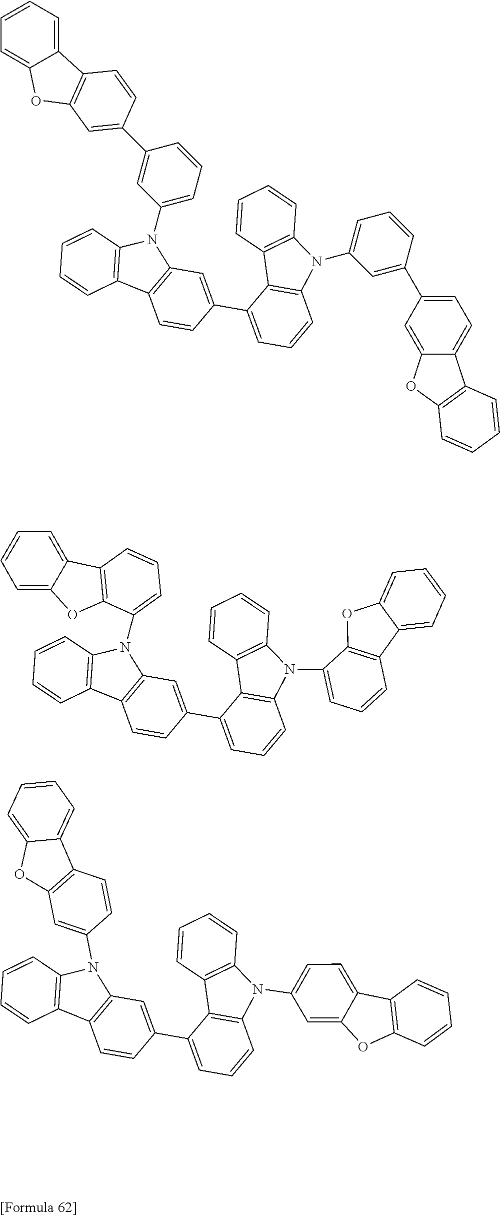 [SMILES: c1cc(-c2ccc3c(c2)oc2ccccc23)cc(-n2c3ccccc3c3ccc(-c4cccc5c4c4ccccc4n5-c4cccc(-c5ccc6c(c5)oc5ccccc56)c4)cc32)c1 ; c1ccc2c(c1)oc1c(-n3c4ccccc4c4ccc(-c5cccc6c5c5ccccc5n6-c5cccc6c5oc5ccccc56)cc43)cccc12 ; c1ccc2c(c1)oc1cc(-n3c4ccccc4c4ccc(-c5cccc6c5c5ccccc5n6-c5ccc6c(c5)oc5ccccc56)cc43)ccc12]

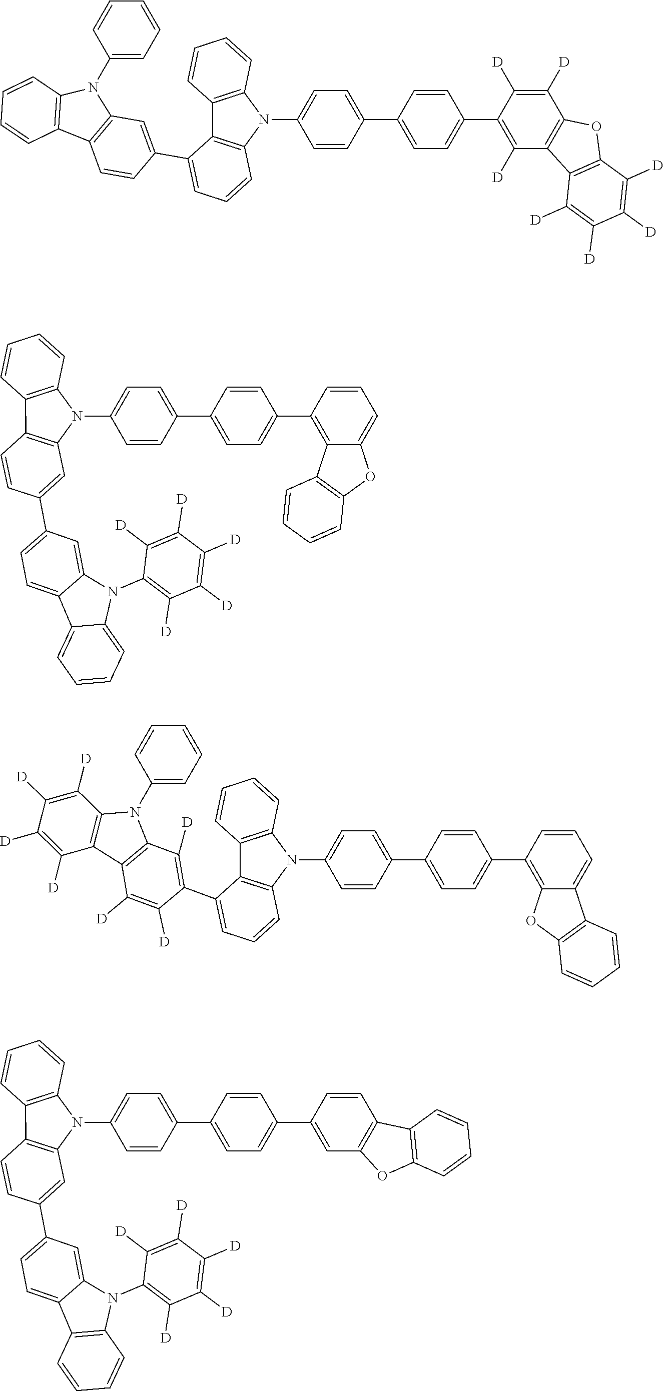

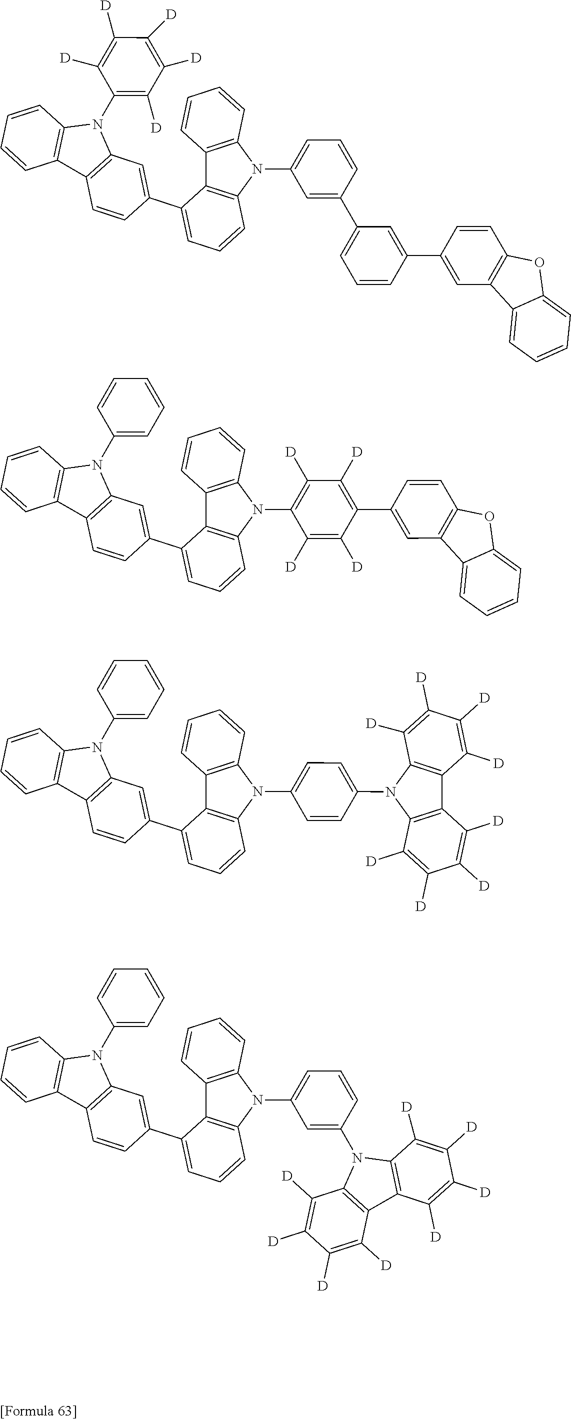 [SMILES: [2H]c1c([2H])c([2H])c(-n2c3ccccc3c3ccc(-c4cccc5c4c4ccccc4n5-c4cccc(-c5cccc(-c6ccc7oc8ccccc8c7c6)c5)c4)cc32)c([2H])c1[2H] ; [2H]c1c([2H])c(-n2c3ccccc3c3c(-c4ccc5c6ccccc6n(-c6ccccc6)c5c4)cccc32)c([2H])c([2H])c1-c1ccc2oc3ccccc3c2c1 ; [2H]c1c([2H])c([2H])c2c(c1[2H])c1c([2H])c([2H])c([2H])c([2H])c1n2-c1ccc(-n2c3ccccc3c3c(-c4ccc5c6ccccc6n(-c6ccccc6)c5c4)cccc32)cc1 ; [2H]c1c([2H])c([2H])c2c(c1[2H])c1c([2H])c([2H])c([2H])c([2H])c1n2-c1cccc(-n2c3ccccc3c3c(-c4ccc5c6ccccc6n(-c6ccccc6)c5c4)cccc32)c1]

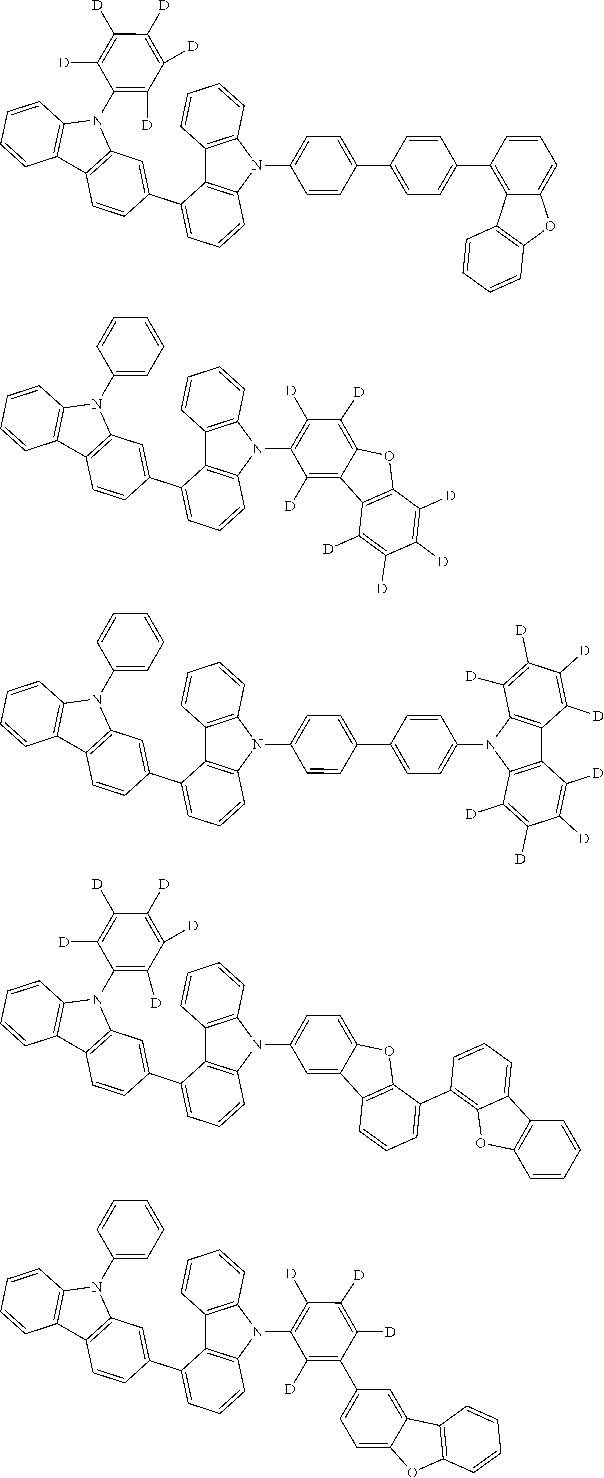

### Compound M2

The emitting layer of the exemplary embodiment contains a delayed fluorescent compound M2.

### Delayed Fluorescence

Delayed fluorescence is explained in “Yuki Hando-tai no Debaisu Bussei (Device Physics of Organic Semiconductors)” (edited by ADACHI, Chihaya, published by Kodansha, on pages 261-268). This document describes that, if an energy difference ΔE13 of a fluorescent material between a singlet state and a triplet state is reducible, a reverse energy transfer from the triplet state to the singlet state, which usually occurs at a low transition probability, would occur at a high efficiency to express thermally activated delayed fluorescence (TADF). Further, a generation mechanism of delayed fluorescence is explained in FIG. 10.38 in the document. The compound M2 of the exemplary embodiment is preferably a compound exhibiting thermally activated delayed fluorescence generated by such a mechanism.

In general, emission of delayed fluorescence can be confirmed by measuring the transient PL (Photo Luminescence).

The behavior of delayed fluorescence can also be analyzed based on the decay curve obtained from the transient PL measurement. The transient PL measurement is a method of irradiating a sample with a pulse laser to excite the sample, and measuring the decay behavior (transient characteristics) of PL emission after the irradiation is stopped. PL emission in TADF materials is classified into a light emission component from a singlet exciton generated by the first PL excitation and a light emission component from a singlet exciton generated via a triplet exciton. The lifetime of the singlet exciton generated by the first PL excitation is on the order of nanoseconds and is very short. Therefore, light emission from the singlet exciton rapidly attenuates after irradiation with the pulse laser.

On the other hand, the delayed fluorescence is gradually attenuated due to light emission from a singlet exciton generated via a triplet exciton having a long lifetime. As described above, there is a large temporal difference between the light emission from the singlet exciton generated by the first PL excitation and the light emission from the singlet exciton generated via the triplet exciton. Therefore, the luminous intensity derived from delayed fluorescence can be determined.

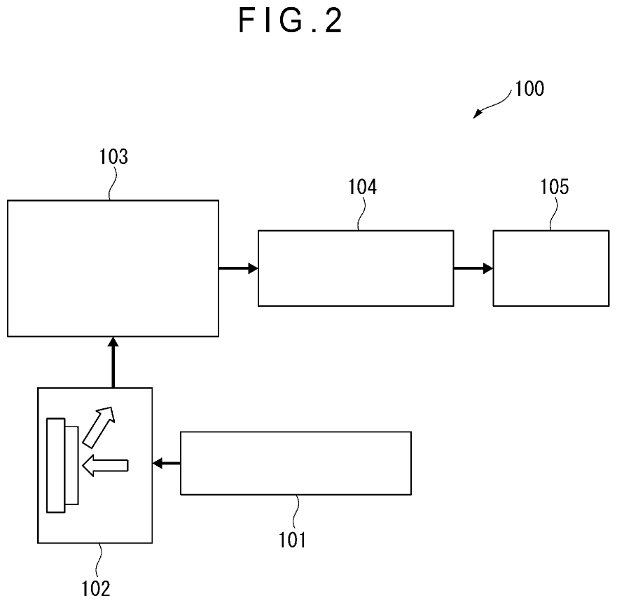

FIG. 2 is a schematic diagram of an exemplary device for measuring the transient PL.

An example of a method of measuring a transient PL using FIG. 2 and an example of behavior analysis of delayed fluorescence will be described.

A transient PL measuring device 100 in FIG. 2 includes: a pulse laser 101 capable of radiating a light having a predetermined wavelength; a sample chamber 102 configured to house a measurement sample; a spectrometer 103 configured to divide a light radiated from the measurement sample; a streak camera 104 configured to provide a two-dimensional image; and a personal computer 105 configured to import and analyze the two-dimensional image. A device for measuring the transient PL is not limited to the device shown in FIG. 2

The sample housed in the sample chamber 102 is obtained by forming a thin film, in which a matrix material is doped with a doping material at a concentration of 12 mass %, on the quartz substrate.

The thin film sample housed in the sample chamber 102 is irradiated with the pulse laser from the pulse laser 101 to excite the doping material. Emission is extracted in a direction of 90 degrees with respect to a radiation direction of the excited light. The extracted emission is divided by the spectrometer 103 to form a two-dimensional image in the streak camera 104. As a result, the two-dimensional image is obtainable in which the ordinate axis represents a time, the abscissa axis represents a wavelength, and a bright spot represents a luminous intensity. When this two-dimensional image is taken out at a predetermined time axis, an emission spectrum in which the ordinate axis represents the luminous intensity and the abscissa axis represents the wavelength is obtainable. Moreover, when this two-dimensional image is taken out at the wavelength axis, a decay curve (transient PL) in which the ordinate axis represents a logarithm of the luminous intensity and the abscissa axis represents the time is obtainable.

For instance, a thin film sample A was prepared as described above from a reference compound H1 as the matrix material and a reference compound D1 as the doping material and was measured in terms of the transient PL.

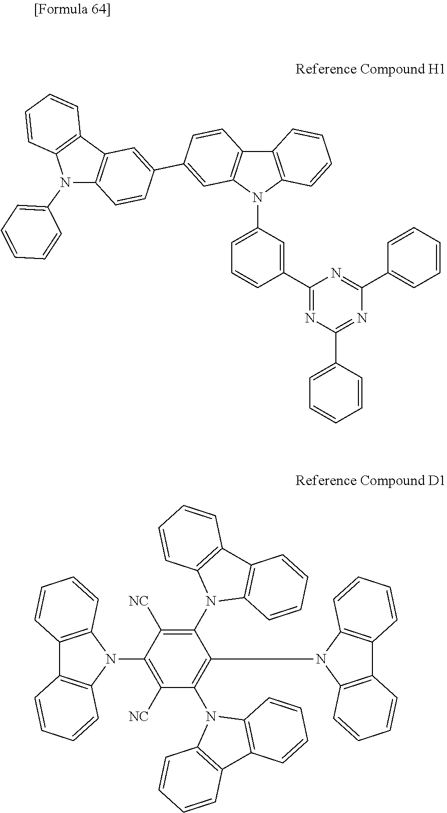 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1 ; N#Cc1c(-n2c3ccccc3c3ccccc32)c(C#N)c(-n2c3ccccc3c3ccccc32)c(-n2c3ccccc3c3ccccc32)c1-n1c2ccccc2c2ccccc21]

The decay curve was analyzed with respect to the above thin film sample A and a thin film sample B. The thin film sample B was produced in the same manner as described above from a reference compound H2 as the matrix material and the reference compound D1 as the doping material.

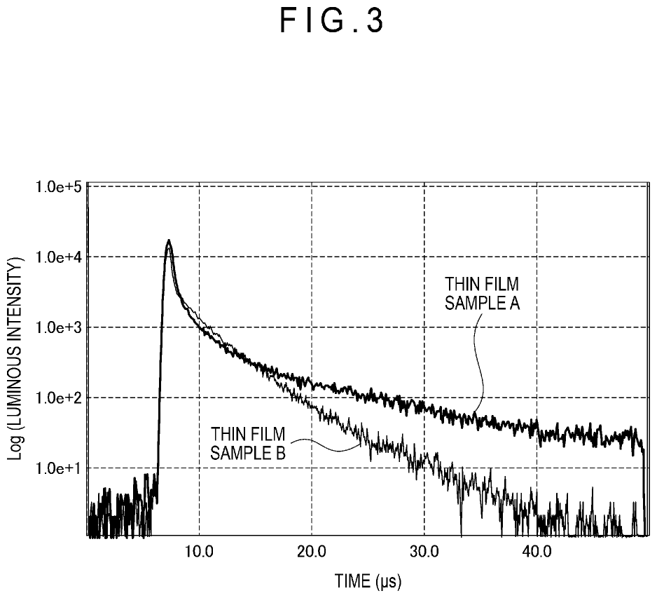

FIG. 3 shows decay curves obtained from transient PL obtained by measuring the thin film samples A and B.

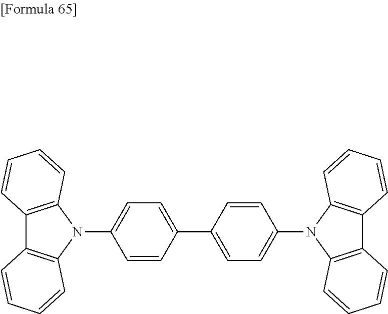 [SMILES: c1ccc2c(c1)c1ccccc1n2-c1ccc(-c2ccc(-n3c4ccccc4c4ccccc43)cc2)cc1]

### Reference Compound H2

As described above, an emission decay curve in which the ordinate axis represents the luminous intensity and the abscissa axis represents the time can be obtained by the transient PL measurement. Based on the emission decay curve, a fluorescence intensity ratio between fluorescence emitted from a singlet state generated by photo-excitation and delayed fluorescence emitted from a singlet state generated by reverse energy transfer via a triplet state can be estimated. In a delayed fluorescent material, a ratio of the intensity of the slowly decaying delayed fluorescence to the intensity of the promptly decaying fluorescence is relatively large.

Specifically, Prompt emission and Delay emission are present as emission from the delayed fluorescent material. Prompt emission is observed promptly when the excited state is achieved by exciting the compound of the exemplary embodiment with a pulse beam (i.e., a beam emitted from a pulse laser) having a wavelength absorbable by the delayed fluorescent material. Delay emission is observed not promptly when the excited state is achieved but after the excited state is achieved.

An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using a device different from one described in Reference Document 1 or one shown in FIG. 2

Herein, a sample produced by the following method is used for measuring delayed fluorescence of the compound M2. For instance, the compound M2 is dissolved in toluene to prepare a dilute solution with an absorbance of 0.05 or less at the excitation wavelength to eliminate the contribution of self-absorption. In order to prevent quenching due to oxygen, the sample solution is frozen and degassed and then sealed in a cell with a lid under an argon atmosphere to obtain an oxygen-free sample solution saturated with argon.

The fluorescence spectrum of the sample solution is measured with a spectrofluorometer FP-8600 (produced by JASCO Corporation), and the fluorescence spectrum of a 9,10-diphenylanthracene ethanol solution is measured under the same conditions. Using the fluorescence area intensities of both spectra, the total fluorescence quantum yield is calculated by an equation (1) in Morris et al. J. Phys. Chem. 80 (1976) 969. An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using a device different from one described in Reference Document 1 or one shown in FIG. 2

In the exemplary embodiment, provided that an amount of Prompt emission of a measurement target compound (compound M2) is denoted by XP and an amount of Delay emission is denoted by XD, a value of XD/XP is preferably 0.05 or more.

The amounts of Prompt emission and Delay emission and a ratio of the amounts thereof in compounds other than the compound M2 herein are measured in the same manner as those of the compound M2.

The compound M2 is preferably a compound represented by a formula (2) or a formula (22) below, more preferably a compound represented by the formula (2).

### Compound Represented by Formula (2)

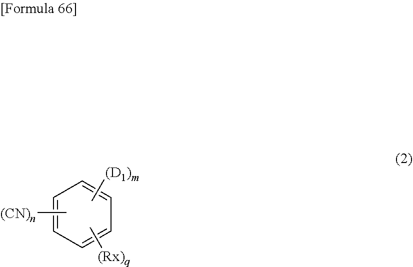

In the formula (2):
n is 1, 2, 3 or 4;
m is 1, 2, 3 or 4;
q is 0, 1, 2, 3 or 4;
m+n+q=6 is satisfied;
CN is a cyano group;
D₁ is a group represented by a formula (2a), (2b) or (2c) below, and when a plurality of D₁ are present, the plurality of D₁ are mutually the same or different;
Rx is a hydrogen atom or a substituent, or at least one combination of combinations of adjacent ones of Rx are mutually bonded to form a ring, and when a plurality of Rx are present, the plurality of Rx are mutually the same or different;
each Rx as a substituent is independently a halogen atom, a substituted or unsubstituted aryl group having 6 to 30 ring carbon atoms, a substituted or unsubstituted heterocyclic group having 5 to 30 ring atoms, a substituted or unsubstituted amino group, a substituted or unsubstituted carbonyl group, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkylsilyl group having 3 to 30 carbon atoms, or a substituted or unsubstituted arylsilyl group having 6 to 60 ring carbon atoms; and
CN, D₁ and Rx are bonded to respective carbon atoms of a six-membered ring.

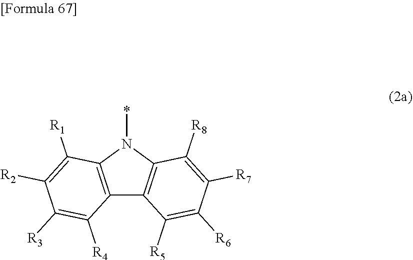

In the formula (2a): R₁ to R₈ are each independently a hydrogen atom or a substituent, or at least one combination of a combination of R₁ and R₂, a combination of R₂ and R₃, a combination of R₃ and R₄, a combination of R₅ and R₆, a combination of R₆ and R₇, or a combination of R₇ and R₈ are mutually bonded to form a ring;

R₁ to R₈ as a substituent are each independently a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkylsilyl group having 3 to 30 carbon atoms, a substituted or unsubstituted arylsilyl group having 6 to 60 ring carbon atoms, a hydroxy group, a substituted or unsubstituted alkoxy group having 1 to 30 carbon atoms, a substituted or unsubstituted alkoxy halide group having 1 to 30 carbon atoms, a substituted or unsubstituted aryloxy group having 6 to 30 ring carbon atoms, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and

* represents a bonding position to a carbon atom in a six-membered ring in the formula (2).

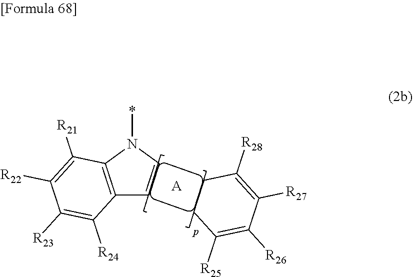

In the formula (2b):
R₂₁ to R₂₈ are each independently a hydrogen atom or a substituent, or at least one combination of a combination of R₂₁ and R₂₂, a combination of R₂₂ and R₂₃, a combination of R₂₃ and R₂₄, a combination of R₂₅ and R₂₆, a combination of R₂₆ and R₂₇, or a combination of R₂₇ and R₂₈ are mutually bonded to form a ring;
R₂₁ to R₂₈ as a substituent each independently represent the same as R₁ to R₈ in the formula (2a);
A represents a cyclic structure represented by a formula (211) or (212) below, and the cyclic structure A is fused with adjacent cyclic structure(s) at any position(s);
p is 1, 2, 3 or 4;
when p is 2, 3 or 4, a plurality of cyclic structures A are mutually the same or different; and
* represents a bonding position to a carbon atom in a six-membered ring in the formula (2).

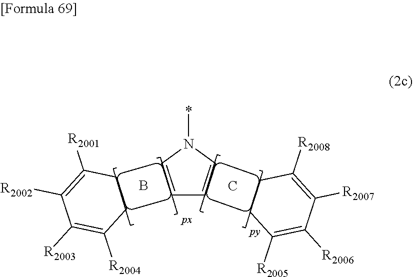

In the formula (2c):
R₂₀₀₁ to R₂₀₀₈ are each independently a hydrogen atom or a substituent, or at least one combination of a combination of R₂₀₀₁ and R₂₀₀₂, a combination of R₂₀₀₂ and R₂₀₀₃, a combination of R₂₀₀₃ and R₂₀₀₄, a combination of R₂₀₀₅ and R₂₀₀₆, a combination of R₂₀₀₆ and R₂₀₀₇, or a combination of R₂₀₀₇ and R₂₀₀₈ are mutually bonded to form a ring;
R₂₀₀₁ to R₂₀₀₈ as a substituent each independently represent the same as R₁ to R₈ as a substituent in the formula (2a);
B represents a cyclic structure represented by the formula (211) or (212), and the cyclic structure B is fused with adjacent cyclic structure(s) at any position(s);
px is 1, 2, 3 or 4;
when px is 2, 3 or 4, a plurality of cyclic structures B are mutually the same or different;
C represents a cyclic structure represented by the formula (211) or (212), and the cyclic structure C is fused with adjacent cyclic structure(s) at any position(s);
py is 1, 2, 3 or 4;
when py is 2, 3 or 4, a plurality of cyclic structures C are mutually the same or different; and
* represents a bonding position to a carbon atom in a six-membered ring in the formula (2).

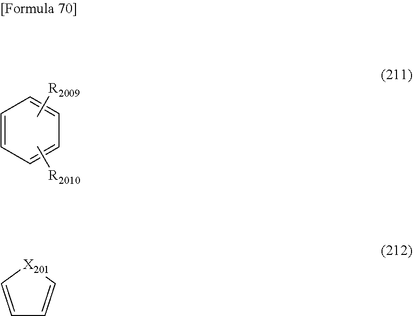

In the formula (211), R₂₀₀₉ and R₂₀₁₀ are each independently a hydrogen atom or a substituent, or bonded to a part of an adjacent cyclic structure to form a ring, or a combination of R₂₀₀₉ and R₂₀₁₀ are mutually bonded to form a ring.

In the formula (212), X₂₀₁ is CR₂₀₁₁R₂₀₁₂, NR₂₀₁₃, a sulfur atom, or an oxygen atom, and R₂₀₁₁, R₂₀₁₂ and R₂₀₁₃ are each independently a hydrogen atom or a substituent, or R₂₀₁₁ and R₂₀₁₂ are mutually bonded to form a ring.

R₂₀₀₉, R₂₀₁₀, R₂₀₁₁, R₂₀₁₂ and R₂₀₁₃ as a substituent each independently represent the same as R₁ to R₈ as a substituent in the formula (2a).

In the formula (211), R₂₀₀₉ and R₂₀₁₀ are each independently bonded to a part of an adjacent cyclic structure to form a ring, which specifically means any of (I) to (IV) below.

In the formula (211), a combination of R₂₀₀₉ and R₂₀₁₀ are mutually bonded to form a ring, which specifically means (V) below.

(I) When the cyclic structures represented by the formula (211) are adjacent to each other, between the two adjacent rings, at least one combination of the following are mutually bonded to form a ring: R₂₀₀₉ of one of the rings and R₂₀₀₉ of the other of the rings; R₂₀₀₉ of one of the rings and R₂₀₁₀ of the other of the rings; or R₂₀₁₀ of one of the rings and R₂₀₁₀ of the other of the rings.

(II) When the cyclic structure represented by the formula (211) and the benzene ring having R₂₅ to R₂₈ in the formula (2b) are adjacent to each other, between the two adjacent rings, at least one combination of the following are mutually bonded to form a ring: R₂₀₀₉ of one of the rings and R₂₅ of the other of the rings; R₂₀₀₉ of one of the rings and R₂₈ of the other of the rings; R₂₀₁₀ of one of the rings and R₂₅ of the other of the rings; or R₂₀₁₀ of one of the rings and R₂₈ of the other of the rings.

(III) When the cyclic structure represented by the formula (211) and the benzene ring having R₂₀₀₁ to R₂₀₀₄ in the formula (2c) are adjacent to each other, between the two adjacent rings, at least one combination of the following are mutually bonded to form a ring: R₂₀₀₉ of one of the rings and R₂₀₀₁ of the other of the rings; R₂₀₀₉ of one of the rings and R₂₀₀₄ of the other of the rings; R₂₀₁₀ of one of the rings and R₂₀₀₁ of the other of the rings; or R₂₀₁₀ of one of the rings and R₂₀₀₄ of the other of the rings.

(IV) When the cyclic structure represented by the formula (211) and the benzene ring having R₂₀₀₅ to R₂₀₀₈ in the formula (2c) are adjacent to each other, between the two adjacent rings, at least one combination of the following are mutually bonded to form a ring: R₂₀₀₉ of one of the rings and R₂₀₀₅ of the other of the rings; R₂₀₀₉ of one of the rings and R₂₀₀₈ of the other of the rings; R₂₀₁₀ of one of the rings and R₂₀₀₅ of the other of the rings; or R₂₀₁₀ of one of the rings and R₂₀₀₈ of the other of the rings.

(V) The combination of R₂₀₀₉ and R₂₀₁₀ of the cyclic structure represented by the formula (211) are mutually bonded to form a ring. In other words, (V) means that the combination of R₂₀₀₉ and R₂₀₁₀, which are bonded to the same ring, are mutually bonded to form a ring.

In the compound M2, it is preferable that Rx is each independently a hydrogen atom, an unsubstituted aryl group having 6 to 30 ring carbon atoms, an unsubstituted heterocyclic group having 5 to 30 ring atoms, or an unsubstituted alkyl group having 1 to 30 carbon atoms, and when Rx is an unsubstituted heterocyclic group having 5 to 30 ring atoms, Rx as an unsubstituted heterocyclic group having 5 to 30 ring atoms is a pyridyl group, pyrimidinyl group, triazinyl group, dibenzofuranyl group, or dibenzothienyl group.

Herein, the triazinyl group refers to a group obtained by excluding one hydrogen atom from 1,3,5-triazine, 1,2,4-triazine, or 1,2,3-triazine.

The triazinyl group is preferably a group obtained by excluding one hydrogen atom from 1,3,5-triazine.

In the compound M2, it is more preferable that Rx is each independently a hydrogen atom, an unsubstituted aryl group having 6 to 30 ring carbon atoms, an unsubstituted dibenzofuranyl group, or an unsubstituted dibenzothienyl group.

In the compound M2 of the exemplary embodiment, it is still more preferable that Rx is a hydrogen atom.

In the compound M2 of the exemplary embodiment, it is preferable that R₁ to R₈, R₂₁ to R₂₈, R₂₀₀₁ to R₂₀₀₈, R₂₀₀₉ to R₂₀₁₀, and R₂₀₁₁ to R₂₀₁₃ as a substituent are each independently an unsubstituted aryl group having 6 to 30 ring carbon atoms, an unsubstituted heterocyclic group having 5 to 30 ring atoms, or an unsubstituted alkyl group having 1 to 30 carbon atoms.

In the compound M2 of the exemplary embodiment, it is preferable that D₁ is a group represented by one of formulae (D-21) to (D-27) below.

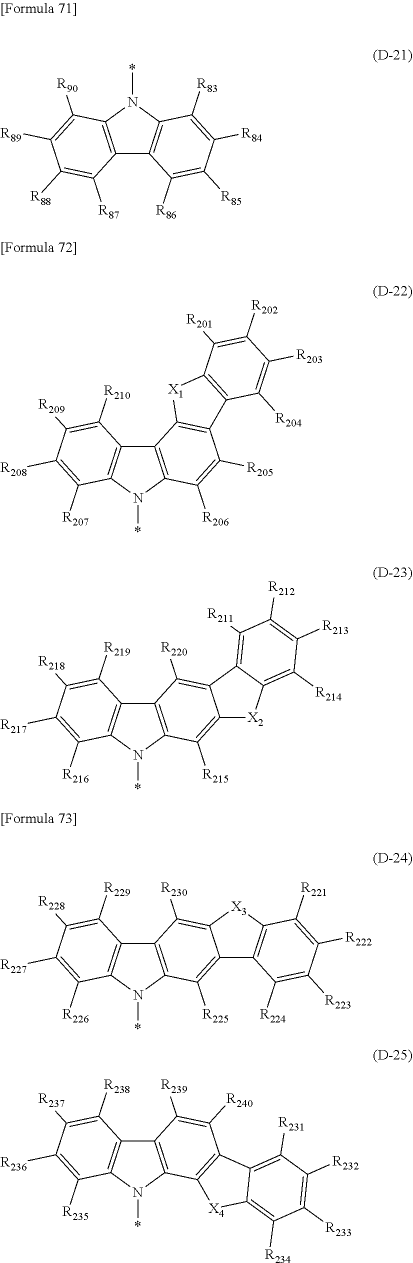

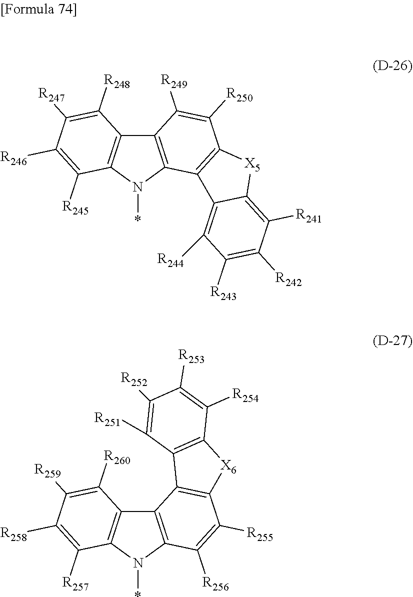

In the formula (D-21), R₈₃ to R₉₀ are each independently a hydrogen atom or a substituent.

In the formulae (D-21) to (D-27):
X₁ to X₆ are each independently an oxygen atom, a sulfur atom, or CR₁₅₁R₁₅₂;
R₁₅₁ and R₁₅₂ are each independently a hydrogen atom or a substituent, or R₁₅₁ and R₁₅₂ are mutually bonded to form a ring;
R₂₀₁ to R₂₆₀ are each independently a hydrogen atom or a substituent, or at least one combination of a combination of R₂₀₁ and R₂₀₂, a combination of R₂₀₂ and R₂₀₃, a combination of R₂₀₃ and R₂₀₄, a combination of R₂₀₅ and R₂₀₆, a combination of R₂₀₇ and R₂₀₈, a combination of R₂₀₈ and R₂₀₉, a combination of R₂₀₉ and R₂₁₀, a combination of R₂₁₁ and R₂₁₂, a combination of R₂₁₂ and R₂₁₃, a combination of R₂₁₃ and R₂₁₄, a combination of R₂₁₆ and R₂₁₇, a combination of R₂₁₇ and R₂₁₈, a combination of R₂₁₈ and R₂₁₉, a combination of R₂₂₁ and R₂₂₂, a combination of R₂₂₂ and R₂₂₃, a combination of R₂₂₃ and R₂₂₄, a combination of R₂₂₆ and R₂₂₇, a combination of R₂₂₇ and R₂₂₈, a combination of R₂₂₈ and R₂₂₉, a combination of R₂₃₁ and R₂₃₂, a combination of R₂₃₂ and R₂₃₃, a combination of R₂₃₃ and R₂₃₄, a combination of R₂₃₅ and R₂₃₆, a combination of R₂₃₆ and R₂₃₇, a combination of R₂₃₇ and R₂₃₈, a combination of R₂₃₉ and R₂₄₀, a combination of R₂₄₁ and R₂₄₂, a combination of R₂₄₂ and R₂₄₃, a combination of R₂₄₃ and R₂₄₄, a combination of R₂₄₅ and R₂₄₆, a combination of R₂₄₆ and R₂₄₇, a combination of R₂₄₇ and R₂₄₈, a combination of R₂₄₉ and R₂₅₀, a combination of R₂₅₁ and R₂₅₂, a combination of R₂₅₂ and R₂₅₃, a combination of R₂₅₃ and R₂₅₄, a combination of R₂₅₅ and R₂₅₆, a combination of R₂₅₇ and R₂₅₈, a combination of R₂₅₈ and R₂₅₉, or a combination of R₂₅₉ and R₂₆₀ are bonded to each other to form a ring;
R₈₃ to R₉₀, R₁₅₁, R₁₅₂ R₂₀₁ to R₂₆₀ as a substituent are each independently a halogen atom, a substituted or unsubstituted aryl group having 6 to 14 ring carbon atoms, a substituted or unsubstituted heterocyclic group having 5 to 14 ring atoms, a substituted or unsubstituted alkyl group having 1 to 6 carbon atoms, a substituted or unsubstituted alkyl halide group having 1 to 6 carbon atoms, a substituted or unsubstituted cycloalkyl group having 3 to 8 ring carbon atoms, a substituted or unsubstituted alkylsilyl group having 3 to 6 carbon atoms, a hydroxy group, a substituted or unsubstituted alkoxy group having 1 to 6 carbon atoms, a substituted or unsubstituted alkoxy halide group having 1 to 6 carbon atoms, a substituted or unsubstituted aryloxy group having 6 to 14 ring carbon atoms, a substituted or unsubstituted arylamino group having 6 to 28 ring carbon atoms, a substituted or unsubstituted alkylamino group having 2 to 12 carbon atoms, a thiol group, a substituted or unsubstituted alkylthio group having 1 to 6 carbon atoms, or a substituted or unsubstituted arylthio group having 6 to 14 ring carbon atoms; and
* represents a bonding position to a carbon atom in a six-membered ring in the formula (2).

In the compound M2 of the exemplary embodiment, it is also more preferable that D₁ is a group represented by the formula (D-21), (D-23), (D-24) or (D-25).

In the compound M2 of the exemplary embodiment, it is also still more preferable that D₁ is a group represented by the formula (D-21), (D-23), or (D-25).

In the compound M2 of the exemplary embodiment, it is preferable that R₈₃ to R₉₀, R₂₀₁ to R₂₆₀, R₁₅₁ and R₁₅₂ are each independently a hydrogen atom, an unsubstituted aryl group having 6 to 14 ring carbon atoms, an unsubstituted heterocyclic group having 5 to 14 ring atoms, or an unsubstituted alkyl group having 1 to 6 carbon atoms.

In the compound M2 of the exemplary embodiment, it is preferable that R₈₃ to R₉₀ and R₂₀₁ to R₂₆₀ are each a hydrogen atom.

In the compound M2 of the exemplary embodiment, it is preferable that R₁₅₁ and R₁₅₂ are each independently an unsubstituted aryl group having 6 to 14 ring carbon atoms, an unsubstituted heterocyclic group having 5 to 14 ring atoms, or an unsubstituted alkyl group having 1 to 6 carbon atoms.

### Compound Represented by Formula (22)

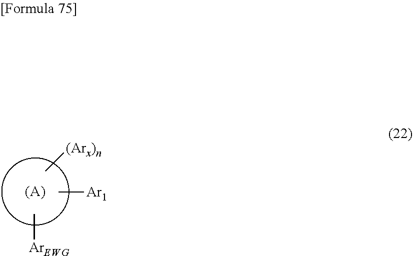

In the formula (22), Ar₁ is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by formulae (1a) to (1j) below.

In the formula (22), Ar_{EWG} is a substituted or unsubstituted heteroaryl group having 5 to 30 ring atoms that includes at least one nitrogen atom in a ring, or an aryl group having 6 to 30 ring carbon atoms that is substituted by at least one cyano group.

In the formula (22), Ar_{X} is each independently a hydrogen atom or a substituent, and Ar_{X} as a substituent is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by the formulae (1a) to (1j).

In the formula (22), n is 0, 1, 2, 3, 4 or 5, and when n is 2, 3, 4 or 5, a plurality of Ar_{X} are mutually the same or different.

In the formula (22), a ring (A) is a substituted or unsubstituted aromatic hydrocarbon ring or a substituted or unsubstituted heterocycle, the ring (A) is a five-membered ring, a six-membered ring, or a seven-membered ring. The ring (A) may be an aromatic hydrocarbon ring or a heterocycle. Ar₁ and Ar_{X} are bonded to respective ones of elements forming the ring (A).

In the formula (22), at least one of Ar or Ar_{X} is a group selected from the group consisting of groups represented by the formulae (1a) to (1j).

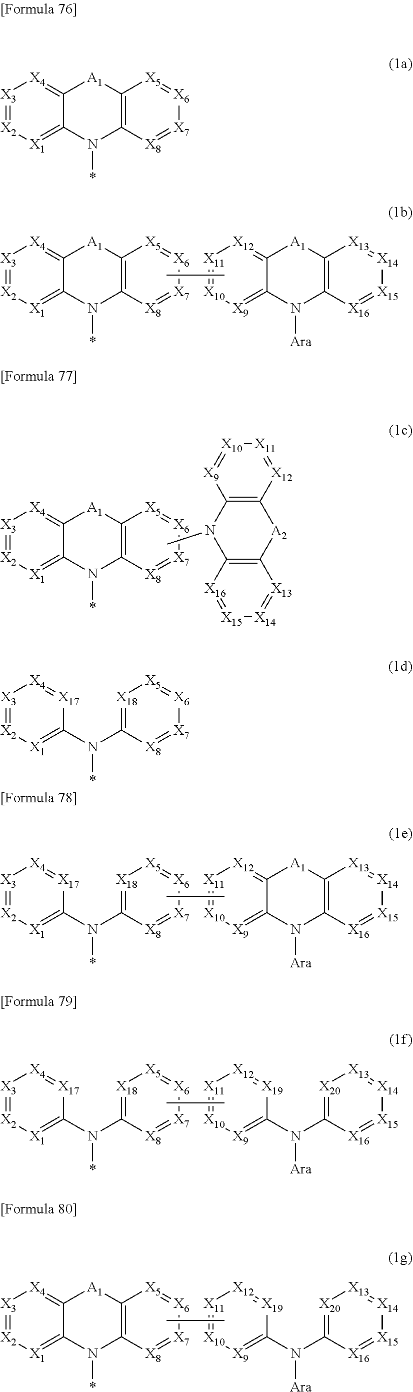

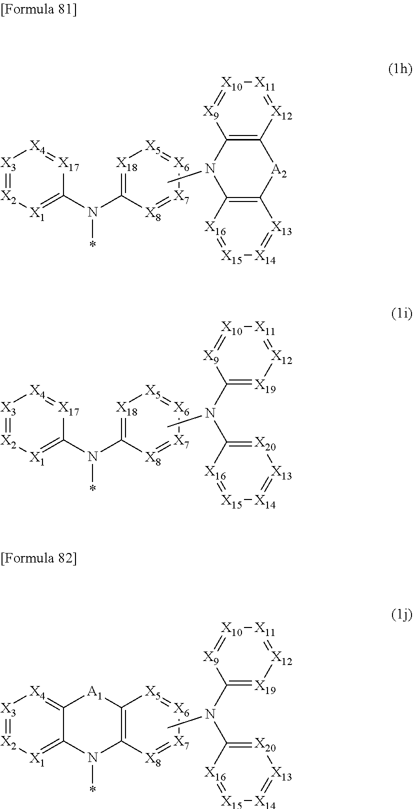

In the formulae (1a) to (1j), X₁ to X₂₀ are each independently a nitrogen atom (N) or a carbon atom bonded with R_{A1} (C—R_{A1}).

In the formula (1b), one of X₅ to X₈ is a carbon atom bonded to one of X₉ to X₁₂, and one of X₉ to X₁₂ is a carbon atom bonded to one of X₅ to X₈.

In the formula (1c), one of X₅ to X₈ is a carbon atom bonded to a nitrogen atom in a ring including A₂.

In the formula (1e), one of X₅ to X₈ and X₁₈ is a carbon atom bonded to one of X₉ to X₁₂, and one of X₉ to X₁₂ is a carbon atom bonded to one of X₅ to X₈ and X₁₈.

In the formula (1f), one of X₅ to X₈ and X₁₈ is a carbon atom bonded to one of X₉ to X₁₂ and X₁₉, and one of X₉ to X₁₂ and X₁₉ is a carbon atom bonded to one of X₅ to X₈ and X₁₈.

In the formula (1g), one of X₅ to X₈ is a carbon atom bonded to one of X₉ to X₁₂ and X₁₉, and one of X₉ to X₁₂ and X₁₉ is a carbon atom bonded to one of X₅ to X₈.

In the formula (1h), one of X₅ to X₈ and X₁₈ is a carbon atom bonded to a nitrogen atom in a ring including A₂.

In the formula (1i), one of X₅ to X₈ and X₁₈ is a carbon atom bonded to a nitrogen atom that links a ring including X₉ to X₁₂ and X₁₉ with a ring including X₁₃ to X₁₆ and X₂₀.

In the formula (1j), one of X₅ to X₈ is a carbon atom bonded to a nitrogen atom that links a ring including X₉ to X₁₂ and X₁₉ with a ring including X₁₃ to X₁₆ and X₂₀.

R_{A1} is each independently a hydrogen atom or a substituent, or at least one combination of combinations of a plurality of R_{A1} are mutually directly bonded to form a ring or bonded via a hetero atom to form a ring.

R_{A1} as a substituent is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group.

When a plurality of R_{A1} as a substituent are present, the plurality of R_{A1} are mutually the same or different.

In the formula (1a), when X₁ to X₈ are each a carbon atom bonded with R_{A1} (C—R_{A1}), a plurality of R_{A1} preferably form no ring.

In the formulae (1a) to (1j), * represents a bonding position to the ring (A).

In the formulae (1a) to (1j), A₁ and A₂ are each independently a single bond, an oxygen atom (O), a sulfur atom (S), C(R₂₀₂₁)(R₂₀₂₂), Si(R₂₀₂₃)(R₂₀₂₄), C(═O), S(═O), SO₂ or N(R₂₀₂₅). R₂₀₂₁ to R₂₀₂₅ are each independently a hydrogen atom or a substituent, and R₂₀₂₁ to R₂₀₂₅ as a substituent are each independently a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group.

In the formulae (1a) to (1j), Ara is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, and a substituted silyl group. Ara is preferably a substituted or unsubstituted aryl group having 6 to 30 ring carbon atoms or a substituted or unsubstituted heterocyclic group having 5 to 30 ring atoms.

The formula (1a) is represented by a formula (1aa) below when A₁ is a single bond, represented by a formula (1ab) below when A₁ is O, represented by a formula (1ac) below when A₁ is S, represented by a formula (1ad) below when A₁ is C(R₂₀₂₁)(R₂₀₂₂), represented by a formula (1ae) below when A₁ is Si(R₂₀₂₃)(R₂₀₂₄), represented by a formula (1af) below when A₁ is C(═O), represented by a formula (1ag) below when A₁ is S(═O), represented by a formula (1ah) below when A₁ is SO₂, and represented by a formula (1ai) below when A₁ is N(R₂₀₂₅). In the formulae (1aa) to (1ai), X₁ to X₈ and R₂₀₂₁ to R₂₀₂₅ represent the same as described above. Linkages between rings via A₁ and A₂ in the formulae (1b), (1c), (1e) and (1g) to (1j) are the same as those in the formulae (1aa) to (1ai). In the formula (1aa), when X₁ to X₈ are each a carbon atom bonded with R_{A1} (C—R_{A1}), a plurality of R_{A1} as a substituent preferably form no ring.

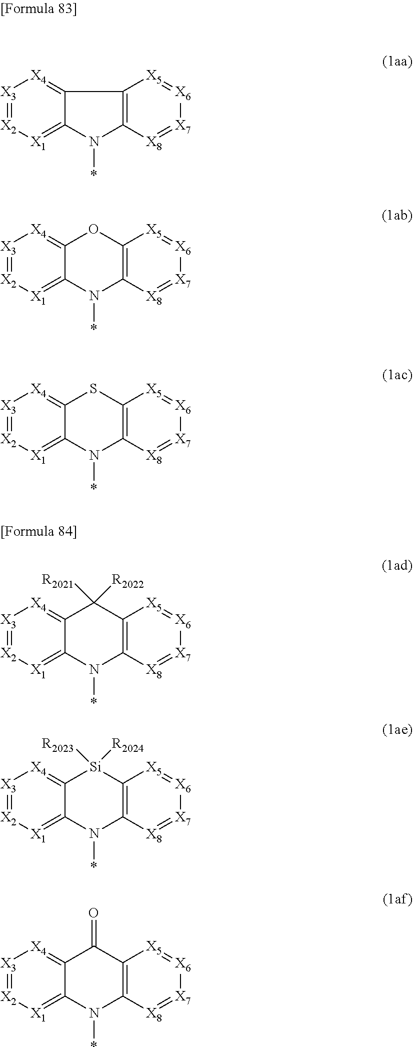

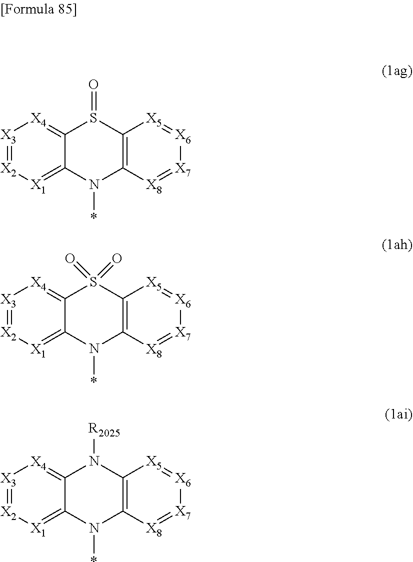

The compound M2 is preferably a compound represented by a formula (221) below.

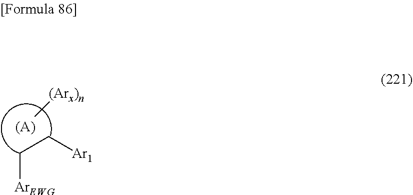

Ar₁, Ar_{EWG}, Ar_{X}, n and a ring (A) in the formula (221) respectively represent the same as Ar₁, Ar_{EWG}, Ar_{X}, n and the ring (A) in the formula (22).

The compound M2 is also preferably a compound represented by a formula (222) below.

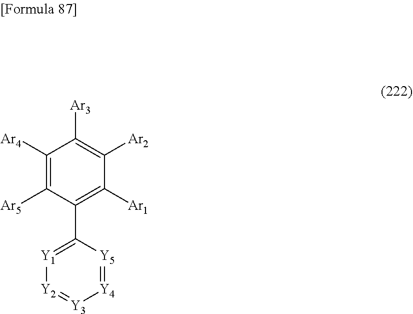

In the formula (222), Y₁ to Y₅ are each independently a nitrogen atom (N), a carbon atom bonded with a cyano group (C—CN), or a carbon atom bonded with R_{A2} (C—R_{A2}), and at least one of Y₁ to Y₅ is N or C—CN. A plurality of R_{A2} are mutually the same or different. R_{A2} is each independently a hydrogen atom or a substituent, and R_{A2} as a substituent is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group.

A plurality of R_{A2} are mutually the same or different.

In the formula (222), Ar₁ represents the same as Ar₁ in the formula (22).

In the formula (222), Ar₂ to Ar₅ are each independently a hydrogen atom or a substituent, and Ar₂ to Ar₅ as a substituent are each independently a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by the formulae (1a) to (1c).

In the formula (222), at least one of Ar₁ to Ar₅ is a group selected from the group consisting of groups represented by the formulae (1a) to (1c).

The compound M2 is also preferably a compound represented by a formula (11aa), (11bb) or (11cc) below.

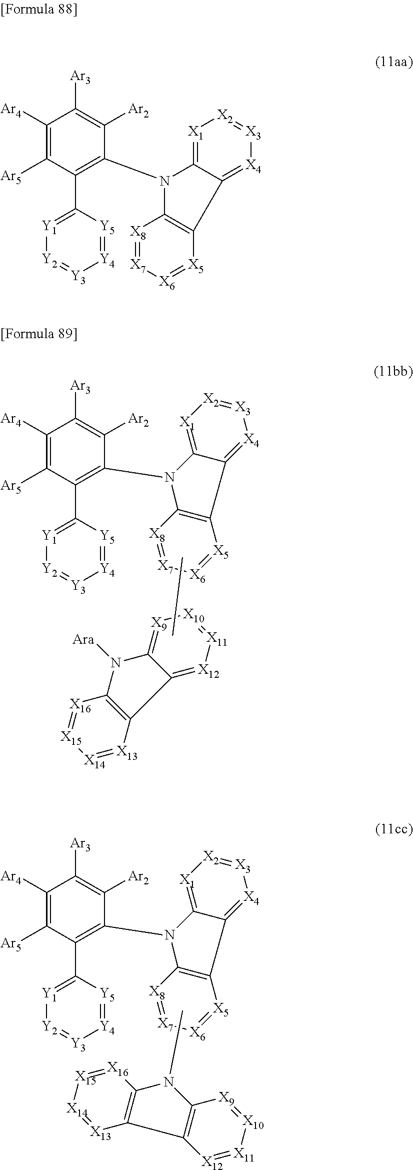

In the formulae (11 aa), (11 bb) and (11 cc), Y₁ to Y₅, R_{A2}, Ar₂ to Ar₅, X₁ to X₁₆, R_{A1} and Ara respectively represent the same as the above-described Y₁ to Y₅, R_{A2}, Ar₂ to Ar₅, X₁ to X₁₆, R_{A1} and Ara.

The compound M2 is exemplified by a compound represented by a formula (23) below.

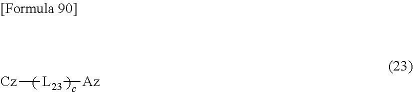

In the formula (23):
Az is a cyclic structure selected from the group consisting of a substituted or unsubstituted pyridine ring, a substituted or unsubstituted pyrimidine ring, a substituted or unsubstituted triazine ring, and a substituted or unsubstituted pyrazine ring;
c is 0, 1, 2, 3, 4 or 5;
when c is 0, Cz and Az are bonded by a single bond;
when c is 1, 2, 3, 4 or 5, L23 is a linking group selected from the group consisting of a substituted or unsubstituted arylene group having 6 to 30 ring carbon atoms, and a substituted or unsubstituted heteroarylene group having 5 to 30 ring atoms;
when c is 2, 3, 4 or 5, a plurality of L₂ are mutually the same or different;
the plurality of L₂ are mutually bonded to form a ring or not bonded; and
Cz is represented by a formula (23a) below.

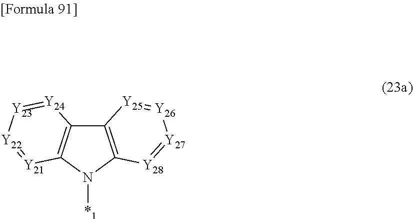

In the formula (23a):
Y₂₁ to Y₂₈ are each independently a nitrogen atom or CR_{A3};
R_{A3} is each independently a hydrogen atom or a substituent, or at least one combination of combinations of a plurality of R_{A3} are mutually bonded to form a ring;
each R_{A3} as a substituent is independently a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group;
a plurality of R_{A3} are mutually the same or different; and
*1 represents a bonding position to a carbon atom in a structure of a linking group represented by L₂₃, or a bonding position to a carbon atom in a cyclic structure represented by Az.
Y₂₁ to Y₂₈ are also preferably CR_{A3}.

In the formula (23), c is preferably 0 or 1.

Cz is also preferably represented by a formula (23b), (23c) or (23d) below.

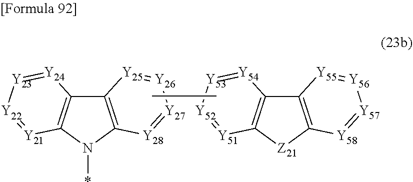

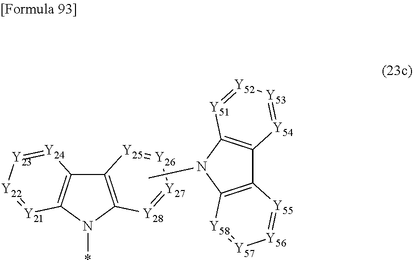

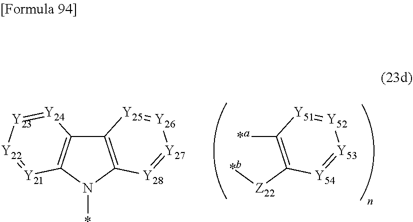

In the formulae (23b), (23c) and (23d), Y₂₁ to Y₂₈ and Y₅₁ to Y₅₈ are each independently a nitrogen atom or CR_{A4}.

In the formula (23b), at least one of Y₂₅ to Y₂₈ is a carbon atom bonded to one of Y₅₁ to Y₅₄, and at least one of Y₅₁ to Y₅₄ is a carbon atom bonded to one of Y₂₅ to Y₂₈.

In the formula (23c), at least one of Y₂₅ to Y₂₈ is a carbon atom bonded to a nitrogen atom in a five-membered ring of a nitrogen-containing fused ring including Y₅₁ to Y₅₈.

In the formula (23d), *a and *b each represent a bonding position to one of Y₂₁ to Y₂₈, at least one of Y₂₅ to Y₂₈ is the bonding position represented by *a, and at least one of Y₂₅ to Y₂₈ is the bonding position represented by *b.

n is 1, 2, 3 or 4;

R_{A4} is each independently a hydrogen atom or a substituent, or at least one combination of combinations of a plurality of R_{A4} are mutually bonded to form a ring; each R_{A4} as a substituent is independently a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group;

a plurality of R_{A4} are mutually the same or different;

Z₂₁ and Z₂₂ are each independently any one selected from the group consisting of an oxygen atom, a sulfur atom, NR₄₅ and CR₄₆R₄₇;

R₄₅ is a hydrogen atom or a substituent;

R₄₆ and R₄₇ are each independently a hydrogen atom or a substituent, or a combination of R₄₆ and R₄₇ are mutually bonded to form a ring;

R₄₅, R₄₆ and R₄₇ as a substituent are each independently a substituent selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, <<nret>>

a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group;

a plurality of R₄₅ are mutually the same or different;

a plurality of R₄₆ are mutually the same or different;

a plurality of R₄₇ are mutually the same or different; and

* represents a bonding position to a carbon atom in a structure of a linking group represented by L₂₃, or a bonding position to a carbon atom in a cyclic structure represented by Az.

Z₂₁ is preferably NR₄₅.

When Z₂₁ is NR₄₅, R₄₅ is preferably a substituted or unsubstituted aryl group having 6 to 30 ring carbon atoms.

Z₂₂ is preferably NR₄₅.

When Z₂₂ is NR₄₅, R₄₅ is preferably a substituted or unsubstituted aryl group having 6 to 30 ring carbon atoms.

Y₅₁ to Y₅₈ are preferably CR_{A4}, provided that at least one of Y₅₁ to Y₅₈ is a carbon atom bonded to a cyclic structure represented by the formula (23a).

It is also preferable that Cz is represented by the formula (23d) and n is 1.

Az is preferably a cyclic structure selected from the group consisting of a substituted or unsubstituted pyrimidine group and a substituted or unsubstituted triazine group.

Az is a cyclic structure selected from the group consisting of a substituted pyrimidine ring and a substituted triazine ring, in which a substituent for each of the substituted pyrimidine ring and the substituted triazine ring is more preferably a group selected from the group consisting of a substituted or unsubstituted aryl group having 6 to 30 ring carbon atoms and a substituted or unsubstituted heteroaryl group having 5 to 30 ring atoms, still more preferably a substituted or unsubstituted aryl group having 6 to 30 ring carbon atoms.

When a pyrimidine ring and a triazine ring as Az have a substituted or unsubstituted aryl group as a substituent, the aryl group preferably has 6 to 20 ring carbon atoms, more preferably 6 to 14 ring carbon atoms, still more preferably 6 to 12 ring carbon atoms.

When Az has a substituted or unsubstituted aryl group as a substituent, the substituent is preferably a substituent selected from the group consisting of a substituted or unsubstituted phenyl group, a substituted or unsubstituted biphenyl group, a substituted or unsubstituted naphthyl group, a substituted or unsubstituted phenanthryl group, a substituted or unsubstituted terphenyl group, and a substituted or unsubstituted fluorenyl group, more preferably a substituent selected from the group consisting of a substituted or unsubstituted phenyl group, a substituted or unsubstituted biphenyl group, and a substituted or unsubstituted naphthyl group.

When Az has a substituted or unsubstituted heteroaryl group as a substituent, the substituent is preferably a substituent selected from the group consisting of a substituted or unsubstituted carbazolyl group, a substituted or unsubstituted dibenzofuranyl group, and a substituted or unsubstituted dibenzothiophenyl group.

It is preferable that R_{A4} is each independently a hydrogen atom or a substituent, and R_{A4} as a substituent is a substituent selected from the group consisting of a substituted or unsubstituted aryl group having 6 to 30 ring carbon atoms and a substituted or unsubstituted heteroaryl group having 5 to 30 ring atoms.

When R_{A4} as a substituent is a substituted or unsubstituted aryl group having 6 to 30 ring carbon atoms, R_{A4} as a substituent is preferably a substituent selected from the group consisting of a substituted or unsubstituted phenyl group, a substituted or unsubstituted biphenyl group, a substituted or unsubstituted naphthyl group, a substituted or unsubstituted phenanthryl group, a substituted or unsubstituted terphenyl group, and a substituted or unsubstituted fluorenyl group, more preferably a substituent selected from the group consisting of a substituted or unsubstituted phenyl group, a substituted or unsubstituted biphenyl group, and a substituted or unsubstituted naphthyl group.

When R_{A4} as a substituent is a substituted or unsubstituted heteroaryl group having 5 to 30 ring atoms, R_{A4} as a substituent is preferably a substituent selected from the group consisting of a substituted or unsubstituted carbazolyl group, a substituted or unsubstituted dibenzofuranyl group, and a substituted or unsubstituted dibenzothiophenyl group.

R₄₅, R₄₆ and R₄₇ as a substituent are preferably each independently a substituent selected from the group consisting of a substituted or unsubstituted aryl group having 6 to 30 ring carbon atoms, a substituted or unsubstituted heteroaryl group having 5 to 30 ring atoms, and a substituted or unsubstituted alkyl group having 1 to 30 carbon atoms.

### Manufacturing Method of Compound M2 in Embodiment

The compound M2 of the exemplary embodiment can be manufactured by a known method.

### Specific Examples of Compound M2

Specific examples of the compound M2 in the exemplary embodiment includes compounds below. However, the invention is by no means limited to the specifically listed compounds.

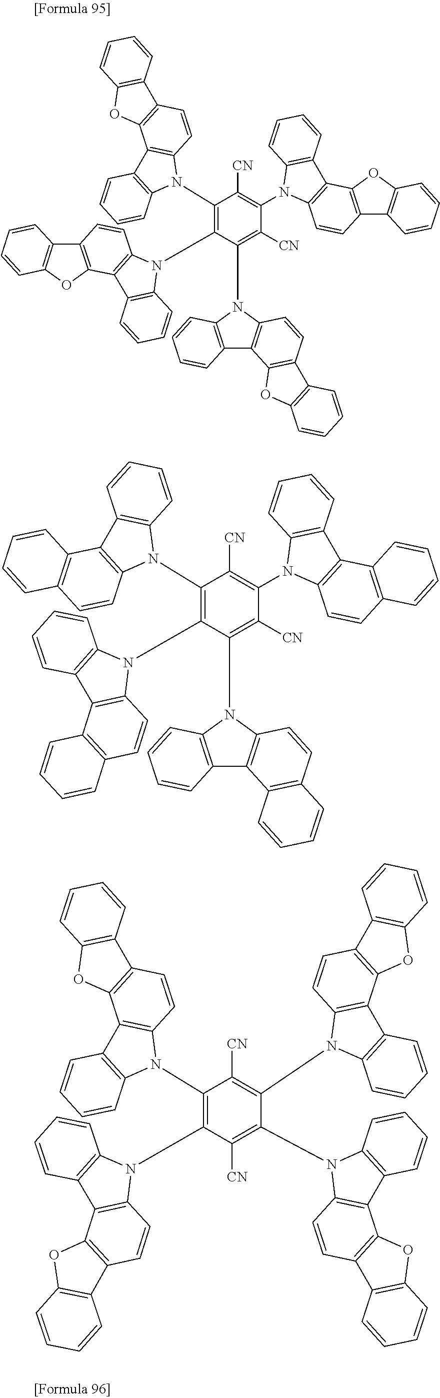

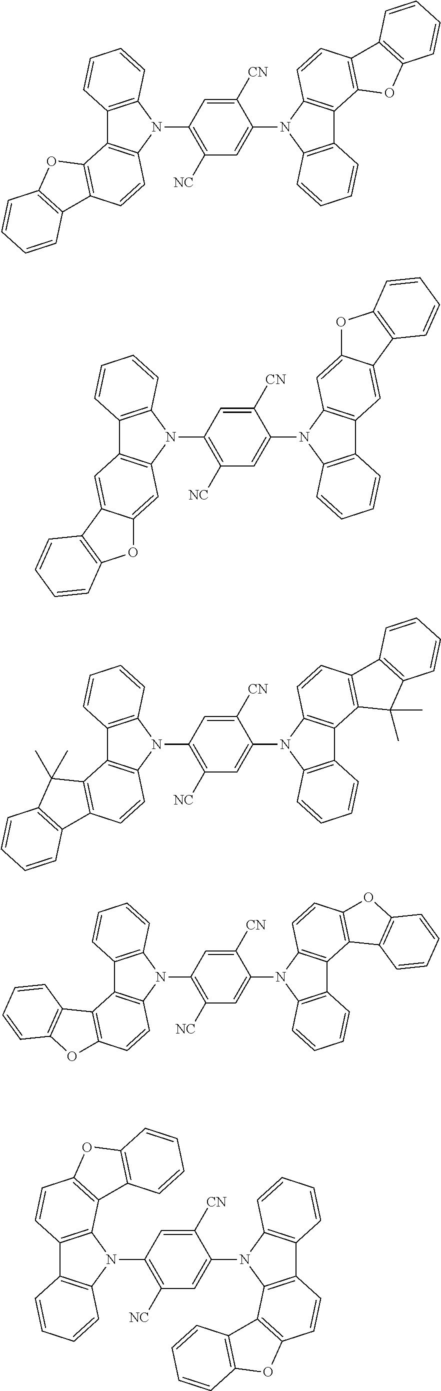 [SMILES: N#Cc1cc(-n2c3ccccc3c3c4oc5ccccc5c4ccc32)c(C#N)cc1-n1c2ccccc2c2c3oc4ccccc4c3ccc21 ; N#Cc1cc(-n2c3ccccc3c3cc4c(cc32)oc2ccccc24)c(C#N)cc1-n1c2ccccc2c2cc3c(cc21)oc1ccccc13 ; CC1(C)c2ccccc2-c2ccc3c(c21)c1ccccc1n3-c1cc(C#N)c(-n2c3ccccc3c3c4c(ccc32)-c2ccccc2C4(C)C)cc1C#N ; N#Cc1cc(-n2c3ccccc3c3c4c(ccc32)oc2ccccc24)c(C#N)cc1-n1c2ccccc2c2c3c(ccc21)oc1ccccc13 ; N#Cc1cc(-n2c3ccccc3c3ccc4oc5ccccc5c4c32)c(C#N)cc1-n1c2ccccc2c2ccc3oc4ccccc4c3c21]

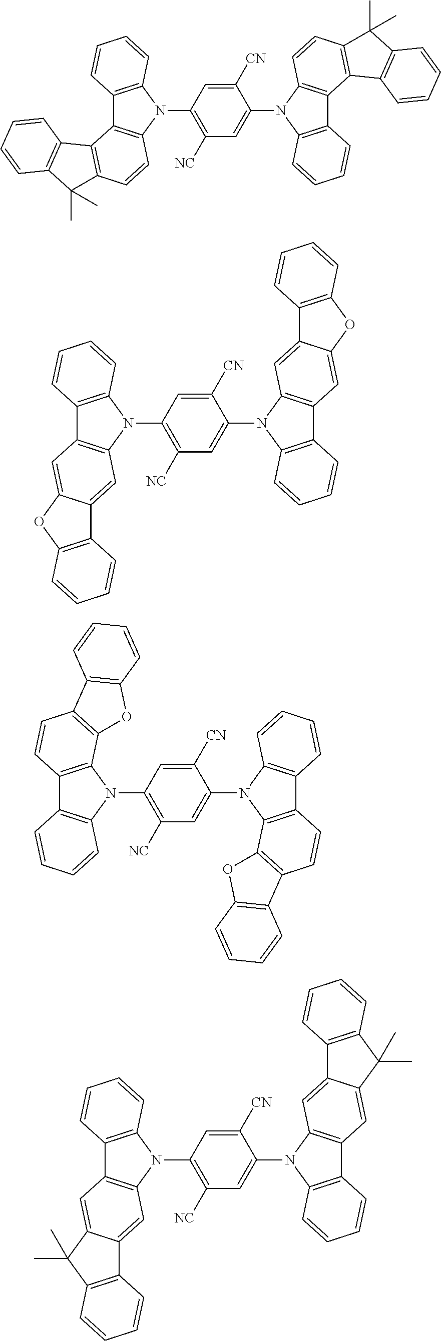

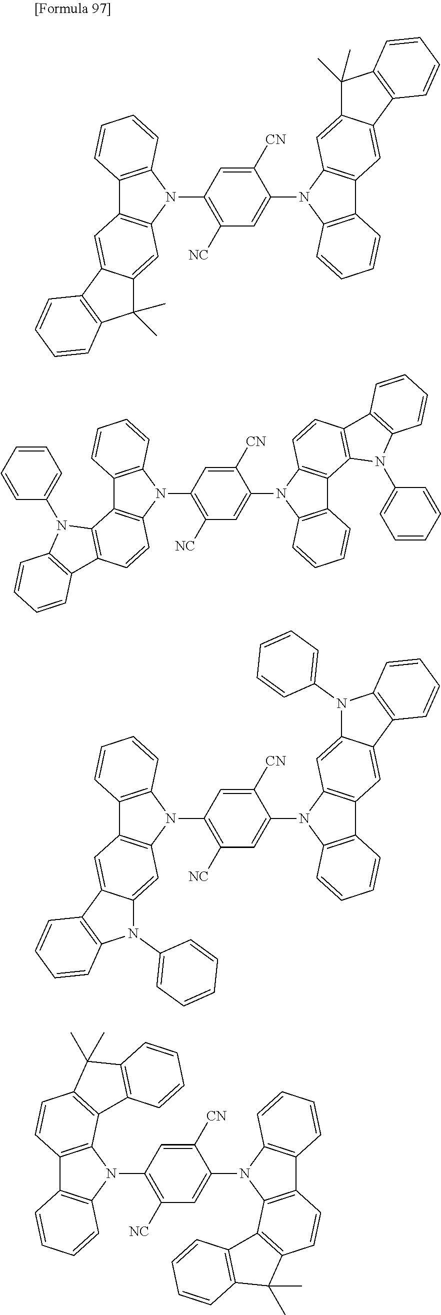 [SMILES: CC1(C)c2ccccc2-c2cc3c4ccccc4n(-c4cc(C#N)c(-n5c6ccccc6c6cc7c(cc65)C(C)(C)c5ccccc5-7)cc4C#N)c3cc21 ; N#Cc1cc(-n2c3ccccc3c3c2ccc2c4ccccc4n(-c4ccccc4)c23)c(C#N)cc1-n1c2ccccc2c2c1ccc1c3ccccc3n(-c3ccccc3)c12 ; N#Cc1cc(-n2c3ccccc3c3cc4c5ccccc5n(-c5ccccc5)c4cc32)c(C#N)cc1-n1c2ccccc2c2cc3c4ccccc4n(-c4ccccc4)c3cc21 ; CC1(C)c2ccccc2-c2c1ccc1c3ccccc3n(-c3cc(C#N)c(-n4c5ccccc5c5ccc6c(c54)-c4ccccc4C6(C)C)cc3C#N)c21]

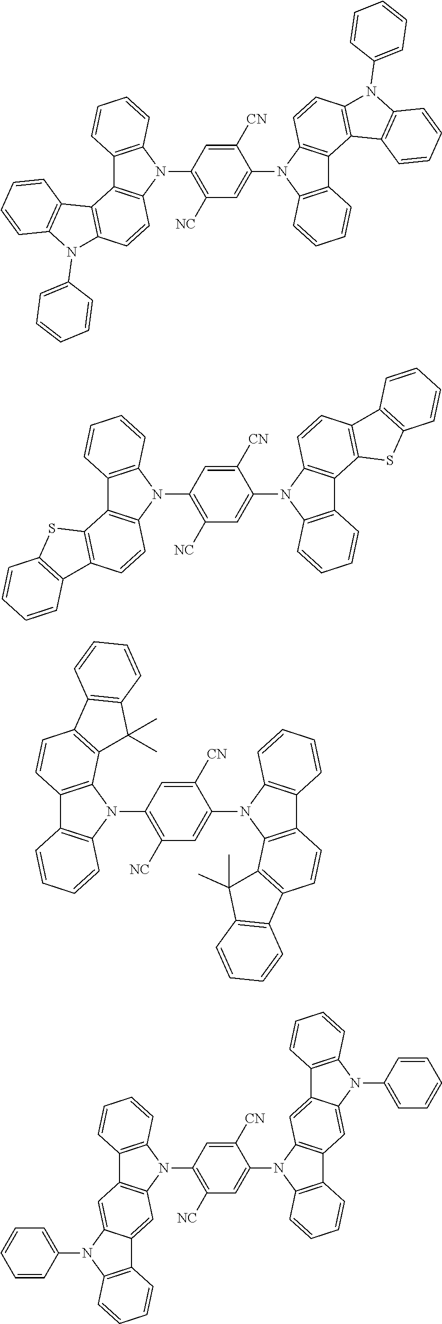 [SMILES: N#Cc1cc(-n2c3ccccc3c3c4c5ccccc5n(-c5ccccc5)c4ccc32)c(C#N)cc1-n1c2ccccc2c2c3c4ccccc4n(-c4ccccc4)c3ccc21 ; N#Cc1cc(-n2c3ccccc3c3c4sc5ccccc5c4ccc32)c(C#N)cc1-n1c2ccccc2c2c3sc4ccccc4c3ccc21 ; CC1(C)c2ccccc2-c2ccc3c4ccccc4n(-c4cc(C#N)c(-n5c6ccccc6c6ccc7c(c65)C(C)(C)c5ccccc5-7)cc4C#N)c3c21 ; N#Cc1cc(-n2c3ccccc3c3cc4c(cc32)c2ccccc2n4-c2ccccc2)c(C#N)cc1-n1c2ccccc2c2cc3c(cc21)c1ccccc1n3-c1ccccc1]

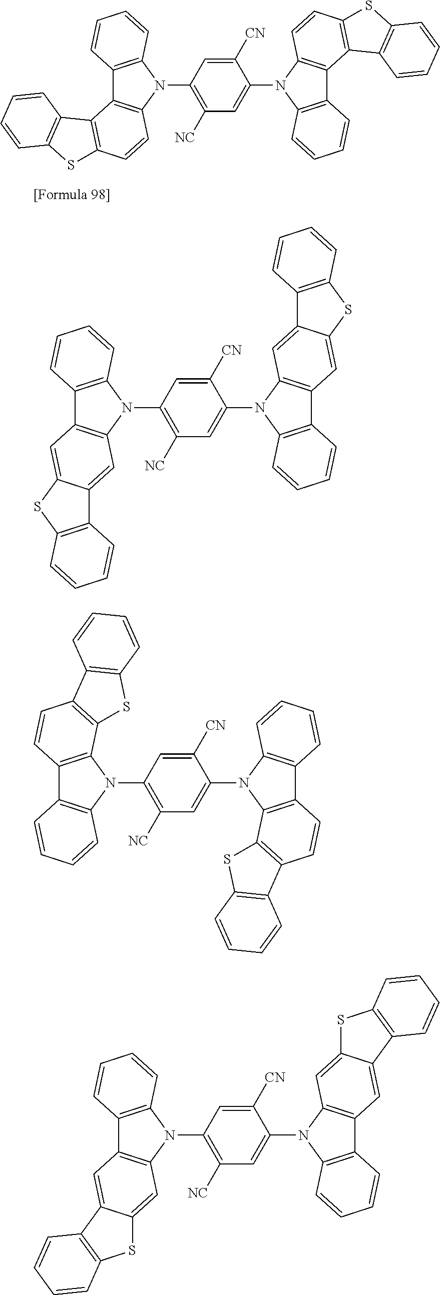

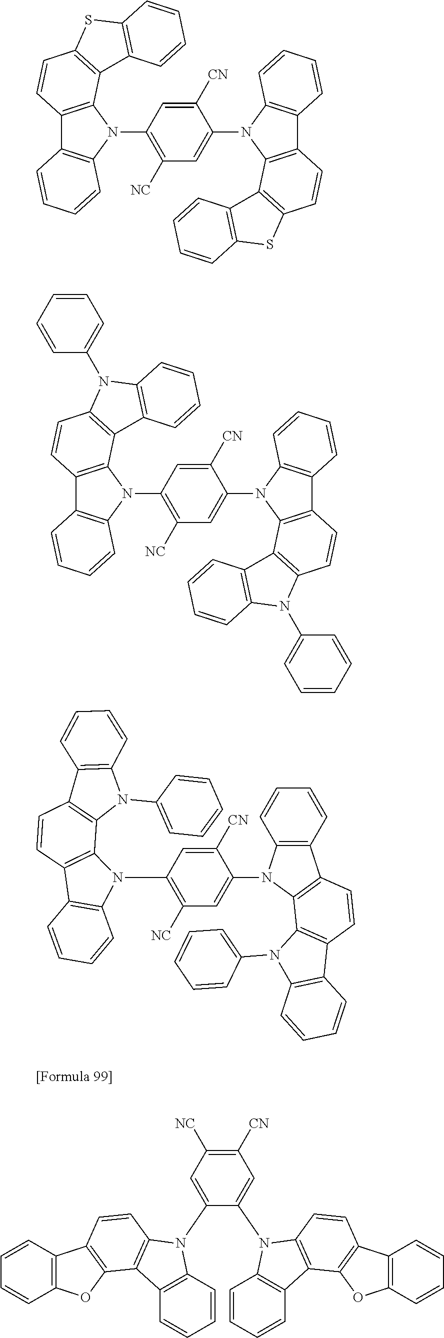 [SMILES: N#Cc1cc(-n2c3ccccc3c3ccc4sc5ccccc5c4c32)c(C#N)cc1-n1c2ccccc2c2ccc3sc4ccccc4c3c21 ; N#Cc1cc(-n2c3ccccc3c3ccc4c(c5ccccc5n4-c4ccccc4)c32)c(C#N)cc1-n1c2ccccc2c2ccc3c(c4ccccc4n3-c3ccccc3)c21 ; N#Cc1cc(-n2c3ccccc3c3ccc4c5ccccc5n(-c5ccccc5)c4c32)c(C#N)cc1-n1c2ccccc2c2ccc3c4ccccc4n(-c4ccccc4)c3c21 ; N#Cc1cc(-n2c3ccccc3c3c4oc5ccccc5c4ccc32)c(-n2c3ccccc3c3c4oc5ccccc5c4ccc32)cc1C#N]

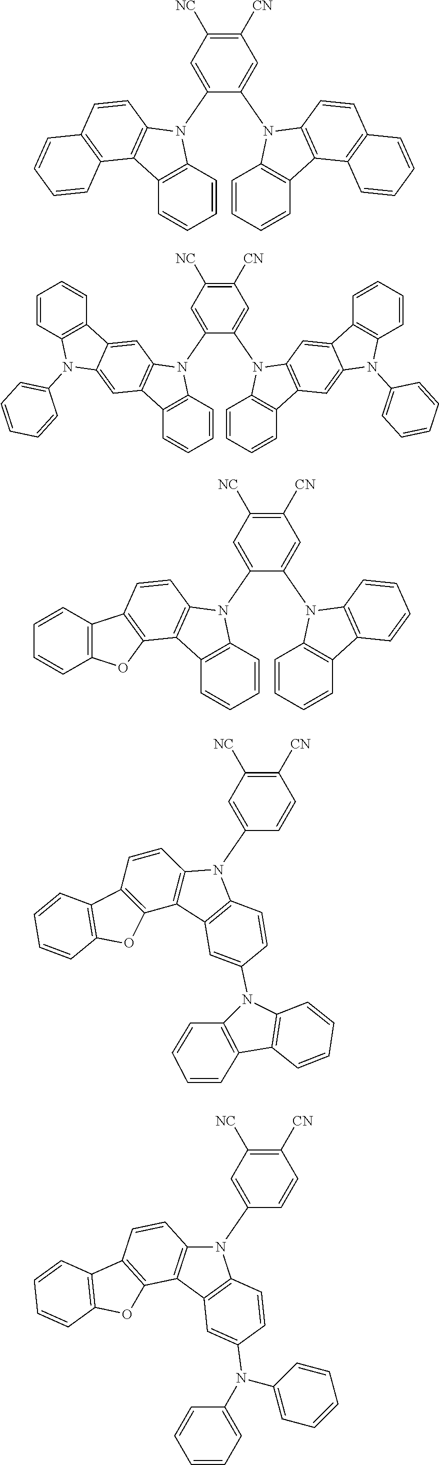

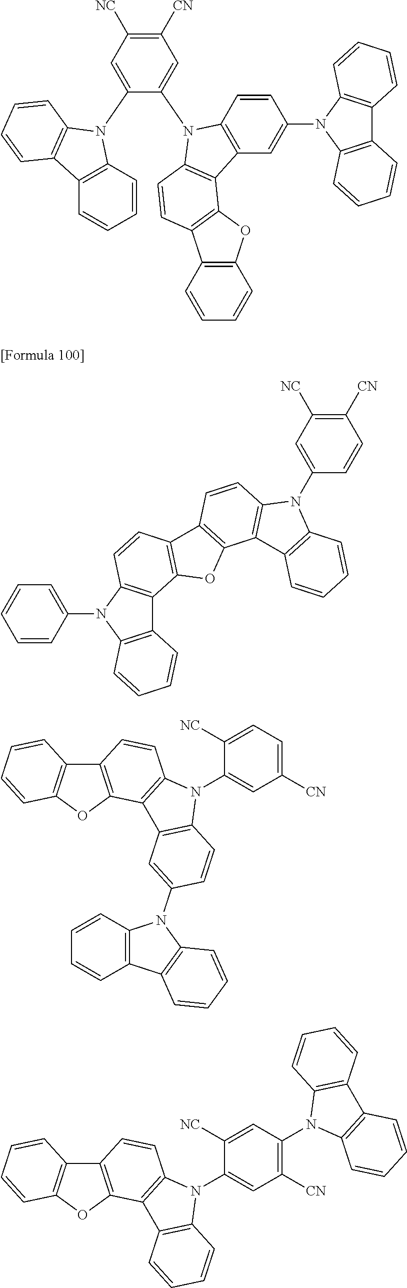 [SMILES: N#Cc1cc(-n2c3ccccc3c3ccccc32)c(-n2c3ccc(-n4c5ccccc5c5ccccc54)cc3c3c4oc5ccccc5c4ccc32)cc1C#N ; N#Cc1ccc(-n2c3ccccc3c3c4oc5c(ccc6c5c5ccccc5n6-c5ccccc5)c4ccc32)cc1C#N ; N#Cc1ccc(C#N)c(-n2c3ccc(-n4c5ccccc5c5ccccc54)cc3c3c4oc5ccccc5c4ccc32)c1 ; N#Cc1cc(-n2c3ccccc3c3c4oc5ccccc5c4ccc32)c(C#N)cc1-n1c2ccccc2c2ccccc21]

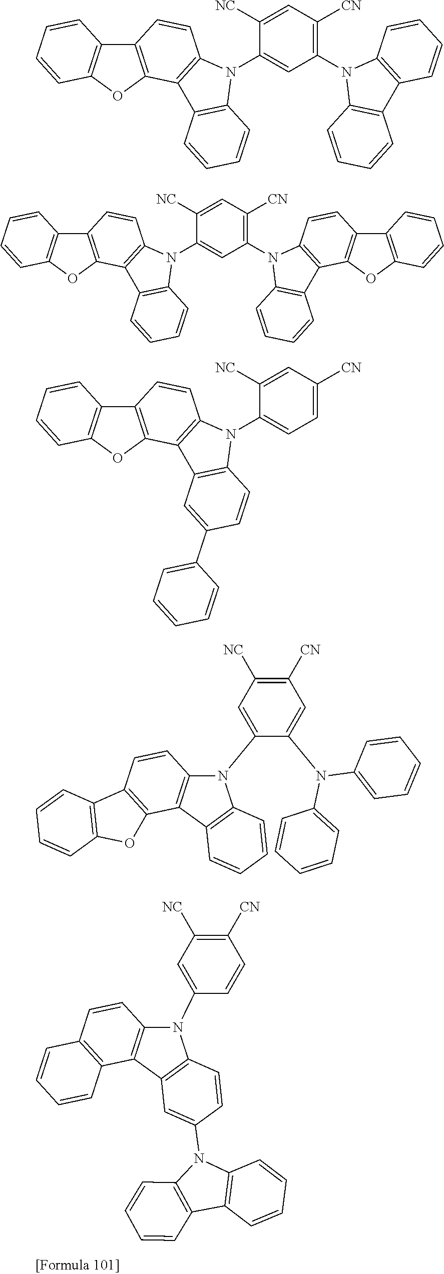

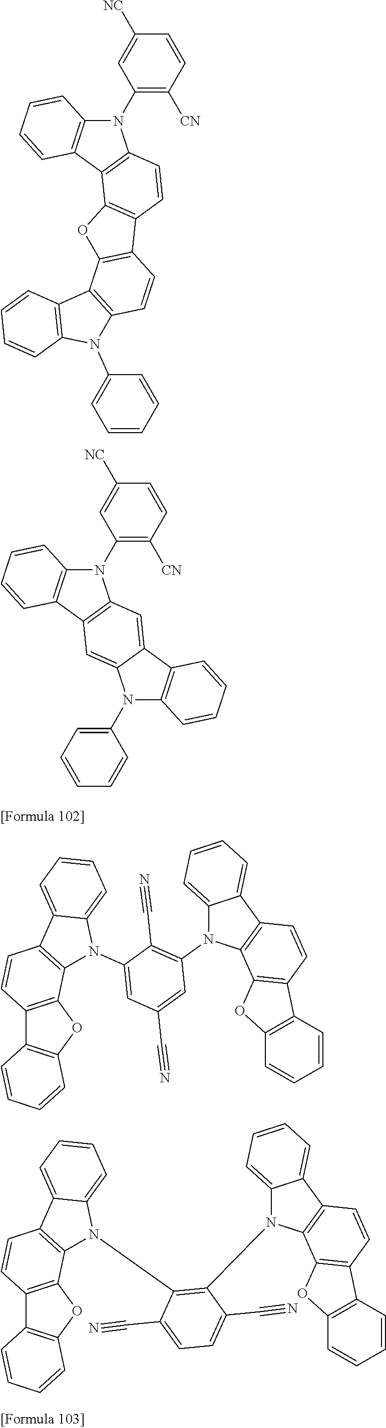

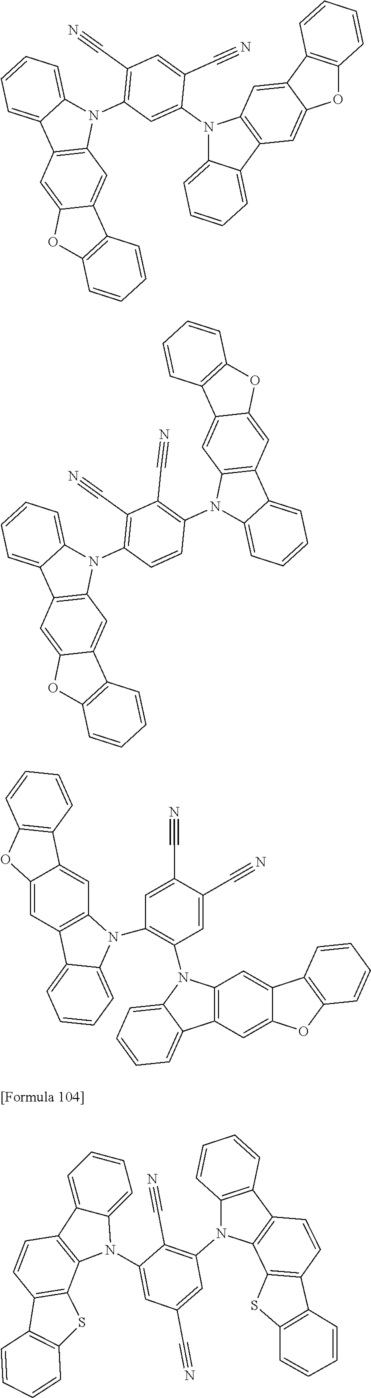 [SMILES: N#Cc1cc(C#N)c(-n2c3ccccc3c3cc4oc5ccccc5c4cc32)cc1-n1c2ccccc2c2cc3oc4ccccc4c3cc21 ; N#Cc1c(-n2c3ccccc3c3cc4oc5ccccc5c4cc32)ccc(-n2c3ccccc3c3cc4oc5ccccc5c4cc32)c1C#N ; N#Cc1cc(-n2c3ccccc3c3cc4oc5ccccc5c4cc32)c(-n2c3ccccc3c3cc4oc5ccccc5c4cc32)cc1C#N ; N#Cc1cc(-n2c3ccccc3c3ccc4c5ccccc5sc4c32)c(C#N)c(-n2c3ccccc3c3ccc4c5ccccc5sc4c32)c1]

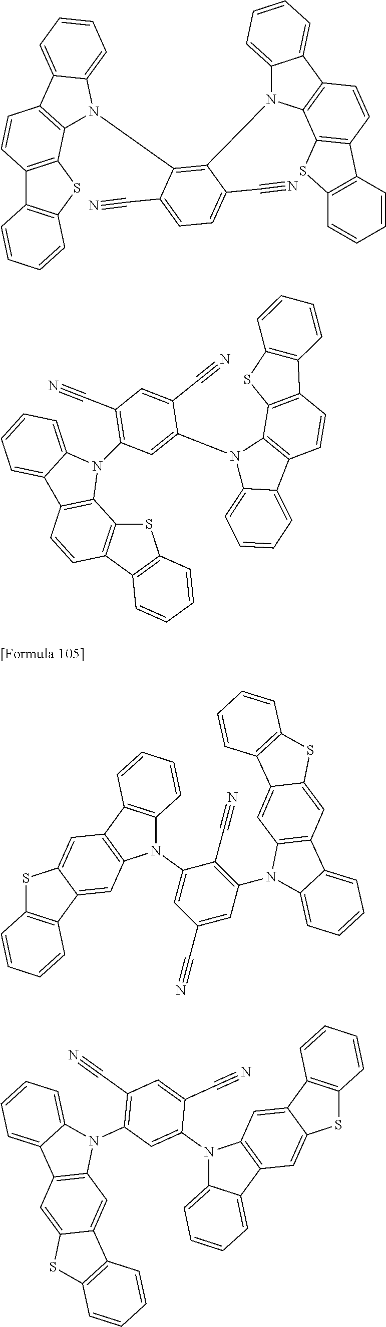 [SMILES: N#Cc1ccc(C#N)c(-n2c3ccccc3c3ccc4c5ccccc5sc4c32)c1-n1c2ccccc2c2ccc3c4ccccc4sc3c21 ; N#Cc1cc(C#N)c(-n2c3ccccc3c3ccc4c5ccccc5sc4c32)cc1-n1c2ccccc2c2ccc3c4ccccc4sc3c21 ; N#Cc1cc(-n2c3ccccc3c3cc4sc5ccccc5c4cc32)c(C#N)c(-n2c3ccccc3c3cc4sc5ccccc5c4cc32)c1 ; N#Cc1cc(C#N)c(-n2c3ccccc3c3cc4sc5ccccc5c4cc32)cc1-n1c2ccccc2c2cc3sc4ccccc4c3cc21]

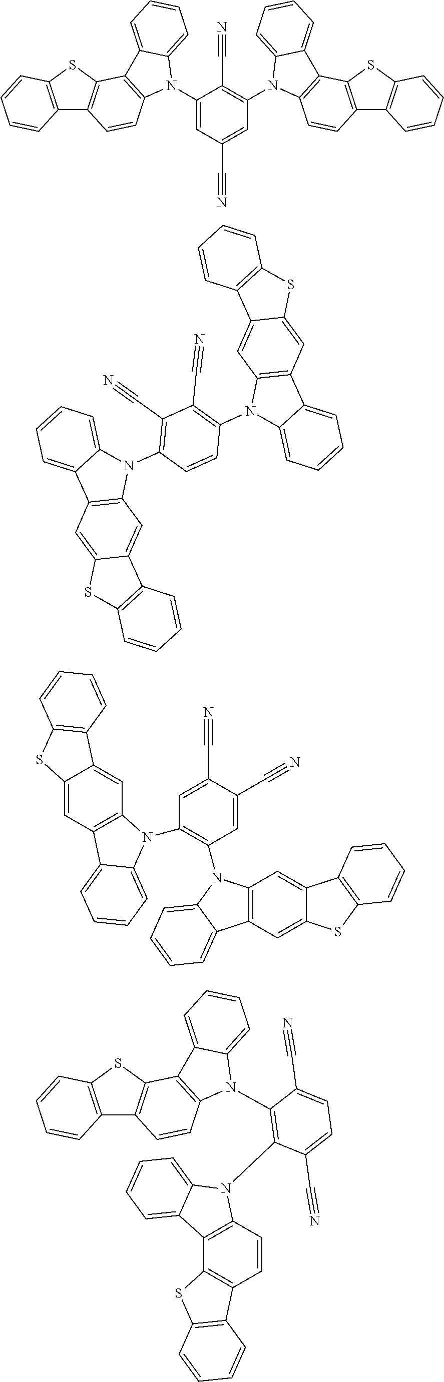

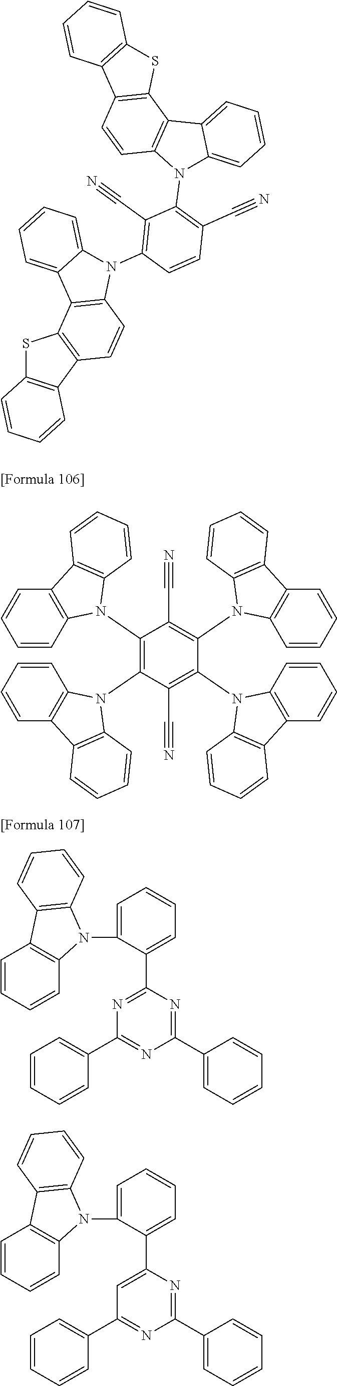

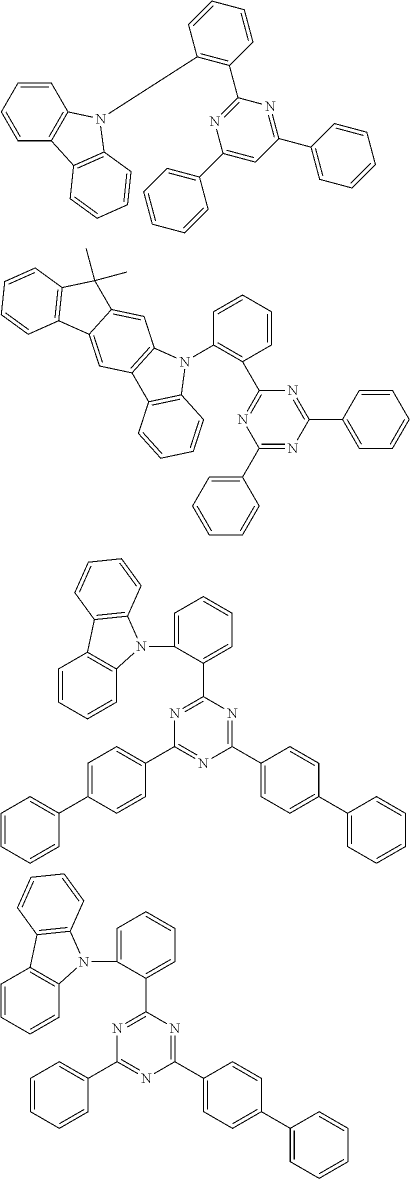

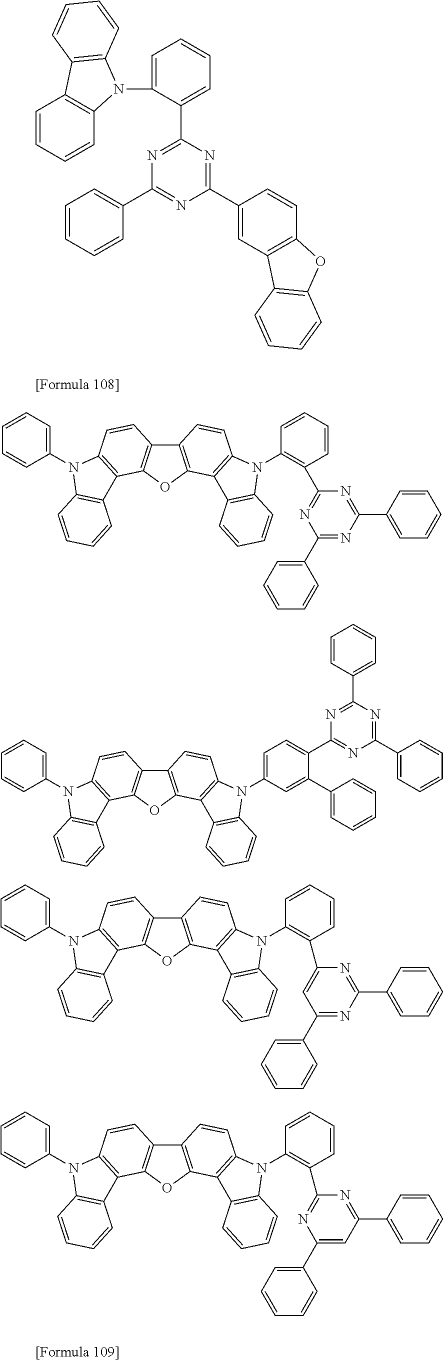

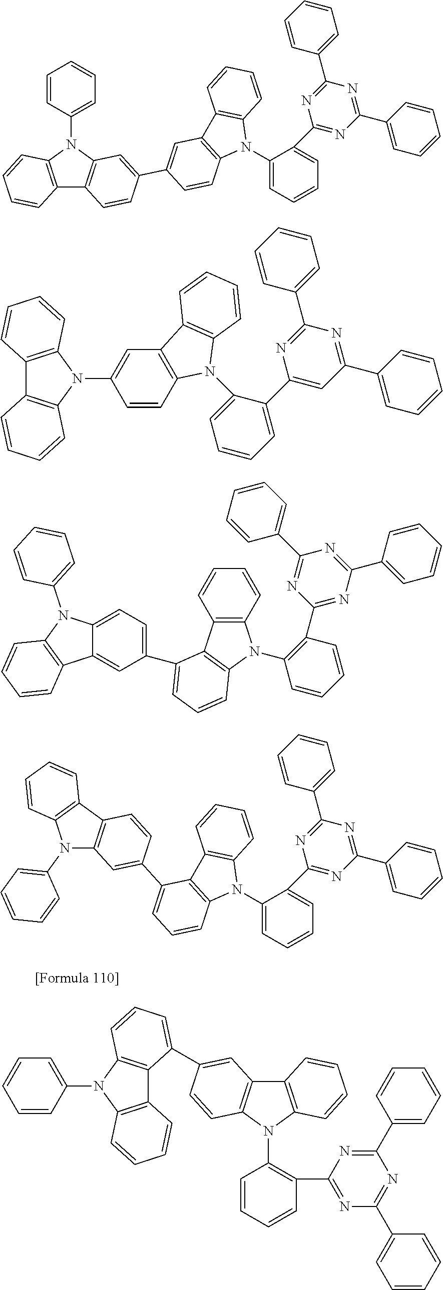

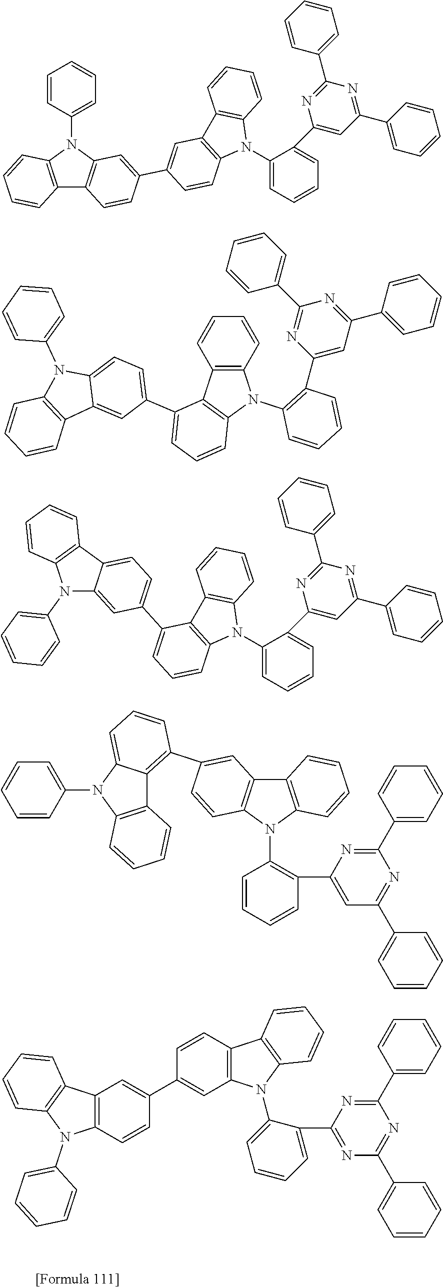

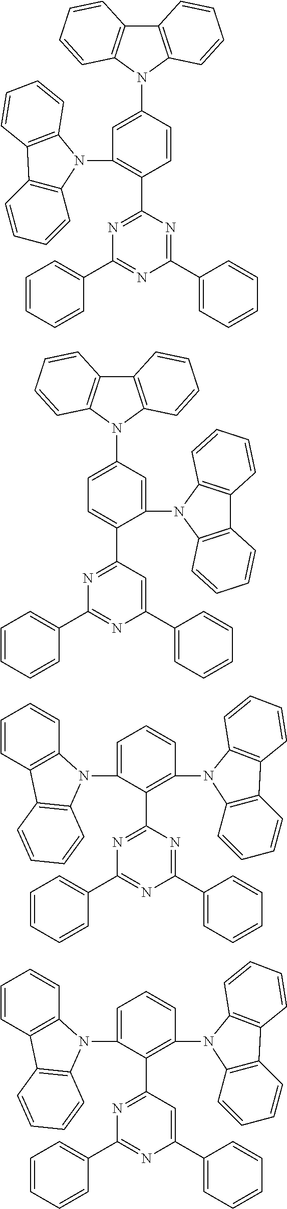

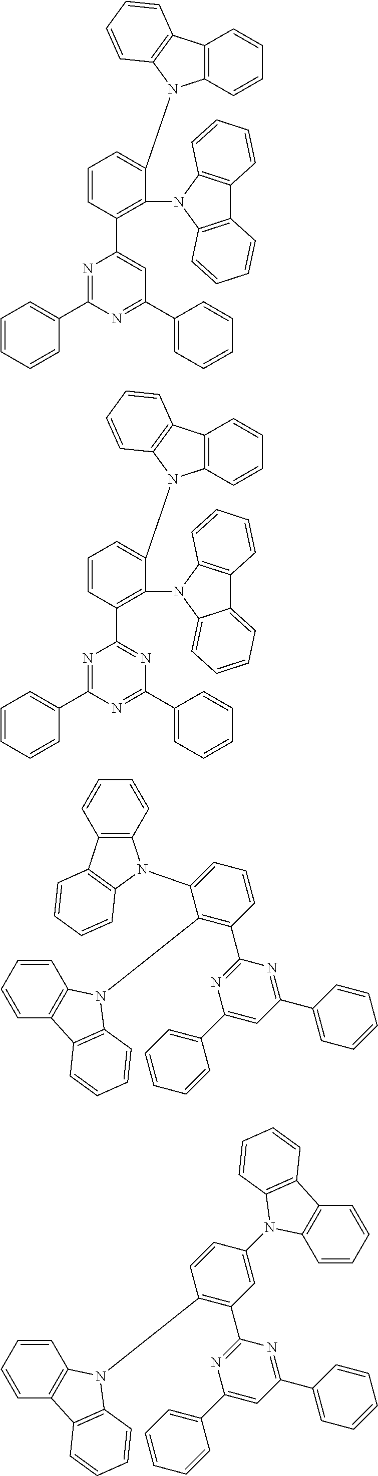

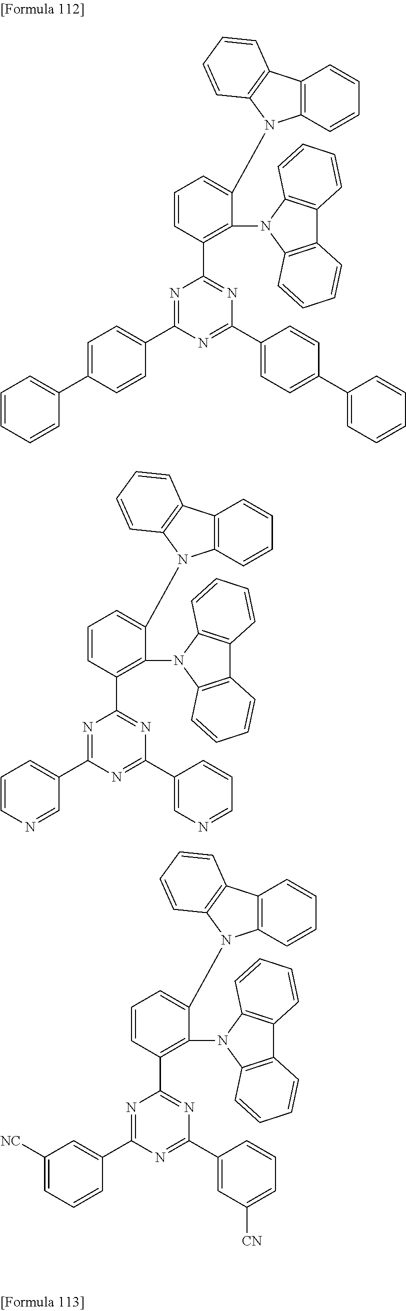

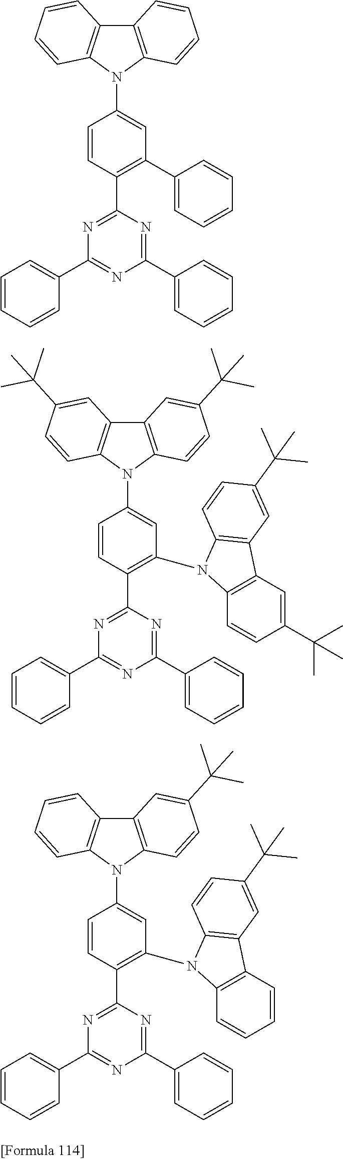

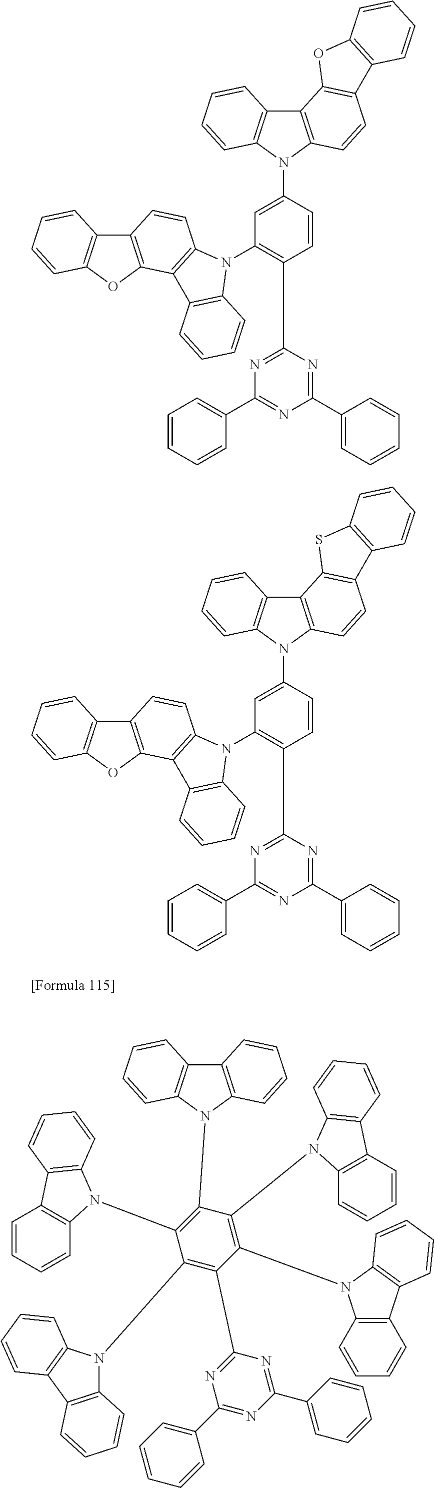

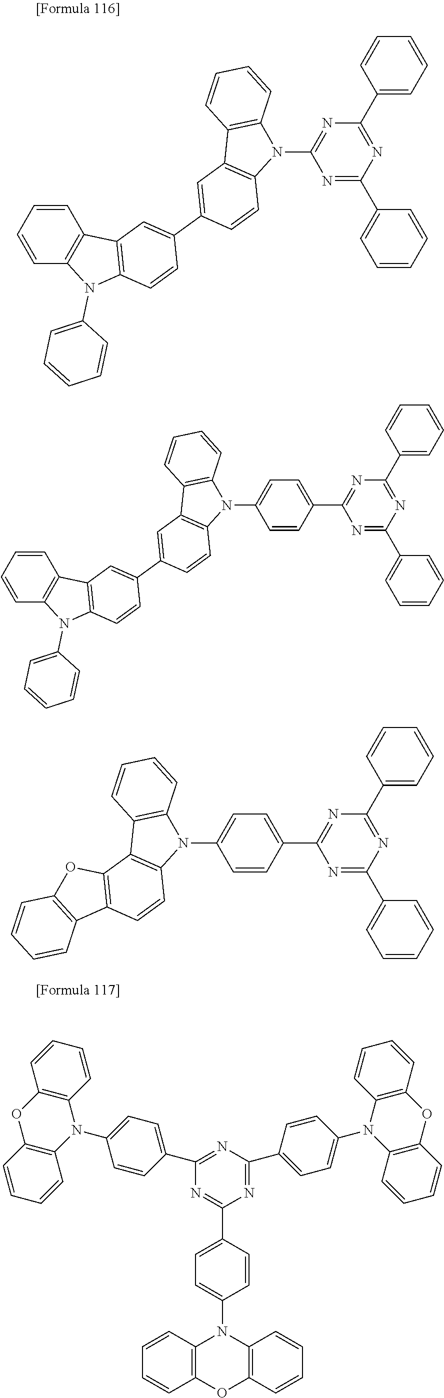 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2nc(-c3ccccc3)nc(-c3ccc(-n4c5ccccc5c5c6oc7ccccc7c6ccc54)cc3)n2)cc1 ; c1ccc2c(c1)Oc1ccccc1N2c1ccc(-c2nc(-c3ccc(N4c5ccccc5Oc5ccccc54)cc3)nc(-c3ccc(N4c5ccccc5Oc5ccccc54)cc3)n2)cc1]

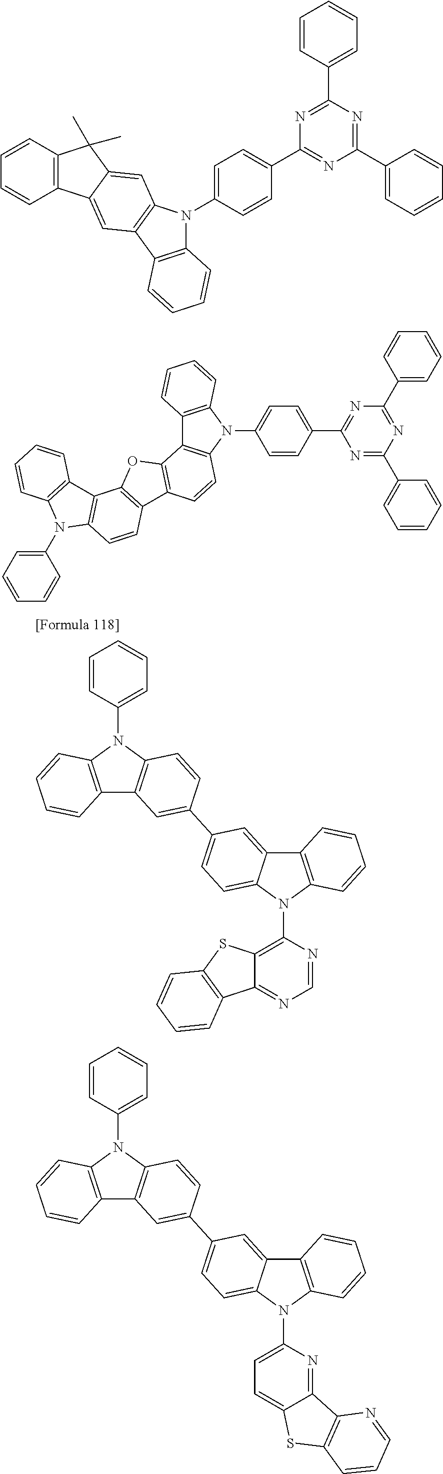

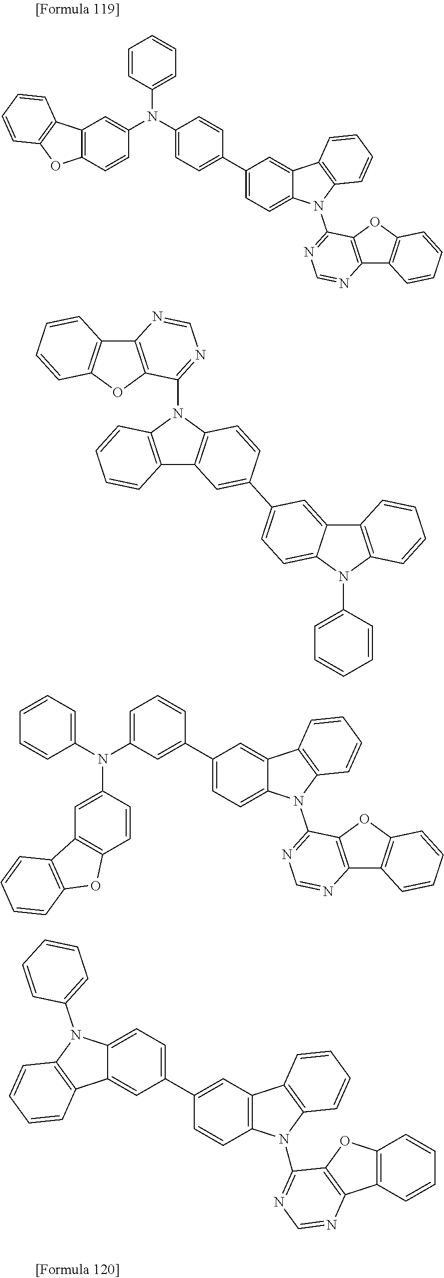

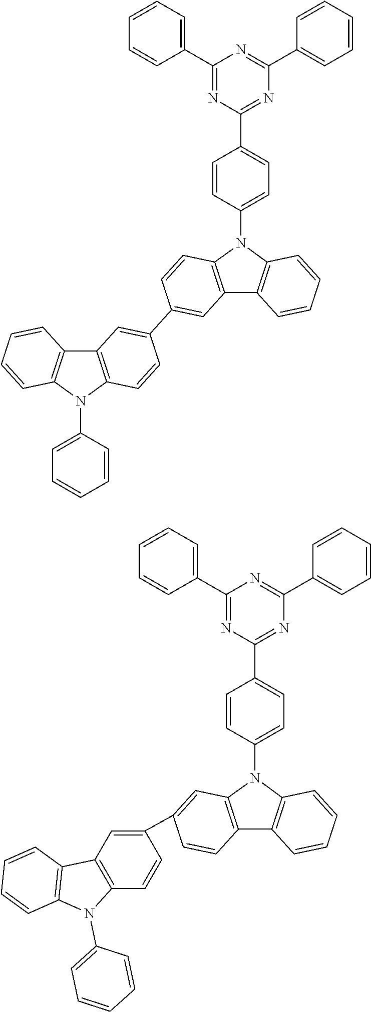 [SMILES: c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2nc(-c3ccccc3)nc(-c3ccc(-n4c5ccccc5c5ccc(-c6ccc7c(c6)c6ccccc6n7-c6ccccc6)cc54)cc3)n2)cc1]

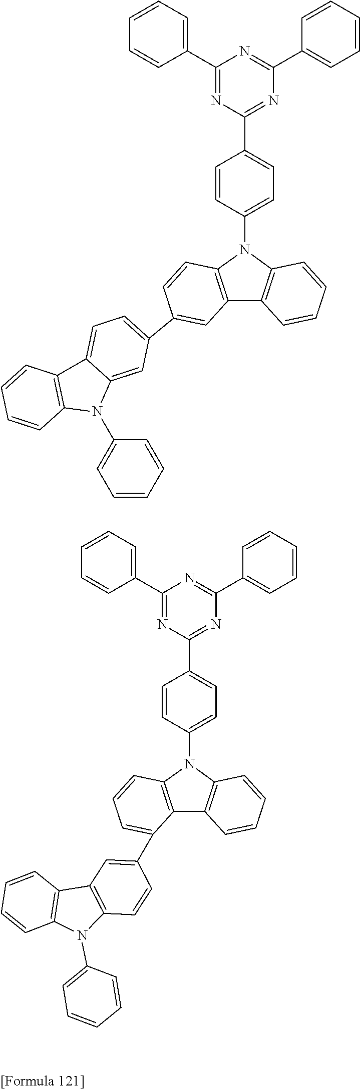

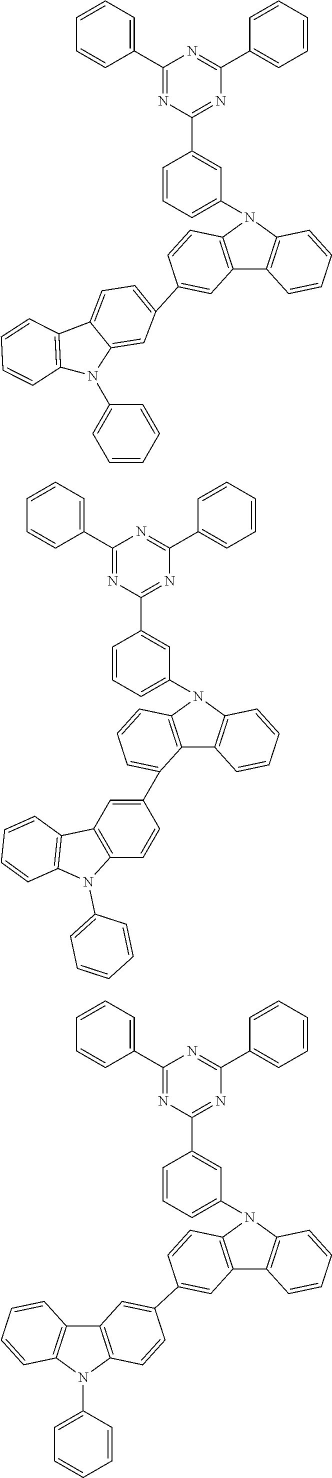

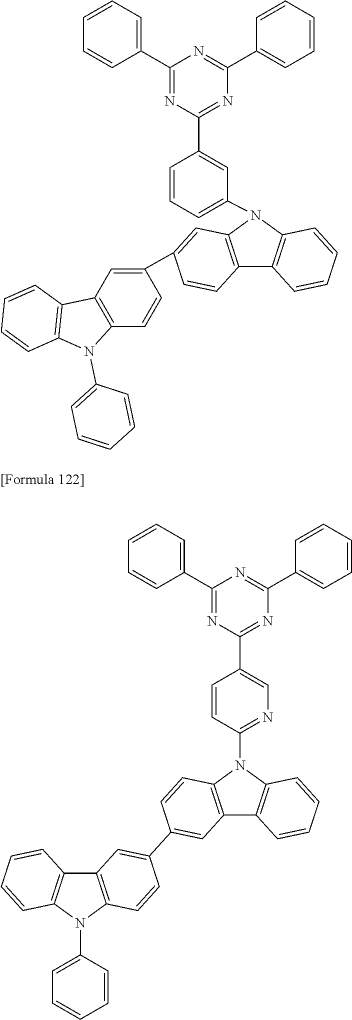 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1 ; c1ccc(-c2nc(-c3ccccc3)nc(-c3ccc(-n4c5ccccc5c5cc(-c6ccc7c(c6)c6ccccc6n7-c6ccccc6)ccc54)nc3)n2)cc1]

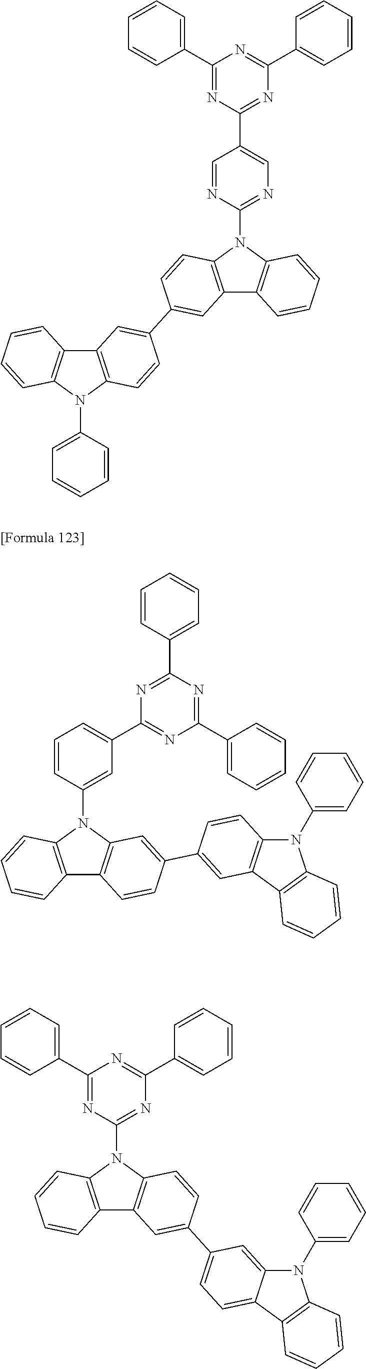 [SMILES: c1ccc(-c2nc(-c3ccccc3)nc(-c3cnc(-n4c5ccccc5c5cc(-c6ccc7c(c6)c6ccccc6n7-c6ccccc6)ccc54)nc3)n2)cc1 ; c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1 ; c1ccc(-c2nc(-c3ccccc3)nc(-n3c4ccccc4c4cc(-c5ccc6c7ccccc7n(-c7ccccc7)c6c5)ccc43)n2)cc1]

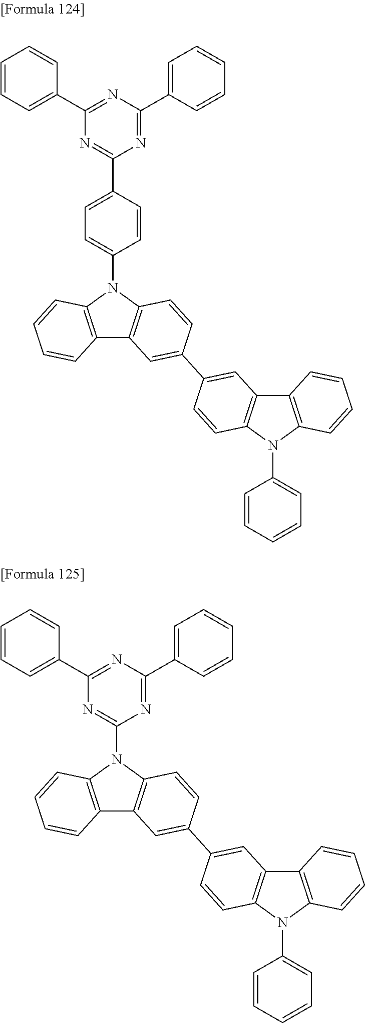 [SMILES: c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2nc(-c3ccccc3)nc(-n3c4ccccc4c4cc(-c5ccc6c(c5)c5ccccc5n6-c5ccccc5)ccc43)n2)cc1]

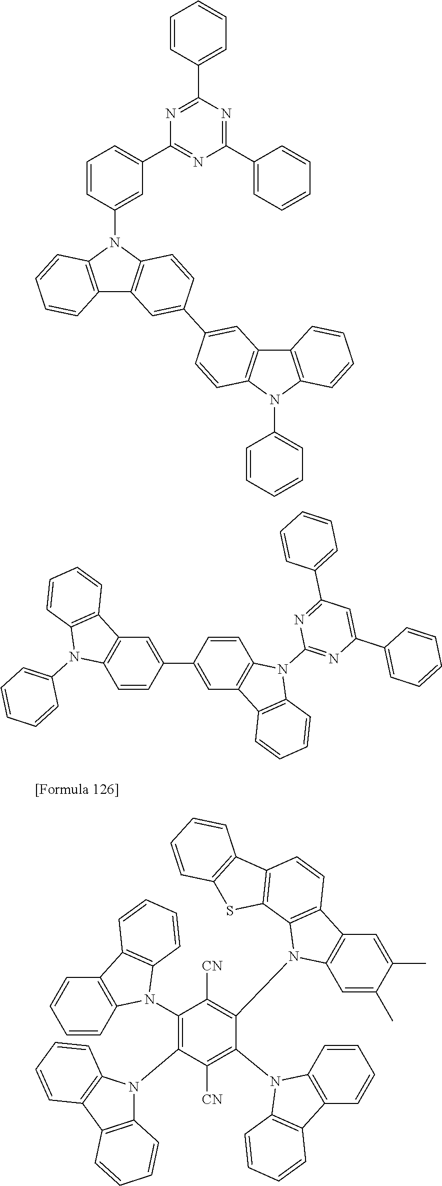

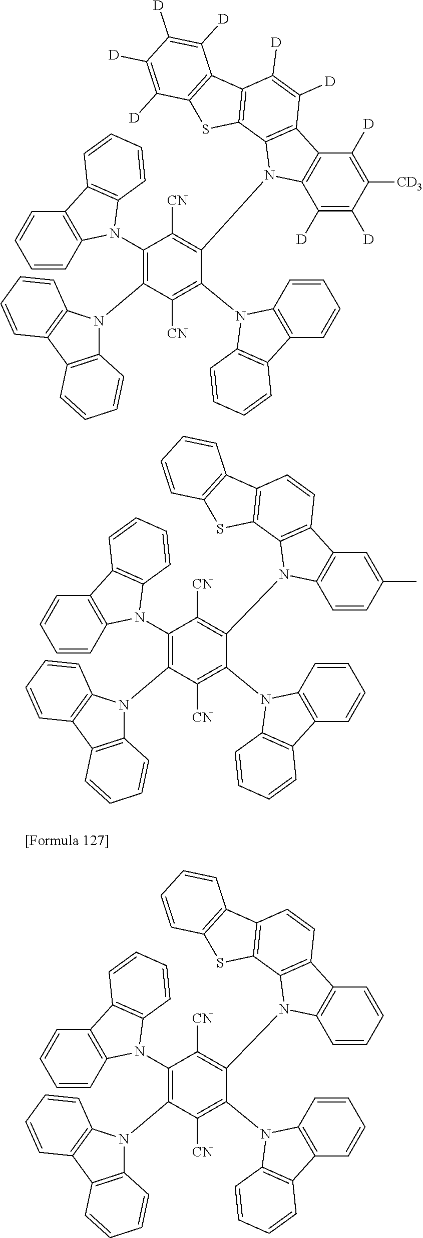

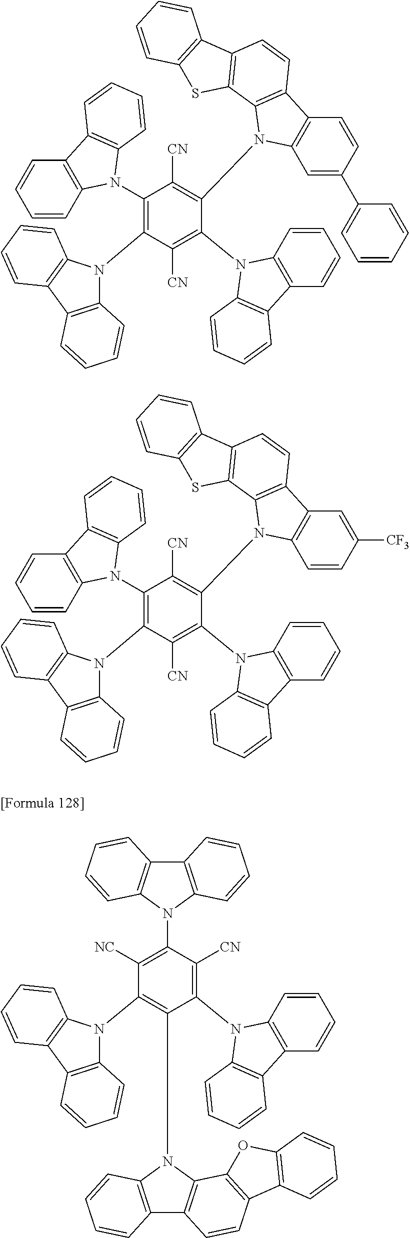 [SMILES: N#Cc1c(-n2c3ccccc3c3ccccc32)c(-n2c3ccccc3c3ccccc32)c(C#N)c(-n2c3cc(-c4ccccc4)ccc3c3ccc4c5ccccc5sc4c32)c1-n1c2ccccc2c2ccccc21 ; N#Cc1c(-n2c3ccccc3c3ccccc32)c(-n2c3ccccc3c3ccccc32)c(C#N)c(-n2c3ccc(C(F)(F)F)cc3c3ccc4c5ccccc5sc4c32)c1-n1c2ccccc2c2ccccc21 ; N#Cc1c(-n2c3ccccc3c3ccccc32)c(C#N)c(-n2c3ccccc3c3ccccc32)c(-n2c3ccccc3c3ccc4c5ccccc5oc4c32)c1-n1c2ccccc2c2ccccc21]

Relationship between Compound M3 and Compound M2 in Emitting Layer

In the organic EL device according to the exemplary embodiment, a singlet energy S₁(M3) of the compound M3 and a singlet energy S₁(M2) of the compound M2 satisfy a relationship of a numerical formula (Numerical Formula 1) below.

S₁(M3)>S₁(M2) (Numerical Formula 1)

An energy gap T_{77K}(M3) at 77K of the compound M3 is preferably larger than an energy gap T_{77K}(M2) at 77K of the compound M2. In other words, a relationship of a numerical formula (Numerical Formula 11) below is preferably satisfied.

T_{77K}(M3)>T_{77K}(M2) (Numerical Formula 11)

When the organic EL device according to the exemplary embodiment emits light, it is preferable that the compound M3 does not mainly emit light in the emitting layer.

Relationship between Triplet Energy and Energy Gap at 77K

Here, a relationship between a triplet energy and an energy gap at 77K will be described. In the exemplary embodiment, the energy gap at 77K is different from a typical triplet energy in some aspects.

The triplet energy is measured as follows. First, a solution in which a compound (measurement target) is dissolved in an appropriate solvent is encapsulated in a quartz glass tube to prepare a sample. A phosphorescence spectrum (ordinate axis: phosphorescent luminous intensity, abscissa axis: wavelength) of the sample is measured at a low temperature (77K). A tangent is drawn to the rise of the phosphorescence spectrum close to the short-wavelength region. The triplet energy is calculated by a predetermined conversion equation based on a wavelength value at an intersection of the tangent and the abscissa axis.

Herein, among the compounds of the exemplary embodiment, the thermally activated delayed fluorescent compound is preferably a compound having a small ΔST. When ΔST is small, intersystem crossing and inverse intersystem crossing are likely to occur even at a low temperature (77K), so that the singlet state and the triplet state coexist. As a result, the spectrum to be measured in the same manner as the above includes emission from both the singlet state and the triplet state. Although it is difficult to distinguish from which state, the singlet state or the triplet state, light is emitted, the value of the triplet energy is basically considered dominant.

Accordingly, in the exemplary embodiment, the triplet energy is measured by the same method as a typical triplet energy T, but a value measured in the following manner is referred to as an energy gap T77K in order to differentiate the measured energy from the typical triplet energy in a strict meaning. The measurement target compound is dissolved in EPA (diethylether:isopentane:ethanol=5:5:2 in volume ratio) at a concentration of 10 μmol/L, and the obtained solution is put in a quartz cell to provide a measurement sample. A phosphorescence spectrum (ordinate axis: phosphorescent luminous intensity, abscissa axis: wavelength) of the sample is measured at a low temperature (77K). A tangent is drawn to the rise of the phosphorescence spectrum close to the short-wavelength region. An energy amount is calculated by a conversion equation (F1) below based on a wavelength value λ_{edge}[nm] at an intersection of the tangent and the abscissa axis and is defined as an energy gap T_{77K} at 77K.

T_{77K} [eV]=1239.85/λ_{edge} Conversion Equation (F1):

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 (manufactured by Hitachi High-Technologies Corporation) is usable. Any device for phosphorescence measurement is usable. A combination of a cooling unit, a low temperature container, an excitation light source and a light-receiving unit may be used for phosphorescence measurement.

### Singlet Energy S₁

A method of measuring a singlet energy S₁ with use of a solution (occasionally referred to as a solution method) is exemplified by a method below.

A toluene solution of a measurement target compound at a concentration of 10 μmol/L is prepared and put in a quartz cell. An absorption spectrum (ordinate axis: absorption intensity, abscissa axis: wavelength) of the thus-obtained sample is measured at a normal temperature (300K). A tangent is drawn to the fall of the absorption spectrum close to the long-wavelength region, and a wavelength value λ_{edge}(nm) at an intersection of the tangent and the abscissa axis is assigned to a conversion equation (F2) below to calculate singlet energy.

S₁ [eV]=1239.85/λ_{edge} Conversion Equation (F2):

Any device for measuring absorption spectrum is usable. For instance, a spectrophotometer (U3310 manufactured by Hitachi, Ltd.) is usable.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

In the exemplary embodiment, a difference (S₁−T_{77K}) between the singlet energy S₁ and the energy gap T_{77K} at 77K is defined as ΔST.

In the exemplary embodiment, a difference ΔST(M2) between the singlet energy S₁(M2) of the compound M2 and the energy gap T_{77K}(M2) at 77K of the compound M2 is preferably less than 0.3 eV, more preferably less than 0.2 eV, still more preferably less than 0.1 eV, and still further more preferably less than 0.01 eV. In other words, ΔST(M2) preferably satisfies a relationship of one of numerical formulae (Numerical Formula 1A) to (Numerical Formula 1D) below.

ΔST(M2)=S₁(M2)−T_{77K}(M2)<0.3 eV (Numerical Formula 1A)

ΔST(M2)=S₁(M2)−T_{77K}(M2)<0.2 eV (Numerical Formula 1B)

ΔST(M2)=S₁(M2)−T_{77K}(M2)<0.1 eV (Numerical Formula 1C)

ΔST(M2)=S₁(M2)−T_{77K}(M2)<0.01 eV (Numerical Formula 1D)

### Film Thickness of Emitting Layer

The film thickness of the emitting layer of the organic EL device in the exemplary embodiment is preferably in a range from 5 nm to 50 nm, more preferably in a range from 7 nm to 50 nm, most preferably in a range from 10 nm to 50 nm. When the film thickness of the emitting layer is 5 nm or more, the formation of the emitting layer and the adjustment of the chromaticity are easy. When the film thickness of the emitting layer is 50 nm or less, an increase in the drive voltage is likely to be reduced.

### Content Ratios of Compounds in Emitting Layer

For instance, content ratios of the compound M2 and the compound M3 in the emitting layer preferably fall within ranges shown below.

The content ratio of the compound M2 is preferably in a range from 10 mass % to 80 mass %, more preferably in a range from 10 mass % to 60 mass %, and still more preferably in a range from 20 mass % to 60 mass %.

The content ratio of the compound M3 is preferably in a range from 20 mass % to 90 mass %, more preferably in a range from 40 mass % to 90 mass %, and still more preferably in a range from 40 mass % to 80 mass %.

It should be noted that the emitting layer of the exemplary embodiment may contain a material other than the compound M2 and the compound M3.

The emitting layer may contain a single type of the compound M2 or may contain two or more types of the compound M2. The emitting layer may contain a single type of the compound M3 or may contain two or more types of the compound M3.

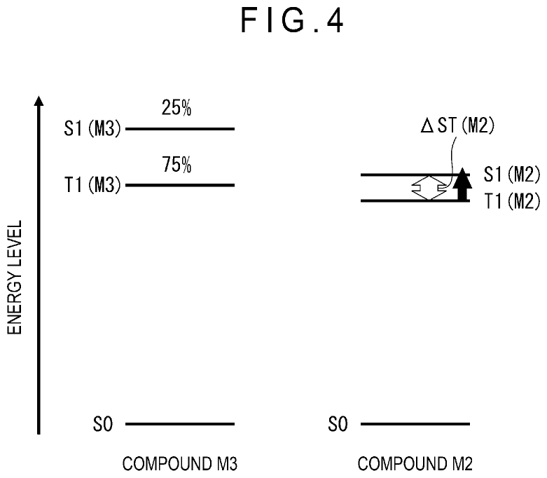

FIG. 4 shows an example of a relationship between energy levels of the compound M3 and the compound M2 in the emitting layer. In FIG. 4, S0 represents a ground state. S₁(M2) represents a lowest singlet state of the compound M2. T1(M2) represents a lowest triplet state of the compound M2. S₁(M3) represents a lowest singlet state of the compound M3. T1(M3) represents a lowest triplet state of the compound M3. As shown in FIG. 4, when a compound having a small ΔST(M2) is used as the compound M2, inverse intersystem crossing from the lowest triplet state T1 to the lowest singlet state S1 can be caused by heat energy.

The inverse intersystem crossing caused in the compound M2 enables light emission from the lowest singlet state S1(M2) of the compound M2 to be observed when the emitting layer does not contain a fluorescent dopant with the lowest singlet state S1 smaller than the lowest singlet state S1(M2) of the compound M2. It is inferred that the internal quantum efficiency can be theoretically raised up to 100% also by using delayed fluorescence by the TADF mechanism.

In the organic EL device according to the exemplary embodiment, the emitting layer contains the delayed fluorescent compound M2 and the compound M3 having a larger singlet energy than the compound M2.

According to the exemplary embodiment, the organic EL device having a high performance, particularly, an improved luminous efficiency is achievable.

The organic EL device according to the exemplary embodiment is usable in an electronic device such as a display device and a light-emitting unit.

An arrangement of the organic EL device will be further described below.

### Substrate

The substrate is used as a support for the organic EL device. For instance, glass, quartz, plastics and the like are usable for the substrate. A flexible substrate is also usable. The flexible substrate is a bendable substrate, which is exemplified by a plastic substrate. Examples of the material for the plastic substrate include polycarbonate, polyarylate, polyethersulfone, polypropylene, polyester, polyvinyl fluoride, polyvinyl chloride, polyimide, and polyethylene naphthalate. Moreover, an inorganic vapor deposition film is also usable.

### Anode

Metal, an alloy, an electrically conductive compound, a mixture thereof, or the like having a large work function (specifically, 4.0 eV or more) is preferably used as the anode formed on the substrate. Specific examples of the material include ITO (Indium Tin Oxide), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the organic layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

The elements belonging to the group 1 or 2 of the periodic table, which are a material having a small work function, specifically, an alkali metal such as lithium (Li) and cesium (Cs), an alkaline earth metal such as magnesium (Mg), calcium (Ca) and strontium (Sr), an alloy containing the alkali metal and the alkaline earth metal (e.g., MgAg, AlLi), a rare earth metal such as europium (Eu) and ytterbium (Yb), and an alloy containing the rare earth metal are usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

### Cathode

It is preferable to use metal, an alloy, an electroconductive compound, a mixture thereof, or the like having a small work function (specifically, 3.8 eV or less) for the cathode. Examples of materials for the cathode include elements belonging to the group 1 or 2 of the periodic table, specifically, an alkali metal such as lithium (Li) and cesium (Cs), an alkaline earth metal such as magnesium (Mg), calcium (Ca) and strontium (Sr), an alloy containing the alkali metal and the alkaline earth metal (e.g., MgAg, AlLi), a rare earth metal such as europium (Eu) and ytterbium (Yb), and an alloy containing the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method and the like.

### Hole Injecting Layer

The hole injecting layer is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the highly hole-injectable substance include: an aromatic amine compound, which is a low-molecule organic compound, such that 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl(abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1); and dipyrazino[2,3-f:20,30-h]quinoxaline-2,3,6,7,10,11-hexacarbonitrile (HAT-CN).

In addition, a high polymer compound (e.g., oligomer, dendrimer and polymer) is usable as the substance exhibiting a high hole injectability. Examples of the high-molecule compound include poly(N-vinylcarbazole) (abbreviation: PVK), poly(4-vinyltiphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine] (abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid) (PAni/PSS) are also usable.

### Hole Transporting Layer

The hole transporting layer is a layer containing a highly hole-transporting substance. An aromatic amine compound, carbazole derivative, anthracene derivative and the like are usable for the hole transporting layer. Specific examples of a material for the hole transporting layer include 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)tiphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(V·s) or more.

For the hole transporting layer, a carbazole derivative such as CBP, 9-[4-(N-carbazolyl)]phenyl-10-phenylanthracene (CzPA), and 9-phenyl-3-[4-(10-phenyl-9-anthryl)phenyl]-9H-carbazole (PCzPA) and an anthracene derivative such as t-BuDNA, DNA, and DPAnth may be used. A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used. It should be noted that the layer containing the substance exhibiting a high hole transportability may be not only a single layer but also a laminate of two or more layers formed of the above substance(s).

When the hole transporting layer includes two or more layers, one of the layers with a larger energy gap is preferably provided closer to the emitting layer. Such a material is exemplified by a compound EBL used in Examples described later.

### Electron Transporting Layer

The electron transporting layer is a layer containing a highly electron-transporting substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinato)aluminum (abbreviation: Almq3), bis(10-hydroxybenzo[h]quinolinato)beryllium (abbreviation: BeBq2), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. In the exemplary embodiment, a benzimidazole compound is preferably usable. The above-described substances mostly have an electron mobility of 10⁻⁶ cm²/(V·s) or more. It should be noted that any substance other than the above substance may be used for the electron transporting layer as long as the substance exhibits a higher electron transportability than the hole transportability. The electron transporting layer may be provided in the form of a single layer or a laminate of two or more layers of the above substance(s).

Further, a high polymer compound is usable for the electron transporting layer. For instance, poly[(9,9-dihexylfluorene-2,7-diyl)-co-(pyridine-3,5-diyl)](abbreviation: PF-Py), poly[(9,9-dioctylfluorene-2,7-diyl)-co-(2,2′-bipyridine-6,6′-diyl)] (abbreviation: PF-BPy) and the like are usable.

### Electron Injecting Layer

The electron injecting layer is a layer containing a highly electron-injectable substance. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

### Layer Formation Method

A method for forming each layer of the organic EL device in the exemplary embodiment is subject to no limitation except for the above particular description. However, known methods of dry film-forming such as vacuum deposition, sputtering, plasma or ion plating and wet film-forming such as spin coating, dipping, flow coating or ink-jet are applicable.

### Film Thickness

A thickness of each of the organic layers in the organic EL device according to the exemplary embodiment is not limited except for the above particular description. In general, the thickness preferably ranges from several nanometers to 1 μm because excessively small film thickness is likely to cause defects (e.g. pin holes) and excessively large thickness leads to the necessity of applying high voltage and consequent reduction in efficiency.

### Second Exemplary Embodiment

An arrangement of an organic EL device according to a second exemplary embodiment of the invention will be described below. In the description of the second exemplary embodiment, the same components as those in the first exemplary embodiment are denoted by the same reference signs and names to simplify or omit an explanation of the components. In the second exemplary embodiment, the same materials and compounds as described in the first exemplary embodiment are usable, unless otherwise specified.

The organic EL device according to the second exemplary embodiment is different from the organic EL device according to the first exemplary embodiment in that the emitting layer further includes a fluorescent compound M1. The second exemplary embodiment is the same as the first exemplary embodiment in other respects.

Specifically, in the second exemplary embodiment, the emitting layer contains the compound M3 represented by the formula (11), (12) or (13) below, a delayed fluorescent compound M2, and a fluorescent compound M1.

In this arrangement, it is preferable that the compound M1 is a dopant material, the compound M2 is a host material, and the compound M3 is not a dopant material.

### Compound M1

The emitting layer of the exemplary embodiment preferably contains the fluorescent compound M1.

The compound M1 of the exemplary embodiment is not a phosphorescent metal complex. The compound M1 is preferably not a heavy-metal complex. The compound M1 is also preferably not a metal complex.

The compound M1 of the exemplary embodiment is preferably a compound not exhibiting thermally activated delayed fluorescence.

A fluorescent material is usable as the compound M1 of the exemplary embodiment. Specific examples of the fluorescent material include a bisarylaminonaphthalene derivative, aryl-substituted naphthalene derivative, bisarylaminoanthracene derivative, aryl-substituted anthracene derivative, bisarylaminopyrene derivative, aryl-substituted pyrene derivative, bisarylamino chrysene derivative, aryl-substituted chrysene derivative, bisarylaminofluoranthene derivative, aryl-substituted fluoranthene derivative, indenoperylene derivative, acenaphthofluoranthene derivative, compound including a boron atom, pyromethene boron complex compound, compound having a pyromethene skeleton, metal complex of the compound having a pyrromethene skeleton, diketopyrrolopyrrole derivative, perylene derivative, and naphthacene derivative.

### Compound Represented by Formula (20)

The compound M1 of the exemplary embodiment is preferably a compound represented by a formula (20) below.

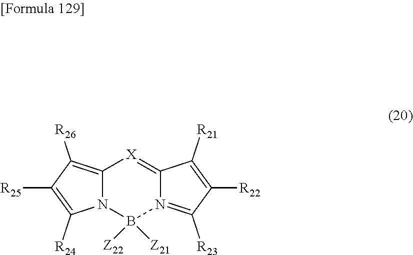

In the formula (20):
X is a nitrogen atom, or a carbon atom bonded to Y;
Y is a hydrogen atom or a substituent;
R₂₁ to R₂₆ are each independently a hydrogen atom or a substituent, or at least one combination of a combination of R₂₁ and R₂₂, a combination of R₂₂ and R₂₃, a combination of R₂₄ and R₂₅, or a combination of R₂₅ and R₂₆ are mutually bonded to form a ring;
Y and R₂₁ to R₂₆ as a substituent are each independently selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, a substituted or unsubstituted alkylthio group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted aralkyl group having 7 to 30 carbon atoms, a substituted or unsubstituted heteroaryl group having 5 to 30 ring atoms, a halogen atom, a carboxy group, a substituted or unsubstituted ester group, a substituted or unsubstituted carbamoyl group, a substituted or unsubstituted amino group, a nitro group, a cyano group, a substituted or unsubstituted silyl group, and a substituted or unsubstituted siloxanyl group;
Z₂₁ and Z₂₂ are each independently a substituent, or Z₂₁ and Z₂₂ are mutually bonded to form a ring;
Z₂₁ and Z₂₂ as a substituent are each independently selected from the group consisting of a halogen atom, a substituted or unsubstituted alkyl group having 1 to 30 carbon atoms, a substituted or unsubstituted alkyl halide group having 1 to 30 carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, and a substituted or unsubstituted aryloxy group having 6 to 30 ring carbon atoms.

When the compound M1 is a fluorescent compound, the compound M1 preferably emits light having a main peak wavelength in a range from 400 nm to 700 nm.

Herein, the main peak wavelength means a peak wavelength of a fluorescence spectrum exhibiting a maximum luminous intensity among fluorescence spectra measured in a toluene solution in which a measurement target compound is dissolved at a concentration ranging from 10⁻⁶ mol/l to 10⁻⁵ mol/l. A spectrophotofluorometer (F-7000 manufactured by Hitachi High-Tech Science Corporation) is used as a measurement device.

The compound M1 preferably exhibits red or green light emission.

Herein, the red light emission refers to light emission whose main peak wavelength of fluorescence spectrum is in a range from 600 nm to 660 nm.

When the compound M1 is a red fluorescent compound, the main peak wavelength of the compound M1 is preferably in a range from 600 nm to 660 nm, more preferably in a range from 600 nm to 640 nm, and still more preferably in a range from 610 nm to 630 nm.

Herein, the green light emission refers to light emission whose main peak wavelength of fluorescence spectrum is in a range from 500 nm to 560 nm.

When the compound M1 is a green fluorescent compound, the main peak wavelength of the compound M1 is preferably in a range from 500 nm to 560 nm, more preferably in a range from 500 nm to 540 nm, and still more preferably in a range from 510 nm to 540 nm.

Herein, the blue light emission refers to light emission whose main peak wavelength of fluorescence spectrum is in a range from 430 nm to 480 nm.

When the compound M1 is a blue fluorescent compound, the main peak wavelength of the compound M1 is preferably in a range from 430 nm to 480 nm, more preferably in a range from 440 nm to 480 nm.

A main peak wavelength of the light emitted from the organic EL device is measured as follows.

Voltage is applied on the organic EL devices such that a current density becomes 10 mA/cm², where spectral radiance spectrum is measured by a spectroradiometer CS-2000 (manufactured by Konica Minolta, Inc.).

A peak wavelength of an emission spectrum, at which the luminous intensity of the resultant spectral radiance spectrum is at the maximum, is measured and defined as the main peak wavelength (unit: nm).

### Manufacturing Method of Compound M1

The compound M1 can be manufactured by a known method.

Specific examples of a compound represented by the formula (20) are shown below. However, the invention is by no means limited to the specifically listed compounds.

A coordinate bond between a boron atom and a nitrogen atom in a pyrromethene skeleton is shown by various means such as a solid line, a broken line, an arrow, and omission. Herein, the coordinate bond is shown by a solid line or a broken line, or the description of the coordinate bond is omitted.

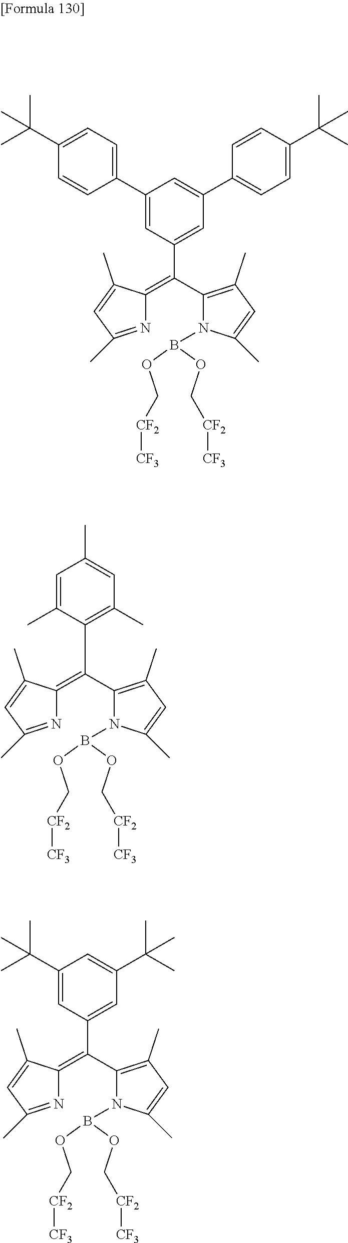

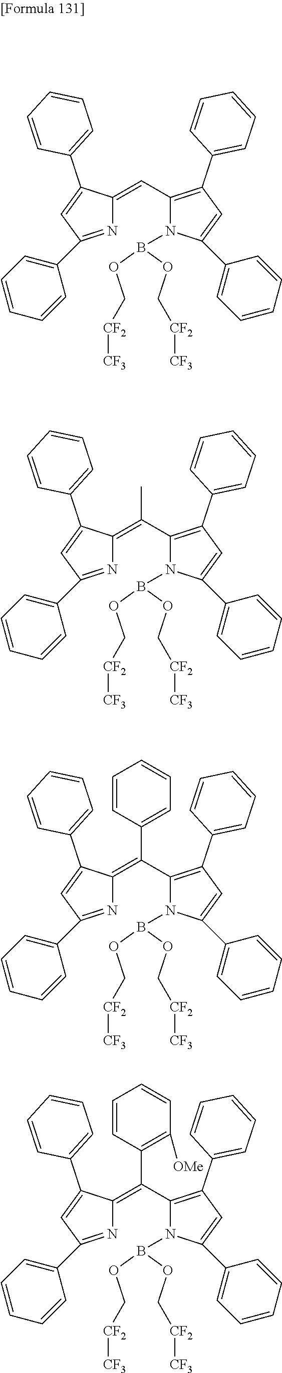 [SMILES: FC(F)(F)C(F)(F)COB(OCC(F)(F)C(F)(F)F)n1c(-c2ccccc2)cc(-c2ccccc2)c1/C=C1\N=C(c2ccccc2)C=C1c1ccccc1 ; C/C(=C1/N=C(c2ccccc2)C=C1c1ccccc1)c1c(-c2ccccc2)cc(-c2ccccc2)n1B(OCC(F)(F)C(F)(F)F)OCC(F)(F)C(F)(F)F ; FC(F)(F)C(F)(F)COB(OCC(F)(F)C(F)(F)F)n1c(-c2ccccc2)cc(-c2ccccc2)c1/C(=C1\N=C(c2ccccc2)C=C1c1ccccc1)c1ccccc1 ; COc1ccccc1/C(=C1/N=C(c2ccccc2)C=C1c1ccccc1)c1c(-c2ccccc2)cc(-c2ccccc2)n1B(OCC(F)(F)C(F)(F)F)OCC(F)(F)C(F)(F)F]

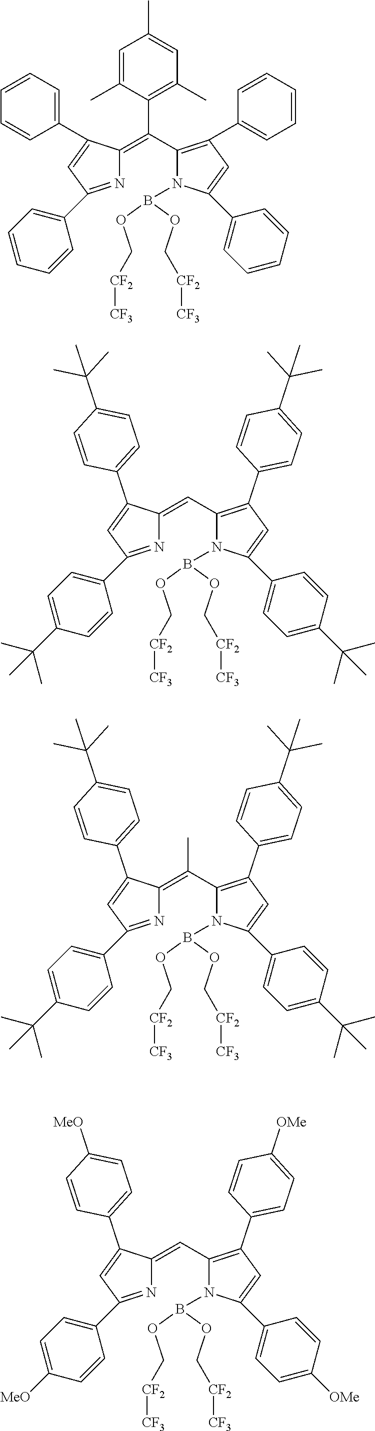

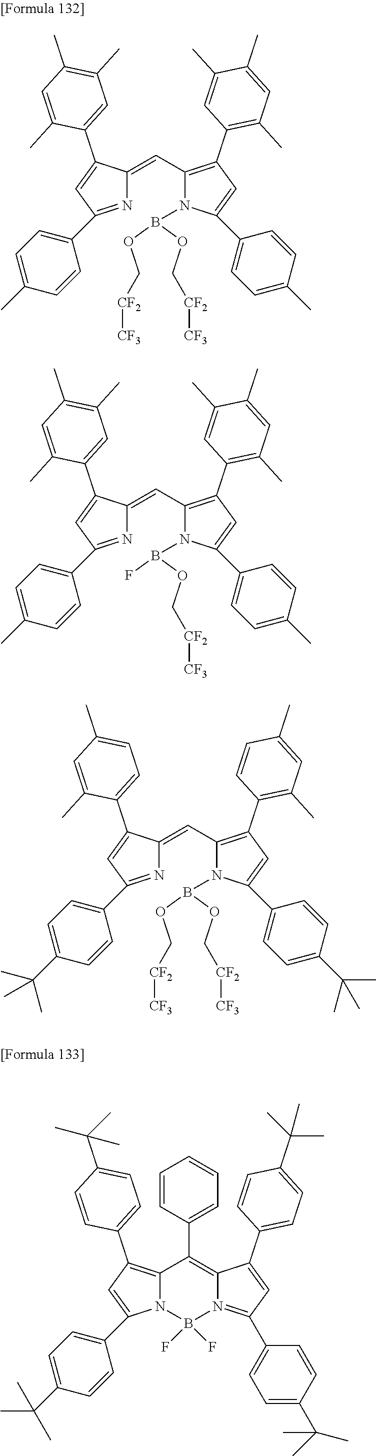

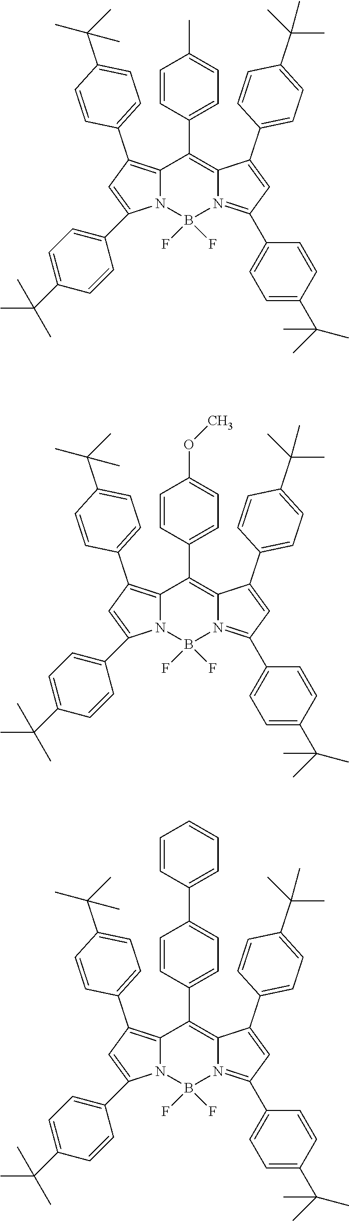

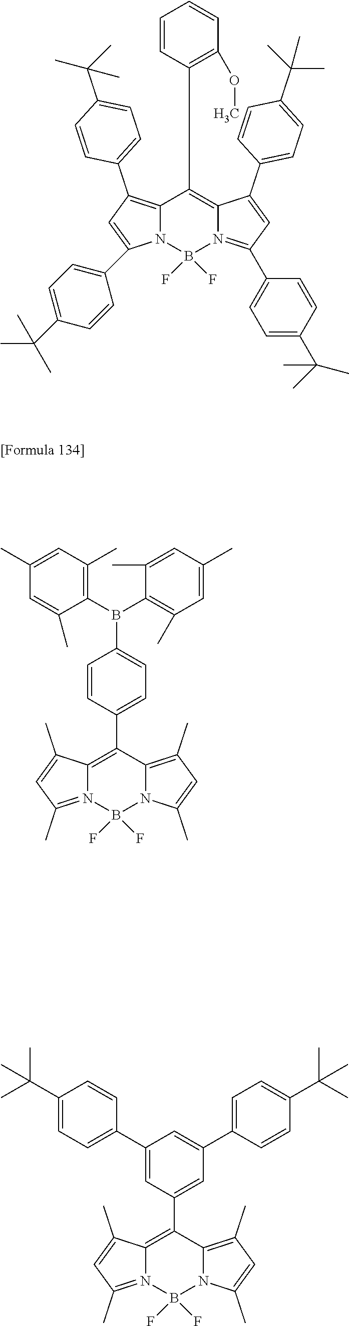

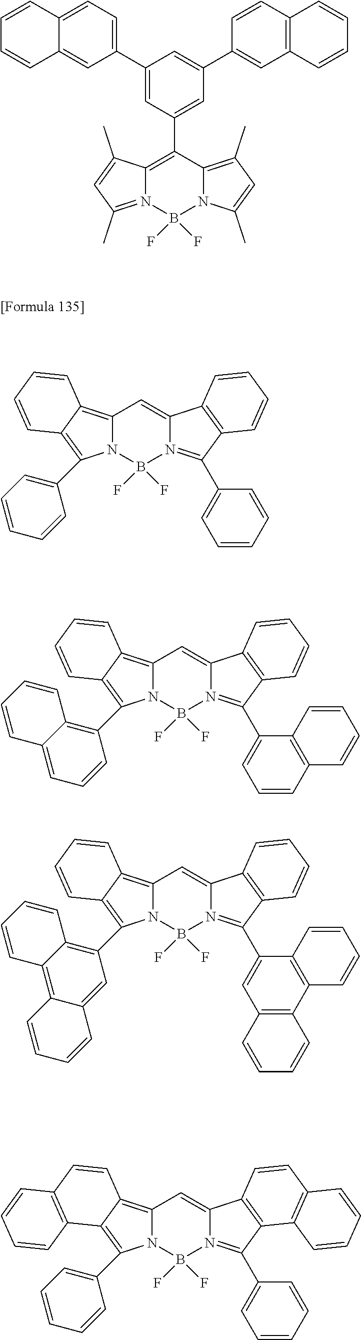

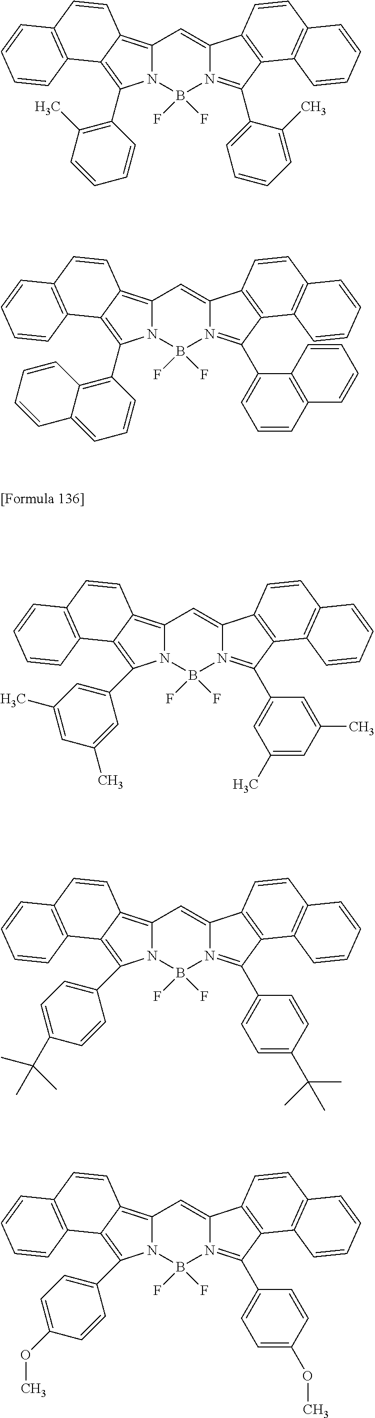

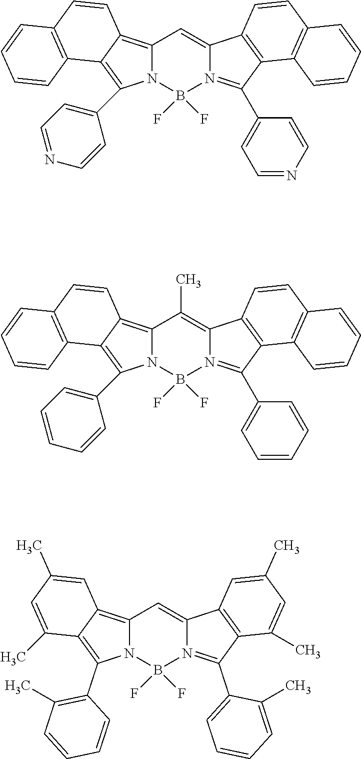

Relationship between Compound M3, Compound M2 and Compound M1 in Emitting Layer

In the organic EL device according to the exemplary embodiment, a singlet energy S₁(M1) of the compound M1 and a singlet energy S₁(M2) of the compound M2 satisfy a relationship of a numerical formula (Numerical Formula 2) below.

S₁(M2)>S₁(M1) (Numerical Formula 2)

Moreover, the singlet energy S1(M3) of the compound M3 is preferably larger than the singlet energy S1(M1) of the compound M1.

S₁(M3)>S₁(M1) (Numerical Formula 2A)

The singlet energy S₁(M3) of the compound M3, the singlet energy S₁(M2) of the compound M2, and the singlet energy S₁(M1) of the compound M1 preferably satisfy a relationship of a numerical formula (Numerical Formula 2B) below.

S₁(M3)>S₁(M2)>S₁(M1) (Numerical Formula 2B)

It is preferable that mainly the fluorescent compound M1 emits light in the emitting layer when the organic EL device of the exemplary embodiment emits light.

The organic EL device according to the exemplary embodiment preferably emits red light or green light.

### Content Ratios of Compounds in Emitting Layer

Content ratios of the compound M3, the compound M2, and the compound M1 in the emitting layer preferably fall, for instance, within a range below.

The content ratio of the compound M3 is preferably in a range from 10 mass % to 80 mass %.

The content ratio of the compound M2 is preferably in a range from 10 mass % to 80 mass %, more preferably in a range from 10 mass % to 60 mass %, and still more preferably in a range from 20 mass % to 60 mass %.

The content ratio of the compound M1 is preferably in a range from 0.01 mass % to 10 mass %, more preferably in a range from 0.01 mass % to 5 mass %, and still more preferably in a range from 0.01 mass % to 1 mass %.

The upper limit of a total of the content ratios of the compound M3, the compound M2, and the compound M1 in the emitting layer is 100 mass %. It should be noted that the emitting layer of the exemplary embodiment may further contain material(s) other than the compounds M3, M2 and M1.

The emitting layer may contain a single type of the compound M3 or may contain two or more types of the compound M3. The emitting layer may contain a single type of the compound M2 or may contain two or more types of the compound M2. The emitting layer may contain a single type of the compound M1 or may contain two or more types of the compound M1.

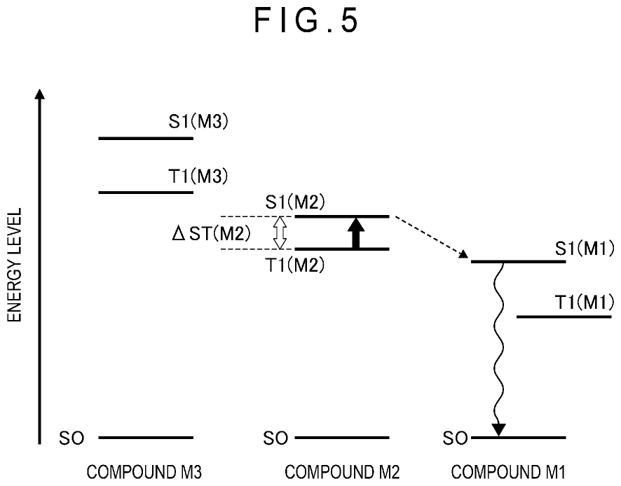

FIG. 5 shows an example of a relationship between energy levels of the compound M3, the compound M2, and the compound M1 in the emitting layer. In FIG. 5, S0 represents a ground state. S₁(M1) represents a lowest singlet state of the compound M1. T1(M1) represents a lowest triplet state of the compound M1. S1(M2) represents a lowest singlet state of the compound M2. T1(M2) represents a lowest triplet state of the compound M2. S1(M3) represents a lowest singlet state of the compound M3. T1(M3) represents a lowest triplet state of the compound M3. A dashed arrow directed from S1(M2) to S1(M1) in FIG. 5 represents Forster energy transfer from the lowest singlet state of the compound M2 to the lowest singlet state of the compound M1.

As shown in FIG. 5, when a compound having a small ΔST(M2) is used as the compound M2, inverse intersystem crossing from the lowest triplet state T1(M2) to the lowest singlet state S1(M2) can be caused by a heat energy. Subsequently, Förster energy transfer from the lowest singlet state S1(M2) of the compound M2 to the compound M1 occurs to generate the lowest singlet state S1(M1). Consequently, fluorescence from the lowest singlet state S1(M1) of the compound M1 can be observed. It is inferred that the internal quantum efficiency can be theoretically raised up to 100% also by using delayed fluorescence by the TADF mechanism.

The organic EL device according to the second exemplary embodiment contains the delayed fluorescent compound M2, the compound M3 having the singlet energy larger than that of the compound M2, and the compound M1 having the singlet energy smaller than that of the delayed fluorescent compound M2 in the emitting layer.

According to the second exemplary embodiment, the organic EL device having a high performance, particularly, an improved luminous efficiency is achievable.

The organic EL device according to the second exemplary embodiment is usable in an electronic device such as a display device and a light-emitting unit.

### Third Exemplary Embodiment

### Compound

A compound of a third exemplary embodiment is a compound represented by a formula (121) or (122) below. A compound falling under the formula (121) or (122) among the specific examples of the compound M3 of the first exemplary embodiment is also used as a specific example of the compound of the third exemplary embodiment.

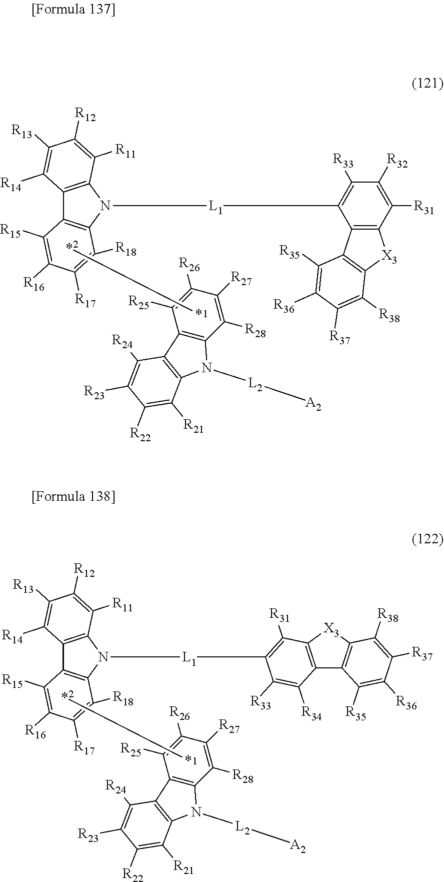

In the formulae (121) and (122):
A₂ is a substituted or unsubstituted aryl group having 6 to 30 ring carbon atoms, or a substituted or unsubstituted heterocyclic group having 5 to 30 ring atoms;
L₁ and L₂ are each independently a single bond, a substituted or unsubstituted arylene group having 6 to 30 ring carbon atoms, a substituted or unsubstituted divalent heterocyclic group having 5 to 30 ring atoms, a group obtained by bonding two groups selected from the group consisting of a substituted or unsubstituted arylene group having 6 to 30 ring carbon atoms and a substituted or unsubstituted divalent heterocyclic group having 5 to 30 ring atoms, or a group obtained by bonding three groups selected from the group consisting of a substituted or unsubstituted arylene group having 6 to 30 ring carbon atoms and a substituted or unsubstituted divalent heterocyclic group having 5 to 30 ring atoms;
the two groups are mutually the same or different;
the three groups are mutually the same or different;
R₁₁ to R₁₈ are each independently a hydrogen atom or a substituent, or at least one combination of a combination of R₁₁ and R₁₂, a combination of R₁₂ and R₁₃, a combination of R₁₃ and R₁₄, a combination of R₁₅ and R₁₆, a combination of R₁₆ and R₁₇, or a combination of R₁₇ and R₁₈ are mutually bonded to form a ring;
R₂₁ to R₂₈ are each independently a hydrogen atom or a substituent, or at least one combination of a combination of R₂₁ and R₂₂, a combination of R₂₂ and R₂₃, a combination of R₂₃ and R₂₄, a combination of R₂₅ and R₂₆, a combination of R₂₆ and R₂₇, or a combination of R₂₇ and R₂₈ are mutually bonded to form a ring;
one of carbon atoms bonded to R₁₅, R₁₇ and R₁₈ is bonded to *2 and one of carbon atoms bonded to R₂₅, R₂₇ and R₂₈ is bonded to *1;
a carbon atom bonded to R₁₇ and a carbon atom bonded to R₂₇ are not simultaneously bonded to *2 and *1, respectively;
X₃ is an oxygen atom or a sulfur atom;
R₃₁ to R₃₈ are each independently a hydrogen atom or a substituent, or at least one combination of a combination of R₃₁ and R₃₂, a combination of R₃₂ and R₃₃, a combination of R₃₃ and R₃₄, a combination of R₃₅ and R₃₆, a combination of R₃₆ and R₃₇, or a combination of R₃₇ and R₃₈ are mutually bonded to form a ring;
R₁₁ to R₁₈, R₂₁ to R₂₈, and R₃₁ to R₃₈ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, a group represented by —N(Rz)₂, a thiol group, a substituted or unsubstituted alkylthio group having 1 to 30 carbon atoms, a substituted or unsubstituted aralkyl group having 7 to 30 ring carbon atoms, a substituted germanium group, a substituted phosphine oxide group, a nitro group, a substituted boryl group, or a substituted or unsubstituted arylthio group having 6 to 30 ring carbon atoms;
Rz is a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms;
two Rz in —N(Rz)₂ are mutually the same or different.

In the formulae (121) and (122): when a carbon atom bonded to R₁₅ is bonded to *2, a hydrogen atom represented by R₁₅, a substituent represented by R₁₅, and a ring formed by involving R₁₅ are not present.

In the formulae (121) and (122): when a carbon atom bonded to R₁₇ is bonded to *2, a hydrogen atom represented by R₁₇, a substituent represented by R₁₇, and a ring formed by involving R₁₇ are not present.

In the formulae (121) and (122): when a carbon atom bonded to R₁₈ is bonded to *2, a hydrogen atom represented by R₁₈, a substituent represented by R₁₈, and a ring formed by involving R₁₈ are not present.

In the formulae (121) and (122): when a carbon atom bonded to R₂₅ is bonded to *1, a hydrogen atom represented by R₂₅, a substituent represented by R₂₅, and a ring formed by involving R₂₅ are not present.

In the formulae (121) and (122): when a carbon atom bonded to R₂₇ is bonded to *1, a hydrogen atom represented by R₂₇, a substituent represented by R₂₇, and a ring formed by involving R₂₇ are not present.

In the formulae (121) and (122): when a carbon atom bonded to R₂₈ is bonded to *1, a hydrogen atom represented by R₂₈, a substituent represented by R₂₈, and a ring formed by involving R₂₈ are not present.

In the formulae (121) and (122): it is preferable that a combination of R₁₁ and R₁₂, a combination of R₁₂ and R₁₃, a combination of R₁₃ and R₁₄, a combination of R₁₅ and R₁₆, a combination of R₁₆ and R₁₇, and a combination of R₁₇ and R₁₈ are not mutually bonded.

In the formulae (121) and (122): it is preferable that a combination of R₂₁ and R₂₂, a combination of R₂₂ and R₂₃, a combination of R₂₃ and R₂₄, a combination of R₁₅ and R₁₆, a combination of R₂₆ and R₂₇, and a combination of R₂₇ and R₂₈ are not mutually bonded.

In the formulae (121) and (122): it is preferable that a combination of R₃₁ and R₃₂, a combination of R₃₂ and R₃₃, a combination of R₃₃ and R₃₄, a combination of R₃₅ and R₃₆, a combination of R₃ and R₃₇, and a combination of R₃₇ and R₃₈ are not mutually bonded.

The compound of the third exemplary embodiment is preferably a compound represented by the formula (121).

In the compound of the third exemplary embodiment, A₂ is preferably a substituted or unsubstituted aryl group having 6 to 18 ring carbon atoms or a substituted or unsubstituted heterocyclic group having 5 to 18 ring atoms.

In the compound of the third exemplary embodiment, A₂ is more preferably a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, or a group represented by the formula (14).

In the compound of the third exemplary embodiment, it is preferable that L₂ is a single bond and A₂ is a substituted or unsubstituted aryl group having 6 to 18 ring carbon atoms or a substituted or unsubstituted heterocyclic group having 5 to 18 ring atoms.

In the compound of the third exemplary embodiment, it is more preferable that L₂ is a single bond and A₂ is a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, or a group represented by the formula (14).

In the compound of the third exemplary embodiment, it is still more preferable that L₂ is a single bond and A₂ is an unsubstituted phenyl group.

In the compound of the third exemplary embodiment, it is preferable that R₁₁ to R₁₈, R₂₁ to R₂₈ and R₃₁ to R₃₈ are each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms, and R₃₉ is a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the compound of the third exemplary embodiment, it is more preferable that R₁₁ to R₁₈, R₂₁ to R₂₈ and R₃₁ to R₃₈ are each independently a hydrogen atom or a substituted or unsubstituted aryl group having 6 to 30 ring carbon atoms, and R₃₉ is a substituted or unsubstituted aryl group having 6 to 30 ring carbon atoms.

In the compound of the third exemplary embodiment, it is still more preferable that R₁₁ to R₁₈, R₂₁ to R₂₈ and R₃₁ to R₃₈ are each a hydrogen atom and R₃₉ is a substituted or unsubstituted phenyl group.

In the compound of the third exemplary embodiment, it is preferable that L₁ and L₂ are each independently a single bond or a substituted or unsubstituted arylene group having 6 to 30 ring carbon atoms.

In the compound of the third exemplary embodiment, it is more preferable that L₁ and L₂ are each independently a substituted or unsubstituted phenylene group, a substituted or unsubstituted parabiphenylene group, or a substituted or unsubstituted paraterphenylene group.

In the compound of the third exemplary embodiment, it is still more preferable that L₁ and L₂ are each independently a substituted or unsubstituted parabiphenylene group or a substituted or unsubstituted paraterphenylene group.

In the compound of the third exemplary embodiment, it is also preferable that L₁ and L₂ are each independently a single bond or at least one group selected from the group consisting of groups represented by the formulae (L1) to (L7).

In the compound of the third exemplary embodiment, it is also more preferable that L₂ is a single bond or a group represented by one of the formulae (L3), (L4) and (L6).

In the compound of the third exemplary embodiment, a substituent for “substituted or unsubstituted” group is each independently preferably a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon, <<nret>>

more preferably an unsubstituted aryl group having 6 to 30 ring carbon atoms, an unsubstituted heterocyclic group having 5 to 30 ring atoms, or an unsubstituted alkyl group having 1 to 30 carbon atoms.

The compound of the third exemplary embodiment corresponds to a compound of an example of the compound M3 described in the first exemplary embodiment. Therefore, specific examples of the compound of the third exemplary embodiment are also shown in the examples of the compound M3 described in the first exemplary embodiment.

### Organic EL Device

An organic EL device in an exemplary arrangement according to the third exemplary embodiment contains the compound of the third exemplary embodiment (compound represented by the formula (121) or (122)) in at least one of the organic layer(s). An organic EL device in another exemplary arrangement according to the third exemplary embodiment contains the compound of the third exemplary embodiment (compound represented by the formula (121) or (122)) in the emitting layer.

An organic EL device in still another exemplary arrangement according to the third exemplary embodiment is an organic EL device in which the compound M3 in the organic EL device according to the first exemplary embodiment is replaced by the compound of the third exemplary embodiment (compound represented by the formula (121) or (122)).

An organic EL device in a further exemplary arrangement according to the third exemplary embodiment is an organic EL device in which the compound M3 in the organic EL device according to the second exemplary embodiment is replaced by the compound of the third exemplary embodiment (compound represented by the formula (121) or (122)).

The compound according to the third exemplary embodiment enables a high performance, for instance, improvement in luminous efficiency of the organic EL device.

Accordingly, the organic EL device in the exemplary arrangement according to the third exemplary embodiment also has a high performance, for instance, a high luminous efficiency.

### Fourth Exemplary Embodiment

A compound of a fourth exemplary embodiment is a compound represented by one of formulae (131) to (134) below. A compound falling under one of the formulae (131) to (134) among the specific examples of the compound M3 of the first exemplary embodiment is used as a specific example of the compound of the fourth exemplary embodiment.

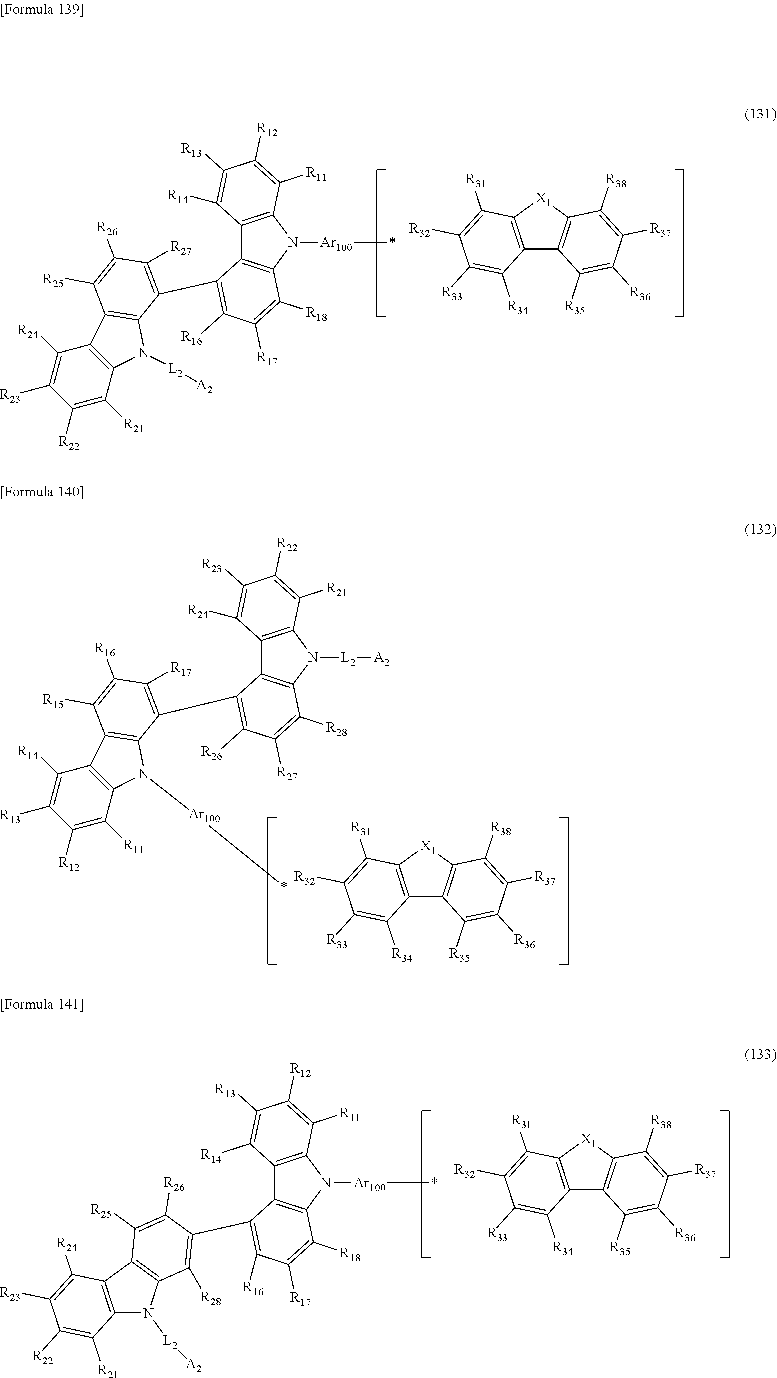

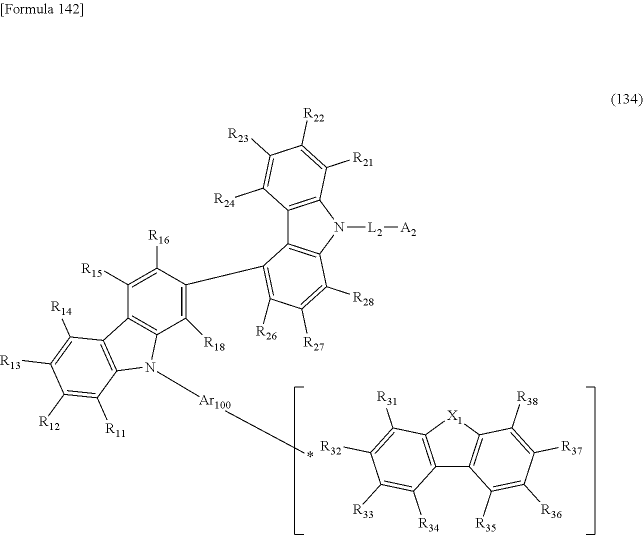

In the formulae (131) to (134):
A₂ is a substituted or unsubstituted aryl group having 6 to 30 ring carbon atoms, or a substituted or unsubstituted heterocyclic group having 5 to 30 ring atoms;
Ar₁₀₀ is a substituted or unsubstituted arylene group having 6 to 30 ring carbon atoms;
L₂ is a single bond, a substituted or unsubstituted arylene group having 6 to 30 ring carbon atoms, a substituted or unsubstituted divalent heterocyclic group having 5 to 30 ring atoms, a group obtained by bonding two groups selected from the group consisting of a substituted or unsubstituted arylene group having 6 to 30 ring carbon atoms and a substituted or unsubstituted divalent heterocyclic group having 5 to 30 ring atoms, or a group obtained by bonding three groups selected from the group consisting of a substituted or unsubstituted arylene group having 6 to 30 ring carbon atoms and a substituted or unsubstituted divalent heterocyclic group having 5 to 30 ring atoms;
the two groups are mutually the same or different;
the three groups are mutually the same or different;
R₁₁ to R₁₈ are each independently a hydrogen atom or a substituent, or at least one combination of a combination of R₁₁ and R₁₂, a combination of R₁₂ and R₁₃, a combination of R₁₃ and R₁₄, a combination of R₁₅ and R₁₆, a combination of R₁₆ and R₁₇, or a combination of R₁₇ and R₁₈ are mutually bonded to form a ring;
R₂₁ to R₂₈ are each independently a hydrogen atom or a substituent, or at least one combination of a combination of R₂₁ and R₂₂, a combination of R₂₂ and R₂₃, a combination of R₂₃ and R₂₄, a combination of R₂₅ and R₂₆, a combination of R₂₆ and R₂₇, or a combination of R₂₇ and R₂₈ are mutually bonded to form a ring;
X₁ is an oxygen atom, a sulfur atom, or NR₃₉;
R₃₉ is a substituent;
R₃₁ to R₃₈ are each independently a hydrogen atom or a substituent, or at least one combination of a combination of R₃₁ and R₃₂, a combination of R₃₂ and R₃₃, a combination of R₃₃ and R₃₄, a combination of R₃₅ and R₃₆, a combination of R₃₆ and R₃₇, or a combination of R₃₇ and R₃₈ are mutually bonded to form a ring;
one selected from carbon atoms bonded to R₃₁ to R₃₈ and a nitrogen atom bonded to R₃₉ is bonded to *;
R₁₁ to R₁₈, R₂₁ to R₂₈, and R₃₁ to R₃₉ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, a group represented by —N(Rz)₂, a thiol group, a substituted or unsubstituted alkylthio group having 1 to 30 carbon atoms, a substituted or unsubstituted aralkyl group having 7 to 30 ring carbon atoms, a substituted germanium group, a substituted phosphine oxide group, a nitro group, a substituted boryl group, or a substituted or unsubstituted arylthio group having 6 to 30 ring carbon atoms;
Rz is a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms;
two Rz in —N(Rz)₂ are mutually the same or different;
when Ar₁₀₀ has a substituent, the substituent is each independently a halogen atom, a cyano group, an unsubstituted aryl group having 6 to 30 ring carbon atoms, an unsubstituted heterocyclic group having 5 to 30 ring atoms, an unsubstituted alkyl group having 1 to 30 carbon atoms, an unsubstituted alkyl halide group having 1 to 30 carbon atoms, an unsubstituted alkenyl group having 2 to 30 carbon atoms, an unsubstituted alkynyl group having 2 to 30 carbon atoms, an unsubstituted alkylsilyl group having 3 to 30 carbon atoms, an unsubstituted arylsilyl group having 6 to 60 ring carbon atoms, an unsubstituted arylphosphoryl group having 6 to 60 ring carbon atoms, a hydroxy group, an unsubstituted alkoxy group having 1 to 30 carbon atoms, an unsubstituted aryloxy group having 6 to 30 ring carbon atoms, a thiol group, an unsubstituted alkylthio group having 1 to 30 carbon atoms, an unsubstituted aralkyl group having 7 to 30 ring carbon atoms, a substituted germanium group, a substituted phosphine oxide group, a nitro group, a substituted boryl group, or an unsubstituted arylthio group having 6 to 30 ring carbon atoms.

In the formulae (131) to (134): when a carbon atom bonded to R₃₁ is bonded to *, a hydrogen atom represented by R₃₁, a substituent represented by R₃₁, and a ring formed by involving R₃₁ are not present.

In the formulae (131) to (134), when a carbon atom bonded to R₃₂ is bonded to *, a hydrogen atom represented by R₃₂, a substituent represented by R₃₂, and a ring formed by involving R₃₂ are not present.

In the formulae (131) to (134), when a carbon atom bonded to R₃₃ is bonded to *, a hydrogen atom represented by R₃₃, a substituent represented by R₃₃, and a ring formed by involving R₃₃ are not present.

In the formulae (131) to (134), when a carbon atom bonded to R₃₄ is bonded to *, a hydrogen atom represented by R₃₄, a substituent represented by R₃₄, and a ring formed by involving R₃₄ are not present.

In the formulae (131) to (134), when a carbon atom bonded to R₃₅ is bonded to *, a hydrogen atom represented by R₃₅, a substituent represented by R₃₅, and a ring formed by involving R₃₅ are not present.

In the formulae (131) to (134), when a carbon atom bonded to R₃₆ is bonded to *, a hydrogen atom represented by R₃₆, a substituent represented by R₃₆, and a ring formed by involving R₃₆ are not present.

In the formulae (131) to (134), when a carbon atom bonded to R₃₇ is bonded to *, a hydrogen atom represented by R₃₇, a substituent represented by R₃₇, and a ring formed by involving R₃₇ are not present.

In the formulae (131) to (134), when a carbon atom bonded to R₃₈ is bonded to *, a hydrogen atom represented by R₃₈, a substituent represented by R₃₈, and a ring formed by involving R₃₈ are not present.

In the formulae (131) to (134), when a nitrogen atom bonded to R₃₉ is bonded to *, a substituent represented by R₃₉ is not present.

In the formulae (131) to (134): it is preferable that a combination of R₁₁ and R₁₂, a combination of R₁₂ and R₁₃, a combination of R₁₃ and R₁₄, a combination of R₁₅ and R₁₆, a combination of R₁₆ and R₁₇, and a combination of R₁₇ and R₁₈ are not mutually bonded.

In the formulae (131) to (134): it is preferable that a combination of R₂₁ and R₂₂, a combination of R₂₂ and R₂₃, a combination of R₂₃ and R₂₄, a combination of R₂₅ and R₂₆, a combination of R₂₆ and R₂₇, and a combination of R₂₇ and R₂₈ are not mutually bonded.

In the formulae (131) to (134): it is preferable that a combination of R₃₁ and R₃₂, a combination of R₃₂ and R₃₃, a combination of R₃₃ and R₃₄, a combination of R₃₅ and R₃₆, a combination of R₃₆ and R₃₇, and a combination of R₃₇ and R₃₈ are not mutually bonded.

In the compound of the fourth exemplary embodiment, A₂ is preferably a substituted or unsubstituted aryl group having 6 to 18 ring carbon atoms or a substituted or unsubstituted heterocyclic group having 5 to 18 ring atoms. In the compound of the fourth exemplary embodiment, A₂ is more preferably a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, or a group represented by the formula (14).

In the compound of the fourth exemplary embodiment, it is preferable that L₂ is a single bond and A₂ is a substituted or unsubstituted aryl group having 6 to 18 ring carbon atoms or a substituted or unsubstituted heterocyclic group having 5 to 18 ring atoms.

In the compound of the fourth exemplary embodiment, it is more preferable that L₂ is a single bond and A₂ is a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, or a group represented by the formula (14).

In the compound of the fourth exemplary embodiment, it is still more preferable that L₂ is a single bond and A₂ is an unsubstituted phenyl group.

In the compound of the fourth exemplary embodiment, X₁ is preferably NR₃₉.

In the compound of the fourth exemplary embodiment, it is preferable that X₁ is preferably NR₃₉ and a nitrogen atom bonded to R₃₉ is bonded to *.

In the compound of the fourth exemplary embodiment, it is preferable that R₁₁ to R₁₈, R₂₁ to R₂₈ and R₃₁ to R₃₈ are each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms, and R₃₉ is a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the compound of the fourth exemplary embodiment, it is more preferable that R₁₁ to R₁₈, R₂₁ to R₂₈ and R₃₁ to R₃₈ are each independently a hydrogen atom or a substituted or unsubstituted aryl group having 6 to 30 ring carbon atoms, and R₃₉ is a substituted or unsubstituted aryl group having 6 to 30 ring carbon atoms.

In the compound of the fourth exemplary embodiment, it is still more preferable that R₁₁ to R₁₈, R₂₁ to R₂₈ and R₃₁ to R₃₈ are each a hydrogen atom and R₃₉ is a substituted or unsubstituted phenyl group.

In the compound of the fourth exemplary embodiment, it is preferable that Ar₁₀₀ and L₂ are each independently a single bond or a substituted or unsubstituted arylene group having 6 to 30 ring carbon atoms.

In the compound of the fourth exemplary embodiment, it is more preferable that Ar₁₀₀ and L₂ are each independently a substituted or unsubstituted phenylene group, a substituted or unsubstituted parabiphenylene group, or a substituted or unsubstituted paraterphenylene group.

In the compound of the fourth exemplary embodiment, it is still more preferable that Ar₁₀₀ and L₂ are each independently a substituted or unsubstituted parabiphenylene group or a substituted or unsubstituted paraterphenylene group.

In the compound of the fourth exemplary embodiment, it is also preferable that Ar₁₀₀ and L₂ are each independently a single bond or at least one group selected from the group consisting of groups represented by the formulae (L1) to (L7).

In the compound of the fourth exemplary embodiment, it is also more preferable that L₂ is a single bond or a group represented by one of the formulae (L3), (L4) and (L6).

In the compound of the fourth exemplary embodiment, a substituent for “substituted or unsubstituted” group is each independently preferably a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon, more preferably an unsubstituted aryl group having 6 to 30 ring carbon atoms, an unsubstituted heterocyclic group having 5 to 30 ring atoms, or an unsubstituted alkyl group having 1 to 30 carbon atoms.

The compound of the fourth exemplary embodiment corresponds to a compound of an example of the compound M3 described in the first exemplary embodiment. Therefore, specific examples of the compound of the fourth exemplary embodiment are also shown in the examples of the compound M3 described in the first exemplary embodiment.

### Organic EL Device

An organic EL device in an exemplary arrangement according to the fourth exemplary embodiment contains the compound of the fourth exemplary embodiment (compound represented by one of the formulae (131) to (134)) in at least one of the organic layer(s). An organic EL device in another exemplary arrangement according to the fourth exemplary embodiment contains the compound of the fourth exemplary embodiment (compound represented by one of the formulae (131) to (134)) in the emitting layer.

An organic EL device in still another exemplary arrangement according to the fourth exemplary embodiment is an organic EL device in which the compound M3 in the organic EL device according to the first exemplary embodiment is replaced by the compound of the fourth exemplary embodiment (compound represented by one of the formulae (131) to (134)).

An organic EL device in a further exemplary arrangement according to the fourth exemplary embodiment is an organic EL device in which the compound M3 in the organic EL device according to the second exemplary embodiment is replaced by the compound of the fourth exemplary embodiment (compound represented by one of the formulae (131) to (134)).

The compound according to the fourth exemplary embodiment enables a high performance, for instance, improvement in luminous efficiency of the organic EL device.

Accordingly, the organic EL device in the exemplary arrangement according to the fourth exemplary embodiment also has a high performance, for instance, a high luminous efficiency.

### Fifth Exemplary Embodiment

### Compound

A compound of a fifth exemplary embodiment is a compound represented by a formula (141), (142) or (143) below. A compound falling under the formula (141), (142) or (143) among the specific examples of the compound M3 of the first exemplary embodiment is used as a specific example of the compound of the fifth exemplary embodiment.

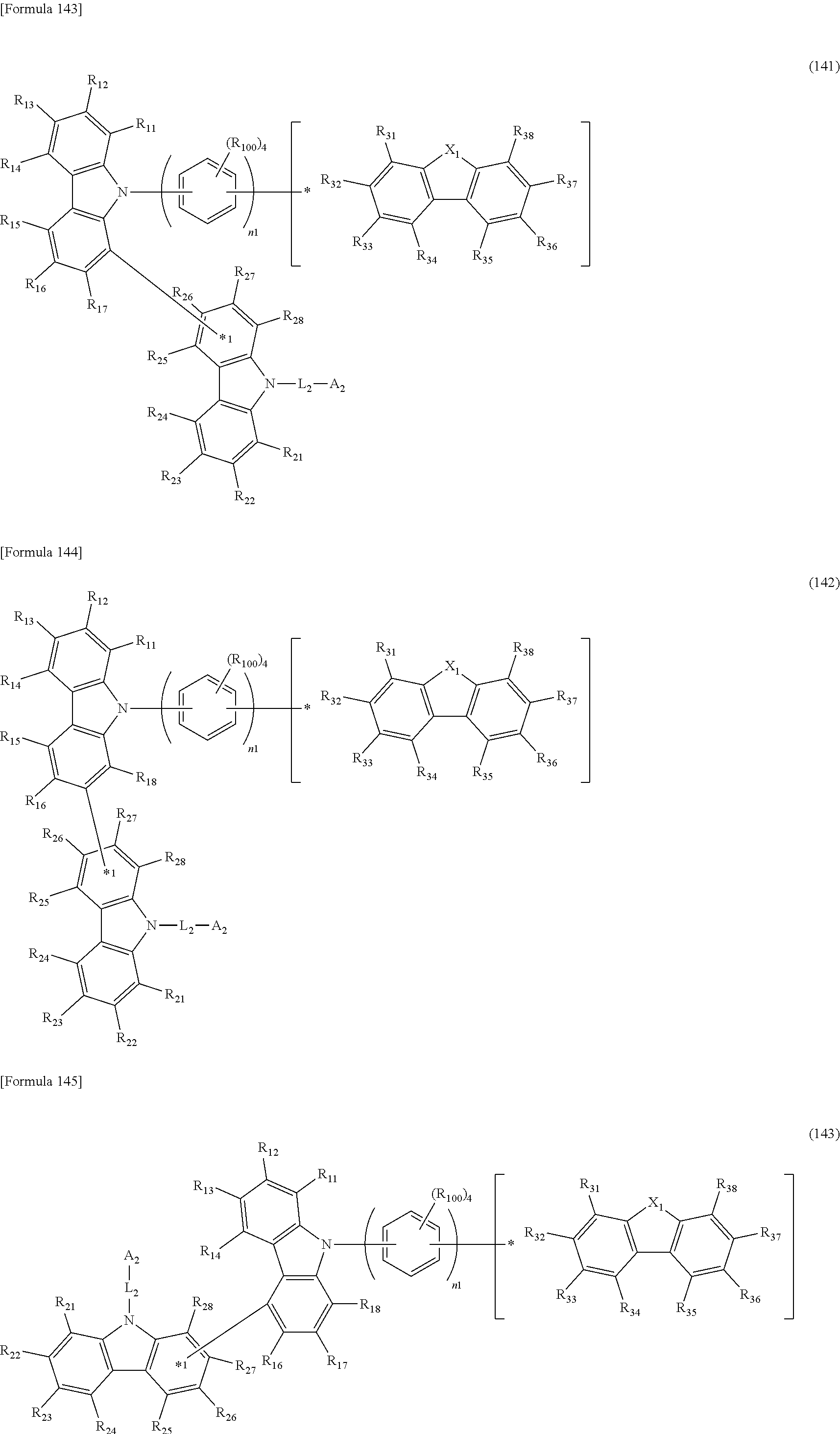

In the formulae (141) to (143):
A₂ is a substituted or unsubstituted aryl group having 6 to 30 ring carbon atoms, or a substituted or unsubstituted heterocyclic group having 5 to 30 ring atoms;
L₂ are each independently a single bond, a substituted or unsubstituted arylene group having 6 to 30 ring carbon atoms, a substituted or unsubstituted divalent heterocyclic group having 5 to 30 ring atoms, a group obtained by bonding two groups selected from the group consisting of a substituted or unsubstituted arylene group having 6 to 30 ring carbon atoms and a substituted or unsubstituted divalent heterocyclic group having 5 to 30 ring atoms, or a group obtained by bonding three groups selected from the group consisting of a substituted or unsubstituted arylene group having 6 to 30 ring carbon atoms and a substituted or unsubstituted divalent heterocyclic group having 5 to 30 ring atoms; the two groups are mutually the same or different;
the three groups are mutually the same or different;
R₁₀₀ is each independently a hydrogen atom or a substituent;
n1 is 2, 3, or 4;
four R₁₀₀ are mutually the same or different;
R₁₁ to R₁₈ are each independently a hydrogen atom or a substituent, or at least one combination of a combination of R₁₁ and R₁₂, a combination of R₁₂ and R₁₃, a combination of R₁₃ and R₁₄, a combination of R₁₅ and R₁₆, a combination of R₁₆ and R₁₇, or a combination of R₁₇ and R₁₈ are mutually bonded to form a ring;
R₂₁ to R₂₈ are each independently a hydrogen atom or a substituent, or at least one combination of a combination of R₂₁ and R₂₂, a combination of R₂₂ and R₂₃, a combination of R₂₃ and R₂₄, a combination of R₂₅ and R₂₆, a combination of R₂₆ and R₂₇, or a combination of R₂₇ and R₂₈ are mutually bonded to form a ring;
one of carbon atoms bonded to R₂₅, R₂₇ and R₂₈ is bonded to *1;
X₁ is an oxygen atom, a sulfur atom, or NR₃₉;
R₃₉ is a substituent;
R₃₁ to R₃₈ are each independently a hydrogen atom or a substituent, or at least one combination of a combination of R₃₁ and R₃₂, a combination of R₃₂ and R₃₃, a combination of R₃₃ and R₃₄, a combination of R₃₅ and R₃₆, a combination of R₃₆ and R₃₇, or a combination of R₃₇ and R₃₈ are mutually bonded to form a ring;
one selected from carbon atoms bonded to R₃₁ to R₃₈ and a nitrogen atom bonded to R₃₉ is bonded to *;
R₁₀₀, R₁₁ to R₁₈, R₂₁ to R₂₈ and R₃₁ to R₃₉ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, a group represented by —N(Rz)₂, a thiol group, a substituted or unsubstituted alkylthio group having 1 to 30 carbon atoms, a substituted or unsubstituted aralkyl group having 7 to 30 ring carbon atoms, a substituted germanium group, a substituted phosphine oxide group, a nitro group, a substituted boryl group, or a substituted or unsubstituted arylthio group having 6 to 30 ring carbon atoms;
Rz is a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms; two Rz in —N(Rz)₂ are mutually the same or different;
R₁₀₀ is not a group represented by —N(Rz)₂;
when R₁₀₀ has a substituent, the substituent is each independently a halogen atom, a cyano group, an unsubstituted aryl group having 6 to 30 ring carbon atoms, an unsubstituted heterocyclic group having 5 to 30 ring atoms, an unsubstituted alkyl group having 1 to 30 carbon atoms, an unsubstituted alkyl halide group having 1 to 30 carbon atoms, an unsubstituted alkenyl group having 2 to 30 carbon atoms, an unsubstituted alkynyl group having 2 to 30 carbon atoms, an unsubstituted alkylsilyl group having 3 to 30 carbon atoms, an unsubstituted arylsilyl group having 6 to 60 ring carbon atoms, an unsubstituted arylphosphoryl group having 6 to 60 ring carbon atoms, a hydroxy group, an unsubstituted alkoxy group having 1 to 30 carbon atoms, an unsubstituted aryloxy group having 6 to 30 ring carbon atoms, a thiol group, an unsubstituted alkylthio group having 1 to 30 carbon atoms, an unsubstituted aralkyl group having 7 to 30 ring carbon atoms, a substituted germanium group, a substituted phosphine oxide group, a nitro group, a substituted boryl group, or an unsubstituted arylthio group having 6 to 30 ring carbon atoms.

In the formulae (141) to (143): when a carbon atom bonded to R₃₁ is bonded to *, a hydrogen atom represented by R₃₁, a substituent represented by R₃₁, and a ring formed by involving R₃₁ are not present.

In the formulae (141) to (143), when a carbon atom bonded to R₃₂ is bonded to *, a hydrogen atom represented by R₃₂, a substituent represented by R₃₂, and a ring formed by involving R₃₂ are not present.

In the formulae (141) to (143), when a carbon atom bonded to R₃₃ is bonded to *, a hydrogen atom represented by R₃₃, a substituent represented by R₃₃, and a ring formed by involving R₃₃ are not present.

In the formulae (141) to (143), when a carbon atom bonded to R₄ is bonded to *, a hydrogen atom represented by R₃₄, a substituent represented by R₃₄, and a ring formed by involving R₃₄ are not present.

In the formulae (141) to (143), when a carbon atom bonded to R₃₅ is bonded to *, a hydrogen atom represented by R₃₅, a substituent represented by R₃₅, and a ring formed by involving R₃₅ are not present.

In the formulae (141) to (143), when a carbon atom bonded to R₃₆ is bonded to *, a hydrogen atom represented by R₃₆, a substituent represented by R₃₆, and a ring formed by involving R₃₆ are not present.

In the formulae (141) to (143), when a carbon atom bonded to R₃₇ is bonded to *, a hydrogen atom represented by R₃₇, a substituent represented by R₃₇, and a ring formed by involving R₃₇ are not present.

In the formulae (141) to (143), when a carbon atom bonded to R₃₈ is bonded to *, a hydrogen atom represented by R₃₈, a substituent represented by R₃₈, and a ring formed by involving R₃₈ are not present.

In the formulae (141) to (143), when a nitrogen atom bonded to R₃₉ is bonded to *, a substituent represented by R₃₉ is not present.

In the formulae (141) to (143), when a carbon atom bonded to R₂₅ is bonded to *1, a hydrogen atom represented by R₂₅, a substituent represented by R₂₅, and a ring formed by involving R₂₅ are not present.

In the formulae (141) to (143), when a carbon atom bonded to R₂₇ is bonded to *1, a hydrogen atom represented by R₂₇, a substituent represented by R₂₇, and a ring formed by involving R₂₇ are not present.

In the formulae (141) to (143): when a carbon atom bonded to R₂₈ is bonded to *1, a hydrogen atom represented by R₂₈, a substituent represented by R₂₈, and a ring formed by involving R₂₈ are not present.

The compound of the fifth exemplary embodiment is preferably a compound represented by a formula (141A), (142A) or (143A) below.

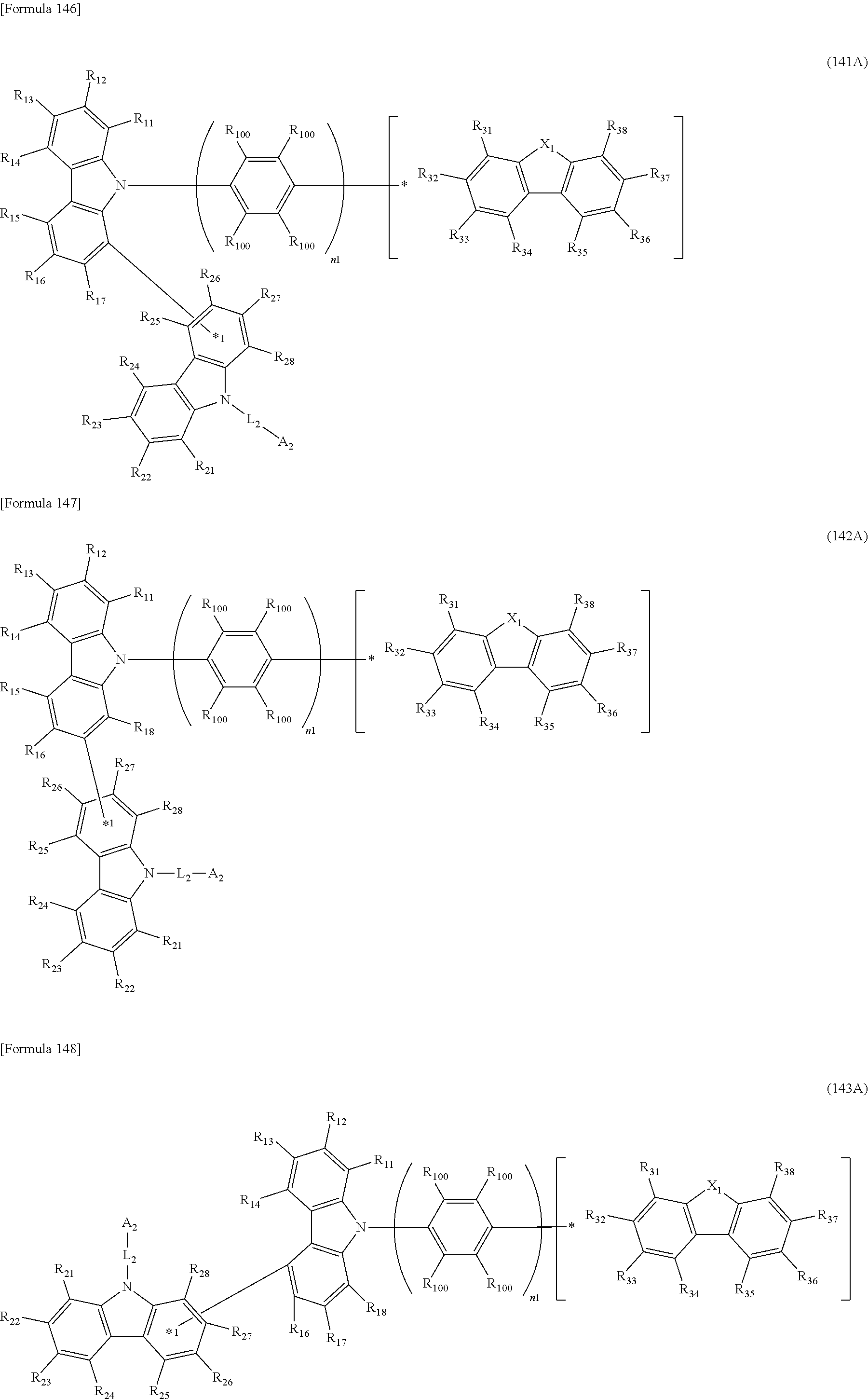

In the formulae (141A), (142A) and (143A): A₂, L₂, X₁, R₁₁ to R₁₈, R₂₁ to R₂₈, R₃₁ to R₃₉, R₁₀₀ and n1 each independently represent the same as A₂, L₂, X₁, R₁₁ to R₁₈, R₂₁ to R₂₈, R₃₁ to R₃₉, R₁₀₀ and n1 in the formulae (141) to (143); four R₁₀₀ are mutually the same or different; one of carbon atoms bonded to R₂₅, R₂₇ and R₂₈ is bonded to *1, and one selected from carbon atoms bonded to R₃₁ to R₃₈ and a nitrogen atom bonded to R₃₉ is bonded to *.

In the formulae (141A), (142A) and (143A): when a carbon atom bonded to R₃₁ is bonded to *, a hydrogen atom represented by R₃₁, a substituent represented by R₃₁, and a ring formed by involving R₃₁ are not present.

In the formulae (141A), (142A) and (143A): when a carbon atom bonded to R₃₂ is bonded to *, a hydrogen atom represented by R₃₂, a substituent represented by R₃₂, and a ring formed by involving R₃₂ are not present.

In the formulae (141A), (142A) and (143A): when a carbon atom bonded to R₃₃ is bonded to *, a hydrogen atom represented by R₃₃, a substituent represented by R₃₃, and a ring formed by involving R₃₃ are not present.

In the formulae (141A), (142A) and (143A): when a carbon atom bonded to R₃₄ is bonded to *, a hydrogen atom represented by R₃₄, a substituent represented by R₃₄, and a ring formed by involving R₃₄ are not present.

In the formulae (141A), (142A) and (143A): when a carbon atom bonded to R₃₅ is bonded to *, a hydrogen atom represented by R₃₅, a substituent represented by R₃₅, and a ring formed by involving R₃₅ are not present.

In the formulae (141A), (142A) and (143A): when a carbon atom bonded to R₃₆ is bonded to *, a hydrogen atom represented by R₃₆, a substituent represented by R₃₆, and a ring formed by involving R₃₆ are not present.

In the formulae (141A), (142A) and (143A): when a carbon atom bonded to R₃₇ is bonded to *, a hydrogen atom represented by R₃₇, a substituent represented by R₃₇, and a ring formed by involving R₃₇ are not present.

In the formulae (141A), (142A) and (143A): when a carbon atom bonded to R₃₈ is bonded to *, a hydrogen atom represented by R₃₈, a substituent represented by R₃₈, and a ring formed by involving R₃₈ are not present.

In the formulae (141A), (142A) and (143A), when a nitrogen atom bonded to R₃₉ is bonded to *, a substituent represented by R₃₉ is not present.

In the formulae (141A), (142A) and (143A): when a carbon atom bonded to R₂₅ is bonded to *1, a hydrogen atom represented by R₂₅, a substituent represented by R₂₅, and a ring formed by involving R₂₅ are not present.

In the formulae (141A), (142A) and (143A): when a carbon atom bonded to R₂₇ is bonded to *1, a hydrogen atom represented by R₂₇, a substituent represented by R₂₇, and a ring formed by involving R₂₇ are not present.

In the formulae (141A), (142A) and (143A): when a carbon atom bonded to R₂₆ is bonded to *1, a hydrogen atom represented by R₂₆, a substituent represented by R₂₆, and a ring formed by involving R₂₆ are not present.

In the formulae (141) to (143), (141A), (142A) and (143A): it is preferable that a combination of R₁₁ and R₁₂, a combination of R₁₂ and R₁₃, a combination of R₁₃ and R₁₄, a combination of R₁₅ and R₁₈, a combination of R₁₆ and R₁₇, and a combination of R₁₇ and R₁₈ are not mutually bonded.

In the formulae (141) to (143), (141A), (142A) and (143A): it is preferable that a combination of R₂₁ and R₂₂, a combination of R₂₂ and R₂₃, a combination of R₂₃ and R₂₄, a combination of R₂₅ and R₂₆, a combination of R₂₆ and R₂₇, and a combination of R₂₇ and R₂₈ are not mutually bonded.

In the formulae (141) to (143), (141A), (142A) and (143A): it is preferable that a combination of R₃₁ and R₃₂, a combination of R₃₂ and R₃₃, a combination of R₃₃ and R₃₄, a combination of R₃₅ and R₃₆, a combination of R₃₆ and R₃₇, and a combination of R₃₇ and R₃₈ are not mutually bonded.

In the formulae (141) to (143), (141A), (142A) and (143A), n1 is preferably 2 or 3.

In the compound of the fifth exemplary embodiment, A₂ is preferably a substituted or unsubstituted aryl group having 6 to 18 ring carbon atoms or a substituted or unsubstituted heterocyclic group having 5 to 18 ring atoms.

In the compound of the fifth exemplary embodiment, A₂ is more preferably a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, or a group represented by the formula (14).

In the compound of the fifth exemplary embodiment, it is preferable that L₂ is a single bond and A₂ is a substituted or unsubstituted aryl group having 6 to 18 ring carbon atoms or a substituted or unsubstituted heterocyclic group having 5 to 18 ring atoms.

In the compound of the fifth exemplary embodiment, it is more preferable that L₂ is a single bond and A₂ is a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, or a group represented by the formula (14).

In the compound of the fifth exemplary embodiment, it is still more preferable that L₂ is a single bond and A₂ is an unsubstituted phenyl group.

In the compound of the fifth exemplary embodiment, X₁ is preferably NR₃₉.

In the compound of the fifth exemplary embodiment, it is preferable that X₁ is NR₃₉ and a nitrogen atom bonded to R₃₉ is bonded to *.

In the compound of the fifth exemplary embodiment, it is preferable that R₁ to R₁₈, R₂₁ to R₂₈ and R₃₁ to R₃₈ are each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms, and R₃₉ is a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the compound of the fifth exemplary embodiment, it is more preferable that R₁₁ to R₁₈, R₂₁ to R₂₈ and R₃₁ to R₃₈ are each independently a hydrogen atom or a substituted or unsubstituted aryl group having 6 to 30 ring carbon atoms, and R₃₉ is a substituted or unsubstituted aryl group having 6 to 30 ring carbon atoms.

In the compound of the fifth exemplary embodiment, it is still more preferable that R₁₁ to R₁₈, R₂₁ to R₂₈ and R₃₁ to R₃₈ are each a hydrogen atom and R₃₉ is a substituted or unsubstituted phenyl group.

In the compound of the fifth exemplary embodiment, it is preferable that L₂ is each independently a single bond or a substituted or unsubstituted arylene group having 6 to 30 ring carbon atoms.

In the compound of the fifth exemplary embodiment, it is more preferable that L₂ is each independently a substituted or unsubstituted phenylene group, a substituted or unsubstituted parabiphenylene group, or a substituted or unsubstituted paraterphenylene group.

In the compound of the fifth exemplary embodiment, it is still more preferable that L₂ is each independently a substituted or unsubstituted parabiphenylene group or a substituted or unsubstituted paraterphenylene group.

In the compound of the fifth exemplary embodiment, it is also preferable that L₂ is each independently a single bond or at least one group selected from the group consisting of groups represented by the formulae (L1) to (L7).

In the compound of the fifth exemplary embodiment, it is also more preferable that L₂ is a single bond or a group represented by one of the formulae (L3), (L4) and (L6).

In the compound of the fifth exemplary embodiment, a substituent for “substituted or unsubstituted” group is each independently preferably a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon, more preferably an unsubstituted aryl group having 6 to 30 ring carbon atoms, an unsubstituted heterocyclic group having 5 to 30 ring atoms, or an unsubstituted alkyl group having 1 to 30 carbon atoms.

The compound of the fifth exemplary embodiment also corresponds to a compound of an example of the compound M3 described in the first exemplary embodiment. Therefore, specific examples of the compound of the fifth exemplary embodiment are also shown in the examples of the compound M3 described in the first exemplary embodiment.

### Organic EL Device

An organic EL device in an exemplary arrangement according to the fifth exemplary embodiment contains the compound of the fifth exemplary embodiment (compound represented by the formula (141), (142) or (143)) in at least one of the organic layer(s). An organic EL device in another exemplary arrangement according to the fifth exemplary embodiment contains the compound of the fifth exemplary embodiment (compound represented by the formula (141), (142) or (143)) in the emitting layer.

An organic EL device in still another exemplary arrangement according to the fifth exemplary embodiment is an organic EL device in which the compound M3 in the organic EL device according to the first exemplary embodiment is replaced by the compound of the fifth exemplary embodiment (compound represented by the formula (141), (142) or (143)).

An organic EL device in a further exemplary arrangement according to the fifth exemplary embodiment is an organic EL device in which the compound M3 in the organic EL device according to the second exemplary embodiment is replaced by the compound of the fifth exemplary embodiment (compound represented by the formula (141), (142) or (143)).

The compound according to the fifth exemplary embodiment enables a high performance, for instance, improvement in luminous efficiency of the organic EL device.

Accordingly, the organic EL device in the exemplary arrangement according to the fifth exemplary embodiment also has a high performance, for instance, a high luminous efficiency.

The compound of the third exemplary embodiment (compound represented by the formula (121) or (122)), the compound of the fourth exemplary embodiment (compound represented by one of the formulae (131) to (134)), and the compound of the fifth exemplary embodiment (compound represented by the formula (141), (142) or (143)), which are described above, may contain at least one deuterium atom.

An exemplary arrangement in which the compound of the third exemplary embodiment, the compound of the fourth exemplary embodiment, and the compound of the fifth exemplary embodiment contain at least one deuterium atom is exemplified by an exemplary arrangement in which the compound M3, in “the exemplary arrangement in which the compound M3 contains at least one deuterium atom” described in the first exemplary embodiment, is replaced by each of the compound of the third exemplary embodiment, the compound of the fourth exemplary embodiment, and the compound of the fifth exemplary embodiment.

### Sixth Exemplary Embodiment

### Electronic Device

An electronic device according to a sixth exemplary embodiment is installed with any one of the organic EL devices according to the above exemplary embodiments. Examples of the electronic device include a display device and a light-emitting unit. Examples of the display device include a display component (e.g., an organic EL panel module), TV, mobile phone, tablet and personal computer. Examples of the light-emitting unit include an illuminator and a vehicle light.

### Seventh Exemplary Embodiment

### Organic-EL-Device Material

An organic-EL-device material of a seventh exemplary embodiment contains at least one of the compound of the third exemplary embodiment, the compound of the fourth exemplary embodiment, or the compound of the fifth exemplary embodiment.

The organic-EL-device material according to the seventh exemplary embodiment enables a high performance, for instance, improvement in luminous efficiency of the organic EL device.

The organic-EL-device material of the seventh exemplary embodiment may further contain an additional compound. When the organic-EL-device material of the seventh exemplary embodiment further contains an additional compound, the additional compound may be solid or liquid.

### Modification of Embodiment(s)

The scope of the invention is not limited by the above-described exemplary embodiments but includes any modification and improvement as long as such modification and improvement are compatible with the invention.

For instance, the emitting layer is not limited to a single layer, but may be provided by laminating a plurality of emitting layers. When the organic EL device has the plurality of emitting layers, it is only required that at least one of the emitting layers satisfies the conditions described in the above exemplary embodiments. For instance, the rest of the emitting layers may be a fluorescent emitting layer or a phosphorescent emitting layer with use of emission caused by electron transfer from the triplet excited state directly to the ground state.

When the organic EL device includes a plurality of emitting layers, these emitting layers may be mutually adjacently provided, or may form a so-called tandem organic EL device, in which a plurality of emitting units are layered via an intermediate layer.

For instance, a blocking layer may be provided adjacent to at least one of a side of the emitting layer close to the anode or a side of the emitting layer close to the cathode. The blocking layer is preferably provided in contact with the emitting layer to block at least any of holes, electrons, excitons or combinations thereof.

For instance, when the blocking layer is provided in contact with the side of the emitting layer close to the cathode, the blocking layer permits transport of electrons, and blocks holes from reaching a layer provided closer to the cathode (e.g., the electron transporting layer) beyond the blocking layer. When the organic EL device includes the electron transporting layer, the blocking layer is preferably interposed between the emitting layer and the electron transporting layer.

When the blocking layer is provided in contact with the side of the emitting layer close to the anode, the blocking layer permits transport of holes and blocks electrons from reaching a layer provided closer to the anode (e.g., the hole transporting layer) beyond the blocking layer. When the organic EL device includes the hole transporting layer, the blocking layer is preferably interposed between the emitting layer and the hole transporting layer.

Alternatively, the blocking layer may be provided adjacent to the emitting layer so that the excitation energy does not leak out from the emitting layer toward neighboring layer(s). The blocking layer blocks excitons generated in the emitting layer from being transferred to a layer(s) (e.g., the electron transporting layer and the hole transporting layer) closer to the electrode(s) beyond the blocking layer.

The emitting layer is preferably bonded with the blocking layer.

Specific structure, shape and the like of the components in the invention may be designed in any manner as long as an object of the invention can be achieved.

### Other Explanations

Herein, numerical ranges represented by “x to y” represents a range whose lower limit is the value (x) recited before “to” and whose upper limit is the value (y) recited after “to.”

Herein, the phrase “Rx and Ry are mutually bonded to form a ring” means, for instance, that Rx and Ry include a carbon atom, a nitrogen atom, an oxygen atom, a sulfur atom or a silicon atom, the atom(s) included in Rx (a carbon atom, a nitrogen atom, an oxygen atom, a sulfur atom or a silicon atom) and the atom(s) contained in Ry (a carbon atom, a nitrogen atom, an oxygen atom, a sulfur atom or a silicon atom) are bonded via a single bond(s), a double bond(s), a triple bond, and/or a divalent linking group(s) to form a ring having 5 or more ring atoms (specifically, a heterocycle or an aromatic hydrocarbon ring). x represents a number, a character or a combination of a number and a character. y represents a number, a character or a combination of a number and a character.

The divalent linking group is not limited. Examples of the divalent linking group include —O—, —CO—, —CO₂—, —S—, —SO—, —SO₂—, —NH—, —NRa-, and a group provided by a combination of two or more of these linking group.

Specific examples of the heterocyclic ring include a cyclic structure (heterocyclic ring) obtained by removing a bond from a “heteroaryl group Sub₂” exemplarily shown in the later-described “Description of Each Substituent in Formula.” The heterocyclic ring may have a substituent.

Specific examples of the heterocyclic ring include a cyclic structure (heterocyclic ring) obtained by removing a bond from an “aryl group Sub₁” exemplarily shown in the later-described “Description of Each Substituent in Formula.” The aromatic hydrocarbon ring may have a substituent.

Examples of Ra include a substituted or unsubstituted alkyl group Sub₃ having 1 to 30 carbon atoms, a substituted or unsubstituted aryl group Sub₁ having 6 to 30 ring carbon atoms, and a substituted or unsubstituted heteroaryl group Sub₂ having 5 to 30 ring atoms, which are exemplarily shown in the later-described “Description of Each Substituent in Formula.”

Rx and Ry are mutually bonded to form a ring, which means, for instance, that: an atom included in Rx₁ and an atom included in Ry₁ in a molecular structure represented by a formula (E1) below form a ring (cyclic structure) E represented by a formula (E2); an atom included in Rx₁ and an atom included in Ry₁ in a molecular structure represented by a formula (F1) below form a ring F represented by a formula (F2); an atom included in Rx₁ and an atom included in Ry₁ in a molecular structure represented by a formula (G1) below form a ring G represented by a formula (G2); an atom included in Rx₁ and an atom included in Ry₁ in a molecular structure represented by a formula (H1) below form a ring H represented by a formula (H2); and an atom included in Rx₁ and an atom included in Ry₁ in a molecular structure represented by a formula (I1) below form a ring I represented by a formula (I2).

In the formulae (E1) to (I1), * each independently represent a bonding position to another atom in a molecule. The two * in the formulae (E1), (F1), (G1), (H1) and (I1) correspond to two * in the formulae (E2), (F2), (G2), (H2) and (I2), respectively.

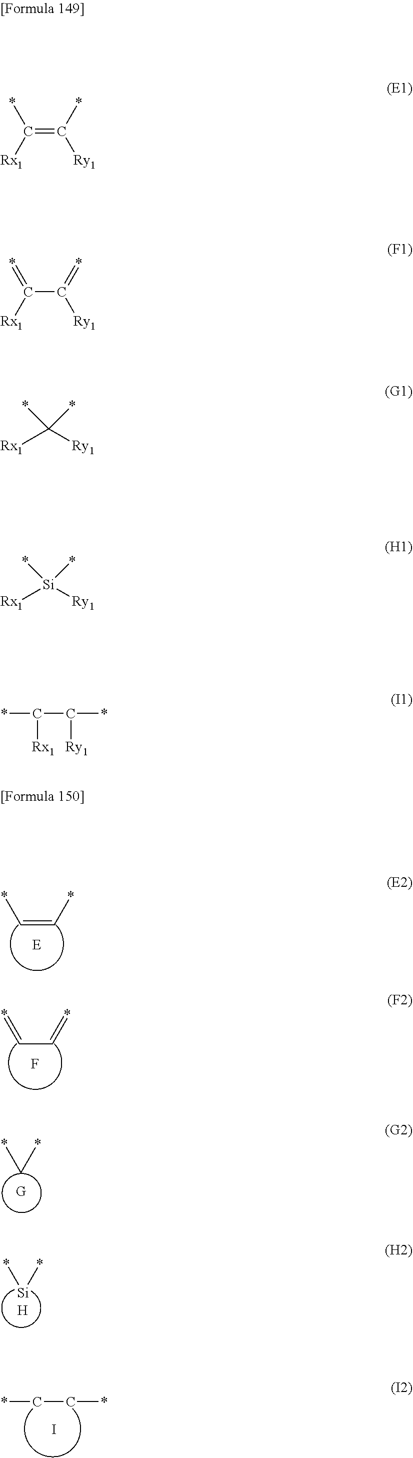

In the molecular structures represented by the formulae (E2) to (I2), E to I each represent a cyclic structure (the ring having 5 or more ring atoms). In the formulae (E2) to (I2), * each independently represent a bonding position to another atom in a molecule. The two * in the formula (E2) correspond to two * in the formula (E1). Similarly, two * in each of the formulae (F2) to (I2) correspond one-to-one to two *in each of the formulae (F1) to (I1).

For instance, in the formula (E1), Rx₁ and Ry₁ are mutually bonded to form a ring E in the formula (E2). When the ring E is an unsubstituted benzene ring, the molecular structure represented by the formula (E1) is the molecular structure represented by the formula (E3). Here, the two * in the formula (E3) each independently correspond to two *in each of the formulae (E1) and (E2).

For instance, in the formula (E1), Rx₁ and Ry₁ are mutually bonded to form a ring E in the formula (E2). When the ring E is an unsubstituted benzene ring, the molecular structure represented by the formula (E1) is the molecular structure represented by the formula (E4). Here, the two * in the formula (E4) each independently correspond to two * in each of the formulae (E1) and (E2). In the formulae (E3) to (E4), * each independently represent a bonding position to another atom in a molecule.

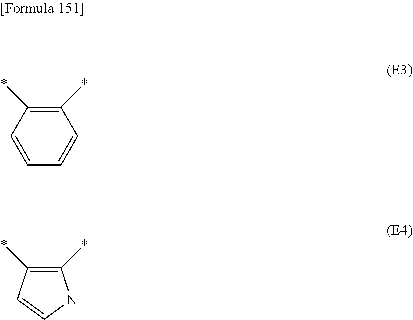 [SMILES: *c1ccccc1* ; *c1cc[nH]c1*]

Herein, the ring carbon atoms refer to the number of carbon atoms among atoms forming a ring of a compound (e.g., a monocyclic compound, fused-ring compound, crosslinking compound, carbon ring compound, and heterocyclic compound) in which the atoms are bonded to each other to form the ring. When the ring is substituted by a substituent(s), carbon atom(s) included in the substituent(s) is not counted in the ring carbon atoms. Unless specifically described, the same applies to the “ring carbon atoms” described later. For instance, a benzene ring has 6 ring carbon atoms, a naphthalene ring has 10 ring carbon atoms, a pyridinyl group has 5 ring carbon atoms, and a furanyl group has 4 ring carbon atoms. When a benzene ring and/or a naphthalene ring is substituted by a substituent (e.g., an alkyl group), the number of carbon atoms of the alkyl group is not counted in the number of the ring carbon atoms. When a fluorene ring is substituted by a substituent (e.g., a fluorene ring) (i.e., a spirofluorene ring is included), the number of carbon atoms of the fluorene ring as a substituent is not counted in the number of the ring carbon atoms of the fluorene ring.

Herein, the ring atoms refer to the number of atoms forming a ring of a compound (e.g., a monocyclic compound, fused-ring compound, crosslinking compound, carbon ring compound, and heterocyclic compound) in which the atoms are bonded to each other to form the ring (e.g., monocyclic ring, fused ring, ring assembly). Atom(s) not forming a ring and atom(s) included in a substituent when the ring is substituted by the substituent are not counted in the number of the ring atoms. Unless specifically described, the same applies to the “ring atoms” described later. For instance, a pyridine ring has six ring atoms, a quinazoline ring has ten ring atoms, and a furan ring has five ring atoms. A hydrogen atom(s) and/or an atom(s) of a substituent which are bonded to carbon atoms of a pyridine ring and/or quinazoline ring are not counted in the ring atoms. When a fluorene ring is substituted by a substituent (e.g., a fluorene ring) (i.e., a spirofluorene ring is included), the number of atoms of the fluorene ring as a substituent is not counted in the number of the ring atoms of the fluorene ring.

### Description of Each Substituent in Formulae Herein

The aryl group (occasionally referred to as an aromatic hydrocarbon group) herein is exemplified by an aryl group Sub₁. The aryl group Sub1 preferably has 6 to 30 ring carbon atoms, more preferably 6 to 20 ring carbon atoms, still more preferably 6 to 14 ring carbon atoms, and still further more preferably 6 to 12 ring carbon atoms.

The aryl group Sub₁ herein is at least one group selected from the group consisting of a phenyl group, biphenyl group, terphenyl group, naphthyl group, anthryl group, phenanthryl group, fluorenyl group, pyrenyl group, chrysenyl group, fluoranthenyl group, benz[a]anthryl group, benzo[c]phenanthryl group, triphenylenyl group, benzo[k]fluoranthenyl group, benzo[g]chrysenyl group, benzo[b]triphenylenyl group, picenyl group, and perylenyl group.

Among the aryl group Sub₁, a phenyl group, biphenyl group, naphthyl group, phenanthryl group, terphenyl group and fluorenyl group are preferable. A carbon atom in a position 9 of each of 1-fluorenyl group, 2-fluorenyl group, 3-fluorenyl group and 4-fluorenyl group is preferably substituted by a substituted or unsubstituted alkyl group Sub₃ or a substituted or unsubstituted aryl group Sub₁ described later herein.

The heteroaryl group (occasionally referred to as a heterocyclic group, heteroaromatic cyclic group or aromatic heterocyclic group) herein is exemplified by a heterocyclic group Sub₂. The heterocyclic group Sub₂ is a group containing, as a hetero atom(s), at least one atom selected from the group consisting of nitrogen, sulfur, oxygen, silicon, selenium atom and germanium atom. The heterocyclic group Sub₂ preferably contains, as a hetero atom(s), at least one atom selected from the group consisting of nitrogen, sulfur and oxygen. The heterocyclic group Sub₂ preferably has 5 to 30 ring atoms, more preferably 5 to 20 ring atoms, and still more preferably 5 to 14 ring atoms.

The heterocyclic group Sub₂ herein are, for instance, at least one group selected from the group consisting of a pyridyl group, pyrimidinyl group, pyrazinyl group, pyridazinyl group, triazinyl group, quinolyl group, isoquinolinyl group, naphthyridinyl group, phthalazinyl group, quinoxalinyl group, quinazolinyl group, phenanthridinyl group, acridinyl group, phenanthrolinyl group, pyrrolyl group, imidazolyl group, pyrazolyl group, triazolyl group, tetrazolyl group, indolyl group, benzimidazolyl group, indazolyl group, imidazopyridinyl group, benzotriazolyl group, carbazolyl group, furyl group, thienyl group, oxazolyl group, thiazolyl group, isoxazolyl group, isothiazolyl group, oxadiazolyl group, thiadiazolyl group, benzofuranyl group, benzothienyl group, benzoxazolyl group, benzothiazolyl group, benzisoxazolyl group, benzisothiazolyl group, benzoxadiazolyl group, benzothiadiazolyl group, dibenzofuranyl group, dibenzothienyl group, piperidinyl group, pyrrolidinyl group, piperazinyl group, morpholyl group, phenazinyl group, phenothiazinyl group, and phenoxazinyl group.

Among the above heterocyclic group Sub₂, a 1-dibenzofuranyl group, 2-dibenzofuranyl group, 3-dibenzofuranyl group, 4-dibenzofuranyl group, 1-dibenzothienyl group, 2-dibenzothienyl group, 3-dibenzothienyl group, 4-dibenzothienyl group, 1-carbazolyl group, 2-carbazolyl group, 3-carbazolyl group, 4-carbazolyl group, and 9-carbazolyl group are more preferable. A nitrogen atom in a position 9 of each of 1-carbazolyl group, 2-carbazolyl group, 3-carbazolyl group and 4-carbazolyl group is preferably substituted by a substituted or unsubstituted aryl group Sub1 or a substituted or unsubstituted heterocyclic group Sub2 described herein.

Herein, the heterocyclic group Sub₂ may be a group derived from any one of partial structures represented by formulae (XY-1) to (XY-18) below.

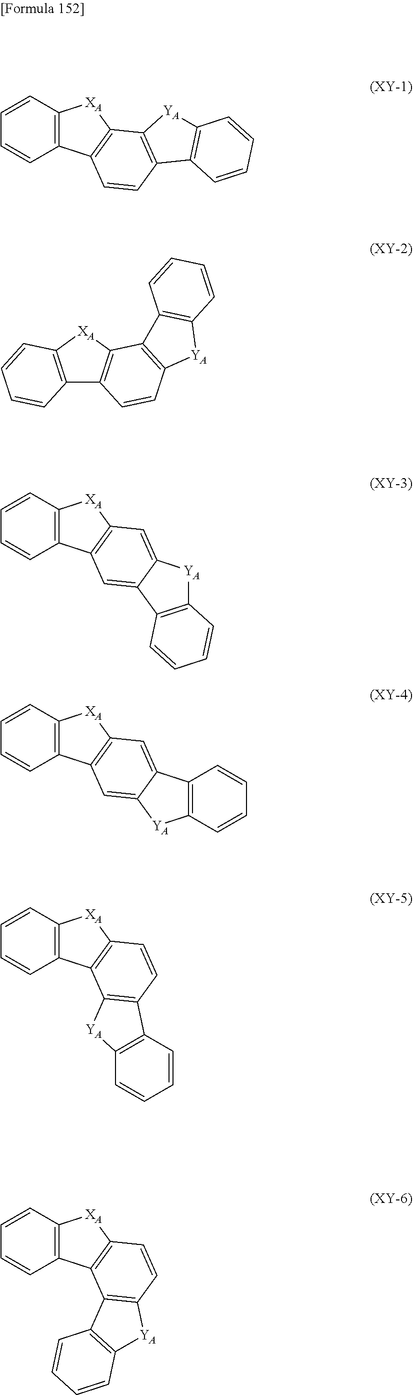

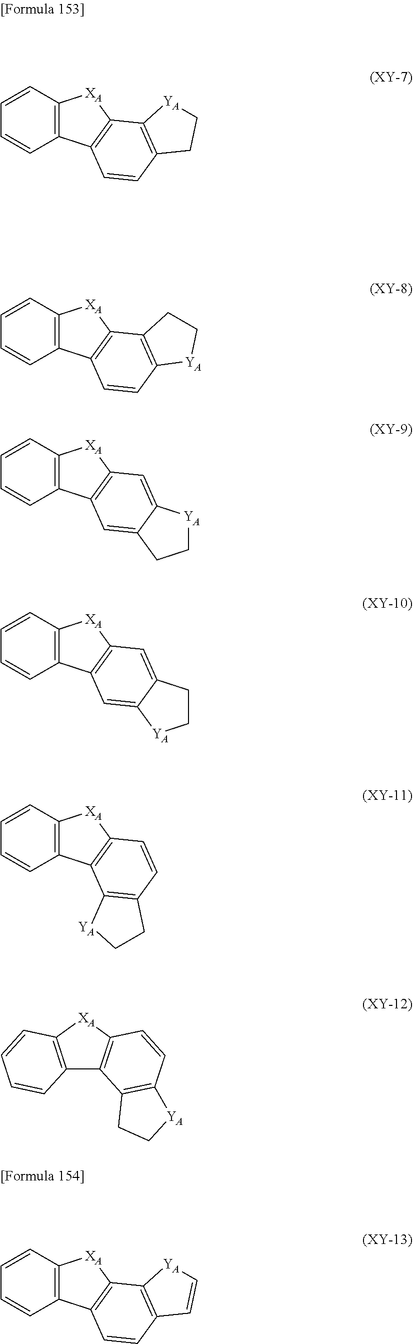

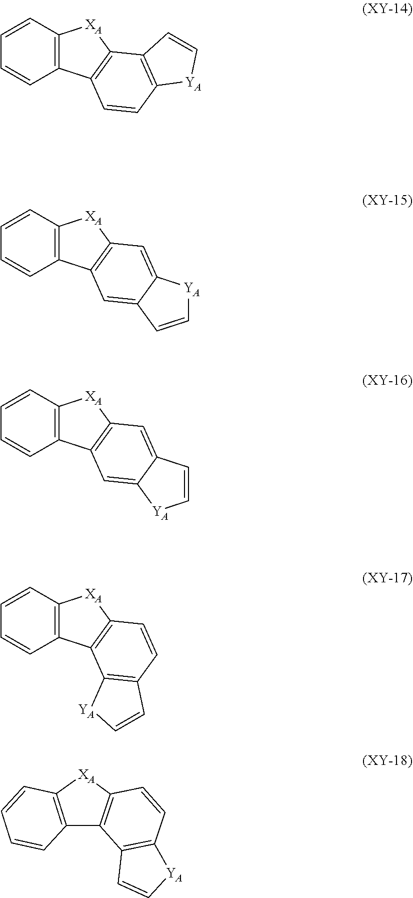

In the formulae (XY-1) to (XY-18), X_{A} and Y_{A} each independently represent a hetero atom, and preferably represent an oxygen atom, sulfur atom, selenium atom, silicon atom or germanium atom. The partial structures represented by the formulae (XY-1) to (XY-18) may each have a bond at any position to provide a heterocyclic group, in which the heterocyclic group may be substituted.

Herein, the heterocyclic group Sub₂ may be a group represented by one of formulae (XY-19) to (XY-22) below. Further, the position of the bond may be changed as needed.

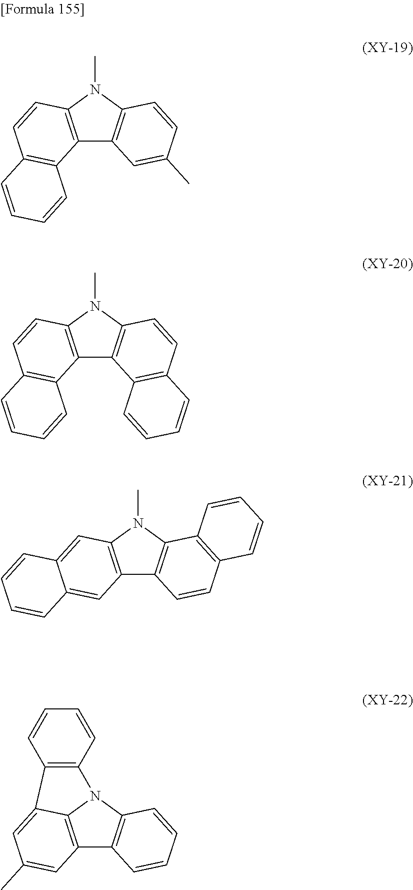

The alkyl group herein may be any one of a linear alkyl group, branched alkyl group and cyclic alkyl group.

The alkyl group herein is exemplified by an alkyl group Sub₃.

The linear alkyl group herein is exemplified by a linear alkyl group Sub₃₁. The branched alkyl group herein is exemplified by a branched alkyl group Sub₃₂.

The cyclic alkyl group herein is exemplified by a cyclic alkyl group Sub₃₃ (also referred to as a cycloalkyl group Sub₃₃).

For instance, the alkyl group Sub₃ is at least one group selected from the group consisting of a linear alkyl group Sub₃₁, branched alkyl group Sub₃₂, and cyclic alkyl group Sub₃₃.

Herein, the linear alkyl group Sub₃₁ or branched alkyl group Sub₃₂ preferably has 1 to 30 carbon atoms, more preferably 1 to 20 carbon atoms, still more preferably 1 to 10 carbon atoms, and still further more preferably 1 to 6 carbon atoms.

Herein, the cycloalkyl group Sub₃₃ preferably has 3 to 30 ring carbon atoms, more preferably 3 to 20 ring carbon atoms, still more preferably 3 to 10 ring carbon atoms, and still further more preferably 5 to 8 ring carbon atoms.

The linear alkyl group Sub₃₁ or branched alkyl group Sub₃₂ herein is exemplified by at least one group selected from the group consisting of a methyl group, ethyl group, n-propyl group, isopropyl group, n-butyl group, s-butyl group, isobutyl group, t-butyl group, n-pentyl group, n-hexyl group, n-heptyl group, n-octyl group, n-nonyl group, n-decyl group, n-undecyl group, n-dodecyl group, n-tridecyl group, n-tetradecyl group, n-pentadecyl group, n-hexadecyl group, n-heptadecyl group, n-octadecyl group, neopentyl group, amyl group, isoamyl group, 1-methylpentyl group, 2-methylpentyl group, 1-pentylhexyl group, 1-butylpentyl group, 1-heptyloctyl group, and 3-methylpentyl group.

The linear alkyl group Sub₃₁ or branched alkyl group Sub₃₂ is still more preferably a methyl group, ethyl group, propyl group, isopropyl group, n-butyl group, s-butyl group, isobutyl group, t-butyl group, n-pentyl group, n-hexyl group, amyl group, isoamyl group and neopentyl group.

The cycloalkyl group Sub₃₃ herein is exemplified by at least one group selected from the group consisting of a cyclopropyl group, cyclobutyl group, cyclopentyl group, cyclohexyl group, 4-metylcyclohexyl group, adamantyl group and norbornyl group. Among the cycloalkyl group Sub₃₃, a cyclopentyl group and a cyclohexyl group are still more preferable.

Herein, an alkyl halide group is exemplified by an alkyl halide group Sub₄. The alkyl halide group Sub₄ is provided by substituting the alkyl group Sub₃ with at least one halogen atom, preferably at least one fluorine atom.

Herein, the alkyl halide group Sub₄ is exemplified by at least one group selected from the group consisting of a fluoromethyl group, difluoromethyl group, trifluoromethyl group, fluoroethyl group, trifluoromethylmethyl group, trifluoroethyl group, and pentafluoroethyl group.

Herein, a substituted silyl group is exemplified by a substituted silyl group Sub₅. The substituted silyl group Sub₅ is exemplified by at least one group selected from the group consisting of an alkylsilyl group Sub₅₁ and an arylsilyl group Sub₅₂.

Herein, the alkylsilyl group Sub₅₁ is exemplified by a trialkylsilyl group Sub₅₁₁ having the above-described alkyl group Sub₃.

The trialkylsilyl group Sub₅₁₁ is exemplified by at least one group selected from the group consisting of a trimethylsilyl group, triethylsilyl group, tri-n-butylsilyl group, tri-n-octylsilyl group, triisobutylsilyl group, dimethylethylsilyl group, dimethylisopropylsilyl group, dimethyl-n-propylsilyl group, dimethyl-n-butylsilyl group, dimethyl-t-butylsilyl group, diethylisopropylsilyl group, vinyl dimethylsilyl group, propyldimethylsilyl group, and triisopropylsilyl group. Three alkyl groups Sub₃ in the trialkylsilyl group Sub₅₁₁ may be mutually the same or different.

Herein, the arylsilyl group Sub₅₂ is exemplified by at least one group selected from the group consisting of a dialkylarylsilyl group Sub₅₂₁, alkyldiarylsilyl group Sub₅₂₂ and triarylsilyl group Sub₅₂₃.

The dialkylarylsilyl group Sub₅₂₁ is exemplified by a dialkylarylsilyl group including two alkyl groups Sub₃ and one aryl group Sub₁. The dialkylarylsilyl group Sub₅₂₁ preferably has 8 to 30 carbon atoms.

The alkyldiarylsilyl group Sub₅₂₂ is exemplified by an alkyldiarylsilyl group including one alkyl group Sub₃ and two aryl groups Sub₁. The alkyldiarylsilyl group Sub₅₂₂ preferably has 13 to 30 carbon atoms.

The triarylsilyl group Sub₅₂₃ is exemplified by a triarylsilyl group including three aryl groups Sub₁. The triarylsilyl group Sub₅₂₃ preferably has 18 to 30 carbon atoms.

Herein, a substituted or unsubstituted alkyl sulfonyl group is exemplified by an alkyl sulfonyl group Sub₆. The alkyl sulfonyl group Sub₆ is represented by —SO₂R_{w}. R_{w} in —SO₂R_{w} represents a substituted or unsubstituted alkyl group Sub₃ described above.

Herein, an aralkyl group (occasionally referred to as an arylalkyl group) is exemplified by an aralkyl group Sub₇. An aryl group in the aralkyl group Sub₇ includes, for instance, at least one of the above-described aryl group Sub₁ or the above-described heteroaryl group Sub₂.

The aralkyl group Sub₇ herein is preferably a group having the aryl group Sub₁ and is represented by —Z₃—Z₄. Z₃ is exemplified by an alkylene group corresponding to the above alkyl group Sub₃. Z₄ is exemplified by the above aryl group Sub₁. In this aralkyl group Sub₇, an aryl moiety has 6 to 30 carbon atoms (preferably 6 to 20 carbon atoms, more preferably 6 to 12 carbon atoms) and an alkyl moiety has 1 to 30 carbon atoms (preferably 1 to 20 carbon atoms, more preferably 1 to 10 carbon atoms, and still more preferably 1 to 6 carbon atoms). The aralkyl group Sub₇ is exemplified by at least one group selected from the group consisting of a benzyl group, 2-phenylpropane-2-yl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

The alkoxy group herein is exemplified by an alkoxy group Sub₈. The alkoxy group Sub₈ is represented by —OZ₁. Z₁ is exemplified by the above alkyl group Sub₃. The alkoxy group Sub₈ preferably has 1 to 30 carbon atoms, more preferably 1 to 20 carbon atoms. The alkoxy group Sub₈ is exemplified by at least one group selected from the group consisting of a methoxy group, ethoxy group, propoxy group, butoxy group, pentyloxy group and hexyloxy group.

Herein, an alkoxy halide group is exemplified by an alkoxy halide group Sub₉. The alkoxy halide group Sub₉ is provided, for instance, by substituting the alkoxy group Sub8 with at least one halogen atom, preferably at least one fluorine atom.

Herein, an aryloxy group (occasionally referred to as an arylalkoxy group) is exemplified by an arylalkoxy group Sub₁₀. An aryl group in the arylalkoxy group Sub₁₀ includes at least one of the aryl group Sub₁ or the heteroaryl group Sub₂.

The arylalkoxy group Sub₁₀ herein is represented by —OZ₂. Z₂ is exemplified by the aryl group Sub₁ or the heteroaryl group Sub₂. The arylalkoxy group Sub₁₀ preferably has 6 to 30 ring carbon atoms, more preferably 6 to 20 ring carbon atoms. The arylalkoxy group Sub₁₀ is exemplified by a phenoxy group.

Herein, a substituted amino group is exemplified by a substituted amino group Sub₁₁. The substituted amino group Sub₁₁ is exemplified by at least one group selected from the group consisting of an arylamino group Sub₁₁₁ and an alkylamino group Sub₁₁₂.

The arylamino group Sub₁₁₁ is represented by —NHR_{V1} or —N(R_{V1})₂. R_{V1} is exemplified by the aryl group Sub₁. Two R_{V1} in —N(R_{V1})₂ are mutually the same or different.

The alkylamino group Sub₁₁₂ is represented by —NHR_{V2} or —N(R_{V2})₂. R_{V2} is exemplified by the alkyl group Sub₃. Two R_{V2} in —N(R_{V2})₂ are mutually the same or different.

Herein, the alkenyl group is exemplified by an alkenyl group Sub₁₂. The alkenyl group Sub₁₂, which is linear or branched, is exemplified by at least one group selected from the group consisting of a vinyl group, propenyl group, butenyl group, oleyl group, eicosapentaenyl group, docosahexaenyl group, styryl group, 2,2-diphenylvinyl group, 1,2,2-triphenylvinyl group, and 2-phenyl-2-propenyl group.

The alkynyl group herein is exemplified by an alkynyl group Sub₁₃. The alkynyl group Sub₁₃ may be linear or branched and is at least one group selected from the group consisting of an ethynyl group, a propynyl group and a 2-phenylethynyl group.

The alkylthio group herein is exemplified by an alkylthio group Sub₁₄.

The alkylthio group Sub₁₄ is represented by —SR_{V3}. R_{V3} is exemplified by the alkyl group Sub₃. The alkylthio group Sub₁₄ preferably has 1 to 30 carbon atoms, more preferably 1 to 20 carbon atoms.

The arylthio group herein is exemplified by an arylthio group Sub₁₅.

The arylthio group Sub₁₅ is represented by —SR_{V4}. R_{V4} is exemplified by the aryl group Sub₁. The arylthio group Sub₁₅ preferably has 6 to 30 ring carbon atoms, more preferably 6 to 20 ring carbon atoms.

Herein, examples of the halogen atom include a fluorine atom, a chlorine atom, a bromine atom and an iodine atom, among which a fluorine atom is preferable.

A substituted phosphino group herein is exemplified by a substituted phosphino group Sub₁₆. The substituted phosphino group Sub₁₆ is exemplified by a phenyl phosphanyl group.

An arylcarbonyl group herein is exemplified by an arylcarbonyl group Sub₁₇. The arylcarbonyl group Sub17 is represented by —COY′. Y′ is exemplified by the aryl group Sub₁. Herein, the arylcarbonyl group Sub₁₇ is exemplified by at least one group selected from the group consisting of a phenyl carbonyl group, diphenyl carbonyl group, naphthyl carbonyl group, and triphenyl carbonyl group.

An acyl group herein is exemplified by an acyl group Sub₁₈. The acyl group Sub₁₈ is represented by —COR′. R′ is exemplified by the alkyl group Sub₃. The acyl group Sub₁₈ herein is exemplified by at least one group selected from the group consisting of an acetyl group and a propionyl group.

A substituted phosphoryl group herein is exemplified by a substituted phosphoryl group Sub₁₉. The substituted phosphoryl group Sub₁₉ is represented by a formula (P) below.

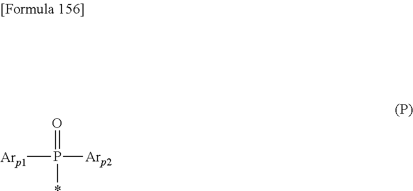

In the formula (P), Ar_{P1} and Ar_{P2} are any one substituent selected from the group consisting of the above alkyl group Sub₃ and the above aryl group Sub₁.

An ester group herein is exemplified by an ester group Sub₂₀. The ester group Sub₂₀ is exemplified by at least one group selected from the group consisting of an alkyl ester group and an aryl ester group.

An alkyl ester group herein is exemplified by an alkyl ester group Sub₂₀₁. The alkyl ester group Sub₂₀₁ is represented by —C(═O)OR^{E}. R^{E} is exemplified by a substituted or unsubstituted alkyl group Sub₃ described above.

An aryl ester group herein is exemplified by an aryl ester group Sub₂₀₂. The aryl ester group Sub₂₀₂ is represented by —C(═O)OR^{Ar}. R^{Ar} is exemplified by a substituted or unsubstituted aryl group Sub₁ described above.

A siloxanyl group herein is exemplified by a siloxanyl group Sub₂₁. The siloxanyl group Sub₂₁ is a silicon compound group through an ether bond. The siloxanyl group Sub₂₁ is exemplified by a trimethylsiloxanyl group.

A carbamoyl group herein is represented by —CONH₂.

A substituted carbamoyl group herein is exemplified by a carbamoyl group Sub₂₂. The carbamoyl group Sub₂₂ is represented by —CONH—Ar^{C} or —CONH—R^{C}. Ar^{C} is exemplified by at least one group selected from the group consisting of a substituted or unsubstituted aryl group Sub₁ described above (preferably 6 to 10 ring carbon atoms) and the above-described heteroaryl group Sub₂ (preferably 5 to 14 ring atoms). Ar^{C} may be a group formed by bonding the aryl group Sub₁ and the heteroaryl group Sub₂.

R^{C} is exemplified by a substituted or unsubstituted alkyl group Sub3 described above (preferably having 1 to 6 carbon atoms).

Herein, “carbon atoms forming a ring (ring carbon atoms)” mean carbon atoms forming a saturated ring, unsaturated ring, or aromatic ring. “Atoms forming a ring (ring atoms)” mean carbon atoms and hetero atoms forming a hetero ring including a saturated ring, unsaturated ring, or aromatic ring.

Herein, a hydrogen atom includes isotope having different numbers of neutrons, specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a deuterium.

Hereinafter, an alkyl group Sub₃ means at least one group of a linear alkyl group Sub₃₁, a branched alkyl group Sub₃₂, or a cyclic alkyl group Sub₃₃ described in “Description of Each Substituent.”

Similarly, a substituted silyl group Sub₅ means at least one group of an alkylsilyl group Sub₅₁ or an arylsilyl group Sub₅₂.

Similarly, a substituted amino group Sub₁₁ means at least one group of an arylamino group Sub₁₁₁ or an alkylamino group Sub₁₁₂.

Herein, a substituent for a “substituted or unsubstituted” group is exemplified by a substituent R_{F1}. The substituent R_{F1} is at least one group selected from the group consisting of an aryl group Sub₁, heteroaryl group Sub₂, alkyl group Sub₃, alkyl halide group Sub₄, substituted silyl group Sub₅, alkylsulfonyl group Sub₆, aralkyl group Sub₇, alkoxy group Sub₈, alkoxy halide group Sub₉, arylalkoxy group Sub₁₀, substituted amino group Sub₁₁, alkenyl group Sub₁₂, alkynyl group Sub₁₃, alkylthio group Sub₁₄, arylthio group Sub₁₅, substituted phosphino group Sub₁₆, arylcarbonyl group Sub₁₇, acyl group Sub₁₈, substituted phosphoryl group Sub₁₉, ester group Sub₂₀, siloxanyl group Sub₂₁, carbamoyl group Sub₂₂, unsubstituted amino group, unsubstituted silyl group, halogen atom, cyano group, hydroxy group, nitro group, and carboxy group.

Herein, the substituent R_{F1} for a “substituted or unsubstituted” group may be a diaryl boron group (Ar_{B1}Ar_{B2}B—). Ar_{B1} and Ar_{B2} are exemplified by the above-described aryl group Sub₁. Ar_{B1} and Ar_{B2} in Ar_{B1}Ar_{B2}B— are the same or different.

Specific examples and preferable examples of the substituent R_{F1} are the same as those of the substituents described in “Description of Each Substituent” (e.g., an aryl group Sub₁, heteroaryl group Sub₂, alkyl group Sub₃, alkyl halide group Sub₄, substituted silyl group Sub₅, alkylsulfonyl group Sub₆, aralkyl group Sub₇, alkoxy group Sub₈, alkoxy halide group Sub₉, arylalkoxy group Sub₁₀, substituted amino group Sub₁₁, alkenyl group Sub₁₂, alkynyl group Sub₁₃, alkylthio group Sub₁₄, arylthio group Sub₁₅, substituted phosphino group Sub₁₆, arylcarbonyl group Sub₁₇, acyl group Sub₁₈, substituted phosphoryl group Sub₁₉, ester group Sub₂₀, siloxanyl group Sub₂₁, and carbamoyl group Sub₂₂).

The substituent R_{F1} for a “substituted or unsubstituted” group may be further substituted by at least one group (hereinafter, also referred to as a substituent R_{F2}) selected from the group consisting of an aryl group Sub₁, heteroaryl group Sub₂, alkyl group Sub₃, alkyl halide group Sub₄, substituted silyl group Sub₅, alkylsulfonyl group Sub₆, aralkyl group Sub₇, alkoxy group Sub₈, alkoxy halide group Sub₉, arylalkoxy group Sub₁₀, substituted amino group Sub₁₁, alkenyl group Sub₁₂, alkynyl group Sub₁₃, alkylthio group Sub₁₄, arylthio group Sub₁₅, substituted phosphino group Sub₁₆, arylcarbonyl group Sub₁₇, acyl group Sub₁₈, substituted phosphoryl group Sub₁₉, ester group Sub₂₀, siloxanyl group Sub₂₁, carbamoyl group Sub₂₂, unsubstituted amino group, unsubstituted silyl group, halogen atom, cyano group, hydroxy group, nitro group, and carboxy group. Moreover, a plurality of substituents R_{F2} may be bonded to each other to form a ring.

“Unsubstituted” for a “substituted or unsubstituted” group means that a group is not substituted by the above-described substituent R_{F1} but bonded with a hydrogen atom.

Herein, “XX to YY carbon atoms” in the description of “substituted or unsubstituted ZZ group having XX to YY carbon atoms” represent carbon atoms of an unsubstituted ZZ group and do not include carbon atoms of the substituent R_{F1} of the substituted ZZ group.

Herein, “XX to YY atoms” in the description of “substituted or unsubstituted ZZ group having XX to YY atoms” represent atoms of an unsubstituted ZZ group and do not include atoms of the substituent R_{F1} of the substituted ZZ group.

The same description as the above applies to “substituted or unsubstituted” in compounds or partial structures thereof described herein.

Herein, when the substituents are bonded to each other to form a ring, the ring is structured to be a saturated ring, an unsaturated ring, an aromatic hydrocarbon ring or a hetero ring.

Herein, examples of the aromatic hydrocarbon group in the linking group include a divalent or multivalent group obtained by eliminating one or more atoms from the above monovalent aryl group Sub₁.

Herein, examples of the heterocyclic group in the linking group include a divalent or multivalent group obtained by eliminating one or more atoms from the above monovalent heteroaryl group Sub₂.

### EXAMPLES

### Compounds

The compound M3 used for manufacturing the organic EL device is shown below.

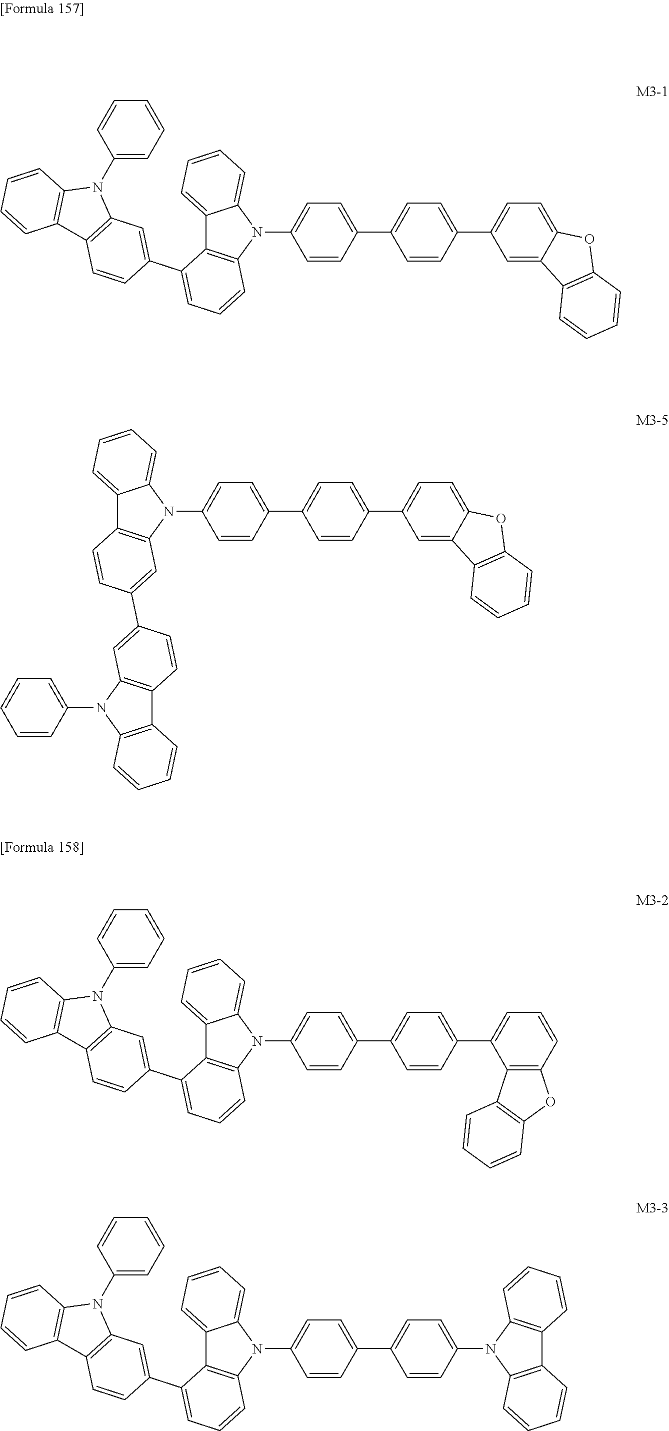 [SMILES: c1ccc(-n2c3ccccc3c3ccc(-c4cccc5c4c4ccccc4n5-c4ccc(-c5ccc(-c6ccc7oc8ccccc8c7c6)cc5)cc4)cc32)cc1 ; c1ccc(-n2c3ccccc3c3ccc(-c4ccc5c6ccccc6n(-c6ccc(-c7ccc(-c8ccc9oc%10ccccc%10c9c8)cc7)cc6)c5c4)cc32)cc1 ; c1ccc(-n2c3ccccc3c3ccc(-c4cccc5c4c4ccccc4n5-c4ccc(-c5ccc(-c6cccc7oc8ccccc8c67)cc5)cc4)cc32)cc1 ; c1ccc(-n2c3ccccc3c3ccc(-c4cccc5c4c4ccccc4n5-c4ccc(-c5ccc(-n6c7ccccc7c7ccccc76)cc5)cc4)cc32)cc1]

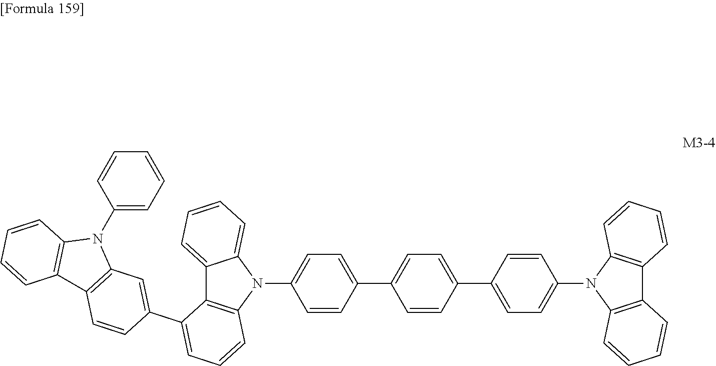 [SMILES: c1ccc(-n2c3ccccc3c3ccc(-c4cccc5c4c4ccccc4n5-c4ccc(-c5ccc(-c6ccc(-n7c8ccccc8c8ccccc87)cc6)cc5)cc4)cc32)cc1]

Structures of compounds used for manufacturing organic EL devices in Comparatives are shown below.

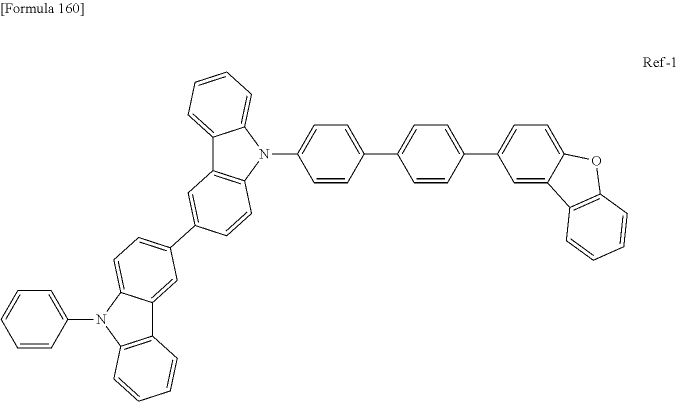 [SMILES: c1ccc(-n2c3ccccc3c3cc(-c4ccc5c(c4)c4ccccc4n5-c4ccc(-c5ccc(-c6ccc7oc8ccccc8c7c6)cc5)cc4)ccc32)cc1]

Structures of other compounds used for manufacturing the organic EL devices in Examples and Comparatives are shown below.

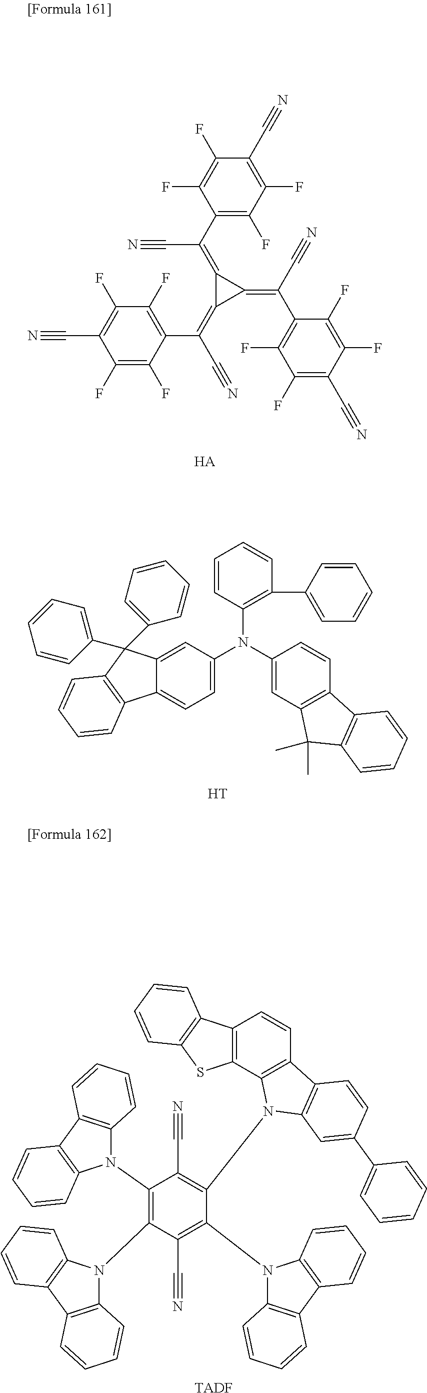

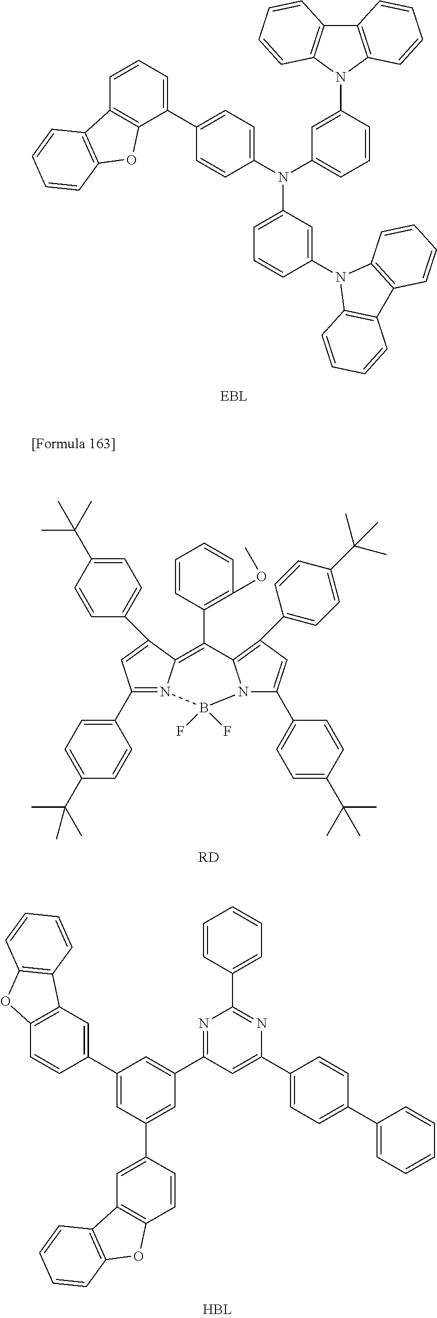

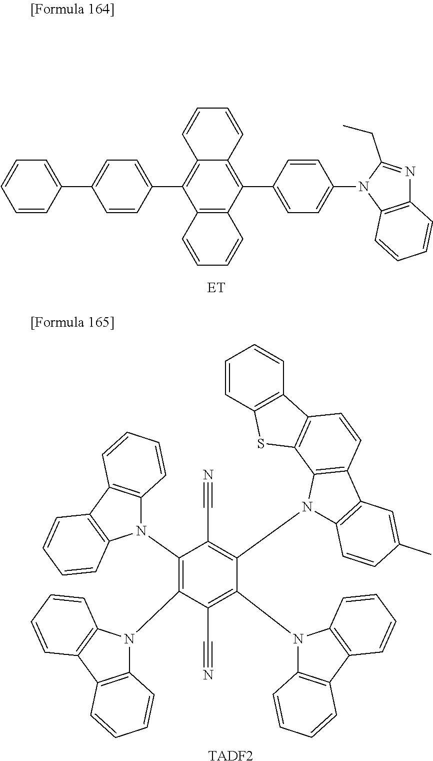

### Manufacture 1 of Organic EL Device

The organic EL devices were manufactured and evaluated as follows.

### Example 1

A glass substrate (size: 25 mm×75 mm×1.1 mm thick, manufactured by Geomatec Co., Ltd.) having an ITO transparent electrode (anode) was ultrasonic-cleaned in isopropyl alcohol for five minutes, and then UV-ozone-cleaned for one minute. The film thickness of ITO was 130 nm.

After the glass substrate having the transparent electrode line was cleaned, the glass substrate was mounted on a substrate holder of a vacuum deposition apparatus. Firstly, a compound HT1 and a compound HA were co-deposited on a surface of the glass substrate where the transparent electrode line was provided in a manner to cover the transparent electrode, thereby forming a 10-nm-thick hole injecting layer. The concentrations of the compound HT and the compound HA in the hole injecting layer were 97 mass % and 3 mass %, respectively.

Next, the compound HT was vapor-deposited on the hole injecting layer to form a 200-nm-thick hole transporting layer.

Next, a compound EBL was vapor-deposited on the hole transporting layer to form a 10-nm-thick electron blocking layer.

Next, a compound RD as the fluorescent compound M1, a compound TADF as the delayed fluorescent compound M2, and a compound M3-1 as the compound M3 were co-deposited on the electron blocking layer to form a 25-nm-thick emitting layer. The concentrations of the compound RD, the compound TADF, and the compound M3-1 in the emitting layer were 1 mass %, 25 mass %, and 74 mass %, respectively.

Next, a compound HBL was vapor-deposited on the emitting layer to form a 10-nm-thick hole blocking layer.

Next, a compound ET was vapor-deposited on the hole blocking layer to form a 30-nm-thick electron transporting layer.

Next, lithium fluoride (LiF) was vapor-deposited on the electron transporting layer to form a 1-nm-thick electron injecting electrode (cathode).

Subsequently, metal aluminum (Al) was vapor-deposited on the electron injectable electrode to form an 80-nm-thick metal Al cathode.

A device arrangement of the organic EL device in Example 1 is roughly shown as follows.

### ITO(130)/HT:HA(10.97%:3%)/HT(200)/EBL(10)/M3-1:TADF:RD(25.74%:25%:1%)/HBL(10)/ET(30)/LiF(1)/Al(80)

Numerals in parentheses represent a film thickness (unit: nm).

The numerals (97%:3%) represented by percentage in the same parentheses indicate a ratio (mass %) between the compound HT and the compound HA in the hole injecting layer. The numerals (74%:25%:1%) represented by percentage in the same parentheses indicate a ratio (mass %) between the compound M3, the compound M2, and the compound M1 in the emitting layer.

### Example 2 and Comparative 1

The organic EL devices in Example 2 and Comparative 1 were manufactured in the same manner as in Example 1 except that the compound M3-1 in the emitting layer in Example 1 was replaced by compounds shown in Table 2.

### Evaluation 1 of Organic EL Device

The organic EL devices manufactured in Examples 1, 2 and Comparative 1 were evaluated as follows. Table 2 shows the results. Although a compound Ref-1 used in Comparative 1 does not correspond to the compound M3, the compound Ref-1 is shown in the same column as the compound M3-1 in Example 1 for convenience.

### Main Peak Wavelength (λp)

Voltage was applied on an organic EL device such that a current density was 10 mA/cm², where spectral radiance spectrum was measured by a spectroradiometer CS-2000 (manufactured by Konica Minolta, Inc.). The main peak wavelength λp (unit: nm) was calculated based on the obtained spectral-radiance spectra.

### Drive Voltage

A voltage (unit: V) was measured when current was applied between the anode and the cathode such that a current density was 10 mA/cm².

### External Quantum Efficiency EQE

Voltage was applied to the organic EL devices such that a current density was 10 mA/cm², where spectral radiance spectrum was measured by a spectroradiometer CS-2000 (produced by Konica Minolta, Inc.). The external quantum efficiency EQE (unit: %) was calculated based on the obtained spectral radiance spectra, assuming that the spectra was provided under a Lambertian radiation.

TABLE 2

| | Emitting Layer | | | | | | | | | | Device Evaluation Results | | |
|---|---|---|---|---|---|---|---|---|---|---|---|---|---|
| | | | | | | | | | | | (@10 mA/cm²) | | |
| | Compound M3 | | | Compound M2 | | | | Compound M1 | | | | Drive | |
| | | S₁ | T_{77K} | | S₁ | ΔST | λ | | S₁ | λ | λp | Voltage | EQE |
| | Type | [eV] | [eV] | Type | [eV] | [eV] | [nm] | Type | [eV] | [nm] | [nm] | [V] | [%] |
| Ex. 1 | M3-1 | 3.36 | 2.70 | TADF | 2.34 | <0.01 | 539 | RD | 2.02 | 609 | 621 | 3.8 | 16.5 |
| Ex. 2 | M3-5 | 3.37 | 2.65 | TADF | 2.34 | <0.01 | 539 | RD | 2.02 | 609 | 621 | 3.7 | 16.4 |
| Comp. 1 | Ref-1 | 3.25 | 2.68 | TADF | 2.34 | <0.01 | 539 | RD | 2.02 | 609 | 621 | 3.8 | 2.6 |

The organic EL devices in Examples 1 and 2 exhibited a significantly improved external quantum efficiency EQE as compared with the organic EL device in Comparative 1 using the compound Ref-1 in place of the compound M3 in the emitting layer.

The compound Ref-1 used in Comparative 1 corresponds to a compound having a biscarbazole structure in Patent Literature 2. It is inferred that a luminous efficiency in Comparative 1 is decreased because an absolute value (4.97 eV) of HOMO energy level of the compound Ref-1 is too small as described in Table 1.

### Manufacture 2 of Organic EL Device —Examples 3 to 6 and Comparative 2

Organic EL devices in Example 3 to 6 and Comparative 2 were manufactured in the same manner as in Example 1 except that the compound M3-1 and the compound TADF in the emitting layer in Example 1 were replaced by compounds shown in Table 3.

### Evaluation 2 of Organic EL Device

The organic EL devices manufactured in Examples 3 to 6 and Comparative 2 were measured for the main peak wavelength λp (unit: nm) and the external quantum efficiency EQE (unit: %) in the same manner as in Example 1. Table 3 shows the results. Although the compound Ref-1 used in Comparative 2 does not correspond to the compound M3, the compound Ref-1 is shown in the same column as the compound M3-1 in Example 3 for convenience.

TABLE 3

| | Emitting Layer | | | | | | | | | | Device Evaluation Results | |
|---|---|---|---|---|---|---|---|---|---|---|---|---|
| | Compound M3 | | | Compound M2 | | | | Compound M1 | | | (@10 mA/cm²) | |
| | | S₁ | T_{77K} | | S₁ | ΔST | λ | | S₁ | λ | λp | EQE |
| | Type | [eV] | [eV] | Type | [eV] | [eV] | [nm] | Type | [eV] | [nm] | [nm] | [%] |
| Ex. 3 | M3-1 | 3.36 | 2.70 | TADF2 | 2.32 | <0.01 | 545 | RD | 2.02 | 609 | 621 | 17.4 |
| Ex. 4 | M3-2 | 3.36 | 2.74 | TADF2 | 2.32 | <0.01 | 545 | RD | 2.02 | 609 | 621 | 18.4 |
| Ex. 5 | M3-3 | 3.37 | 2.76 | TADF2 | 2.32 | <0.01 | 545 | RD | 2.02 | 609 | 621 | 18.1 |
| Ex. 6 | M3-4 | 3.36 | 2.68 | TADF2 | 2.32 | <0.01 | 545 | RD | 2.02 | 609 | 621 | 16.9 |
| Comp. 2 | Ref-1 | 3.25 | 2.68 | TADF2 | 2.32 | <0.01 | 545 | RD | 2.02 | 609 | 621 | 2.6 |

The organic EL devices in Examples 3 to 6 exhibited a significantly improved external quantum efficiency EQE as compared with the organic EL device in Comparative 2 using the compound Ref-1 in place of the compound M3 in the emitting layer.

The compound Ref-1 used in Comparative 2 corresponds to a compound having a biscarbazole structure in Patent Literature 2. It is inferred that a luminous efficiency in Comparative 2 is decreased because an absolute value (4.97 eV) of HOMO energy level of the compound Ref-1 is too small as described in Table 1.

### Evaluation of Compounds

Physical properties of compounds described in Tables 2 and 3 were measured according to the following methods.

### Thermally Activated Delayed Fluorescence (Delayed Fluorescence of Compound TADF)

Delayed fluorescence was checked by measuring transient PL using a device shown in FIG. 2. The compound TADF was dissolved in toluene to prepare a dilute solution with an absorbance of 0.05 or less at the excitation wavelength to eliminate contribution of self-absorption. In order to prevent quenching due to oxygen, the sample solution was frozen and degassed and then sealed in a cell with a lid under an argon atmosphere to obtain an oxygen-free sample solution saturated with argon.

The fluorescence spectrum of the sample solution was measured with a spectrofluorometer FP-8600 (produced by JASCO Corporation), and the fluorescence spectrum of a 9,10-diphenylanthracene ethanol solution was measured under the same conditions. Using the fluorescence area intensities of both spectra, the total fluorescence quantum yield was calculated by an equation (1) in Morris et al. J. Phys. Chem. 80 (1976) 969.

Prompt emission was observed immediately when the excited state was achieved by exciting the compound TADF with a pulse beam (i.e., a beam emitted from a pulse laser) having a wavelength to be absorbed by the compound TADF, and Delay emission was observed not immediately when the excited state was achieved but after the excited state was achieved. The delayed fluorescence in Examples means that an amount of Delay emission is 5% or more with respect to an amount of Prompt emission. Specifically, provided that the amount of Prompt emission is denoted by X_{P} and the amount of Delay emission is denoted by X_{D}, the delayed fluorescence means that a value of X_{D}/X_{P} is 0.05 or more.

An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using a device different from one described in Reference Document 1 or one shown in FIG. 2.

It was confirmed that the amount of Delay emission was 5% or more with respect to the amount of Prompt emission in the compound TADF.

Specifically, the value of X_{D}/X_{P} was 0.05 or more in the compound TADF. A value of X_{D}/X_{P} was 0.05 or more in a compound TADF2.

### Singlet Energy S₁

A singlet energy S₁ of each of the compound M3-1 to the compound M3-5, the compound TADF, the compound TADF2, the compound RD, and Comparative compound Ref-1 was measured according to the above-described solution method.

### Energy Gap T_{77K} at 77K

An energy gap T_{77K} of each of the compound M3-1 to the compound M3-5, the compound TADF, the compound TADF2, and Comparative compound Ref-1 was measured according to the measurement method of the energy gap T_{77K} described in the above “Relationship between Triplet Energy and Energy Gap at 77K.” ΔST was confirmed from the measurement results of T_{77K} and the above values of the singlet energy S₁.

### Main Peak Wavelength λ of Compounds

A main peak wavelength λ of each of the compound RD, the compound TADF, and the compound TADF2 was measured according to the following method.

A toluene solution of a measurement target compound at a concentration of 5 μmol/L was prepared and put in a quartz cell. An emission spectrum (ordinate axis: luminous intensity, abscissa axis: wavelength) of the thus-obtained sample was measured at a normal temperature (300K). In Examples, the emission spectrum was measured using a spectrophotometer manufactured by Hitachi, Ltd. (device name: F-7000). It should be noted that the machine for measuring the emission spectrum is not limited to the machine used herein. A peak wavelength of the emission spectrum exhibiting the maximum luminous intensity was defined as a main peak wavelength λ.

### Synthesis of Compounds

Compounds M3-1 to M3-11, which were the compound M3, were synthesized.

### Synthesis Example 1: Synthesis of Compound M3-1

A synthetic scheme of the compound M3-1 is shown below.

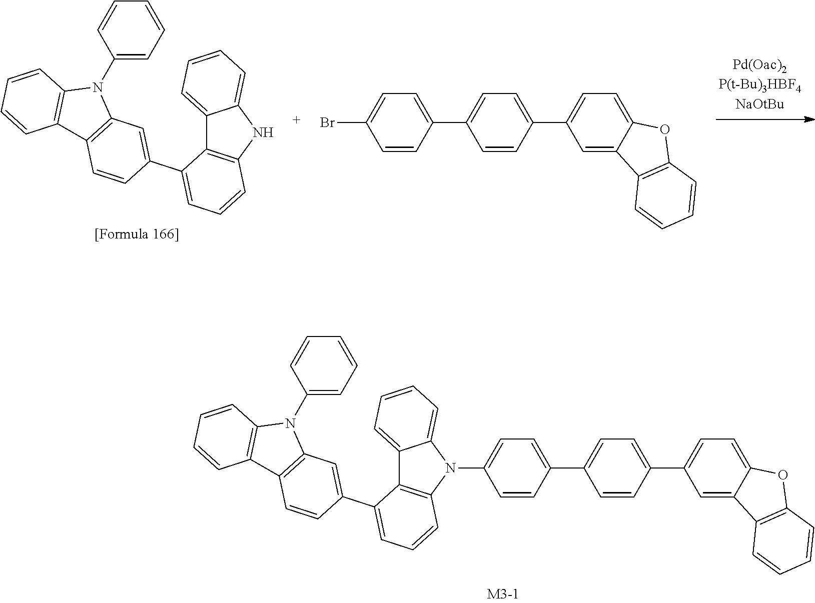 [SMILES: c1ccc(-n2c3ccccc3c3ccc(-c4cccc5[nH]c6ccccc6c45)cc32)cc1 ; Brc1ccc(-c2ccc(-c3ccc4oc5ccccc5c4c3)cc2)cc1 ; c1ccc(-n2c3ccccc3c3ccc(-c4cccc5c4c4ccccc4n5-c4ccc(-c5ccc(-c6ccc7oc8ccccc8c7c6)cc5)cc4)cc32)cc1]

Under nitrogen atmosphere, xylene (100 mL) was added to a mixture of 9-phenyl-9H,9′H-2,4′-bicarbazole (8.17 g, 20.0 mmol), 2-(4′-bromo-[1,1′-biphenyl]-4-yl)dibenzo[b,d]furan (7.99 g, 20.0 mmol), palladium acetate (134.7 mg, 0.60 mmol), tri-tert-butylphosphonium tetrafluoroborate (34.8 mg, 1.20 mmol), and sodium tert-butoxide (5.77 g, 60.0 mmol), and stirred for eight hours at 130 degrees C. After the reaction, a solid was filtrated and recrystallized with toluene to obtain the compound M3-1 (11.8 g, a yield of 81%). The obtained compound was identified as the compound M3-1 by analysis according to Liquid chromatography mass spectrometry (LC-MS).

### Synthesis Example 2: Synthesis of Compound M3-2

A synthetic scheme of a compound M3-2 is shown below.

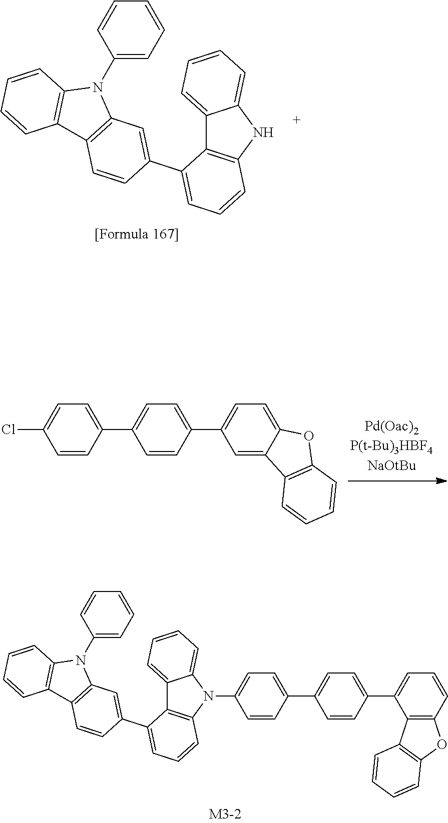 [SMILES: c1ccc(-n2c3ccccc3c3ccc(-c4cccc5[nH]c6ccccc6c45)cc32)cc1 ; Clc1ccc(-c2ccc(-c3ccc4oc5ccccc5c4c3)cc2)cc1 ; c1ccc(-n2c3ccccc3c3ccc(-c4cccc5c4c4ccccc4n5-c4ccc(-c5ccc(-c6cccc7oc8ccccc8c67)cc5)cc4)cc32)cc1]

The compound M3-2 was obtained in the same manner as in Synthesis Example 1 except for using 1-(4′-chloro-[1,1′-biphenyl]-4-yl)dibenzo[b,d]furan in place of 2-(4′-bromo-[1,1′-biphenyl]-4-yl)dibenzo[b,d]furan. A yield was 65%. The obtained compound was identified as the compound M3-2 by analysis according to LC-MS.

### Synthesis Example 3: Synthesis of Compound M3-3

A synthetic scheme of a compound M3-3 is shown below.

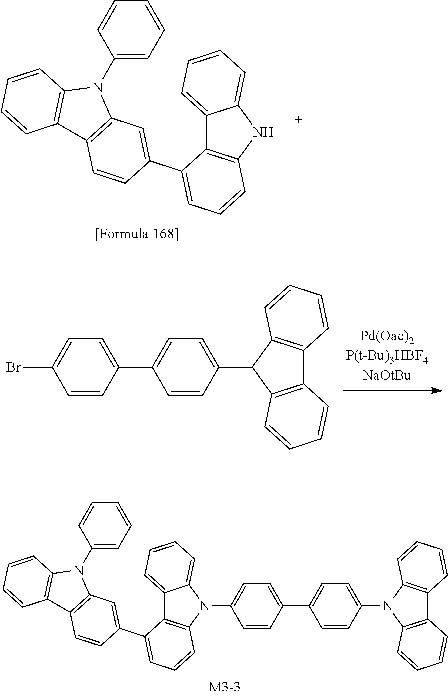 [SMILES: c1ccc(-n2c3ccccc3c3ccc(-c4cccc5[nH]c6ccccc6c45)cc32)cc1 ; Brc1ccc(-c2ccc(C3c4ccccc4-c4ccccc43)cc2)cc1 ; c1ccc(-n2c3ccccc3c3ccc(-c4cccc5c4c4ccccc4n5-c4ccc(-c5ccc(-n6c7ccccc7c7ccccc76)cc5)cc4)cc32)cc1]

The compound M3-3 was obtained in the same manner as in Synthesis Example 1 except for using 9-(4′-bromo-[1,1′-biphenyl]-4-yl)-9H-carbazole in place of 2-(4′-bromo-[1,1′-biphenyl]-4-yl)dibenzo[b,d]furan. A yield was 91%. The obtained compound was identified as the compound M3-3 by analysis according to LC-MS.

### Synthesis Example 4: Synthesis of Compound M3-4

A synthetic scheme of a compound M3-4 is shown below.

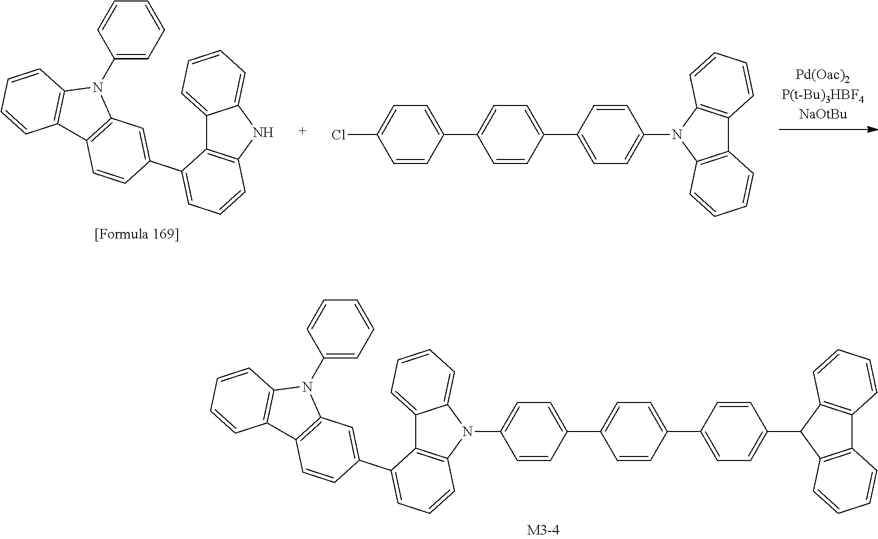 [SMILES: c1ccc(-n2c3ccccc3c3ccc(-c4cccc5[nH]c6ccccc6c45)cc32)cc1 ; Clc1ccc(-c2ccc(-c3ccc(-n4c5ccccc5c5ccccc54)cc3)cc2)cc1 ; c1ccc(-n2c3ccccc3c3ccc(-c4cccc5c4c4ccccc4n5-c4ccc(-c5ccc(-c6ccc(C7c8ccccc8-c8ccccc87)cc6)cc5)cc4)cc32)cc1]

The compound M3-4 was obtained in the same manner as in Synthesis Example 1 except for using 9-(4″-chloro-[1,1′: 4′,1″-terphenyl]-4-yl)-9H-carbazole in place of 2-(4′-bromo-[1,1′-biphenyl]-4-yl)dibenzo[b,d]furan. A yield was 77%. The obtained compound was identified as the compound M3-4 by analysis according to LC-MS.

### Synthesis Example 5: Synthesis of Compound M3-5

A synthetic scheme of a compound M3-5 is shown below.

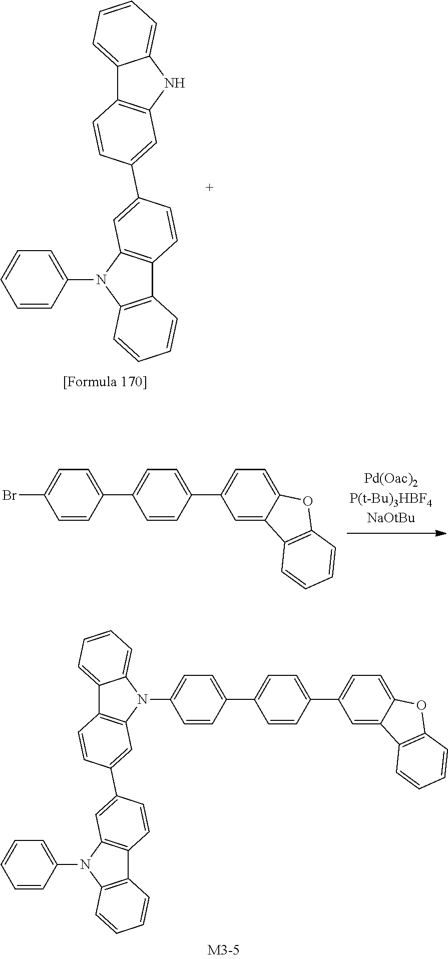 [SMILES: c1ccc(-n2c3ccccc3c3ccc(-c4ccc5c(c4)[nH]c4ccccc45)cc32)cc1 ; Brc1ccc(-c2ccc(-c3ccc4oc5ccccc5c4c3)cc2)cc1 ; c1ccc(-n2c3ccccc3c3ccc(-c4ccc5c6ccccc6n(-c6ccc(-c7ccc(-c8ccc9oc%10ccccc%10c9c8)cc7)cc6)c5c4)cc32)cc1]

The compound M3-5 was obtained in the same manner as in Synthesis Example 1 except for using 9-phenyl-9H,9′H-2,2′-bicarbazole in place of 9-phenyl-9H,9′H-2,4′-bicarbazole. A yield was 49%. The obtained compound was identified as the compound M3-5 by analysis according to LC-MS.

### Synthesis Example 6: Synthesis of Compound M3-6

A synthetic scheme of a compound M3-6 is shown below.

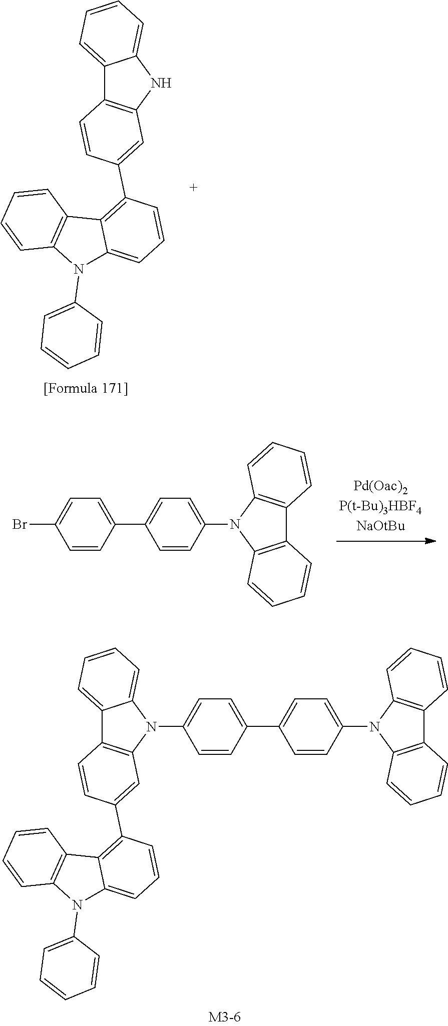 [SMILES: c1ccc(-n2c3ccccc3c3c(-c4ccc5c(c4)[nH]c4ccccc45)cccc32)cc1 ; Brc1ccc(-c2ccc(-n3c4ccccc4c4ccccc43)cc2)cc1 ; c1ccc(-n2c3ccccc3c3c(-c4ccc5c6ccccc6n(-c6ccc(-c7ccc(-n8c9ccccc9c9ccccc98)cc7)cc6)c5c4)cccc32)cc1]

The compound M3-6 was obtained in the same manner as in Synthesis Example 1 except for using 9′-phenyl-9H,9′H-2,4′-bicarbazole in place of 9-phenyl-9H,9′H-2,4′-bicarbazole and using 9-(4′-bromo-[1,1′-biphenyl]-4-yl)-9H-carbazole in place of 2-(4′-bromo-[1,1′-biphenyl]-4-yl)dibenzo[b,d]furan. A yield was 90%. The obtained compound was identified as the compound M3-6 by analysis according to LC-MS.

### Synthesis Example 7: Synthesis of Compound M3-7

A synthetic scheme of a compound M3-7 is shown below.

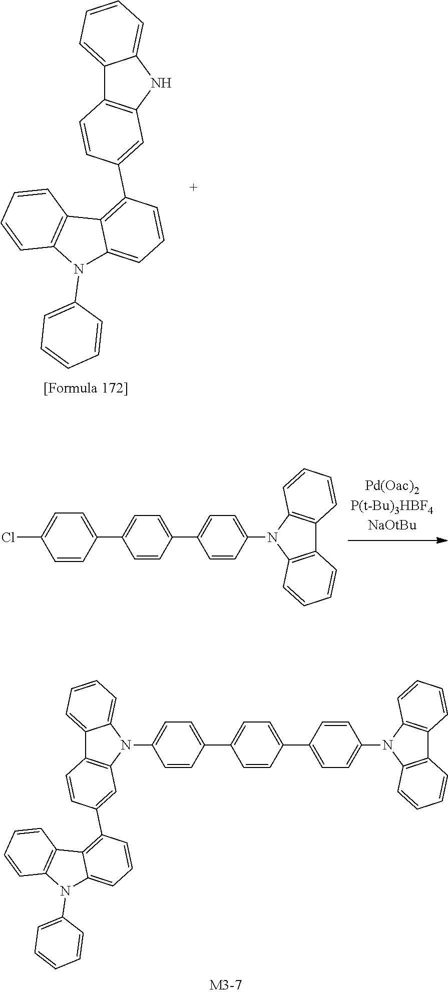 [SMILES: c1ccc(-n2c3ccccc3c3c(-c4ccc5c(c4)[nH]c4ccccc45)cccc32)cc1 ; Clc1ccc(-c2ccc(-c3ccc(-n4c5ccccc5c5ccccc54)cc3)cc2)cc1 ; c1ccc(-n2c3ccccc3c3c(-c4ccc5c6ccccc6n(-c6ccc(-c7ccc(-c8ccc(-n9c%10ccccc%10c%10ccccc%109)cc8)cc7)cc6)c5c4)cccc32)cc1]

The compound M3-7 was obtained in the same manner as in Synthesis Example 1 except for using 9′-phenyl-9H,9′H-2,4′-bicarbazole in place of 9-phenyl-9H,9′H-2,4′-bicarbazole and using 9-(4″-chloro-[1,1′: 4′,1″-terphenyl]-4-yl)-9H-carbazole in place of 2-(4′-bromo-[1,1′-biphenyl]-4-yl)dibenzo[b,d]furan. A yield was 82%. The obtained compound was identified as the compound M3-7 by analysis according to LC-MS.

### Synthesis Example 8: Synthesis of Compound M3-8

A synthetic scheme of a compound M3-8 is shown below.

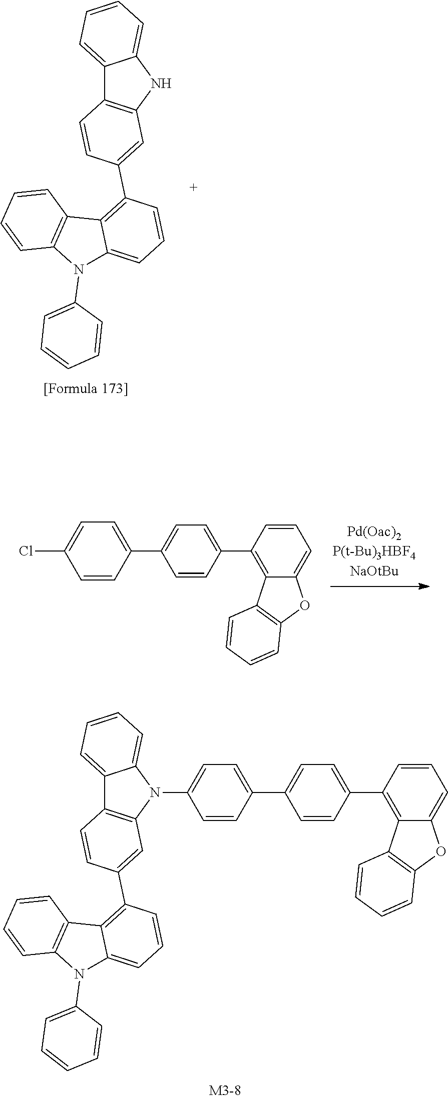 [SMILES: c1ccc(-n2c3ccccc3c3c(-c4ccc5c(c4)[nH]c4ccccc45)cccc32)cc1 ; Clc1ccc(-c2ccc(-c3cccc4oc5ccccc5c34)cc2)cc1 ; c1ccc(-n2c3ccccc3c3c(-c4ccc5c6ccccc6n(-c6ccc(-c7ccc(-c8cccc9oc%10ccccc%10c89)cc7)cc6)c5c4)cccc32)cc1]

The compound M3-8 was obtained in the same manner as in Synthesis Example 1 except for using 9′-phenyl-9H,9′H-2,4′-bicarbazole in place of 9-phenyl-9H,9′H-2,4′-bicarbazole and using 1-(4′-chloro-[1,1′-biphenyl]-4-yl)dibenzo[b,d]furan in place of 2-(4′-bromo-[1,1′-biphenyl]-4-yl)dibenzo[b,d]furan. A yield was 48%. The obtained compound was identified as the compound M3-8 by analysis according to LC-MS.

### Synthesis Example 9: Synthesis of Compound M3-9

A synthetic scheme of a compound M3-9 is shown below.

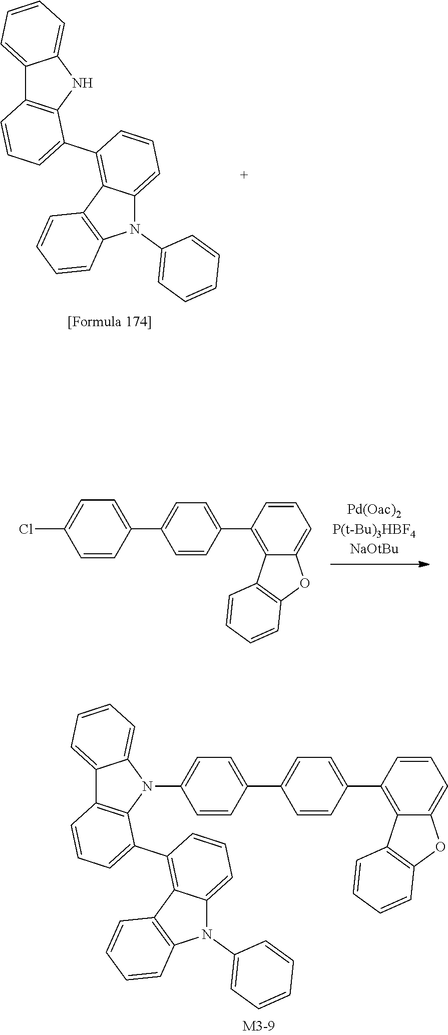 [SMILES: c1ccc(-n2c3ccccc3c3c(-c4cccc5c4[nH]c4ccccc45)cccc32)cc1 ; Clc1ccc(-c2ccc(-c3cccc4oc5ccccc5c34)cc2)cc1 ; c1ccc(-n2c3ccccc3c3c(-c4cccc5c6ccccc6n(-c6ccc(-c7ccc(-c8cccc9oc%10ccccc%10c89)cc7)cc6)c45)cccc32)cc1]

The compound M3-9 was obtained in the same manner as in Synthesis Example 1 except for using 9′-phenyl-9H,9′H-1,4′-bicarbazole in place of 9-phenyl-9H,9′H-2,4′-bicarbazole and using 1-(4′-chloro-[1,1′-biphenyl]-4-yl)dibenzo[b,d]furan in place of 2-(4′-bromo-[1,1′-biphenyl]-4-yl)dibenzo[b,d]furan. A yield was 33%. The obtained compound was identified as the compound M3-9 by analysis according to LC-MS.

### Synthesis Example 10: Synthesis of Compound M3-10

A synthetic scheme of a compound M3-10 is shown below.

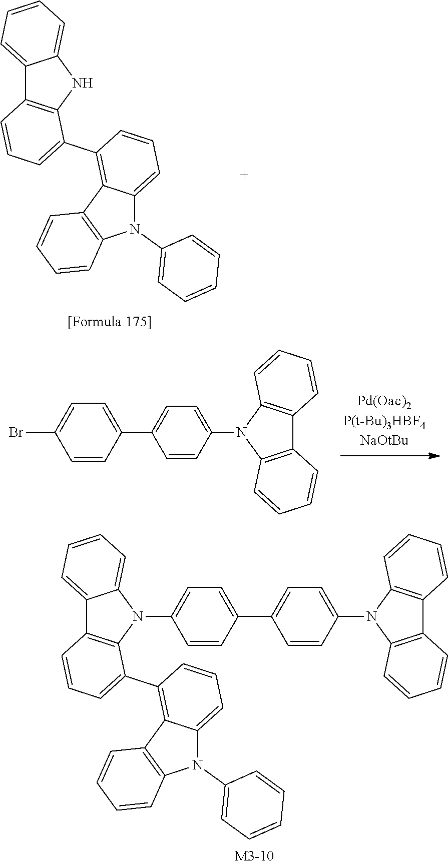 [SMILES: c1ccc(-n2c3ccccc3c3c(-c4cccc5c4[nH]c4ccccc45)cccc32)cc1 ; Brc1ccc(-c2ccc(-n3c4ccccc4c4ccccc43)cc2)cc1 ; c1ccc(-n2c3ccccc3c3c(-c4cccc5c6ccccc6n(-c6ccc(-c7ccc(-n8c9ccccc9c9ccccc98)cc7)cc6)c45)cccc32)cc1]

The compound M3-10 was obtained in the same manner as in Synthesis Example 1 except for using 9′-phenyl-9H,9′H-1,4′-bicarbazole in place of 9-phenyl-9H,9′H-2,4′-bicarbazole and using 9-(4′-bromo-[1,1′-biphenyl]-4-yl)-9H-carbazole in place of 2-(4′-bromo-[1,1′-biphenyl]-4-yl)dibenzo[b,d]furan. A yield was 85%. The obtained compound was identified as the compound M3-10 by analysis according to LC-MS.

### Synthesis Example 11: Synthesis of Compound M3-11

A synthetic scheme of a compound M3-11 is shown below.

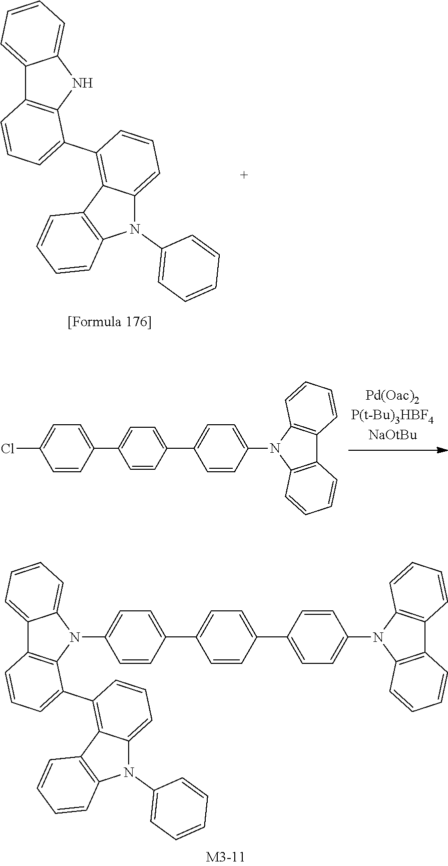 [SMILES: c1ccc(-n2c3ccccc3c3c(-c4cccc5c4[nH]c4ccccc45)cccc32)cc1 ; Clc1ccc(-c2ccc(-c3ccc(-n4c5ccccc5c5ccccc54)cc3)cc2)cc1 ; c1ccc(-n2c3ccccc3c3c(-c4cccc5c6ccccc6n(-c6ccc(-c7ccc(-c8ccc(-n9c%10ccccc%10c%10ccccc%109)cc8)cc7)cc6)c45)cccc32)cc1]

The compound M3-11 was obtained in the same manner as in Synthesis Example 1 except for using 9′-phenyl-9H,9′H-1,4′-bicarbazole in place of 9-phenyl-9H,9′H-2,4′-bicarbazole and using 9-(4″-chloro-[1,1′:4′,1″-terphenyl]-4-yl)-9H-carbazole in place of 2-(4′-bromo-[1,1′-biphenyl]-4-yl)dibenzo[b,d]furan. A yield was 85%. The obtained compound was identified as the compound M3-11 by analysis according to LC-MS.

### EXPLANATION OF CODES

1 . . . organic EL device, 2 . . . substrate, 3 . . . anode, 4 . . . cathode, 5 . . . emitting layer, 6 . . . hole injecting layer, 7 . . . hole transporting layer, 8 . . . electron transporting layer, 9 . . . electron injecting layer

1: An organic electroluminescence device comprising:
an anode;
a cathode;
an emitting layer provided between the anode and the cathode, wherein
the emitting layer comprises a compound M3 represented by a formula (11), a formula (12) or a formula (13) below and a delayed fluorescent compound M2,
the compound M3 and the compound M2 are different in structure, and
a singlet energy S₁(M3) of the compound M3 and a singlet energy S₁(M2) of the compound M2 satisfy a relationship of a numerical formula (Numerical Formula 1) below, 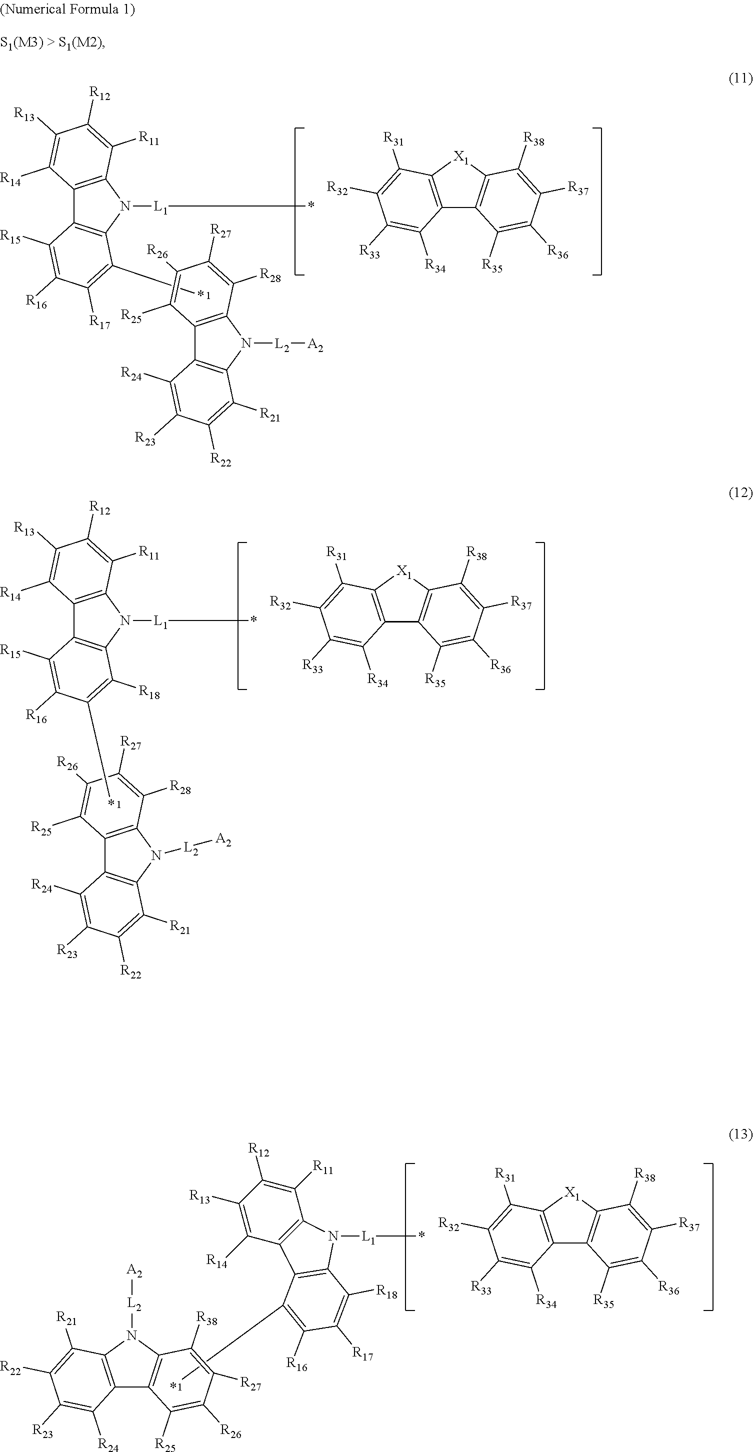
where:
A₂ is a substituted or unsubstituted aryl group having 6 to 30 ring carbon atoms, or a substituted or unsubstituted heterocyclic group having 5 to 30 ring atoms;
L₁ and L₂ are each independently a single bond, a substituted or unsubstituted arylene group having 6 to 30 ring carbon atoms, a substituted or unsubstituted divalent heterocyclic group having 5 to 30 ring atoms, a group obtained by bonding two groups selected from the group consisting of a substituted or unsubstituted arylene group having 6 to 30 ring carbon atoms and a substituted or unsubstituted divalent heterocyclic group having 5 to 30 ring atoms, or a group obtained by bonding three groups selected from the group consisting of a substituted or unsubstituted arylene group having 6 to 30 ring carbon atoms and a substituted or unsubstituted divalent heterocyclic group having 5 to 30 ring atoms;
the two groups are mutually the same or different;
the three groups are mutually the same or different;
R₁₁ to R₁₈ are each independently a hydrogen atom or a substituent, or at least one combination of a combination of R₁₁ and R₁₂, a combination of R₁₂ and R₁₃, a combination of R₁₃ and R₁₄, a combination of R₁₅ and R₁₆, a combination of R₁₆ and R₁₇, or a combination of R₁₇ and R₁₈ are mutually bonded to form a ring;
R₂₁ to R₂₈ are each independently a hydrogen atom or a substituent, or at least one combination of a combination of R₂₁ and R₂₂, a combination of R₂₂ and R₂₃, a combination of R₂₃ and R₂₄, a combination of R₂₅ and R₂₆, a combination of R₂₆ and R₂₇, or a combination of R₂₇ and R₂₈ are mutually bonded to form a ring;
one of carbon atoms bonded to R₂₅, R₂₇ and R₂₈ is bonded to * 1;
X₁ is an oxygen atom, a sulfur atom, or NR₃₉;
R₃₉ is a substituent;
R₃₁ to R₃₈ are each independently a hydrogen atom or a substituent, or at least one combination of a combination of R₃₁ and R₃₂, a combination of R₃₂ and R₃₃, a combination of R₃₃ and R₃₄, a combination of R₃₅ and R₃₆, a combination of R₃₆ and R₃₇, or a combination of R₃₇ and R₃₈ are mutually bonded to form a ring;
one selected from carbon atoms bonded to R₃₁ to R₃₈ and a nitrogen atom bonded to R₃₉ is bonded to *;
R₁₁ to R₁₈, R₂₁ to R₂₈, and R₃₁ to R₃₉ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, a group represented by —N(Rz)₂, a thiol group, a substituted or unsubstituted alkylthio group having 1 to 30 carbon atoms, a substituted or unsubstituted aralkyl group having 7 to 30 ring carbon atoms, a substituted germanium group, a substituted phosphine oxide group, a nitro group, a substituted boryl group, or a substituted or unsubstituted arylthio group having 6 to 30 ring carbon atoms;
Rz is a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms; and
two Rz in —N(Rz)₂ are mutually the same or different.

2: The organic electroluminescence device according to claim 1, wherein
the emitting layer further comprises a fluorescent compound M1, and
a singlet energy S₁(M1) of the compound M1 and a singlet energy S₁(M2) of the compound M2 satisfy a relationship of a numerical formula (Numerical Formula 2) below,
S₁(M2)>S₁(M1) (Numerical Formula 2).

3: The organic electroluminescence device according to claim 1, wherein
a combination of R₁₁ and R₁₂, a combination of R₁₂ and R₁₃, a combination of R₁₃ and R₁₄, a combination of R₁₅ and R₁₆, a combination of R₁₆ and R₁₇, and a combination of R₁₇ and R₁₈ are not mutually bonded.

4: The organic electroluminescence device according to claim 1, wherein
a combination of R₂₁ and R₂₂, a combination of R₂₂ and R₂₃, a combination of R₂₃ and R₂₄, a combination of R₂₅ and R₂₆, a combination of R₂₆ and R₂₇, and a combination of R₂₇ and R₂₈ are not mutually bonded.

5: The organic electroluminescence device according to claim 1, wherein
a combination of R₃₁ and R₃₂, a combination of R₃₂ and R₃₃, a combination of R₃₃ and R₃₄, a combination of R₃₅ and R₃₆, a combination of R₃₆ and R₃₇, and a combination of R₃₇ and R₃₈ are not mutually bonded.

6: The organic electroluminescence device according to claim 1, wherein
R₁₁ to R₁₈, R₂₁ to R₂₈, and R₃₁ to R₃₈ are each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms; and
R₃₉ is a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

7: The organic electroluminescence device according to claim 1, wherein
R₁₁ to R₁₈, R₂₁ to R₂₈, and R₃₁ to R₃₈ are each independently a hydrogen atom, or a substituted or unsubstituted aryl group having 6 to 30 ring carbon atoms, and
R₃₉ is a substituted or unsubstituted aryl group having 6 to 30 ring carbon atoms.

8: The organic electroluminescence device according to claim 1, wherein
R₁₁ to R₁₈, R₂₁ to R₂₈, and R₃₁ to R₃₈ are each a hydrogen atom, and
R₃₉ is a substituted or unsubstituted phenyl group.

9: The organic electroluminescence device according to claim 1, wherein
L₁ and L₂ are each independently a single bond, or a substituted or unsubstituted arylene group having 6 to 30 ring carbon atoms.

10: The organic electroluminescence device according to claim 1, wherein
L₁ and L₂ are each independently a substituted or unsubstituted phenylene group, a substituted or unsubstituted parabiphenylene group, or a substituted or unsubstituted paraterphenylene group.

11: The organic electroluminescence device according to claim 1, wherein
L₁ and L₂ are each independently a substituted or unsubstituted parabiphenylene group, or a substituted or unsubstituted paraterphenylene group.

12: The organic electroluminescence device according to claim 1, wherein X₁ is an oxygen atom or a sulfur atom.

13: The organic electroluminescence device according to claim 1, wherein X₁ is an oxygen atom.

14: The organic electroluminescence device according to claim 1, wherein the compound M3 is a compound represented by one of formulae (111) to (114) below, 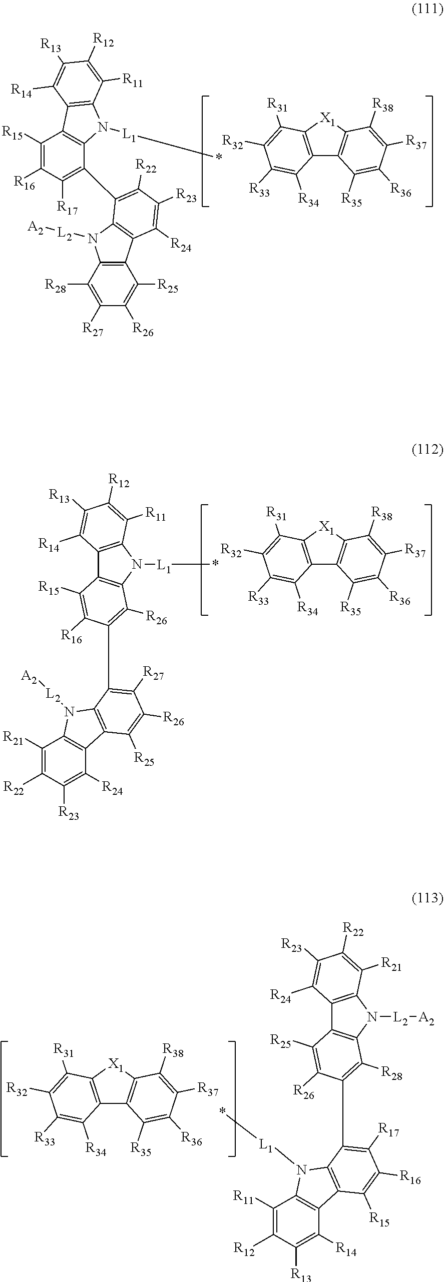 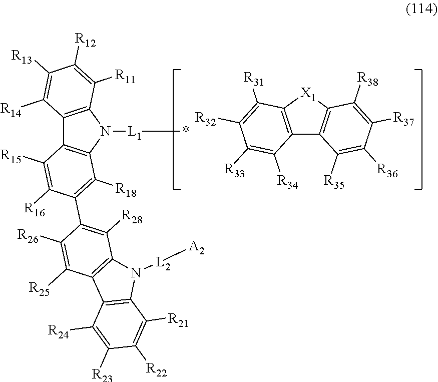
where: A₂, L₁, L₂, X₁, R₁₁ to R₁₈, R₂₁ to R₂₈ and R₃₁ to R₃₉ each independently represent the same as A₂, L₁, L₂, X₁, R₁₁ to R₁₈, R₂₁ to R₂₈ and R₃₁ to R₃₉ in the formulae (11) to (13) and one selected from carbon atoms bonded to R₃₁ to R₃₈ and a nitrogen atom bonded to R₃₉ is bonded to *.

15: The organic electroluminescence device according to claim 1, wherein the compound M3 is a compound represented by one of formulae (115) to (119), 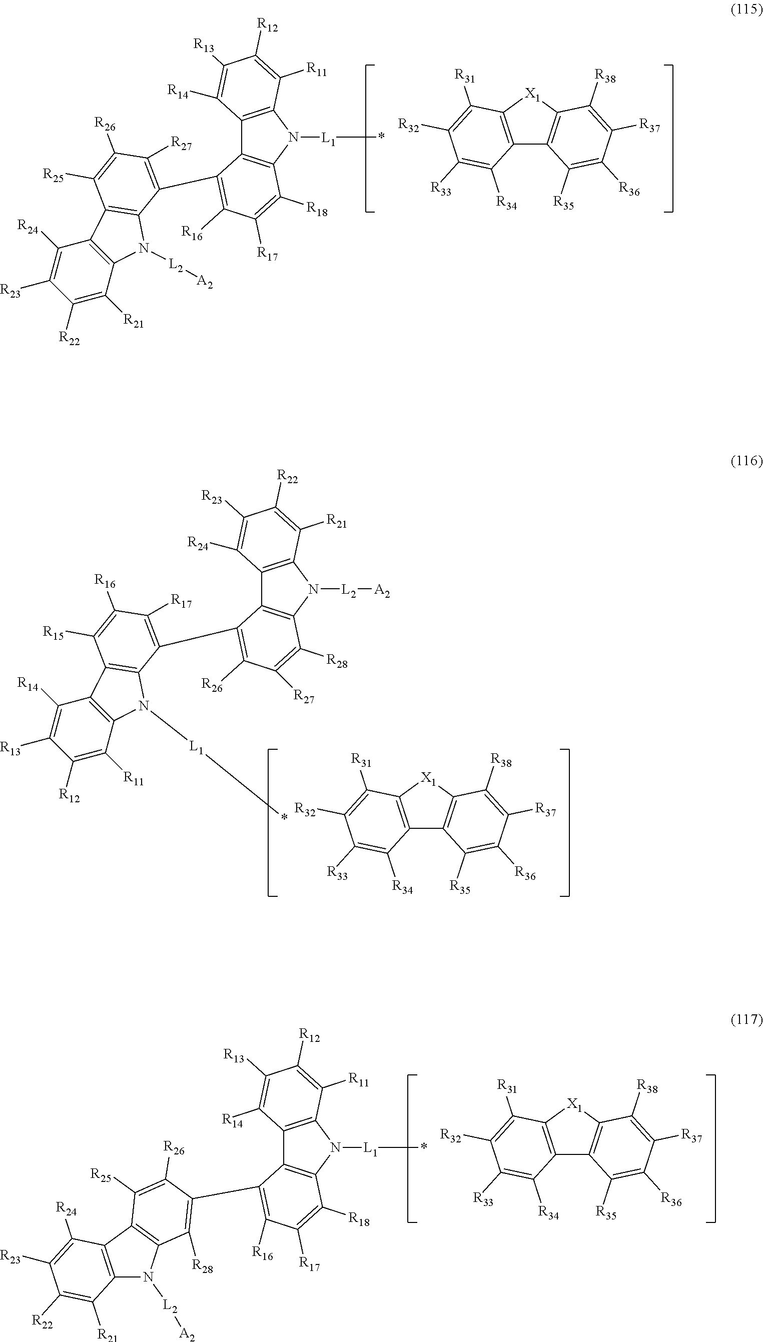 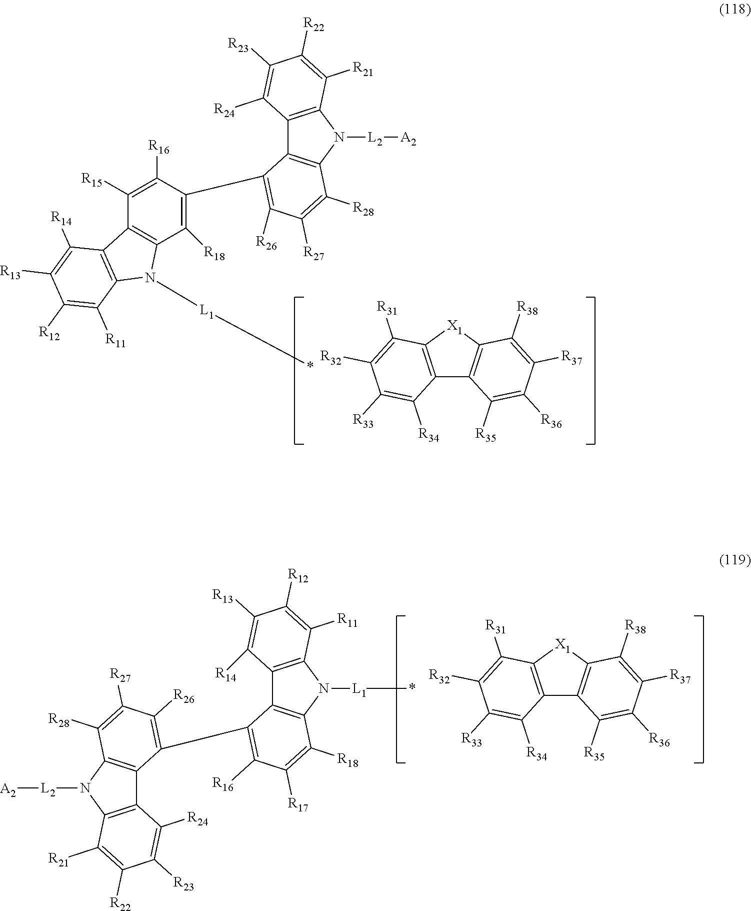
where: A₂, L₁, L₂, X₁, R₁₁ to R₁₈, R₂₁ to R₂₈ and R₃₁ to R₃₉ each independently represent the same as A₂, L₁, L₂, X₁, R₁₁ to R₁₈, R₂₁ to R₂₈ and R₃₁ to R₃₉ in the formulae (11) to (13) and one selected from carbon atoms bonded to R₃₁ to R₃₈ and a nitrogen atom bonded to R₃₉ is bonded to *.

16: The organic electroluminescence device according to claim 15, wherein the compound M3 is a compound represented by one of the formulae (115), (117) and (119).

17: The organic electroluminescence device according to claim 1, wherein the compound M3 is a compound represented by one of formulae (111) to (113) and (115) to (119) below, 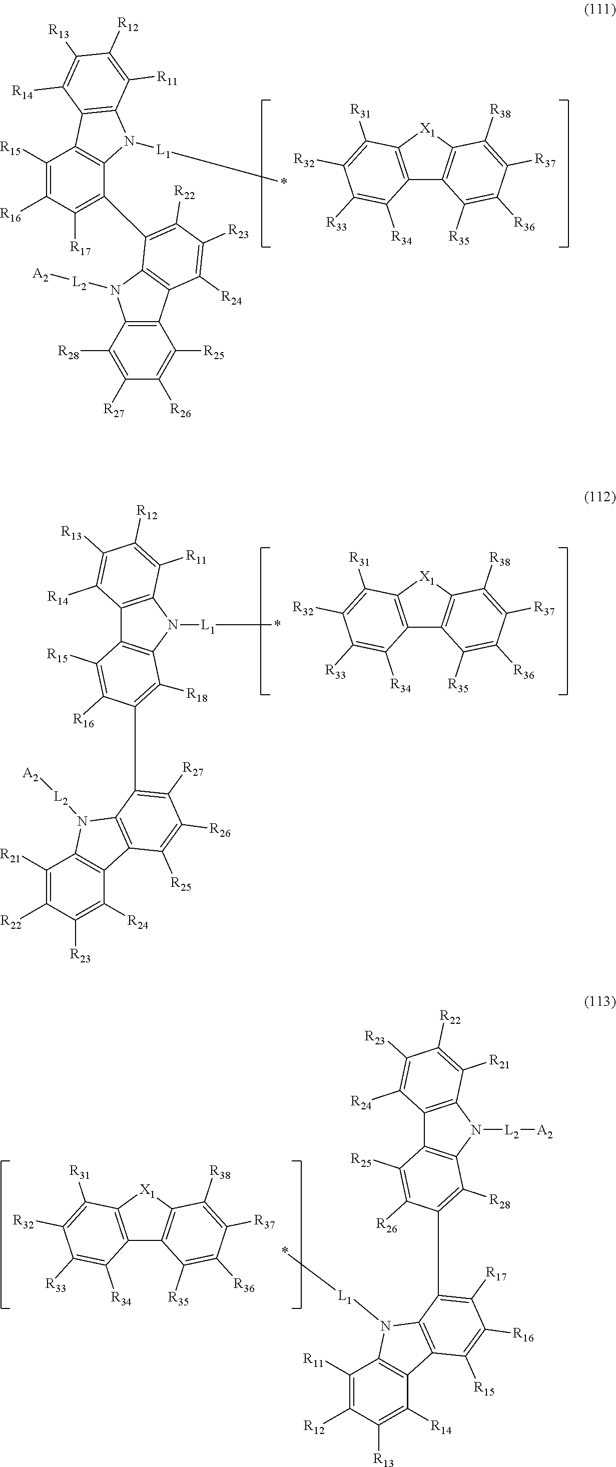 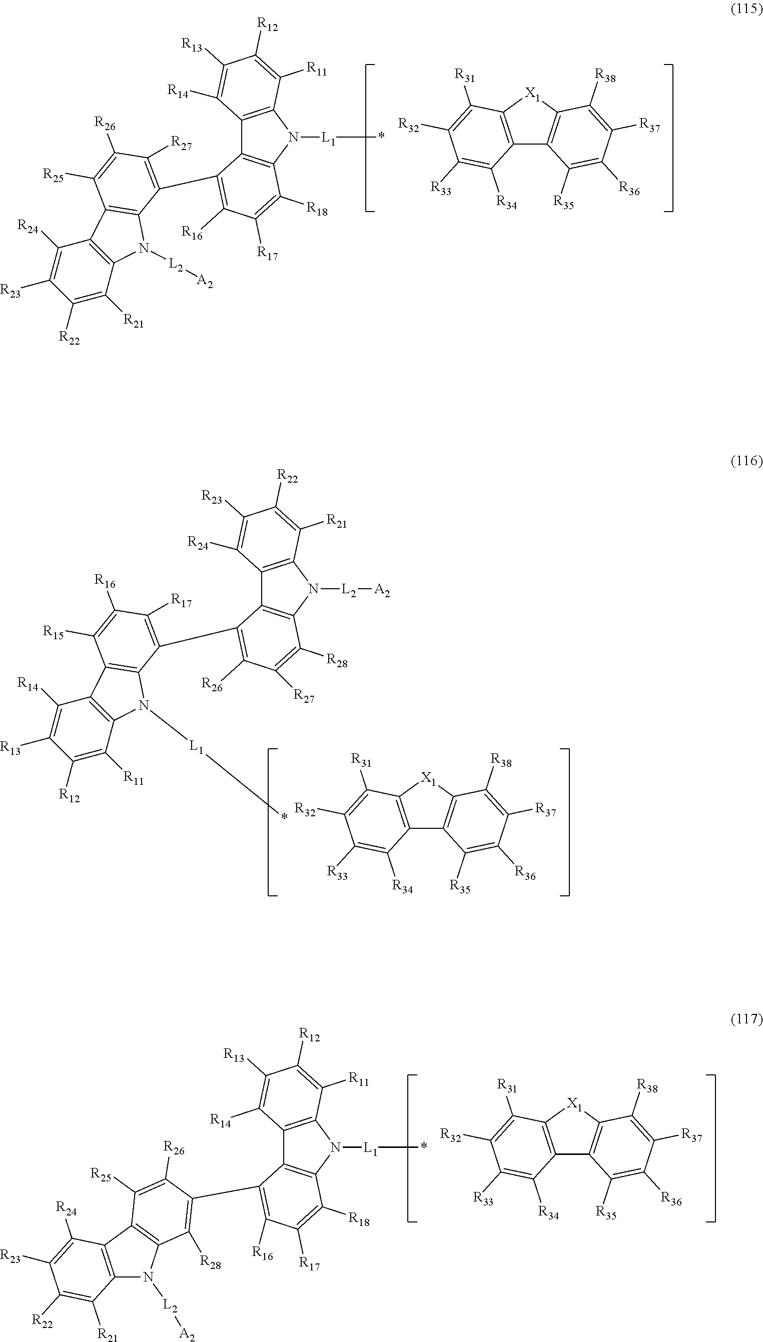 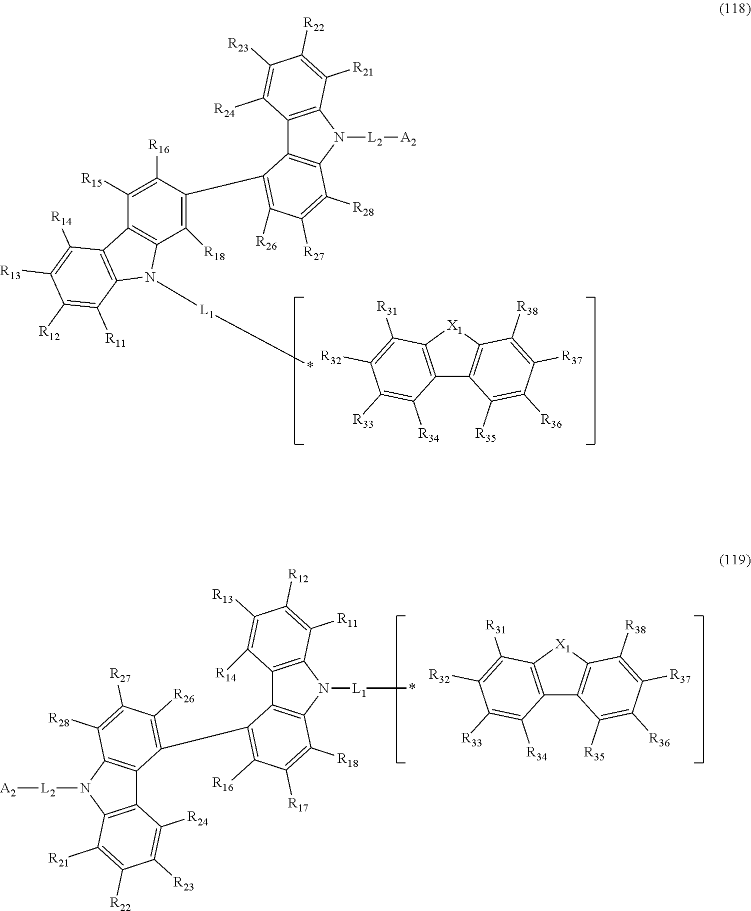
where: A₂, L₁, L₂, X₁, R₁₁ to R₁₈, R₂₁ to R₂₈ and R₃₁ to R₃₉ each independently represent the same as A₂, L₁, L₂, X₁, R₁₁ to R₁₈, R₂₁ to R₂₈ and R₃₁ to R₃₉ in the formulae (11) to (13) and one selected from carbon atoms bonded to R₃₁ to R₃₈ and a nitrogen atom bonded to R₃₉ is bonded to *.

18: The organic electroluminescence device according to claim 17, wherein the compound M3 is a compound represented by one of the formulae (111), (115), (116) and (119).

19: The organic electroluminescence device according to claim 1, wherein the compound M2 is a compound represented by a formula (2) or a formula (22) below, 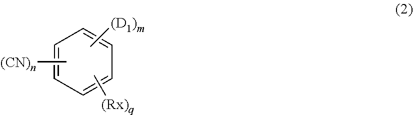
where:
n is 1, 2, 3 or 4;
m is 1, 2, 3 or 4;
q is 0, 1, 2, 3 or 4;
m+n+q=6 is satisfied;
CN is a cyano group;
D₁ is a group represented by a formula (2a), (2b) or (2c) below, and when a plurality of D₁ are present, the plurality of D₁ are mutually the same or different;
Rx is a hydrogen atom or a substituent, or at least one combination of combinations of adjacent ones of Rx are mutually bonded to form a ring, and when a plurality of Rx are present, the plurality of Rx are mutually the same or different; and
each Rx as a substituent is independently a halogen atom, a substituted or unsubstituted aryl group having 6 to 30 ring carbon atoms, a substituted or unsubstituted heterocyclic group having 5 to 30 ring atoms, a substituted or unsubstituted amino group, a substituted or unsubstituted carbonyl group, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkylsilyl group having 3 to 30 carbon atoms, or a substituted or unsubstituted arylsilyl group having 6 to 60 ring carbon atoms; and
CN, D₁ and Rx are bonded to respective carbon atoms of a six-membered ring, 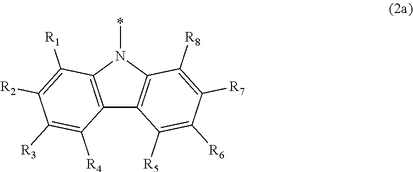
where:
R₁ to R₈ are each independently a hydrogen atom or a substituent, or at least one combination of a combination of R₁ and R₂, a combination of R₂ and R₃, a combination of R₃ and R₄, a combination of R₅ and R₆, a combination of R₆ and R₇, or a combination of R₇ and R₈ are mutually bonded to form a ring;
R₁ to R₈ as a substituent are each independently a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkylsilyl group having 3 to 30 carbon atoms, a substituted or unsubstituted arylsilyl group having 6 to 60 ring carbon atoms, a hydroxy group, a substituted or unsubstituted alkoxy group having 1 to 30 carbon atoms, a substituted or unsubstituted alkoxy halide group having 1 to 30 carbon atoms, a substituted or unsubstituted aryloxy group having 6 to 30 ring carbon atoms, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and
* represents a bonding position to a carbon atom in a six-membered ring in the formula (2), 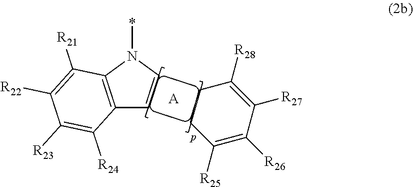
where:
R₂₁ to R₂₈ are each independently a hydrogen atom or a substituent, or at least one combination of a combination of R₂₁ and R₂₂, a combination of R₂₂ and R₂₃, a combination of R₂₃ and R₂₄, a combination of R₂₅ and R₂₆, a combination of R₂₆ and R₂₇, or a combination of R₂₇ and R₂₈ are mutually bonded to form a ring;
R₂₁ to R₂₈ as a substituent each independently represent the same as R₁ to R₈ in the formula (2a);
A represents a cyclic structure represented by a formula (211) or (212) below, and the cyclic structure A is fused with an adjacent cyclic structure at any position;
p is 1, 2, 3 or 4;
when p is 2, 3 or 4, a plurality of cyclic structures A are mutually the same or different; and
* represents a bonding position to a carbon atom in a six-membered ring in the formula (2), 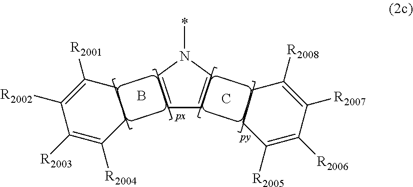
where:
R₂₀₀₁ to R₂₀₀₈ are each independently a hydrogen atom or a substituent, or at least one combination of a combination of R₂₀₀₁ and R₂₀₀₂, a combination of R₂₀₀₂ and R₂₀₀₃, a combination of R₂₀₀₃ and R₂₀₀₄, a combination of R₂₀₀₅ and R₂₀₀₆, a combination of R₂₀₀₆ and R₂₀₀₇, or a combination of R₂₀₀₇ and R₂₀₀₈ are mutually bonded to form a ring;
R₂₀₀₁ to R₂₀₀₈ as a substituent each independently represent the same as R₁ to R₈ as a substituent in the formula (2a);
B represents a cyclic structure represented by the formula (211) or (212), and the cyclic structure B is fused with an adjacent cyclic structure at any position;
px is 1, 2, 3 or 4;
when px is 2, 3 or 4, a plurality of cyclic structures B are mutually the same or different;
C represents a cyclic structure represented by the formula (211) or (212), and the cyclic structure C is fused with an adjacent cyclic structure at any position;
py is 1, 2, 3 or 4;
when py is 2, 3 or 4, a plurality of cyclic structures C are mutually the same or different; and
* represents a bonding position to a carbon atom in a six-membered ring in the formula (2), 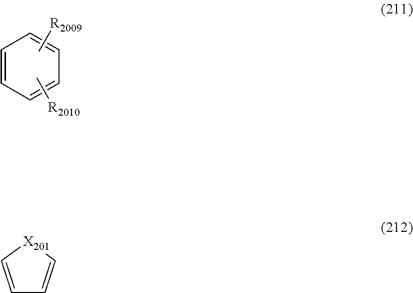
where, in the formula (211): R₂₀₀₉ and R₂₀₁₀ are each independently a hydrogen atom or a substituent, or bonded to a part of an adjacent cyclic structure to form a ring, or a combination of R₂₀₀₉ and R₂₀₁₀ are mutually bonded to form a ring,
in the formula (212): X₂₀₁ is CR₂₀₁₁R₂₀₁₂, NR₂₀₁₃, a sulfur atom, or an oxygen atom, and R₂₀₁₁, R₂₀₁₂ and R₂₀₁₃ are each independently a hydrogen atom or a substituent, or R₂₀₁₁ and R₂₀₁₂ are mutually bonded to form a ring; and
R₂₀₀₉, R₂₀₁₀, R₂₀₁₁, R₂₀₁₂ and R₂₀₁₃ as a substituent each independently represent the same as R₁ to R₈ as a substituent in the formula (2a), 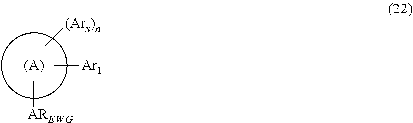
where, in the formula (22):
Ar₁ is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by formulae (1a) to (1j) below;
Ar_{EWG} is a substituted or unsubstituted heteroaryl group having 5 to 30 ring atoms that includes at least one nitrogen atom in a ring, or an aryl group having 6 to 30 ring carbon atoms that is substituted by at least one cyano group;
Ar_{X} is each independently a hydrogen atom or a substituent, and Ar_{X} as a substituent is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by the formulae (1a) to (1j);
n is 0, 1, 2, 3, 4 or 5, and when n is 2, 3, 4 or 5, a plurality of Ar_{X} are mutually the same or different;
a ring (A) is a substituted or unsubstituted aromatic hydrocarbon ring or a substituted or unsubstituted heterocycle, the ring (A) being a five-membered ring, a six-membered ring, or a seven-membered ring, Ar_{EWG}, Ar₁ and Ar_{X} being bonded to respective ones of elements forming the ring (A); and
at least one of Ar₁ or Ar_{X} is a group selected from the group consisting of groups represented by formulae (1a) to (1j) below, 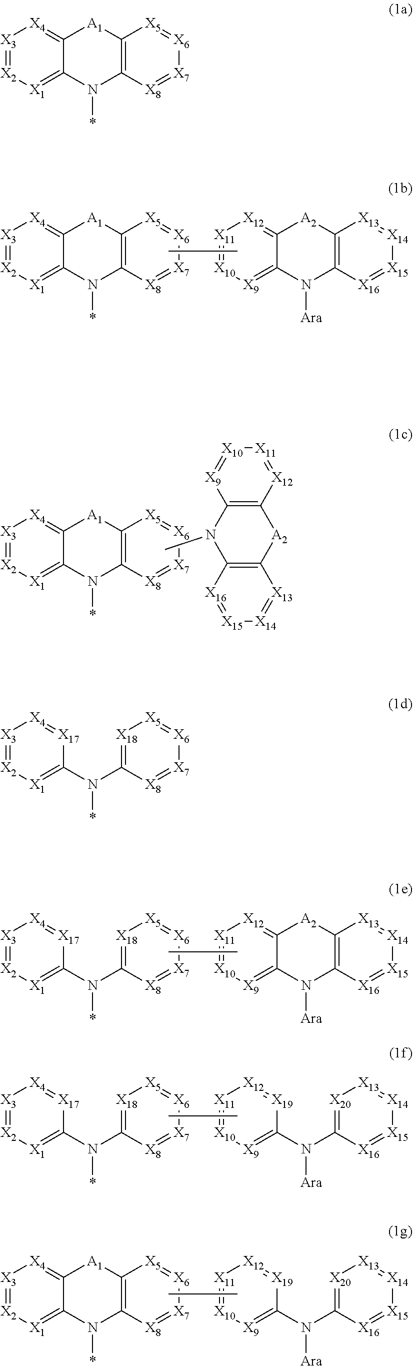 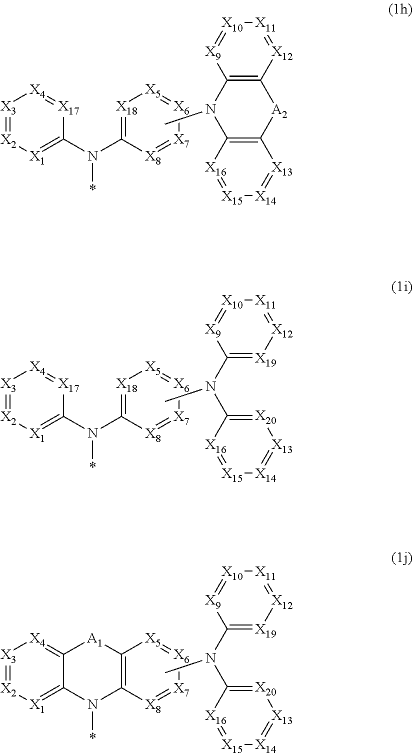
where, in the formulae (1a) to (1j): X₁ to X₂₀ are each independently a nitrogen atom (N) or a carbon atom bonded with R_{A1} (C—R_{A1});
in the formula (1b): one of X₅ to X₈ is a carbon atom bonded to one of X₉ to X₁₂, and one of X₉ to X₁₂ is a carbon atom bonded to one of X₅ to X₈;
in the formula (1c): one of X₅ to X₈ is a carbon atom bonded to a nitrogen atom in a ring including A₂;
in the formula (1e): one of X₅ to X₈ and X₁₈ is a carbon atom bonded to one of X₉ to X₁₂, and one of X₉ to X₁₂ is a carbon atom bonded to one of X₅ to X₈ and X₁₈;
in the formula (1f): one of X₅ to X₈ and X₁₈ is a carbon atom bonded to one of X₉ to X₁₂ and X₁₉, and one of X₉ to X₁₂ and X₁₉ is a carbon atom bonded to one of X₅ to X₈ and X₁₈;
in the formula (1g): one of X₅ to X₈ is a carbon atom bonded to one of X₉ to X₁₂ and X₁₉, and one of X₉ to X₁₂ and X₁₉ is a carbon atom bonded to one of X₅ to X₈;
in the formula (1h): one of X₅ to X₈ and X₁₈ is a carbon atom bonded to a nitrogen atom in a ring including A₂;
in the formula (1i): one of X₅ to X₈ and X₁₈ is a carbon atom bonded to a nitrogen atom that links a ring including X₉ to X₁₂ and X₁₉ with a ring including X₁₃ to X₁₆ and X₂₀;
in the formula (1j): one of X₅ to X₈ is a carbon atom bonded to a nitrogen atom that links a ring including X₉ to X₁₂ and X₁₉ with a ring including X₁₃ to X₁₆ and X₂₀;
R_{A1} is each independently a hydrogen atom or a substituent, or at least one combination of combinations of a plurality of R_{A1} are mutually directly bonded to form a ring or bonded via a hetero atom to form a ring;
R_{A1} as a substituent is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group;
a plurality of R_{A1} as a substituent are mutually the same or different;
in the formulae (1a) to (1j), * represents a bonding position to the ring (A);
in the formulae (1a) to (1j), A₁ and A₂ are each independently a single bond, an oxygen atom (O), a sulfur atom (S), C(R₂₀₂₁)(R₂₀₂₂), Si(R₂₀₂₃)(R₂₀₂₄), C(═O), S(═O), SO₂ or N(R₂₀₂₅); R₂₀₂₁ to R₂₀₂₅ are each independently a hydrogen atom or a substituent, and R₂₀₂₁ to R₂₀₂₅ as a substituent are each independently a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group; and
in the formulae (1a) to (1j), Ara is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, and a substituted silyl group.

20: The organic electroluminescence device according to claim 19, wherein the compound M2 is a compound represented by the formula (2).

21: An electronic device comprising the organic electroluminescence device according to claim 1.

22: A compound represented by a formula (121) or a formula (122) below, 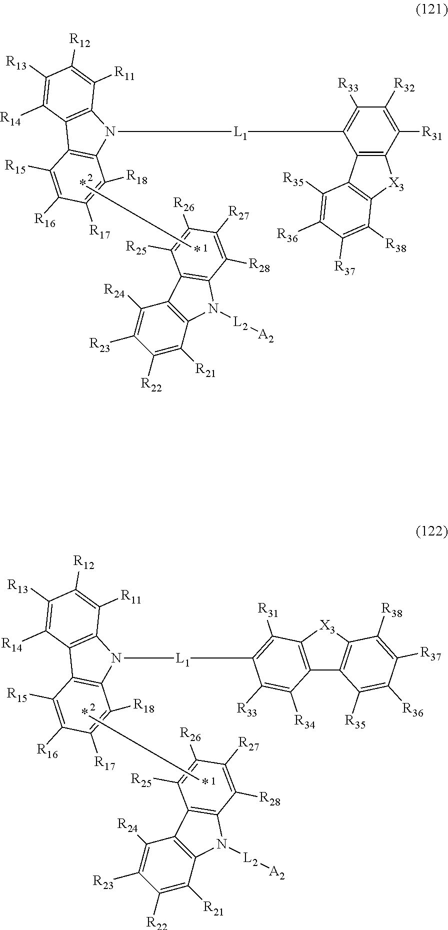
where:
A₂ is a substituted or unsubstituted aryl group having 6 to 30 ring carbon atoms, or a substituted or unsubstituted heterocyclic group having 5 to 30 ring atoms;
L₁ and L₂ are each independently a single bond, a substituted or unsubstituted arylene group having 6 to 30 ring carbon atoms, a substituted or unsubstituted divalent heterocyclic group having 5 to 30 ring atoms, a group obtained by bonding two groups selected from the group consisting of a substituted or unsubstituted arylene group having 6 to 30 ring carbon atoms and a substituted or unsubstituted divalent heterocyclic group having 5 to 30 ring atoms, or a group obtained by bonding three groups selected from the group consisting of a substituted or unsubstituted arylene group having 6 to 30 ring carbon atoms and a substituted or unsubstituted divalent heterocyclic group having 5 to 30 ring atoms;
the two groups are mutually the same or different;
the three groups are mutually the same or different;
R₁₁ to R₁₈ are each independently a hydrogen atom or a substituent, or at least one combination of a combination of R₁₁ and R₁₂, a combination of R₁₂ and R₁₃, a combination of R₁₃ and R₁₄, a combination of R₁₅ and R₁₆, a combination of R₁₆ and R₁₇, or a combination of R₁₇ and R₁₈ are mutually bonded to form a ring;
R₂₁ to R₂₈ are each independently a hydrogen atom or a substituent, or at least one combination of a combination of R₂₁ and R₂₂, a combination of R₂₂ and R₂₃, a combination of R₂₃ and R₂₄, a combination of R₂₅ and R₂₆, a combination of R₂₆ and R₂₇, or a combination of R₂₇ and R₂₈ are mutually bonded to form a ring;
one of carbon atoms bonded to R₁₅, R₁₇ and R₁₈ is bonded to *2 and one of carbon atoms bonded to R₂₅, R₂₇ and R₂₈ is bonded to * 1;
a carbon atom bonded to R₁₇ and a carbon atom bonded to R₂₇ are not simultaneously bonded to *2 and *1, respectively;
X₃ is an oxygen atom or a sulfur atom;
R₃₁ to R₃₈ are each independently a hydrogen atom or a substituent, or at least one combination of a combination of R₃₁ and R₃₂, a combination of R₃₂ and R₃₃, a combination of R₃₃ and R₃₄, a combination of R₃₅ and R₃₆, a combination of R₃₆ and R₃₇, or a combination of R₃₇ and R₃₈ are mutually bonded to form a ring;
R₁₁ to R₁₈, R₂₁ to R₂₈, and R₃₁ to R₃₈ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, a group represented by —N(Rz)₂, a thiol group, a substituted or unsubstituted alkylthio group having 1 to 30 carbon atoms, a substituted or unsubstituted aralkyl group having 7 to 30 ring carbon atoms, a substituted germanium group, a substituted phosphine oxide group, a nitro group, a substituted boryl group, or a substituted or unsubstituted arylthio group having 6 to 30 ring carbon atoms;
Rz is a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms; and
two Rz in —N(Rz)₂ are mutually the same or different.

23: The compound according to claim 22, wherein the compound is represented by the formula (121).

24: A compound represented by one of formulae (131) to (134) below, 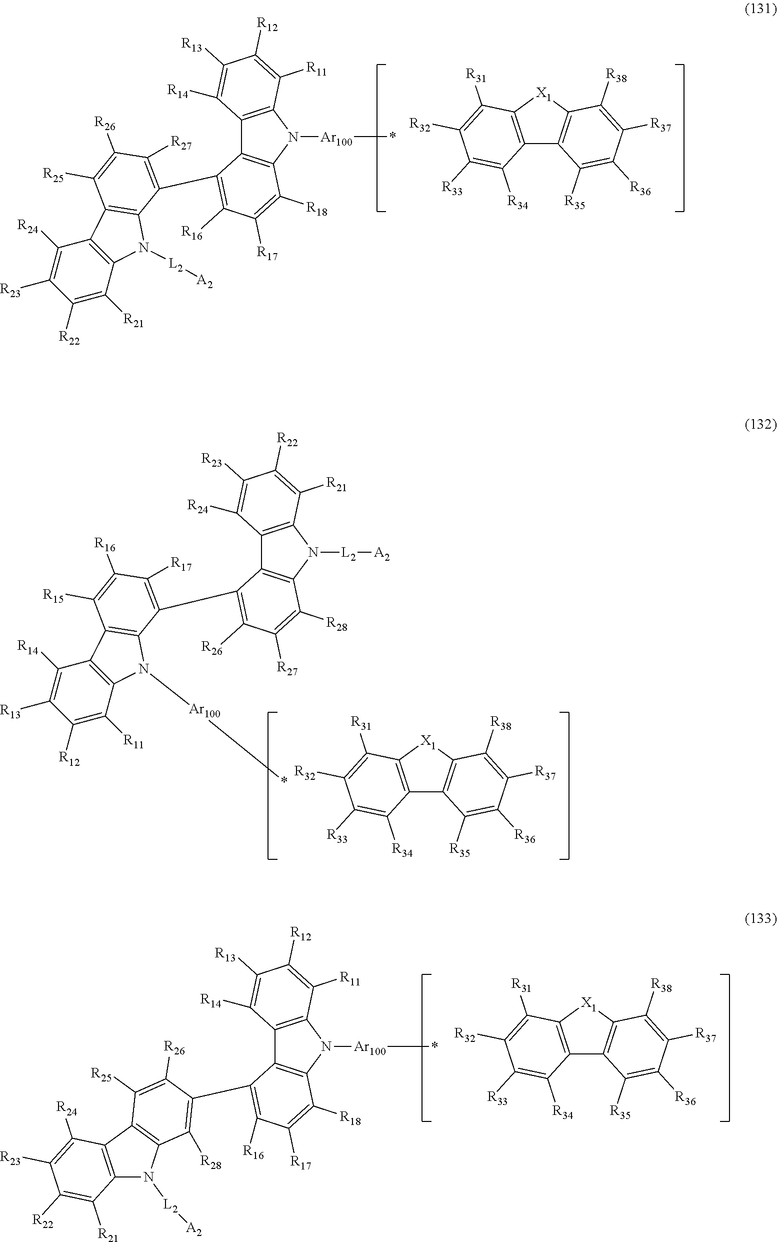 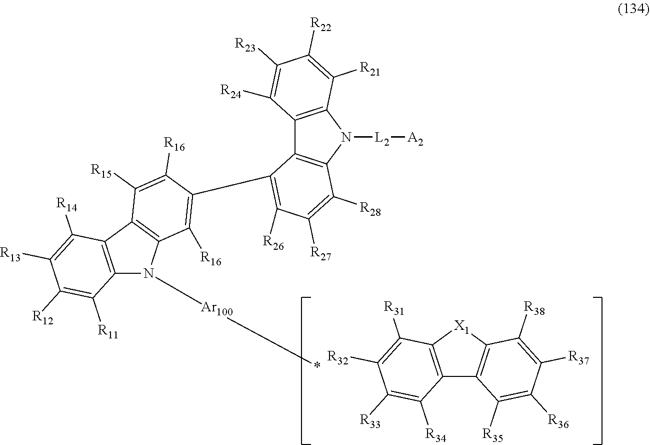
where:
A₂ is a substituted or unsubstituted aryl group having 6 to 30 ring carbon atoms, or a substituted or unsubstituted heterocyclic group having 5 to 30 ring atoms;
Ar₁₀₀ is a substituted or unsubstituted arylene group having 6 to 30 ring carbon atoms,
L₂ is a single bond, a substituted or unsubstituted arylene group having 6 to 30 ring carbon atoms, a substituted or unsubstituted divalent heterocyclic group having 5 to 30 ring atoms, a group obtained by bonding two groups selected from the group consisting of a substituted or unsubstituted arylene group having 6 to 30 ring carbon atoms and a substituted or unsubstituted divalent heterocyclic group having 5 to 30 ring atoms, or a group obtained by bonding three groups selected from the group consisting of a substituted or unsubstituted arylene group having 6 to 30 ring carbon atoms and a substituted or unsubstituted divalent heterocyclic group having 5 to 30 ring atoms;
the two groups are mutually the same or different;
the three groups are mutually the same or different;
R₁₁ to R₁₈ are each independently a hydrogen atom or a substituent, or at least one combination of a combination of R₁₁ and R₁₂, a combination of R₁₂ and R₁₃, a combination of R₁₃ and R₁₄, a combination of R₁₅ and R₁₆, a combination of R₁₆ and R₁₇, or a combination of R₁₇ and R₁₈ are mutually bonded to form a ring;
R₂₁ to R₂₈ are each independently a hydrogen atom or a substituent, or at least one combination of a combination of R₂₁ and R₂₂, a combination of R₂₂ and R₂₃, a combination of R₂₃ and R₂₄, a combination of R₂₅ and R₂₆, a combination of R₂₆ and R₂₇, or a combination of R₂₇ and R₂₈ are mutually bonded to form a ring;
X₁ is an oxygen atom, a sulfur atom, or NR₃₉;
R₃₉ is a substituent;
R₃₁ to R₃₈ are each independently a hydrogen atom or a substituent, or at least one combination of a combination of R₃₁ and R₃₂, a combination of R₃₂ and R₃₃, a combination of R₃₃ and R₃₄, a combination of R₃₅ and R₃₆, a combination of R₃₆ and R₃₇, or a combination of R₃₇ and R₃₈ are mutually bonded to form a ring;
one selected from carbon atoms bonded to R₃₁ to R₃₈ and a nitrogen atom bonded to R₃₉ is bonded to *;
R₁₁ to R₁₈, R₂₁ to R₂₈, and R₃₁ to R₃₉ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, a group represented by —N(Rz)₂, a thiol group, a substituted or unsubstituted alkylthio group having 1 to 30 carbon atoms, a substituted or unsubstituted aralkyl group having 7 to 30 ring carbon atoms, a substituted germanium group, a substituted phosphine oxide group, a nitro group, a substituted boryl group, or a substituted or unsubstituted arylthio group having 6 to 30 ring carbon atoms;
Rz is a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms;
two Rz in —N(Rz)₂ are mutually the same or different; and
when Ar₁₀₀ has a substituent, the substituent is each independently a halogen atom, a cyano group, an unsubstituted aryl group having 6 to 30 ring carbon atoms, an unsubstituted heterocyclic group having 5 to 30 ring atoms, an unsubstituted alkyl group having 1 to 30 carbon atoms, an unsubstituted alkyl halide group having 1 to 30 carbon atoms, an unsubstituted alkenyl group having 2 to 30 carbon atoms, an unsubstituted alkynyl group having 2 to 30 carbon atoms, an unsubstituted alkylsilyl group having 3 to 30 carbon atoms, an unsubstituted arylsilyl group having 6 to 60 ring carbon atoms, an unsubstituted arylphosphoryl group having 6 to 60 ring carbon atoms, a hydroxy group, an unsubstituted alkoxy group having 1 to 30 carbon atoms, an unsubstituted aryloxy group having 6 to 30 ring carbon atoms, a thiol group, an unsubstituted alkylthio group having 1 to 30 carbon atoms, an unsubstituted aralkyl group having 7 to 30 ring carbon atoms, a substituted germanium group, a substituted phosphine oxide group, a nitro group, a substituted boryl group, or an unsubstituted arylthio group having 6 to 30 ring carbon atoms.

25: A compound represented by a formula (141), a formula (142), or a formula (143) below, 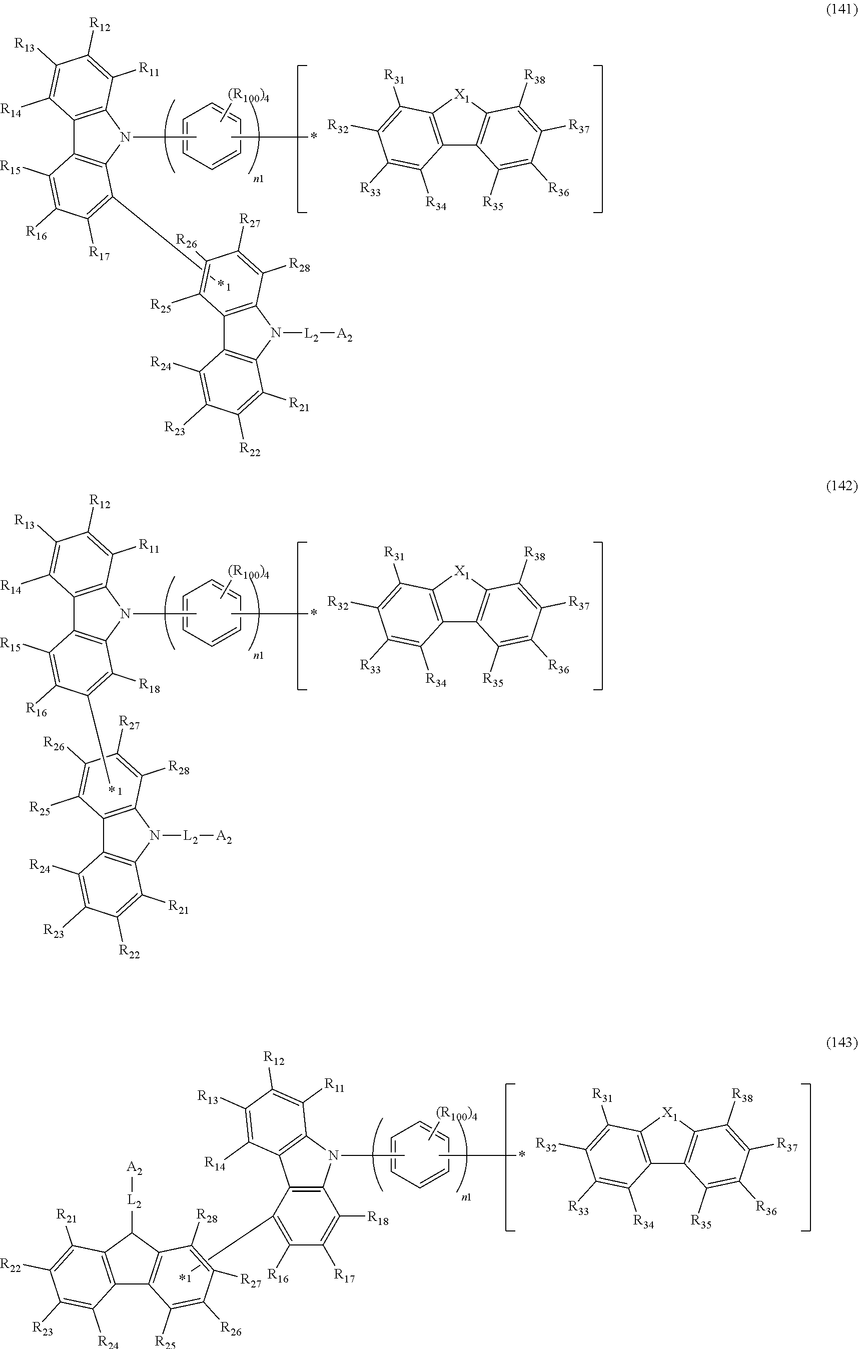
where:
A₂ is a substituted or unsubstituted aryl group having 6 to 30 ring carbon atoms, or a substituted or unsubstituted heterocyclic group having 5 to 30 ring atoms;
L₂ are each independently a single bond, a substituted or unsubstituted arylene group having 6 to 30 ring carbon atoms, a substituted or unsubstituted divalent heterocyclic group having 5 to 30 ring atoms, a group obtained by bonding two groups selected from the group consisting of a substituted or unsubstituted arylene group having 6 to 30 ring carbon atoms and a substituted or unsubstituted divalent heterocyclic group having 5 to 30 ring atoms, or a group obtained by bonding three groups selected from the group consisting of a substituted or unsubstituted arylene group having 6 to 30 ring carbon atoms and a substituted or unsubstituted divalent heterocyclic group having 5 to 30 ring atoms;
the two groups are mutually the same or different;
the three groups are mutually the same or different;
R₁₀₀ is each independently a hydrogen atom or a substituent;
n1 is 2, 3, or 4;
four R₁₀₀ are mutually the same or different;
R₁₁ to R₁₈ are each independently a hydrogen atom or a substituent, or at least one combination of a combination of R₁₁ and R₁₂, a combination of R₁₂ and R₁₃, a combination of R₁₃ and R₁₄, a combination of R₁₅ and R₁₆, a combination of R₁₆ and R₁₇, or a combination of R₁₇ and R₁₈ are mutually bonded to form a ring;
R₂₁ to R₂₈ are each independently a hydrogen atom or a substituent, or at least one combination of a combination of R₂₁ and R₂₂, a combination of R₂₂ and R₂₃, a combination of R₂₃ and R₂₄, a combination of R₂₅ and R₂₆, a combination of R₂₆ and R₂₇, or a combination of R₂₇ and R₂₈ are mutually bonded to form a ring;
one of carbon atoms bonded to R₂₅, R₂₇ and R₂₈ is bonded to * 1;
X₁ is an oxygen atom, a sulfur atom, or NR₃₉;
R₃₉ is a substituent;
R₃₁ to R₃₈ are each independently a hydrogen atom or a substituent, or at least one combination of a combination of R₃₁ and R₃₂, a combination of R₃₂ and R₃₃, a combination of R₃₃ and R₃₄, a combination of R₃₅ and R₃₆, a combination of R₃₆ and R₃₇, or a combination of R₃₇ and R₃₈ are mutually bonded to form a ring;
one selected from carbon atoms bonded to R₃₁ to R₃₈ and a nitrogen atom bonded to R₃₉ is bonded to *;
R₁₀₀, R₁₁ to R₁₈, R₂₁ to R₂₈ and R₃₁ to R₃₉ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, a group represented by —N(Rz)₂, a thiol group, a substituted or unsubstituted alkylthio group having 1 to 30 carbon atoms, a substituted or unsubstituted aralkyl group having 7 to 30 ring carbon atoms, a substituted germanium group, a substituted phosphine oxide group, a nitro group, a substituted boryl group, or a substituted or unsubstituted arylthio group having 6 to 30 ring carbon atoms;
Rz is a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms;
two Rz in —N(Rz)₂ are mutually the same or different; and
R₁₀₀ is not a group represented by —N(Rz)₂;
when R₁₀₀ has a substituent, the substituent is each independently a halogen atom, a cyano group, an unsubstituted aryl group having 6 to 30 ring carbon atoms, an unsubstituted heterocyclic group having 5 to 30 ring atoms, an unsubstituted alkyl group having 1 to 30 carbon atoms, an unsubstituted alkyl halide group having 1 to 30 carbon atoms, an unsubstituted alkenyl group having 2 to 30 carbon atoms, an unsubstituted alkynyl group having 2 to 30 carbon atoms, an unsubstituted alkylsilyl group having 3 to 30 carbon atoms, an unsubstituted arylsilyl group having 6 to 60 ring carbon atoms, an unsubstituted arylphosphoryl group having 6 to 60 ring carbon atoms, a hydroxy group, an unsubstituted alkoxy group having 1 to 30 carbon atoms, an unsubstituted aryloxy group having 6 to 30 ring carbon atoms, a thiol group, an unsubstituted alkylthio group having 1 to 30 carbon atoms, an unsubstituted aralkyl group having 7 to 30 ring carbon atoms, a substituted germanium group, a substituted phosphine oxide group, a nitro group, a substituted boryl group, or an unsubstituted arylthio group having 6 to 30 ring carbon atoms.

26: The compound according to claim 25, wherein the compound is represented by a formula (141A), a formula (142A), or a formula (143A) below, 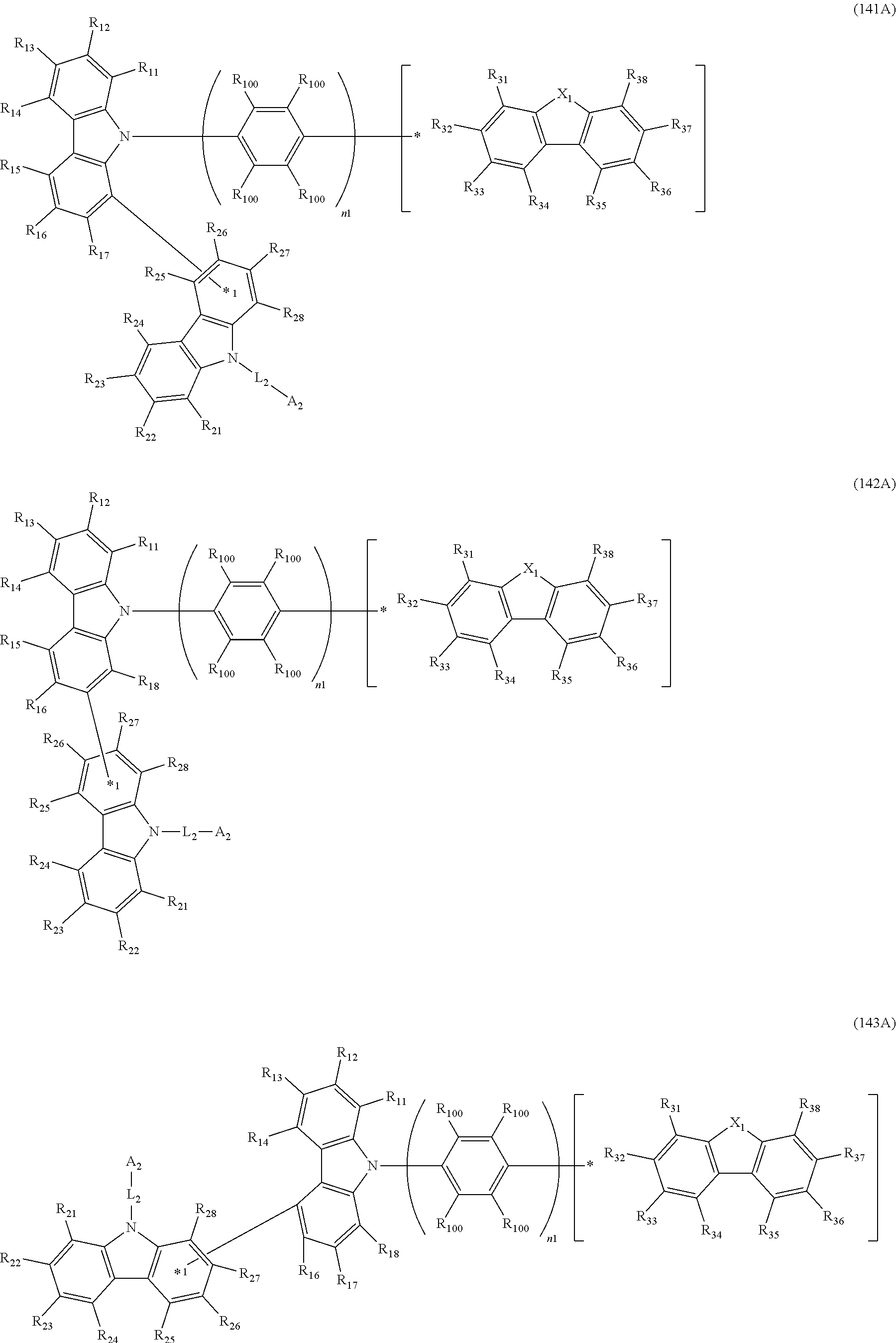
where: A₂, L₂, X₁, R₁₁ to R₁₈, R₂₁ to R₂₈, R₃₁ to R₃₉, R₁₀₀ and n1 each independently represent the same as A₂, L₂, X₁, R₁₁ to R₁₈, R₂₁ to R₂₈, R₃₁ to R₃₉, R₁₀₀ and n1 in the formulae (141) to (143); four R₁₀₀ are mutually the same or different; one of carbon atoms bonded to R₂₅, R₂₇ and R₂₈ is bonded to *1, and one selected from carbon atoms bonded to R₃₁ to R₃₈ and a nitrogen atom bonded to R₃₉ is bonded to *.

27: The compound according to claim 24, wherein X₁ is NR₃₉.

28: The compound according to claim 24, wherein X₁ is NR₃₉ and a nitrogen atom bonded to R₃₉ is bonded to *.

29: An organic-electroluminescence-device material comprising the compound according to claim 22.

30: The compound according to claim 25, wherein X₁ is NR₃₉.

31: The compound according to claim 26, wherein X₁ is NR₃₉.

32: The compound according to claim 25, wherein X₁ is NR₃₉ and a nitrogen atom bonded to R₃₉ is bonded to *.

33: The compound according to claim 26, wherein X₁ is NR₃₉ and a nitrogen atom bonded to R₃₉ is bonded to *.

34: An organic-electroluminescence-device material comprising the compound according to claim 24.

35: An organic-electroluminescence-device material comprising the compound according to claim 25.
